(19) United States
(12) Patent Application Publication
(10) Pub. No.: US 2008/0003202 A1
(43) Pub. Date: Jan. 3, 2008

(54) Modified interferon-beta (IFN-beta) polypeptides

(76) Inventors: Thierry Guyon, Palaiseau (FR); Gilles Borrelly, Combs La Ville (FR); Lila Drittanti, Vigneux-sur-Seine (FR); Manuel Vega, Vigneux-sur-Seine (FR)

Correspondence Address: FISH & RICHARDSON, PC, P.O. BOX 1022, MINNEAPOLIS, MN, 55440-1022, US

(21) Appl. No.: 11/729,266

(22) Filed: Mar. 27, 2007

Related U.S. Application Data
(60) Provisional application No. 60/787,208, filed on Mar. 28, 2006.

(51) Int. Cl. A61K 38/21; C07K 14/00; C12N 1/20; C12N 15/00; C12N 15/11; C12N 5/04; C12N 5/06; C12P 21/04

(52) U.S. Cl. 424/85.6; 435/252.3; 435/320.1; 435/325; 435/410; 435/69.51; 530/351; 536/23.52

(57) ABSTRACT

Provided are modified interferon-beta polypeptides and nucleic acid molecules encoding modified interferon-beta polypeptides and formulations containing the polypeptides and/or nucleic acid molecules. The modified polypeptides exhibit increased protein stability, including increased resistance to proteases. Also provided are methods of treatment by administering modified interferon-beta polypeptides.

### RELATED APPLICATIONS

### Priority

Benefit of priority is claimed under 35 U.S.C. §119(e) to U.S. Provisional Application Ser. No. 60/787,208, to Thierry Guyon, Gilles Borrelly, Lila Drittanti and Manuel Vega, entitled “MODIFIED INTERFERON-β (IFN-β) POLYPEPTIDES,” filed Mar. 28, 2006. The subject matter of this application is incorporated by reference in its entirety.

### Related Applications/Patents

This application is related to U.S. application Ser. No. 11/729,267, to Thierry Guyon, Gilles Borrelly, Lila Drittanti and Manuel Vega, entitled “MODIFIED INTERFERON-β (IFN-β) POLYPEPTIDES,” filed the same day herewith, and to International PCT Application Serial No. PCT/EP2007/002700, to Thierry Guyon, Gilles Borrelly, Lila Drittanti and Manuel Vega, entitled “MODIFIED INTERFERON-β (IFN-β) POLYPEPTIDES,” filed Mar. 27, 2007 both of which also claim priority to U.S. Provisional Application Ser. No. 60/787,208, to Thierry Guyon, Gilles Borrelly, Lila Drittanti and Manuel Vega, entitled “MODIFIED INTERFERON-β (IFN-β) POLYPEPTIDES,” filed Mar. 28, 2006.

This application also is related to U.S. application Ser. No. 11/176,830, to Rene Gantier, Thierry Guyon, Manuel Vega and Lila Drittanti, entitled “RATIONAL EVOLUTION OF CYTOKINES FOR HIGHER STABILITY, THE CYTOKINES AND ENCODING NUCLEIC ACID MOLECULES,” filed Jul. 6, 2005 and published as U.S. Application No. US 2006-0020116, which is a continuation of U.S. application Ser. No. 10/658,834, to Rene Gantier, Thierry Guyon, Manuel Vega and Lila Drittanti entitled “RATIONAL EVOLUTION OF CYTOKINES FOR HIGHER STABILITY, THE CYTOKINES AND ENCODING NUCLEIC ACID MOLECULES,” filed Sep. 8, 2003 and published as U.S. Application No. US-2004-0132977-A1. This application also is related to U.S. application Ser. No. 11/706,088, to Rene Gantier, Thierry Guyon, Manuel Vega and Lila Drittanti, entitled “RATIONAL EVOLUTION OF CYTOKINES FOR HIGHER STABILITY, THE CYTOKINES AND ENCODING NUCLEIC ACID MOLECULES,” filed Feb. 13, 2007, which is a divisional application of U.S. application Ser. No. 10/658,834, to Rene Gantier, Thierry Guyon, Manuel Vega and Lila Drittanti entitled “RATIONAL EVOLUTION OF CYTOKINES FOR HIGHER STABILITY, THE CYTOKINES AND ENCODING NUCLEIC ACID MOLECULES.”

This application also is related to U.S. application Ser. No. 11/196,067, to Rene Gantier, Thierry Guyon, Hugo Cruz Ramos, Manuel Vega and Lila Drittanti entitled “RATIONAL DIRECTED PROTEIN EVOLUTION USING TWO-DIMENSIONAL RATIONAL MUTAGENESIS SCANNING,” filed Aug. 2, 2005 and published as U.S. Application No. US-2006-0020396-A1, which is a continuation of U.S. application Ser. No. 10/658,355, to Rene Gantier, Thierry Guyon, Hugo Cruz Ramos, Manuel Vega and Lila Drittanti entitled “RATIONAL DIRECTED PROTEIN EVOLUTION USING TWO-DIMENSIONAL RATIONAL MUTAGENESIS SCANNING,” filed Sep. 8, 2003 and published as U.S. Application No. US 2005-0202438.

This application also is related to U.S. application Ser. No. 10/658,834, filed Sep. 8, 2003, and to published International PCT Application WO 2004/022593, to Rene Gantier, Thierry Guyon, Manuel Vega and Lila Drittanti entitled, “RATIONAL EVOLUTION OF CYTOKINES FOR HIGHER STABILITY, THE CYTOKINES AND ENCODING NUCLEIC ACID MOLECULES.” This application also is related to U.S. application Ser. No. 10/658,355, filed Sep. 8, 2003, and to International PCT Application WO 2004/022747, to Rene Gantier, Thierry Guyon, Hugo Cruz Ramos, Manuel Vega and Lila Drittanti entitled “RATIONAL DIRECTED PROTEIN EVOLUTION USING TWO-DIMENSIONAL RATIONAL MUTAGENESIS SCANNING.”

The subject matter of each of the above-noted applications, provisional applications and international applications is incorporated by reference in its entirety.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy # 1 and Copy # 2), the contents of which are incorporated by reference in their entirety. The computer-readable file on each of the aforementioned compact discs, created on Mar. 26, 2007 is identical, 935 kilobytes in size, and titled 924BSEQ.001.txt.

### FIELD OF THE INVENTION

Modified Interferon-β (IFN-β) polypeptides that have pre-selected modified properties compared to unmodified or wild-type proteins, and nucleic acid molecules encoding these proteins are provided. The polypeptides can be used for treatment and diagnosis.

### BACKGROUND

Effective delivery of therapeutic proteins for clinical use is a major challenge to pharmaceutical science. Once in the blood stream, these proteins are constantly eliminated from circulation within a short time by different physiological processes, involving metabolism as well as clearance using normal pathways for protein elimination, such as (glomerular) filtration in the kidneys or proteolysis in blood. Once in the luminal gastrointestinal tract, these proteins are constantly digested by luminal proteases. The latter is often the limiting process affecting the half-life of proteins used as therapeutic agents in per-oral administration and either intravenous or intramuscular injection. The problems associated with these routes of administration of proteins are well known and various strategies have been used in attempts to solve them.

A protein family that has been the focus of clinical work and effort to improve its administration and bio-assimilation is the cytokine family, including the interferon family. Interferon molecules are grouped in the heterogeneous family of cytokines, originally identified on the basis of their ability to induce cellular resistance to viral infections (Diaz et al., J. Interferon Cytokine Res., 16: 179-180 (1996)). Type I interferons, referred to as interferons α/β, include many members of the interferon α family (interferon α1, α2, ω and τ) as well as interferon β. The type II interferon-γ is different from type I in its particular mechanisms that regulate its production. Whereas the production of interferons α/β is most efficiently induced in many types of cells upon viral infection, interferon-γ is produced mainly in cells of hematopoietic system, such as T-cells or natural killer cells, upon stimulation by antigens or cytokines, respectively. These two interferon systems are functionally non-redundant in the anti-viral defense host.

Interferons, as well as many other cytokines, are important therapeutics. Naturally occurring variants can have undesirable side effects as well as the problems of administration, bioavailability and short half-life. IFN-β has been well established as a pharmaceutical for humans and other animals. Because of its instability in the blood stream and under storage conditions, therapeutic protocols require frequent and repeated administration. Hence, there is a need to improve properties of IFN-β for its use as a biotherapeutic. Therefore, among the objects herein, it is an object to provide modified IFN-β polypeptides that have improved therapeutic properties and/or activities.

### SUMMARY

Provided herein are modified IFN-β polypeptides that have improved properties, particularly therapeutic properties and/or activities. Modified IFN-β polypeptides provided herein exhibit increased protein stability compared to an unmodified IFN-β polypeptide or an IFN-β that does not have such modifications or corresponding modifications. Modified IFN-β polypeptides provided herein that exhibit increased protein stability display, among other parameters, increased protein half-life in vivo or in vitro compared to an unmodified IFN-β polypeptide. Increased protein stability of a modified IFN-β provided herein can be manifested in a variety of ways, such as increased resistance to digestion by proteases and/or increased conformational stability.

Therapeutic use of IFN-β is well established for human and other animals. Because of its instability in the bloodstream, as well as under storage conditions, therapy with IFN-β can require frequent and repeated applications. The modified IFN-β polypeptides provided herein are mutant variants of IFN-β that display improved protein stability. These variants possess increased protein half-life, including, for example, increased stability in the bloodstream, following oral administration, and/or under storage conditions. Such increased stability includes stability as assessed by resistance to blood, intestinal or any other proteases and/or increased thermal tolerance and/or tolerance to pH and/or other potentially denaturing and stability disrupting conditions.

Modified IFN-β polypeptides provided herein that exhibit increased protein stability include IFN-β polypeptides modified at any number of residues whereby the targeted activity or property that is modified, such as protease resistance, is modified, and such that at least one activity, typically a therapeutic activity, is retained at a level, so as, for example, to permit formulation of the IFN-β polypeptide at an effective dosage for treatment. In general, the modified IFN-β polypeptides include 1 or 2 modifications, but can include such modifications in addition to modifications that alter other properties. Hence, included are modified IFN-β in which a total of 1, 2, 3, 4, 5, 6, 7, 8, 9, 10, 11, 12, 13, 14, 15, 16, 17, 18, 19, or 20 positions are modified compared to an unmodified IFN-β polypeptide. Modified IFN-β polypeptides include mature forms (i.e. the polypeptide whose sequence is set forth in SEQ ID NO. 1) and precursor forms (i.e. the polypeptide whose sequence is set forth in SEQ ID NO. 2). Modification is with reference to a wildtype human IFN-β polypeptide that includes a sequence of amino acids set forth in SEQ ID NO:1 or SEQ ID NO:2, respectively, and also includes modification relative to allelic or species variant or an isoform of an IFN-β polypeptide set forth in SEQ ID NO:1 or SEQ ID NO:3. Allelic and species variants can have 40%, 50%, 60%, 70%, 80%, 90%, 91%, 92%, 93%, 94%, 95%, 96%, 97%, 98%, or 99% sequence identity to the polypeptide set forth in SEQ ID NO:1, excluding any amino acid modification thereof. Modified loci are identified with reference to the amino acid numbering of a unmodified mature IFN-β polypeptide whose sequence of amino acids is set forth in SEQ ID NO:1. Corresponding positions on a particular polypeptide readily can be determined, such as by alignment of unchanged residues. The modified IFN-β polypeptide exhibits increased protein stability compared to the unmodified IFN-β polypeptide. Typically, the modified IFN-β polypeptide also retains one or more activities and/or properties of the unmodified IFN-β polypeptide.

Provided herein are modified IFN-β polypeptides containing an amino acid modification at a position corresponding to amino acid residues L5 or L6 of a mature IFN-β polypeptide set forth in SEQ ID NO:1, that also contains a further amino acid modification at another position. For example, such a modified IFN-β polypeptide has 2, 3, 4, 5, 6, 7, 8, 9, 10, 11, 12, 13, 14, 15, 16, 17, 18, 19, or 20 modifications. Modified IFN-β polypeptides with modifications at positions L5 or L6 include mature forms (i.e. the polypeptide whose sequence is set forth in SEQ ID NO. 1) and precursor forms (i.e. the polypeptide whose sequence is set forth in SEQ ID NO. 2). The amino acid modification at position L5 or L6 is a replacement of leucine (L) by any of aspartic acid (D), glutamine (Q), asparagines (N), or glutamic acid (E). In one example, the further amino acid replacement is at positions corresponding to any of amino acid positions M1, Y3, L5, L6, F8, L9, Q10, R11, S12, S13, N14, F15, Q16, C17, Q18, K19, L20, L21, W22, Q23, L24, N25, R27, L28, E29, Y30, C31, L32, K33, D34, R35, M36, F38, D39, P41, E42, E43, K45, L47, Q48, Q49, F50, Q51, K52, E53, D54, L57, Y60, E61, M62, L63, Q64, F67, F70, R71, Q72, D73, G78, W79, N80, E81, T82, I83, E85, N86, L87, L88, A89, N90, V91, Y92, Q94, I95, H97, L98, K99, V101, L102, E103, E104, K105, L106, E107, K108, E109, D110, R113, K115, L116, M117, L120, L122, K123, R124, Y125, Y126, R128, L130, Y132, L133, K134, K136, E137, Y138, W143, R147, E149, L151, R152, F154, Y155, F156, R159, L160, Y163, L164, and R165 of a mature IFN-β polypeptide set forth in SEQ ID NO:1. For example, such further replacements include M1V (i.e., replacement of M by V at a position corresponding to amino acid position 1 of mature IFN-β (e.g., SEQ ID NO:1), M1I, M1T, M1A, M1Q, M1D, M1E, M1K, M1N, M1R, M1S, M1C, Y3I, Y3H, L5V, L5I, L5T, L5Q, L5H, L5A, L5D, L5E, L5K, L5R, L5N, L5S, L6D, L6E, L6K, L6N, L6Q, L6R, L6S, L6T, L6C, L6I, L6V, L6H, L6A, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, R11H, R11Q, R11D, S12D, S12E, S12K, S12R, S13D, S13E, S13K, S13N, S13Q, S13R, S13T, S13C, N14D, N14E, N14K, N14Q, N14R, N14S, N14T, F15I, F15V, F15D, F15E, F15K, F15R, Q16D, Q16E, Q16K, Q16N, Q16R, Q16S, Q16T, Q16C, C17D, C17E, C17K, C17N, C17R, C17S, C17T, Q18H, Q18S, Q18T, Q18N, K19N, K19Q, K19T, K19S, K19H, L20I, L20V, L20H, L20A, L20N, L20Q, L20R, L20S, L20T, L20D, L20E, L20K, L21I, L21V, L21T, L21Q, L21H, L21A, W22S, W22H, W22D, W22E, W22K, W22R, Q23D, Q23E, Q23K, Q23R, Q23H, Q23S, Q23T, Q23N, L24I, L24V, L24T, L24Q, L24H, L24A, L24D, L24E, L24K, L24R, N25H, N25S, N25Q, R27H, R27Q, L28V, L28I, L28T, L28Q, L28H, L28A, E29Q, E29H, E29N, Y30H, Y30I, L32V, L32I, L32T, L32Q, L32H, L32A, K33Q, K33T, K33S, K33H, K33N, D34N, D34Q, D34G, R35H, R35Q, M36V, M36I, M36T, M36Q, M36A, F38I, F38V, D39N, D39Q, D39H, D39G, P41A, P41S, E42N, E42Q, E42H, E43K, E43Q, E43H, E43N, K45D, K45N, K45Q, K45T, K45S, K45H, L47V, L47I, L47T, L47Q, L47H, L47A, Q48H, Q48S, Q48T, Q48N, Q49H, Q49S, Q49T, Q49N, F50I, F50V, Q51H, Q51S, Q51T, Q51N, K52Q, K52T, K52S, K52H, K52D, K52N, E53R, E53Q, E53H, E53N, D54K, D54Q, D54N, D54G, L57I, L57V, L57T, L57Q, L57H, L57A, Y60H, Y60I, E61K, E61Q, E61H, E61N, M62I, M62V, M62T, M62Q, M62A, L63I, L63V, L63T, L63Q, L63H, L63A, Q64H, Q64S, Q64T, Q64N, F67I, F67V, F70I, F70V, R71H, R71Q, Q72H, Q72S, Q72T, Q72N, D73Q, D73H, D73G, D73N, G78D, G78E, G78K, G78R, W79H, W79S, N80D, N80E, N80K, N80R, E81Q, E81H, E81K, E81N, T82D, T82E, T82K, T82R, I83D, I83E, I83K, I83R, I83N, I83Q, I83S, I83T, E85Q, E85H, E85K, E85N, N86D, N86E, N86K, N86R, N86Q, N86S, N86T, L87D, L87E, L87K, L87R, L87N, L87Q, L87S, L87T, L87I, L87V, L87H, L87A, L88I, L88V, L88T, L88Q, L88H, L88A, A89D, A89E, A89K, A89R, N90D, N90E, N90K, N90Q, N90R, N90S, N90T, N90C, V91D, V91E, V91K, V91N, V91Q, V91R, V91S, V91T, V91C, Y92H, Y92I, Q94D, Q94E, Q94K, Q94N, Q94R, Q94S, Q94T, Q94C, I95D, I95E, I95K, I95N, I95Q, I95R, I95S, I95T, H97D, H97E, H97K, H97N, H97Q, H97R, H97S, H97T, H97C, L98D, L98E, L98K, L98N, L98Q, L98R, L98S, L98T, L98C, L98I, L98V, L98H, L98A, K99N, K99Q, K99T, K99S, K99H, V101D, V101E, V101K, V101N, V101Q, V101R, V101S, V101T, V101C, L102I, L102V, L102T, L102Q, L102H, L102A, E103K, E103N, E103Q, E103H, E104Q, E104H, E104R, E104N, K105Q, K105T, K105S, K105H, K105D, K105N, L106I, L106V, L106T, L106Q, L106H, L106A, E107Q, E107H, E107R, E107N, K108D, K108N, K108Q, K108T, K108S, K108H, E109H, E109Q, E109R, E109N, D110K, D110N, D110Q, D110H, D110G, F111I, F111V, R113H, R113Q, R113E, K115D, K115Q, K115N, K115S, K115H, L116V, L 116I, L116T, L116Q, L116H, L116A, M117I, M117V, M117T, M117Q, M117A, L120V, L120I, L120T, L120Q, L120H, L120A, L122I, L122V, L122T, L122Q, L122H, L122A, K123Q, K123T, K123S, K123H, K123N, R124D, R124E, R124H, R124Q, Y125H, Y125I, Y126H, Y126I, R128H, R128Q, L130V, L130I, L130T, L130Q, L130H, L130A, Y132H, Y132I, L133I, L133V, L133T, L133Q, L133H, L133A, K134Q, K134T, K134S, K134H, K134N, K136N, K136Q, K136T, K136S, K136H, E137Q, E137H, E137N, Y138H, Y138I, W143H, W143S, R147H, R147Q, E149Q, E149H, E149N, L151I, L151V, L151T, L151Q, L151H, L151A, R152D, R152H, R152Q, F154I, F154V, Y155H, Y155I, F156I, F156V, R159H, R159Q, L160I, L160V, L160T, L160Q, L160H, L160A, Y163H, Y163I, L164I, L164V, L164T, L164Q, L164H, L164A, R165D, R165H, and R165Q.

In one example, exemplary amino acid modifications of an IFN-β polypeptide include amino acid modifications of any of L5D/L6E (i.e., replacement of L by D at a position corresponding to amino acid position 5 and replacement of L by E at a position corresponding to amino acid position 6, of mature IFN-β (e.g., SEQ ID NO:1), L5E/Q0D, L5Q/M36I, L6E/L47I, L5E/K108S, L5E/L6E, L5D/Q10D, L5N/M36I, L6Q/L47I, L5D/K108S, L5N/L6E, L5Q/Q10D, L6E/M36I, L5E/N86Q, L5Q/K108S, L5Q/L6E, L5N/Q10D, L6Q/M36I, L5D/N86Q, L5N/K108S, L5D/L6Q, L6E/Q10D, L5E/L47I, L5Q/N86Q, L6E/L6Q, L6Q/Q10D, L5D/L47I, L6Q/K108S, L5N/L6Q, L6E/M36I, L5Q/L47I, L6E/N86Q, L5Q/L6Q, L5D/M36I, L5N/L47I, L6Q/N86Q, L6E/K108S, and L5N/N86Q. For example, a modified IFN-β polypeptide provided herein has a sequence of amino acids set forth in any of SEQ ID NOS:88-125, or a biologically active portion thereof.

In some examples, a modified IFN-β polypeptide containing a modification corresponding to position L5 or L6 of a mature IFN-β polypeptide set forth in SEQ ID NO:1, and that also contains a further amino acid modification, exhibits increased protein stability and retains one of more activities of the unmodified IFN-β polypeptide. Generally, increased protein stability is increased protein half-life in vitro or in vivo. Increased protein stability of the modified IFN-β polypeptide is the result only of modification to the primary sequence of the IFN-β polypeptide. In some cases, a modified IFN-β polypeptide provided herein also can include a further amino acid modification that contributes to deimmunization, glycosylation, or PEGylation of the polypeptide such that a modified polypeptide provided herein can be glycosylated or conjugated to a polyethylene glycol (PEG) moiety.

The increased protein stability exhibited by an IFN-β polypeptide can be manifested as increased protease resistance or increased conformational stability. For example, such an exemplary IFN-β polypeptide can include modifications corresponding to L5D/L47I (SEQ ID NO:115), L5D/L6Q (SEQ ID NO:108), or L6Q/K108S (SEQ ID NO:117).

Increased protein stability of an IFN-β polypeptide can result from, for example, increased resistance to proteolysis by proteases or a protease that occur/occurs in serum, blood, saliva, digestive fluids, and/or in vitro. For example, the proteases include, any of pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, luminal pepsin, microvillar endopeptidase, dipeptidyl peptidase, enteropeptidase, hydrolase, NS3, factor Xa, Granzyme B, thrombin, plasmin, urokinase, tPA and PSA. In some examples, a modified IFN-β polypeptide that contains an amino acid modification corresponding to amino acid positions L5 or L6 of mature IFN-β set forth in SEQ ID NO:1, and that contains a further amino acid modification, exhibits increased protein stability due to increased protease resistance to gelatinase B. Exemplary of those modified IFN-β polypeptides are those with modifications corresponding to L6E/K108S, L5Q/K108S, L5E/K108S, L5N/Q10D, and L5N/K108S of a mature IFN-β polypeptide.

Provided herein are modified IFN-β polypeptides containing one or more amino acid modification corresponding to any of Y3I (i.e., replacement of Y by I at a position corresponding to amino acid position 3 of mature IFN-β (e.g., SEQ ID NO:1), Y3H, L6I, L6V, L6H, L6A, R11D, Q18S, Q18N, Q18H, Q18T, K19N, L20I, L20V, L20H, L20A, L21I, L21V, L21T, L21Q, L21H, L21A, Q23H, Q23S, Q23T, Q23N, L24I, L24V, L24T, L24Q, L24H, L24A, E29N, K33N, D34N, D34Q, D34G, F38I, F38V, D39N, P41A, P41S, E42N, E43N, E43K, E43Q, E43H, K45D, K45N, Q48S, Q48T, Q48N, Q49H, Q49S, Q49T, Q49N, F50I, F50V, Q51H, Q51S, Q51T, Q51N, K52D, K52N, E53N, E53R, E53Q, E53H, D54K, D54N, D54G, D54Q, L57I, L57T, L57Q, L57H, Y60I, Y60H, E61K, E61H, E61N, E61Q, M62I, M62V, M62T, M62Q, L63I, L63V, L63T, L63Q, L63H, L63A, Q64H, Q64S, Q64T, Q64N, F70I, F70V, Q72H, Q72S, Q72T, Q72N, D73N, W79H, W79S, E81K, E81N, E85N, E85K, L87I, L87V, L87H, L87A, L88I, L88V, L88T, L88Q, L88H, L88A, L98I, L98V, L98H, L98A, K99N, L102I, L102V, L102T, L102Q, L102H, L102A, E103N, E103K, E104N, E104R, K105D, K105N, L106I, L106V, L106T, L106Q, L106H, L106A, E107N, E107R, K108D, K108N, E109R, E109N, D110K, D110N, R113E, K115D, K115N, K115S, K115H, K115Q, M117I, M117V, M117T, M117Q, M117A, L122I, L122V, L122T, L122Q, L122H, L122A, K123N, R124D, R124E, Y125I, Y125H, Y126I, Y126H, Y132I, Y132H, L133I, L133V, L133T, L133Q, L133H, L133A, K134N, K136N, E137N, W143H, W143S, R147H, R147Q, E149H, E149N, E149Q, L151I, L151V, L151T, L151Q, L151H, L151A, R152D, F154V, F154I, F156I, L160I, L160V, L160T, L160Q, L160H, L160A, L164I, L164V, L164T, L164Q, L164H, L164A, and R165D. SEQ ID NOS: of exemplary modified IFN-β polypeptides are set forth in any of SEQ ID NOS: 4-68, 71-82, 84-87, 134-153, 519, 520, 534-557, 559, 560, 562-564, 566-606, and 608-650, or a biologically active portion thereof.

Such a modification of an IFN-β polypeptide at one or more positions set forth above can be in a mature human IFN-β polypeptide of SEQ ID NO:1, or its precursor form set forth in SEQ ID NO:2. Modification also be can in a recombinant IFN-β polypeptide set forth in SEQ ID NO:3. It also is understood that amino acid modification of an IFN-β polypeptide can be in an allelic, species, or isoform variant of SEQ ID NO:1, where the allelic or species variant has 40%, 50%, 60%, 70%, 80%, 90%, 91%, 92%, 93%, 94%, 95%, 96%, 97%, 98%, or 99% sequence identity to the polypeptide set forth in SEQ ID NO:1, excluding the modified positions.

Exemplary amino acid modifications correspond to any of Y3I, Q18S, Q18N, K19N, L20I, L20V, K33N, D34N, P41A, P41S, E42N, E43N, K45D, K45N, Q48S, Q48T, Q49H, Q49S, Q49T, F50I, F50V, Q51H, Q51S, Q51T, Q51N, K52D, K52N, E53N, D54K, D54N, D54G, L57I, Y60I, E61K, E61H, E61N, L63I, Q64H, Q64S, Q64T, F70I, F70V, Q72H, Q72S, E85N, L88I, L88V, L98I, L98V, K99N, E103N, E104N, K105D, K105N, L106I, L106V, E107N, E109N, K115D, K115N, K115S, K115H, K123N, Y125I, Y126I, Y132I, K134N, K136N, R147H, R147Q, E149H, E149N, L151I, and F154V of the mature IFN-β polypeptide set forth in SEQ ID NO:1. In some instances where the unmodified IFN-β is the polypeptide set forth in SEQ ID NO:3, exemplary amino acid modifications correspond to any of Y3I, Q18S, Q18N, K19N, L20I, L20V, L21I, L21V, K33N, D34N, K33N, P41A, P41S, E42N, E43N, K45D, K45N, Q48H, Q48S, Q48T, Q49H, Q49S, Q49T, F50I, F50V, Q51H, Q51S, Q51T, Q51N, K52D, K52N, E53N, D54G, L57I, Y60I, E61K, E61H, E61N, M62I, M62V, Q64H, Q64S, Q64T, F70I, F70V, Q72H, Q72S, E85N, L88I, L88V, L98I, L98V, K99N, E103N, E104N, K105D, K105N, L106I, L106V, E107N, E109N, K115D, K115N, K115S, K115H, M117I, M117V, L122I, L122V, K123N, Y125I, Y126I, Y132I, K134N, Y136N, R147H, R147Q, E149H, E149N, L151I, L154V, and L160V, with reference to amino acid positions of a mature IFN-β polypeptide set forth in SEQ ID NO:1.

Such modifications of an IFN-β polypeptide typically include 1, 2, 3, 4, 5, 6, 7, 8, 9, 10, 11, 12, 13, 14, 15, 16, 17, 18, 19, or 20 modifications. IFN-β polypeptides that can be modified include mature forms (i.e. the polypeptide whose sequence is set forth in SEQ ID NO. 1) and precursor forms (i.e. the polypeptide whose sequence is set forth in SEQ ID NO. 2). A IFN-β polypeptide containing any one or more amino acid modifications such as is set forth above, can contain a further amino acid modification at amino acid positions corresponding to positions M1, Y3, L5, L6, F8, L9, Q10, R11, S12, S13, N14, F15, Q16, C17, Q18, K19, L20, L21, W22, Q23, L24, N25, R27, L28, E29, Y30, L32, K33, D34, R35, M36, F38, D39, E42, E43, K45, L47, Q48, Q49, K52, E53, D54, L57, Y60, E61, M62, L63, Q64, F67, R71, Q72, D73, G78, W79, N80, E81, T82, I83, E85, N86, L87, L88, A89, N90, V91, Y92, Q94, I95, H97, L98, K99, V101, L102, E103, E104, K105, L106, E107, K108, E109, D110, F111, R113, K115, L116, M117, L120, L122, K123, R124, Y125, Y126, R128, L130, Y132, L133, K134, N136, E137, Y138, W143, E149, L151, R152, F154, Y155, F156, R159, L160, Y163, L164, and R165 of a mature IFN-β polypeptide set forth in SEQ ID NO:1. For examples, amino acid replacements at any one of the further amino acid positions can include replacements of any of M1C (i.e. replacement of M by C at a position corresponding to amino acid position 1 of mature IFN-β (SEQ ID NO:1), M1D, M1E, M1K, M1N, M1R, M1S, M1V, M1I, M1T, M1A, M1Q, Y3H, L5V, L5I, L5T, L5Q, L5H, L5A, L5D, L5E, L5K, L5R, L5N, L5S, L6I, L6V, L6H, L6A, L6D, L6E, L6K, L6N, L6Q, L6R, L6S, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, R11D, R11H, R11Q, S12D, S12E, S12K, S12R, S13D, S13E, S13K, S13N, S13Q, S13R, S13T, S13C, N14D, N14E, N14K, N14Q, N14R, N14S, N14T, F15D, F15E, F15K, F15R, F15I, F15V, Q16D, Q16E, Q16K, Q16N, Q16R, Q16S, Q16C, Q16T, C17D, C17E, C17K, C17N, C17Q, C17R, C17S, C17T, Q18H, Q18T, K19Q, K19T, K19S, K19H, L20H, L20A, L20N, L20Q, L20R, L20S, L20T, L20D, L20E, L20K, L21I, L21V, L21T, L21Q, L21H, L21A, W22D, W22E, W22K, W22R, W22S, W22H, Q23H, Q23S, Q23T, Q23N, Q23D, Q23E, Q23K, Q23R, L24I, L24V, L24T, L24Q, L24H, L24A, L24D, L24E, L24K, L24R, N25H, N25S, N25Q, R27H, R27Q, L28V, L28I, L28T, L28Q, L28H, L28A, E29N, E29Q, E29H, Y30H, Y30I, L32V, L32I, L32T, L32Q, L32H, L32A, K33Q, K33T, K33S, K33H, D34Q, D34G, R35H, R35Q, M36V, M36I, M36T, M36Q, M36A, F38I, F38V, D39N, D39Q, D39H, D39G, E42Q, E42H, E43K, E43Q, E43H, K45Q, K45T, K45S, K45H, L47V, L47I, L47T, L47Q, L47H, L47A, Q48N, Q49N, K52Q, K52T, K52S, K52H, E53R, E53Q, E53H, D54Q, L57V, L57T, L57Q, L57H, L57A, Y60H, E61Q, M62I, M62V, M62T, M62Q, M62A, L63V, L63T, L63Q, L63H, L63A, Q64N, F67I, F67V, R71H, R71Q, Q72N, D73N, D73H, D73G, D73Q, G78D, G78E, G78K, G78R, W79H, W79S, W79D, W79E, W79K, W79R, N80D, N80E, N80K, N80R, E81K, E81N, E81Q, E81H, T82D, T82E, T82K, T82R, I83D, I83E, I83K, I83R, I83N, I83Q, I83S, I83T, E85K, E85Q, E85H, N86D, N86E, N86K, N86R, N86Q, N86S, N86T, L87I, L87V, L87H, L87A, L87D, L87E, L87K, L87R, L87N, L87Q, L87S, L87T, L88T, L88Q, L88H, L88A, A89D, A89E, A89K, A89R, N90D, N90E, N90K, N90Q, N90R, N90S, N90T, N90C, V91D, V91E, V91K, V91N, V91Q, V91R, V91S, V91T, V91C, Y92H, Y92I, Q94D, Q94E, Q94K, Q94N, Q94R, Q94S, Q94T, Q94C, I95D, I95E, I95K, I95N, I95Q, I95R, I95S, I95T, H97D, H97E, H97K, H97N, H97Q, H97R, H97S, H97T, H97C, L98H, L98A, L98D, L98E, L98K, L98N, L98Q, L98R, L98S, L98T, L98C, K99Q, K99T, K99S, K99H, V101D, V101E, V101K, V101N, V101Q, V101R, V101S, V101T, V101C, L102I, L102V, L102T, L102Q, L102H, L102A, E103K, E103Q, E103H, E104R, E104Q, E104H, K105Q, K105T, K105S, K105H, L106T, L106Q, L106H, L106A, E107R, E107Q, E107H, K108D, K108N, K108Q, K108T, K108S, K108H, E109R, E109Q, E109H, D110K, D110N, D110Q, D110H, D110G, F111I, F111V, R113E, R113H, R113Q, K115Q, L116V, L116I, L116T, L116Q, L116H, L116A, M117I, M117V, M117T, M117Q, M117Q, M117A, L120V, L120I, L120T, L120Q, L120H, L120A, L122I, L122V, L122T, L122Q, L122H, L122A, K123Q, K123T, K123S, K123H, R124D, R124E, R124H, R124Q, Y125H, Y126H, R128H, R128Q, L130V, L130I, L130T, L130Q, L130H, L130A, Y132H, L133I, L133V, L133T, L133Q, L133H, L133A, K134Q, K134T, K134S, K134H, K136Q, K136T, K136S, K136H, E137N, E137Q, E137H, Y138H, Y138I, W143H, W143S, E149Q, L151V, L151T, L151Q, L151H, L151A, R152D, R152H, R152Q, F154I, Y155H, Y155I, F156I, F156V, R159H, R159Q, L160I, L160V, L160T, L160Q, L160H, L160A, Y163H, Y163I, L164I, L164V, L164T, L164Q, L164H, L164A, R165D, R165Q and R165H of a mature IFN-β polypeptide set forth in SEQ ID NO:1.

A modified IFN-β polypeptide containing one or more amino acid modification exhibits increased protein stability and retains one of more activities of the unmodified IFN-β polypeptide. Generally, increased protein stability is increased protein half-life in vitro or in vivo. Increased protein stability of the modified IFN-β polypeptide is the result only of modification to the primary sequence of the IFN-β polypeptide. In some cases, a modified IFN-β polypeptide provided herein also can include a further amino acid modification that contributes to deimmunization, glycosylation, or PEGylation of the polypeptide such that a modified polypeptide provided herein can be glycosylated or conjugated to a polyethylene glycol (PEG) moiety.

In some examples, an IFN-β polypeptide containing one or more amino acid modification exhibits increased protein stability manifested as increased protease resistance, increased conformational stability, or a combination thereof. For example, the one or more amino acid modifications can correspond to any of Y3H, Y3I, L6I, L6V, L6H, L6A, K19N, Q18S, Q18N, Q18H, Q18T, L20I, L20V, L20H, L20A, L21I, L21V, L21T, L21Q, L21H, L21A, Q23H, Q23S, Q23T, Q23N, L24I, L24V, L24T, L24Q, L24H, L24A, K33N, E29N, D34N, D34Q, D34G, F38I, F38V, D39N, P41A, P41S, E42N, E43Q, E43H, E43N, K45N, Q48N, Q48H, Q48S, Q48T, Q49N, Q49H, Q49S, Q49T, F50I, F50V, Q51H, Q51S, Q51T, Q51N, K52N, E53N, E53Q, E53H, D54N, D54Q, D54G, L57I, L57V, L57T, L57Q, L57H, Y60I, Y60H, E61H, E61N, E61Q, M62I, M62V, M62T, M62Q, L63I, L63V, L63T, L63Q, L63H, Q64N, Q64H, Q64S, Q64T, F70I, F70V, Q72H, Q72S, Q72T, Q72N, D73N, W79H, W79S, E81N, E85N, L87I, L87V, L87H, L87A, L88I, L88V, L88T, L88Q, L88H, L88A, L98I, L98V, L98H, L98A, K99N, L102I, L102V, L102T, L102Q, L102H, L102A, E103N, E104N, K105N, L106I, L106V, L106T, L106Q, L106H, L106A, E107N, K108N, E109N, D110N, K115N, K115S, K115H, K115Q, M117I, M117V, M117T, M117Q, M117A, L122I, L122V, L122T, L122Q, L122H, L122A, K123N, Y125I, Y125H, Y126I, Y126H, Y132I, Y132H, L133I, L133V, L133T, L133Q, L133H, K134N, K136N, E137N, W143H. W143S, R147H, R147Q, E149H, E149N, E149Q, L151I, L151V, L151T, L151Q, L151H, L151A, F154I, F154V, F156I, L160I, L160V, L160T, L160Q, L160H, L160A, L164T, L164Q, L164H, L164A L164I, and L164V of a mature IFN-β polypeptide set forth in SEQ ID NO:1, where increased protein stability is manifested as increased protease resistance. Exemplary SEQ ID NOS of modified IFN-β polypeptides exhibiting increased protease resistance are set forth in any of SEQ ID NOS: 4-68, 71-82, 84-87, 534-557, 559-606, and 608-650, or a biologically active portion thereof.

A modified IFN-β polypeptide provided herein that contains one or more amino acid modifications and exhibits increased protein stability manifested as increased resistance to a protease also can contain any one or more further amino acid modification at amino acid positions corresponding to positions M1, Y3, L5, L6, F8, L9, Q10, R11, S12, S13, N14, F15, Q16, C17, Q18, K19, L20, L21, W22, Q23, L24, N25, R27, L28, E29, Y30, L32, K33, D34, R35, M36, F38, D39, E42, E43, K45, L47, Q48, Q49, K52, E53, D54, L57, Y60, E61, M62, L63, Q64, F67, R71, Q72, D73, G78, W79, N80, E81, T82, I83, E85, N86, L87, L88, A89, N90, V91, Y92, Q94, I95, H97, L98, K99, V101, L102, E103, E104, K105, L106, E107, K108, E109, D110, F111, R113, K115, L116, M117, L122, K123, R124, Y125, Y126, R128, L130, Y132, L133, K134, K136, E137, Y138, W143, E149, L151, R152, Y155, F156, R159, L160, Y163, L164, and R165 of a mature IFN-β polypeptide set forth in SEQ ID NO:1. For examples, amino acid replacements at any one of the further amino acid positions can include replacements of any of M1V (i.e. replacement of M by V at a position corresponding to amino acid position 1 of mature IFN-β (SEQ ID NO:1), M1I, M1T, M1A, M1Q, M1D, M1E, M1K, M1N, M1R, M1S, M1C, Y3H, L5V, L5I, L5T, L5Q, L5H, L5A, L5D, L5E, L5K, L5R, L5N, L5S, L6H, L6A, L6I, L6V, L6D, L6E, L6K, L6N, L6Q, L6R, L6S, L6T, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, R11H, R11Q, S12D, S12E, S12K, S12R, S13D, S13E, S13K, S13N, S13Q, S13R, S13T, S13C, N14D, N14E, N14K, N14Q, N14R, N14S, N14T, F15I, F15V, F15D, F15E, F15K, F15R, Q16D, Q16E, Q16K, Q16N, Q16R, Q16S, Q16T, Q16C, C17D, C17E, C17K, C17N, C17R, C17S, C17T, Q18H, Q18T, K19Q, K19T, K19S, K19H, L20H, L20A, L20N, L20Q, L20R, L20S, L20T, L20D, L20E, L20K, L21I, L21V, L21T, L21Q, L21H, L21A, W22S, W22H, W22D, W22E, W22K, W22R, Q23H, Q23S, Q23T, Q23N, Q23D, Q23E, Q23K, Q23R, L24T, L24Q, L24H, L24I, L24V, L24D, L24E, L24K, L24R, N25H, N25S, N25Q, R27H, R27Q, L28V, L28I, L28T, L28Q, L28H, L28A, E29N, E29Q, E29H, Y30H, Y30I, L32V, L32I, L32T, L32Q, L32H, L32A, K33Q, K33T, K33S, K33H, D34Q, D34G, R35H, R35Q, M36V, M36I, M36T, M36Q, M36A, F38I, F38V, D39N, D39Q, D39H, D39G, E42Q, E42H, E43Q, E43H, K45Q, K45T, K45S, K45T, L47V, L47I, L47T, L47Q, L47H, L47A, Q48N, Q49N, K52Q, K52T, K52S, K52H, E53Q, E53H, D54Q, L57T, L57Q, L57H, L57A, L57V, Y60H, E61Q, M62I, M62V, M62T, M62Q, M62A, L63T, L63Q, L63H, L63A, L63V, Q64N, F67I, F67V, R71H, R71Q, Q72T, Q72N, D73N, D73Q, D73H, D73G, G78D, G78E, G78K, G78R, W79H, W79S, N80D, N80E, N80K, N80R, E81N, E81Q, E81H, T82D, T82E, T82K, T82R, I83D, I83E, I83K, I83R, I83N, I83Q, I83S, I83T, E85 Q, E85H, N86D, N86E, N86K, N86R, N86Q, N86S, N86T, L87H, L87A, L88T, L88Q, L88H, L88A, L87I, L87V, L87D, L87E, L87K, L87R, L87N, L87Q, L87S, L87T, A89D, A89E, A89K, A89R, N90D, N90E, N90K, N90Q, N90R, N90S, N90T, N90C, V91D, V91E, V91K, V91N, V91Q, V91R, V91S, V91T, V91C, Y92H, Y92I, Q94D, Q94E, Q94K, Q94N, Q94R, Q94S, Q94T, Q94C, I95D, I95E, I95K, I95N, I95Q, 195R, I95S, I95T, H97D, H97E, H97K, H97N, H97Q, H97R, H97S, H97T, H97C, L98H, L98A, L98D, L98E, L98K, L98N, L98Q, L98R, L98S, L98T, L98C, K99Q, K99T, K99S, K99H, V101D, V101E, V101K, V101N, V101Q, V101R, V101S, V101T, V101C, L102T, L102Q, L102H, L102A, L102I, L102V, E103Q, E103H, E104Q, E104H, K105Q, K105T, K105S, K105H, L106T, L106Q, L106H, L106A, E107Q, E107H, K108N, K108Q, K108T, K108S, K108H, E109H, E109Q, D110N, D110Q, D110H, D110G, F111I, F111V, R113H, R113Q, K115Q, L116V, L116I, L116T, L116Q, L116H, L116A, M117I, M117V, M117T, M117Q, M117A, L122I, L122V, L122T, L122Q, L122H, L122A, K123Q, K123T, K123S, K123H, R124H, R124Q, Y125H, Y126H, R128H, R128Q, L130V, L130I, L130T, L130Q, L130H, L130A, L133T, L133Q, L133H, L133A, Y132I, K134Q, K134T, K134S, K134H, K136Q, K136T, K136S, K136H, E137N, E137Q, E137H, Y138H, Y138I, W143H, W143S, E149Q, L151T, L151Q, L151H, L151A, L151V, R152H, R152Q, F154V, F154I, Y155H, Y155I, F156I, F156V, R159H, R159Q, L160V, L160T, L160Q, L160H, L160A, L160I, Y163H, Y163I, L164T, L164Q, L164H, L164A, L164I, L164V, R165H, and R165Q.

Increased protease resistance of a modified IFN-β polypeptide containing one or more amino acid modifications can occur in serum, blood, saliva, digestive fluids, or in vitro when exposed to one or more proteases. Exemplary of proteases for which an IFN-β polypeptide is resistant are any of pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, luminal pepsin, microvillar endopeptidase, dipeptidyl peptidase, enteropeptidase, hydrolase, NS3, factor Xa, Granzyme B, thrombin, plasmin, urokinase, tPA and PSA.

In some examples, a modified IFN-β polypeptide containing one or more amino acid modifications exhibits increased protein stability due to increased protease resistance to gelatinase B. A modified IFN-β polypeptide that is resistant to gelatinase B includes amino acid modifications at positions containing any one or more amino acid of Phenylalanine (F), Leucine (L), Glutamic Acid (E), Tyrosine (Y), and Glutamine (Q). Exemplary amino acid modifications in an IFN-β polypeptide to confer resistance to gelatinase B correspond to modifications of any of Y3I, Y3H, L6I, L6V, L6H, L6A, Q18S, Q18N, Q18H, Q18T, L20I, L20V, L20H, L20A, L21I, L21V, L21T, L21Q, L21H, L21A, Q23H, Q23S, Q23T, Q23N, L24I, L24V, L24T, L24Q, L24H, L24A, E29N, F38I, F38V, E42N, E43N, E43Q, E43H, Q48H, Q48S, Q48T, Q48N, Q49H, Q49S, Q49T, Q49N, F50I, F50V, Q51H, Q51S, Q51T, Q51N, E53Q, E53H, E53N, L57I, L57V, L57T, L57Q, L57H, Y60H, Y60I, E61H, E61N, E61Q, L63I, L63V, L63T, L63Q, L63H, Q64H, Q64S, Q64T, Q64N, F70I, F70V, Q72H, Q72S, Q72T, Q72N, E81N, E85N, L87I, L87V, L87H, L87A, L88I, L88V, L88T, L88Q, L88H, L88A, L98I, L98V, L98H, L98A, L102I, L102V, L102T, L102Q, L102H, L102A, E103N, E104N, L106I, L106V, L106T, L106Q, L106H, L106A, E107N, E109N, Y125I, Y125H, Y126I, Y126H, Y132I, Y132H, L133I, L133V, L133T, L133Q, L133H, E137N, E149H, E149N, E149Q, L151I, L151V, L151T, L151Q, L151H, L151A, F154I, F154V, F156I, L160I, L160V, L160T, L160Q, L160H, L160A, L164I, L164V, L164H, L164A, L164T, and L164Q of a mature IFN-β polypeptide set forth in SEQ ID NO:1. SEQ ID NOS of exemplary modified IFN-β polypeptides that are modified to be resistant to gelatinase B are set forth in any of SEQ ID NOS: 4-13, 16, 17, 20-27, 30-36, 39-42, 45-54, 61-68, 75-82, 84-87, 537-547, 551, 555-557, 562-564, 567-576, 578-583, 585-589, 591, 604-606, and 610-650, or a biologically active portion thereof.

A modified IFN-β polypeptide provided herein that contains one or more amino acid modifications and exhibits increased protein stability manifested as increased protease resistance to gelatinase B also can contain any one or more further amino acid modification at amino acid positions corresponding to positions Y3, L5, L6, F8, L9, Q10, F15, Q16, Q18, L20, L21, Q23, L28, E29, Y30, L32, F38, E42, E43, L47, Q48, Q49, E53, L57, Y60, E61, L63, Q64, F67, Q72, E81, E85, L87, L88, Y92, Q94, L98, L102, E103, E104, L106, E107, E109, F111, L116, L120, Y125, Y126, L130, Y132, L133, E137, Y138, E149, L151, F154, F156, L160, Y163, and L164 of a mature IFN-β polypeptide set forth in SEQ ID NO:1. For examples, amino acid replacements at any one of the further amino acid positions can include replacements of any of Y3H (i.e. replacement of Y by H at a position corresponding to amino acid position 3 of mature IFN-β (SEQ ID NO:1)), L5V, L5I, L5T, L5Q, L5H, L5A, L5D, L5E, L5K, L5R, L5N, L5S, L6I, L6V, L6H, L6A, L6D, L6E, L6K, L6N, L6Q, L6R, L6S, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, F15I, F15V, F15D, F15E, F15K, F15R, Q16D, Q16E, Q16K, Q16N, Q16R, Q16S, Q16T, Q16C, Q18H, Q18T, L20H, L20A, L20N, L20Q, L20R, L20S, L20T, L20D, L20E, L20K, L21I, L21V, L21T, L21Q, L21H, L21A, Q23H, Q23S, Q23T, Q23N, Q23D, Q23E, Q23K, Q23R, L28V, L28I, L28T, L28Q, L28H, L28A, E29N, E29Q, E29H, Y30H, Y30I, L32V, L32I, L32T, L32Q, L32H, L32A, F38I, F38V, E42Q, E42H, E43Q, E43H, L47V, L47I, L47T, L47Q, L47H, L47A, Q48N, Q49N, E53Q, E53H, L57V, L57T, L57Q, L57H, L57A, Y60H, E61Q, L63V, L63T, L63Q, L63H, L63A, Q64N, F67I, F67V, Q72T, Q72N, E81N, E81Q, E81H, E85Q, E85H, L87I, L87V, L87H, L87A, L87D, L87E, L87, L87R, L87N, L87Q, L87S, L87T, L88T, L88Q, L88H, L88A, Y92H, Y92I, Q94D, Q94E, Q94K, Q94N, Q94R, Q94S, Q94T, Q94C, L98H, L98A, L98D, L98E, L98K, L98N, L98Q, L98R, L98S, L98T, L98C, L102I, L102V, L102T, L102Q, L102H, L102A, E103Q, E103H, E104Q, E104H, L106T, L106Q, L106H, L106A, E107Q, E107H, E109H, E109Q, F111I, F111V, L116V, L116I, L116T, L116Q, L116H, L116A. L116V, L116I, L116T, L116Q, L116H, L116A, Y125H, Y126H, L130V, L130I, L130T, L130Q, L130H, L130A, Y132H, L133I, L133V, L133T, L133Q, L133H, L133A, E137N, E137Q, E137H, Y138H, Y138I, E149Q, L151V, L151T, L151Q, L151H, L151A, F154I, F156I, F156V, L160I, L160V, L160T, L160Q, L160H, L160A, Y163H, Y163I, L164I, L164V, L164T, L164Q, L164H, and L164A of a mature IFN-β polypeptide set forth in SEQ ID NO:1.

In other examples, a modified IFN-β polypeptide exhibits increase protein stability manifested as increased conformational stability. Such a polypeptide can contain any one or more amino acid modifications corresponding to modification of any one or more of R11D, E43K, K45D, K52D, E53R, D54K, E61K, E81K, E85K, E103K, E104R, K105D, E107R, E109R, D110K, R113E, K115Q, K115D, R124D, R124E, R152D, and R165D in a mature IFN-β polypeptide set forth in SEQ ID NO:1. Exemplary sequences of such polypeptides are set forth in any one of SEQ ID NOS: 56, and 134-153 or a biologically active portion thereof.

Increased conformational stability exhibited by a modified IFN-β polypeptide containing one or more amino acid modification provided herein can be due to a change in the isoelectric point of the polypeptide. For example, the isoelectric point of a modified IFN-β polypeptide is increased due to replacement of one or more of a Glutamic Acid (E) or an Aspartic Acid (D) with a Lysine or an Arginine (R). Exemplary of modified IFN-β polypeptides provided herein exhibiting increased protein stability manifested as increased conformational stability due to a modification that increases the isoelectric point of the polypeptide are polypeptides with one or more amino acid modification corresponding to E43K, E53R, D54K, E61K, E81K, E85K, E103K, E104R, E107R, E109R, and D110K of a mature IFN-β polypeptide set forth in SEQ ID NO:1.

In other examples, increased conformational stability of an IFN-β polypeptide provided herein is due to amino acid modifications that decrease the isoelectric point of the polypeptide. For example, the isoelectric point of a modified IFN-β polypeptide is decreased due to replacement of one or more of a Lysine (K) or an Arginine (R) with a Glutamine (Q), Glutamic Acid (E) or Aspartic Acid (D). Exemplary of modified IFN-β polypeptides provided herein exhibiting increased protein stability manifested as increased conformational stability due to a modification that decreases the isoelectric point of the polypeptide are polypeptides with one or more amino acid modification corresponding to K115Q, R11D, K45D, K52D, K105D, K108D, R113E, K115D, R124D, R124E, R152D, and R165D of a mature IFN-β polypeptide set forth in SEQ ID NO:1.

A modified IFN-β polypeptide provided herein that has a decreased isoelectric point compared to an unmodified IFN-β polypeptide also can contain one or more further amino acid modification at amino acid positions corresponding to positions R11, K45, K52, K105, K108, R113, K115Q, R124, R152, and R165 of a mature IFN-β polypeptide set forth in SEQ ID NO:1. For example, amino acid replacements at any one of the further amino acid positions can include replacements of any of R11Q (i.e. replacement of R by Q at a position corresponding to amino acid position 11 of mature IFN-β (SEQ ID NO:1), R11D, K45Q, K52Q, K105Q, K108Q, K108D, R113Q, R113E, K115Q, R124Q, R124D, R124E, R152Q, R152D, R165Q, and R165D of a mature IFN-β polypeptide set forth in SEQ ID NO:1.

Provided herein are modified IFN-β polypeptides containing two or more amino acid modification corresponding to modifications at any two or more positions of Y3, L6, R11, Q18, K19, L20, L21, Q23, L24, E29, K33, D34, F38, D39, P41, E42, E43, K45, Q48, Q49, F50, Q51, K52, E53, D54, L57, Y60, E61, M62, L63, Q64, F70, Q72, D73, W79, E81, E85, L87, L88, L98, K99, L102, E103, E104, K105, L106, E107, K108, E109, D110, R113, K115, M117, L122, K123, R124, Y125, Y126, Y132, L133, K134, K136, E137, W143, R147, E149, L151, R152, F154, F156, L160, L164, and R165 of a mature IFN-β polypeptide set forth in SEQ ID NO:1. For example, amino acid replacements at any two or more of the amino acid positions can include replacements of any of Y3 I (i.e. replacement of Y by I at a position corresponding to amino acid position 3 of mature IFN-β (SEQ ID NO:1)), Y3H, L6I, L6V, L6H, L6A, R11D, Q18H, Q18S, Q18T, Q18N, K19N, L20I, L20V, L20H, L20A, L21I, L21V, L21T, L21Q, L21H, L21A, Q23H, Q23S, Q23T, Q23N, L24I, L24V, L24T, L24Q, L24H, L24A, E29N, K33N, D34N, D34Q, D34G, F38I, F38V, D39N, P41A, P41S, E42N, E43K, E43Q, E43H, E43N, K45D, K45N, Q48H, Q48S, Q48T, Q48N, Q49H, Q49S, Q49T, Q49N, F50I, F50V, Q51H, Q51S, Q51T, Q51N, K52D, K52N, E53R, E53Q, E53H, E53N, D54G, L57I, L57V, L57T, L57Q, L57H, L57A, Y60H, Y60I, E61K, E61Q, E61H, E61N, M62I, M62V, M62T, M62Q, M62H, M62A, L63I, L63V, L63T, L63Q, L63H, L63A, Q64H, Q64S, Q64T, Q64N, F70I, F70V, Q72H, Q72S, Q72T, Q72N, D73N, W79H, W79S, E81K, E81N, E85K, E85N, L87I, L87V, L87H, L87A, L88I, L88V, L88T, L88Q, L88H, L88A, L98I, L98V, L98H, L98A, K99N, L102I, L102V, L102T, L102Q, L102H, L102A, E103K, E103N, E104R, E104N, K105D, K105N, L106I, L106V, L106T, L106Q, L106H, L106A, E107R, E107N, K108D, K108N, E109R, E109N, D110K, D110N, R113E, K115D, K115Q, K115N, K115S, K115H, M117I, M117V, M117T, M117Q, M117A, L122I, L122V, L122T, L122Q, L122H, L122A, K123N, R124D, R124E, Y125H, Y125I, Y126H, Y126I, Y132H, Y132I, L133I, L133V, L133T, L133Q, L133H, L133A, K134N, K136N, E137N, W143H, W143S, R147H, R147Q, E149Q, E149H, E149N, L151I, L151V, L151T, L151Q, L151H, L151A, R152D, F154I, F154V, F156I, F156V, L160I, L160V, L160T, L160Q, L160H, L160A, L164I, L164V, L164T, L164Q, L164H, L164A, R165D of a mature IFN-β polypeptide set forth in SEQ ID NO:1.

Such a modification of an IFN-β polypeptide at two or more positions set forth above can be in a mature human IFN-β polypeptide of SEQ ID NO:1, or its precursor form set forth in SEQ ID NO:2. Modification also be can in a recombinant IFN-β polypeptide set forth in SEQ ID NO:3. It also is understood that amino acid modification of an IFN-β polypeptide can be in an allelic, species, or isoform variant of SEQ ID NO:1, where the allelic or species variant has 40%, 50%, 60%, 70%, 80%, 90%, 91%, 92%, 93%, 94%, 95%, 96%, 97%, 98%, or 99% sequence identity to the polypeptide set forth in SEQ ID NO:1, excluding the modified positions.

Such a modified IFN-β polypeptide provided herein that contains two or more amino acid modifications exhibits increased protein stability, and typically also retains its activity. Generally, increased protein stability is increased protein half-life in vitro or in vivo. Increased protein stability of the modified IFN-β polypeptide is the result only of modification to the primary sequence of the IFN-β polypeptide. In some cases, a modified IFN-β polypeptide provided herein also can include a further amino acid modification that contributes to deimmunization, glycosylation, or PEGylation of the polypeptide such that a modified polypeptide provided herein can be glycosylated or conjugated to a polyethylene glycol (PEG) moiety. Also, a modified IFN-β polypeptide provided herein containing two or more amino acid modifications exhibits increased protein stability manifested as protease resistance, increased conformational stability, or any combination thereof.

A modified IFN-β polypeptide provided herein that contains two or more amino acid modifications set forth above can have 2, 3, 4, 5, 6, 7, 8, 9, 10, 11, 12, 13, 14, 15, 16, 17, 18, 19, or 20 modifications. IFN-β polypeptides that can be modified by two or mutations include mature forms (i.e. the polypeptide whose sequence is set forth in SEQ ID NO. 1) and precursor forms (i.e. the polypeptide whose sequence is set forth in SEQ ID NO. 2). An IFN-β polypeptide containing any two or more amino acid modifications such as is set forth above, can contain a further amino acid modification at amino acid positions corresponding to positions M1, L5, L6, F8, L9, Q10, R11, S12, S13, N14, F15, Q16, C17, K19, L20, W22, Q23, L24, N25, R27, L28, E29, Y30, L32, K33, R35, M36, D39, E42, K45, L47, K52, F67, R71, D73, G78, W79, N80, E81, T82, I83, E85, N86, L87, A89, N90, V91, Y92, Q94, I95, H97, L98, K99, V10, E103, E104, K105, E107, K108, E109, D110, F111, R113, L116, L120, K123, R124, R128, L130, K134, K136, E137, Y138, R152, Y155, R159, Y163, and R165 of a mature IFN-β polypeptide set forth in SEQ ID NO:1. For example, amino acid replacements at any one of the further amino acid positions can include replacements of any of M1V (i.e. replacement of M by V at a position corresponding to amino acid position 1 of mature IFN-β (SEQ ID NO:1), M1V, M1I, M1T, M1A, M1Q, M1D, M1E, M1K, M1N, M1R, M1S, M1C, L5V, L5I, L5T, L5Q, L5H, L5A, L5D, L5E, L5K, L5R, L5N, L5S, L6D, L6E, L6K, L6N, L6Q, L6R, L6S, L6T, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, R11H, R11Q, S12D, S12E, S12K, S12R, S13D, S13E, S13K, S13N, S13Q, S13R, S13T, S13C, N14D, N14E, N14K, N14Q, N14R, N14S, N14T, F15I, F15V, F15D, F15E, F15K, F15R, Q16D, Q16E, Q16K, Q16N, Q16R, Q16S, Q16T, Q16C, C17D, C17E, C17K, C17N, C17R, C17S, C17T, K19Q, K19T, K19S, K19H, L20N, L20Q, L20R, L20S, L20T, L20D, L20E, L20K, W22S, W22H, W22D, W22E, W22K, W22R, Q23D, Q23E, Q23K, Q23R, L24D, L24E, L24K, L24R, N25H, N25S, N25Q, R27H, R27Q, L28V, L28I, L28T, L28Q, L28H, L28A, E29Q, E29H, Y30H, Y30I, L32V, L32I, L32T, L32Q, L32H, L32A, K33Q, K33T, K33S, K33H, R35H, R35Q, M36V, M36I, M36T, M36Q, M36A, D39Q, D39H, D39G, E42Q, E42H, K45Q, K45T, K45S, K45T, L47V, L47I, L47T, L47Q, L47H, L47A, K52Q, K52T, K52S, K52H, F67I, F67V, R71H, R71Q, D73Q, D73H, D73G, G78D, G78E, G78K, G78R, N80D, N80E, N80K, N80R, E81Q, E81H, T82D, T82E, T82K, T82R, I83D, I83E, I83K, I83R, I83N, I83Q, I83S, I83T, E85Q, E85H, N86D, N86E, N86K, N86R, N86Q, N86S, N86T, L87D, L87E, L87K, L87R, L87N, L87Q, L87S, L87T, A89D, A89E, A89K, A89R, N90D, N90E, N90K, N90Q, N90R, N90S, N90T, N90C, V91D, V91E, V91K, V91N, V91Q, V91R, V91S, V91T, V91C, Y92H, Y92I, Q94D, Q94E, Q94K, Q94N, Q94R, Q94S, Q94T, Q94C, I95D, I95E, I95K, I95N, I95Q, I95R, I95S, I95T, H97D, H97E, H97K, H97N, H97Q, H97R, H97S, H97T, H97C, L98D, L98E, L98K, L98N, L98Q, L98R, L98S, L98T, L98C, K99Q, K99T, K99S, K99H, V101D, V101E, V101K, V101N, V101Q, V101R, V101S, V101T, V101C, E103Q, E103H, E104Q, E104H, K105Q, K105T, K105S, K105H, E107Q, E107H, K108Q, K108T, K108S, K108H, E109H, E109Q, D110Q, D110H, D110G, F111I, F111V, R113H, R113Q, L116V, L116I, L116T, L1116Q, L116H, L116A, L120V, L120I, L120T, L120Q, L120H, L120A, K123Q, K123T, K123S, K123H, R124H, R124Q, R128H, R128Q, L130V, L130I, L130T, L130Q, L130H, L130A, K134Q, K134T, K134S, K134H, K136Q, K136T, K136S, K136H, E137Q, E137H, Y138H, Y138I, R152H, R152Q, Y155H, Y155I, R159H, R159Q, Y163H, Y163I, R165H, and R165Q of a mature IFN-β polypeptide.

Provided herein are modified IFN-β polypeptides exhibiting increased conformational stability containing two or more amino acid modifications corresponding to modifications at any two or more positions of M1, L5, L6, F8, L9, Q10, R11, S12, S13, N14, F15, Q16, C17, L20, W22, Q23, L24, E43, K45, K52, E53, D54, E61, G78, W79, N80, E81, T82, I83, E85, N86, L87, A89, N90, V91, Q94, I95, H97, L98, V101, E103, E104, K105, E107, K108, E109, D110, R113, K115, R124, R152, and R165 of a mature IFN-β polypeptide set forth in SEQ ID NO:1. For example, amino acid replacements at any two or more of the amino acid positions can include replacements of any of M1E (i.e. replacement of M by E at a position corresponding to amino acid position 1 of mature IFN-β (SEQ ID NO:1)), M1D, M1K, M1R, M1N, M1Q, M1S, M1T, M1C, L5E, L5D, L5K, L5R, L5N, L5Q, L5S, L5T, L6C, F8E, F8D, F8K, F8R, L9E, L9D, L9K, L9R, L9N, L9Q, L9S, L9T, Q10C, Q10E, Q10D, Q10K, Q10R, Q10N, Q10S, Q10T, R11Q, R11D, S12E, S12D, S12K, S12R, S13E, S13D, S13K, S13R, S13N, S13Q, S13T, S13C, N14E, N14D, N14K, N14R, N14Q, N14S, N14T, F15E, F15D, F15K, F15R, Q16E, Q16D, Q16K, Q16R, Q16N, Q16S, Q16T, Q16C, C17E, C17D, C17K, C17R, C17N, C17Q, C17S, C17T, L20E, L20D, L20K, L20R, L20N, L20Q, L20S, L20T, W22E, W22D, W22K, W22R, Q23E, Q23D, Q23K, Q23R, L24E, L24D, L24K, L24R, E43K, K45Q, K45D, K52Q, K52D, E53R, D54K, E61K, G78E, G78D, G78K, G78R, W79E, W79D, W79K, W79R, N80E, N80D, N80K, N80R, E81K, T82E, T82D, T82K, T82R, I83E, I83D, I183K, I83R, I83N, I83Q, I83S, I83T, E85K, N86E, N86D, N86K, N86R, N86Q, N86S, N86T, L87E, L87D, L87K, L87R, L87N, L87Q, L87S, L87T, A89E, A89D, A89K, A89R, N90E, N90D, N90K, N90R, N90Q, N90S, N90T, N90C, V91E, V91D, V91K, V91R, V91N, V91Q, V91S, V91T, V91C, Q94E, Q94D, Q94K, Q94R, Q94N, Q94S, Q94T, Q94C, I95E, I95D, I95K, I95R, I95N, I95Q, I95S, I95T, H97E, H97D, H97K, H97R, H97N, H97Q, H97S, H97T, H97C, L98E, L98D, L98K, L98R, L98N, L98Q, L98S, L98T, L98C, V101C, V101E, V101D, V101K, V101R, V101N, V101Q, V101S, V101T, E103K, E104R, K105Q, K105D, E107R, K108Q, K108D, E109R, D110K, R113Q, R113E, K115Q, K115D, R124Q, R124D, R124E, R152Q, R152D, R165Q, and R165D of a mature IFN-β polypeptide set forth in SEQ ID NO:1.

Such a modification of an IFN-β polypeptide at two or more positions set forth above that confers increased conformational stability can be in a mature human IFN-β polypeptide of SEQ ID NO:1, or its precursor form set forth in SEQ ID NO:2. Modification also be can in a recombinant IFN-β polypeptide set forth in SEQ ID NO:3. It also is understood that amino acid modification of an IFN-β polypeptide can be in an allelic, species, or isoform variant of SEQ ID NO:1, where the allelic or species variant has 40%, 50%, 60%, 70%, 80%, 90%, 91%, 92%, 93%, 94%, 95%, 96%, 97%, 98%, or 99% sequence identity to the polypeptide set forth in SEQ ID NO:1, excluding the modified positions.

In one example, a modified IFN-β polypeptide that exhibits increased conformational stability due to two or more amino acid modifications contains 2, 3, 4, 5, 6, 7, 8, 9, 10, 11, 12, 13, 14, 15, 16, 17, 18, 19, or 20 modifications. IFN-β polypeptides that can be modified to increase conformational stability include mature forms (i.e. the polypeptide whose sequence is set forth in SEQ ID NO. 1) and precursor forms (i.e. the polypeptide whose sequence is set forth in SEQ ID NO. 2). Typically, the increased conformational stability of the polypeptide is due to modifications only in the primary sequence of the polypeptide. A modified IFN-β polypeptide provided herein, however, also can contain one or more additional amino acid modification contributing to deimmunization, glycosylation, or PEGylation of the polypeptide. In some instances, a modified polypeptide provided herein containing two or more amino acid modifications that confer increased conformational stability also are glycosylated or are conjugated to a polyethylene glycol (PEG) moiety.

Conformational stability of a modified IFN-β polypeptide provided herein can be due to the addition of charges to regions in helices A and C. Such a modified IFN-β polypeptide provided herein can contain two or more amino acid modifications such as any two or more modifications corresponding to any of L5E, L5D, L5K, L5R, F8E, F8D, F8K, F8R, L9E, L9D, L9K, L9R, S12E, S12D, S12K, S12R, F15E, F15D, F15K, F15R, Q16E, Q16D, Q16K, Q16R, L20E, L20D, L20K, L20R, W22E, W22D, W22K, W22R, Q23E, Q23D, Q23K, Q23R, L24E, L24D, L24K, L24R, G78E, G78D, G78K, G78R, W79E, W79D, W79K, W79R, N80E, N80D, N80K, N80R, T82E, T82D, T82K, T82R, 183E, I83D, I83K, I83R, N86E, N86D, N86K, N86R, L87E, L87D, L87K, L87R, A89E, A89D, A89K, and A89R of a mature IFN-β polypeptide set forth in SEQ ID NO:1.

Conformational stability of a modified IFN-β polypeptide provided herein also can be due to increased polar interactions between helices A and C. Such a modified IFN-β polypeptide provided herein can contain two or more amino acid modifications such as any two or more modifications corresponding to any of M1E, M1D, M1K, M1R, M1N, M1Q, M1S, M1T, L5E, L5D, L5K, L5R, L5N, L5Q, L5S, L5T, L6E, L6D, L6K, L6R, L6N, L6Q, L6S, L6T, L9E, L9D, L9K, L9R, L9N, L9Q, L9S, L9T, Q10E, Q10D, Q10K, Q10R, Q10N, Q10S, Q10T, S13E, S13D, S13K, S13R, S13N, S13Q, S13T, N14E, N14D, N14K, N14R, N14Q, N14S, N14T, Q16E, Q16D, Q16K, Q16R, Q16N, Q16S, Q16T, C17E, C17D, C17K, C17R, C17N, C17Q, C17S, C17T, L20E, L20D, L20K, L20R, L20N, L20Q, L20S, L20T, I83E, I83D, I83K, I83R, I83N, I83Q, I83S, I83T, N86E, N86D, N86K, N86R, N86Q, N86S, N86T, L87E, L87D, L87K, L87R, L87N, L87Q, L87S, L87T, N90E, N90D, N90K, N90R, N90Q, N90S, N90T, V91E, V91D, V91K, V91R, V91N, V91Q, V91S, V91T, Q94E, Q94D, Q94K, Q94R, Q94N, Q94S, Q94T, I95E, I95D, I95K, I95R, I95N, I95Q, I95S, I95T, H97E, H97D, H97K, H97R, H97N, H97Q, H97S, H97T, L98E, L98D, L98K, L98R, L98N, L98Q, L98S, L98T, V101E, V101D, V101K, V101R, V101N, V101Q, V101S, and V101T of a mature IFN-β polypeptide set forth in SEQ ID NO:1.

In addition, conformational stability of a modified IFN-β polypeptide provided herein can be due to the introduction of a disulfide bridge in the IFN-β polypeptide. Such a modified IFN-β polypeptide provided herein can contain two or more amino acid modifications such as any two or more modifications corresponding to any of M1C, L6C, Q10C, S13C, Q16C, N90C, V91C, Q94C, H97C, L98C, and V101C of a mature IFN-β polypeptide set forth in SEQ ID NO:1. Disulfide bridges can be formed between the following positions: C1-C101, C6-C98, C16-C90, C10-C97, C10-C98, C13-C94. Exemplary sequences of modified IFN-β polypeptides containing a disulfide bridge are set forth in any of SEQ ID NOS: 126-128, 130, 132, and 133 or a biologically active portion thereof.

Alteration of the isoelectric point of an IFN-β polypeptide also increases the conformational stability of modified IFN-β polypeptides provided herein. Such a modified IFN-β polypeptide provided herein exhibiting an increased isoelectric point can contain two or more amino acid modifications such as any two or more modifications corresponding to any of E43K, E53R, D54K, E61K, E81K, E85K, E103K, E104R, E107R, E109R, and D110K. In another example, a modified IFN-β polypeptide provided herein exhibiting a decreased isoelectric point can contain two or more amino acid modifications such as any two or more modifications corresponding to any of R11D, R11Q, K45D, K45Q, K52D, K52Q, K105D, K105Q, K108D, K108Q, R113E, R113Q, K115D, K115Q, R124D, R124Q, R124E, R152D, R152Q, R165Q, and R165D of a mature IFN-β polypeptide set forth in SEQ ID NO:1.

Provided herein are any of the above modified IFN-β polypeptides, wherein the one or more amino acid modifications are selected from among natural amino acids, non-natural amino acids and a combination of natural and non-natural amino acids. The modified IFN-β polypeptide can be a naked polypeptide chain. Modified IFN-β polypeptide is a polypeptide that can include one or more modifications provided herein and one or more additional amino acid modifications that reduce the immunogenicity of the polypeptide. Methods for effecting modification of polypeptides to reduce immunogenicity are known in the art.

In some examples, the modified IFN-β polypeptide is a polypeptide complex in which the IFN-β polypeptide is pegylated, albuminated, and/or glycosylated.

Provided herein are any of the above modified IFN-β polypeptides, further containing one or more pseudo-wild type mutations. In one embodiment, the pseudo-wild-type mutations include, but are not limited to, one or more of insertions, deletions or replacements of the amino acid residue(s) of the unmodified IFN-β polypeptide.

Provided herein are any of the above modified IFN-β that exhibit increased resistance to proteolysis by one or more proteases. In one embodiment, the increased resistance to proteolysis is to one or more proteases that occur in serum, blood, saliva, digestive fluids and/or in vitro. The increased resistance to proteolysis is exhibited by the modified IFN-β when it is administered intravenously, orally, nasally, pulmonarily, or is present in the digestive tract. Such modified IFN-β polypeptides exhibit increased resistance to proteolysis by one or more proteases compared to the unmodified IFN-β. Exemplary proteases include, but are not limited to, gelatinase A, gelatinase B, pepsin, trypsin, trypsin (Arg blocked), trypsin (Lys blocked), clostripain, endoproteinase Asp-N, chymotrypsin, cyanogen bromide, iodozobenzoate, Myxobacter P., Armillaria, luminal pepsin, microvillar endopeptidase, dipeptidyl peptidase, enteropeptidase and hydrolase.

Provided herein are modified IFN-β polypeptides that exhibit increased conformational stability. Increased conformational stability can confer on a polypeptide increased thermal tolerance, increased tolerance to pH, and/or increased tolerance to a denaturating agent. In some instances, the modified IFN-β has increased thermal tolerance at a temperature from at or about 20° C. to at or about 45° C. In a particular example, the modified IFN-β has increased thermal tolerance at a body temperature of a subject (e.g., at or about 37° C.).

Provided herein are any of the above modified IFN-β polypeptides, in which the increased protein stability is manifested as an increased half-life in vivo or in vitro. In one example, the increased stability is manifested as an increased half-life when administered to a subject. In another example, the modified IFN-β has a half-life increased by at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450% and at least 500% or more compared to the half-life of unmodified IFN-β. In other examples, the modified IFN-β also has a half-life increased by at least 1.5 times, 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times and 1000 times, or more times when compared to the half-life of unmodified IFN-β.

Provided herein are any of the above modified IFN-β polypeptides exhibiting increased activity compared to the unmodified IFN-β. Provided herein are any of the above modified IFN-β polypeptides exhibiting decreased activity compared to the unmodified IFN-β. Activity can be assessed, for example, by measuring cell proliferation in vitro, measuring anti-viral activity in vitro or in vivo, measuring natural killer cell activation, or measuring markers of IFN-β activity. The results of such assays correlate with an in vivo activity and hence a biological activity.

In one example, any of the above modified IFN-β polypeptides exhibits increased protease resistance, increased conformational stability (assessed, for example, as increased thermal tolerance), or any combinations thereof. In some cases, the modified IFN-β exhibits increased resistance to proteolysis and exhibits decreased thermal tolerance compared to the unmodified IFN-β. Alternatively, in another case, the modified IFN-β exhibits increased thermal tolerance and exhibits decreased resistance to proteolysis compared to the unmodified IFN-β. In still other instances, the modified IFN-β polypeptide exhibits increased resistance to proteolysis and increased thermal tolerance.

Provided herein are any of the above modified IFN-β polypeptides that is a precursor polypeptide containing a signal peptide. In one example, the signal sequence is amino acids 1-21 of the sequence of amino acids set forth in SEQ ID NO: 2. Provided herein are any of the above modified IFN-β polypeptides that do not have a signal peptide. Such a modified IFN-β polypeptide is a mature polypeptide. In cases, the modified IFN-β polypeptides provided herein are secreted. Such a secreted polypeptide had a signal sequence that was processed prior to secretion, and possibly also contains other post-translational modifications, such as for example, glycosylation.

It is understood that modifications are with reference to the amino acid numbering of SEQ ID NO:1. Modifications contemplated include, however, mature IFN-β polypeptide of SEQ ID NO:1 as well as in its precursor form set forth in SEQ ID NO:2, and in a form of IFN-β set forth in SEQ ID NO:3. Additionally, corresponding loci on other species of IFN-β polypeptides and allelic variants readily can be identified. Furthermore, shortened or lengthened variants with insertions or deletions of amino acids, particularly at either terminus that retain an activity readily can be prepared and the loci for corresponding mutations identified.

In one example, provided herein is a modified cytokine structural homolog of a modified IFN-β as described herein containing one or more amino acid replacements in the cytokine structural homolog at positions corresponding to the 3-dimensional-structurally-similar positions within the 3-D structure of the modified IFN-β.

Provided herein are libraries (collections) of modified IFN-β polypeptides containing two, three, four, five, six, 10, 50, 100, 200 or more modified IFN-β polypeptides as described herein.

Provided herein are nucleic acid molecules containing a sequence of nucleotides encoding a modified IFN-β polypeptide as described herein. Provided herein are libraries (collections) of nucleic acid molecules comprising a plurality of the molecules as described herein.

Provided herein are vectors comprising the nucleic acid molecules. In one embodiment, the vectors are in a eukaryotic cell, a prokaryotic cell, an insect cell, a mammalian cell, etc. In some examples, the vectors are in a bacterial cell, a Chinese hamster ovary cell or an algal cell. Also provided herein are libraries containing a plurality of the vectors.

Provided herein are methods for expressing a modified IFN-β polypeptide comprising: i) introducing a nucleic acid encoding a modified IFN-β or a vector containing a nucleic acid encoding a modified IFN-β into a cell, and ii) culturing the cell under conditions in which the encoded modified IFN-β is expressed. In one embodiment, the nucleic acid or vectors are in a eukaryotic cell, a prokaryotic cell, an insect cell, a mammalian cell, etc. In some examples, the nucleic acid or vectors are in a bacterial cell, a Chinese hamster ovary cell or an algal cell. In one embodiment, the modified IFN-β is glycosylated.

Provided herein are pharmaceutical compositions including any of the modified IFN-β polypeptides described herein. In some examples, the modified IFN-β polypeptide containing composition also contains a pharmaceutically acceptable excipient, such as a binding agent, a filler, a lubricant, a disintegrant and a wetting agent.

In one example, the pharmaceutical compositions provided herein are formulated for oral, nasal or pulmonary administration. In a particular example, the pharmaceutical compositions are formulated for oral administration. In some instances, the modified IFN-β polypeptide in the pharmaceutical formulation exhibits increased half-life in the gastrointestinal tract under conditions selected from exposure to saliva, exposure to proteases in the gastrointestinal tract and exposure to low pH conditions compared to an unmodified IFN-β cytokine. Proteases include, but are not limited to one or more of a luminal pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, luminal pepsin, microvillar endopeptidase, dipeptidyl peptidase, enteropeptidase, hydrolase, NS3, factor Xa, Granzyme B, thrombin, plasmin, urokinase, tPA and PSA.

Provided in the pharmaceutical compositions herein are modified IFN-β polypeptides, wherein the modification includes removal of proteolytic digestion sites or increasing the conformational stability of the protein structure. In one example, the modified IFN-β in the pharmaceutical composition exhibits increased protein half-life or bioavailability in the gastrointestinal tract. Protein half-life can be increased in an amount of at least 10%, at least 20%, at least 30%, at least 40%, at least 50%, at least 50%, at least 60%, at least 70%, at least 80%, at least 90%, at least 100%, at least 150%, at least 200%, at least 250%, at least 300%, at least 350%, at least 400%, at least 450% or at least 500% or more compared to the half-life of wild-type protein. Alternatively, protein half-life can be increased in an amount of at least 6 times, at least 7 times, at least 8 times, at least 9 times, at least 10 times, at least 20 times, at least 30 times, at least 40 times, at least 50 times, at least 60 times, at least 70 times, at least 80 times, at least 90 times, at least 100 times, at least 200 times, at least 300 times, at least 400 times, at least 500 times, at least 600 times, at least 700 times, at least 800 times, at least 900 times or at least 1000 times or more compared to an unmodified protein.

Provided herein are pharmaceutical compositions prepared that do not contain added protease inhibitors, such as a Bowman-Birk inhibitor, a conjugated Bowman-Birk inhibitor, aprotinin and camostat.

Provided herein are pharmaceutical compositions formulated for oral administration in a form such as a liquid, a pill, a tablet or a capsule. In one example, the pill or tablet is chewable. In another example, the pill or tablet dissolves when exposed to saliva on the tongue or in the mouth. In an additional example, the capsule or tablet is a gastro-resistant capsule or tablet. In still other instances, the pharmaceutical composition in the capsule is in liquid form. In an example where the pharmaceutical composition is a liquid, the liquid can be, for example, a solution, a syrup or a suspension. In another example, the pharmaceutical composition in the capsule is in lyophilized form.

Provided herein are pharmaceutical compositions formulated for controlled release of the modified IFN-β polypeptide. In one such example, the pharmaceutical composition is in the form of a tablet or a lozenge. Lozenges deliver the modified IFN-β to the mucosa of the mouth, the mucosa of the throat or the gastrointestinal tract. Additionally, the lozenge can be formulated with an excipient, such as among anhydrous crystalline maltose and magnesium stearate.

Provided herein are pharmaceutical compositions formulated without protective compounds. In one such embodiment, the modified IFN-β exhibits resistance to gastrointestinal proteases including, but not limited to, gelatinase B.

Pharmaceutical compositions can be further formulated with one or more pharmaceutically-acceptable additives, such as a suspending agent, an emulsifying agent, a non-aqueous vehicle, and/or a preservative.

Provided herein are pharmaceutical compositions of nucleic acid molecules encoding any of the modified IFN-β polypeptides described herein or a vector containing a nucleic acid molecule encoding any of the modified IFN-β polypeptides described herein and a pharmaceutically acceptable excipient.

Provided herein are methods of treating a subject exhibiting symptoms of or having IFN-β-mediated disease or condition or disease or condition that is responsive to the administration of IFN-β by administering any of the pharmaceutical compositions described herein. Also provided herein are uses of a pharmaceutical composition provided herein for the treatment of an IFN-β-mediated disease or condition or disease or condition that is responsive to the administration of IFN-β. Also provided herein are uses of a modified IFN-β provided herein in the manufacture of a pharmaceutical composition for the treatment of an IFN-β-mediated disease or condition or disease or condition that is responsive to the administration of IFN-β. In one example, the IFN-β-mediated disease or condition or disease or condition that is responsive to the administration of IFN-β includes, but is not limited to viral infection, a proliferative disorder, an autoimmune disease, and an inflammatory disorder. In such an example where the disease to be treated is an autoimmune disease, the disease or condition can be, but is not limited to, any one of multiple sclerosis, rheumatoid arthritis, chronic viral hepatitis, hepatitis A, hepatitis B, and myocardial viral infection. In such another example where the disease to be treated is a proliferative disorder, the disease or condition can be, but is not limited to, a cancer or bone disorder. Exemplary of cancers to be treated with a pharmaceutical composition provided herein include uveal, melanoma, colon cancer, liver cancer, or metastatic cancer. Exemplary of a bone disorder is osteoporosis or osteopenia. In such a further example, where the disease to be treated is an inflammatory disorder, the disease or condition can be, but is not limited to, any of asthma, Guillain-Barre syndrome, and inflammatory bowel disease such as for example, ulcerative colitis or Crohn's disease. In an additional example, where the disease is a viral infection, the infection can be, but is not limited to, chronic viral hepatitis or myocardial infection.

Exemplary of diseases to be treated is multiple sclerosis. In one example, treatment can be by administration of a modified polypeptide provided herein that exhibits increased resistance to gelatinase B. Such modified polypeptides have a sequence of amino acids set forth in any of SEQ ID NOS: 4-11, 16, 17, 20-27, 30-36, 39-42, 45-54, 61-70, 75-87, 157, 158, 163-168, 173, 174, 180-185, 190-193, 198, 199, 204, 205, 209, 210, 213-224, 233-238, 247-250, 266-279, 282, 283, 295-310, 328-358, 377-387, 396-403, 408-411, 447-454, 474-479, 497-504, 540-542, 547, 551, 555-558, 562-576, 578-583, 585-589, 591, 604-607, 610-614, 616-650, 652, 653, 655, 656, and 658 as described herein, or a biologically active fragment thereof.

Provided herein are articles of manufacture including, but not limited to, packaging material and a pharmaceutical composition of a modified IFN-β polypeptide described herein contained within the packaging material. In a particular embodiment, the pharmaceutical composition packaged within the article of manufacture is effective for treatment of an IFN-β-mediated disease or disorder, and the packaging material includes a label that indicates that the modified IFN-β is used for treatment of an IFN-β-mediated disease or disorder.

Provided herein are kits including a pharmaceutical composition of a modified IFN-β polypeptide as described herein, a device for administration of the modified IFN-β polypeptide and optionally instructions for administration.

Provided herein are methods for producing a modified target protein, having an evolved predetermined property, wherein the evolved predetermined property is increased protein stability manifested as any one of increased protease resistance or increased conformational stability. In such examples, the increased protein stability of the IFN-β polypeptide that is evolved is due to amino acid modifications, such that only the primary sequence of the polypeptide is modified to confer the property. In one example, a method of increasing protein stability involves the step of introducing one or more amino acid modification that leads to the removal of proteolytic digestion sites such that the polypeptide exhibits increased protease resistance where the amino acid modifications are chosen from any one or more of Y3H, Y3I, L6I, L6V, L6H, L6A, Q18H, Q18S, Q18T, Q18N, K19N, L20I, L20V, L20H, L20A, L21I, L21V, L21T, L21Q, L21H, L21A, Q23H, Q23S, Q23T, Q23N, L24I, L24V, L24T, L24Q, L24H, L24A, E29N, K33N, D34N, D34Q, D34G, F38I, F38V, D39N, P41A, P41S, E42N, E43Q, E43H, E43N, K45N, Q48H, Q48S, Q48T, Q48N, Q49H, Q49S, Q49T, Q49N, F50I, F50V, Q51H, Q51S, Q51T, Q51N, K52N, E53Q, E53H, E53N, D54N, D54Q, D54G, L57I, L57V, L57T, L57Q, L57H, L57A, Y60H, Y60I, E61Q, E61H, E61N, M62I, M62V, M62T, M62Q, M62A, L63I, L63V, L63T, L63Q, L63H, L63A, Q64H, Q64S, Q64T, Q64N, F70I, F70V, Q72H, Q72S, Q72T, Q72N, D73N, W79H, W79S, E81N, E85N, L87I, L87V, L87H, L87A, L88I, L88V, L88T, L88Q, L88H, L88A, L98I, L98V, L98H, L98A, K99N, L102I, L102V, L102T, L102Q, L102H, L102A, E103N, E104N, K105N, L106I, L106V, L106T, L106Q, L106H, L106A, E107N, K108N, E109N, D110N, K115N, K115Q, K115S, K115H, M117I, M117V, M117T, M117Q, M117A, L122I, L122V, L122T, L122Q, L122H, L122A, K123N, Y125H, Y125I, Y126H, Y126I, Y132H, Y132I, L133I, L133V, L133T, L133Q, L133H, L133A, K134N, K136N, E137N, W143H, W143S, R147H, R147Q, E149Q, E149H, E149N, L151I, L151V, L151T, L151Q, L151H, L151A, F154I, F154V, F156I, F156V, L160I, L160V, L160T, L160Q, L160H, L160A, L164I, L164V, L164T, L164Q, L164H, and L164A of a mature IFN-β polypeptide set forth in SEQ ID NO:1.

In a particular example, a method of increasing protein stability involves the step of introducing one or more amino acid modification that leads to the removal of proteolytic digestion sites recognized by gelatinase B such that the polypeptide exhibits increased protease resistance to gelatinase B where the amino acid modifications are chosen from any one or more of Y3H, Y3I, L5V, L5I, L5T, L5Q, L5H, L5A, L5D, L5E, L5K, L5R, L5N, L5S, L6I, L6V, L6H, L6A, L6D, L6E, L6K, L6N, L6Q, L6R, L6S, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, F15I, F15V, F15D, F15E, F15K, F15R, Q16D, Q16E, Q16K, Q16N, Q16R, Q16S, Q16T, Q16C, Q18H, Q18S, Q18T, Q18N, L20I, L20V, L20H, L20A, L20N, L20Q, L20R, L20S, L20T, L20D, L20E, L20K, L21I, L21V, L21T, L21Q, L21H, L21A, Q23H, Q23S, Q23T, Q23N, Q23D, Q23E, Q23K, Q23R, L28V, L28I, L28T, L28Q, L28H, L28A, E29N, E29Q, E29H, Y30H, Y30I, L32V, L32I, L32T, L32Q, L32H, L32A, F38I, F38V, E42N, E42Q, E42H, E43Q, E43H, E43N, L47V, L47I, L47T, L47Q, L47H, L47A, Q48H, Q48S, Q48T, Q48N, Q49H, Q49S, Q49T, Q49N, F50I, F50V, Q51H, Q51S, Q51T, Q51N, E53Q, E53H, E53N, L57I, L57V, L57T, L57Q, L57H, L57A, Y60H, Y60I, E61Q, E61H, E61N, L63I, L63V, L63T, L63Q, L63H, L63A, Q64H, Q64S, Q64T, Q64N, F67I, F67V, F70I, F70V, Q72H, Q72S, Q72T, Q72N, E81N, E81Q, E81H, E85N, E85Q, E85H, L87I, L87V, L87H, L87A, L87D, L87E, L87, L87R, L87N, L87Q, L87S, L87T, L88I, L88V, L88T, L88Q, L88H, L88A, Y92H, Y92I, Q94D, Q94E, Q94K, Q94N, Q94R, Q94S, Q94T, Q94C, L98I, L98V, L98H, L98A, L98D, L98E, L98K, L98N, L98Q, L98R, L98S, L98T, L98C, L102I, L102V, L102T, L102Q, L102H, L102A, E103N, E103Q, E103H, E104N, E104Q, E104H, L106I, L106V, L106T, L106Q, L106H, L106A, E107N, E107Q, E107H, E109N, E109H, E109Q, F111I, F111V, L116V, L116I, L116T, L116Q, L116H, L116A. L116V, L116I, L116T, L116Q, L116H, L116A, Y125H, Y125I, Y126H, Y126I, L130V, L130I, L130T, L130Q, L130H, L130A, Y132H, Y132I, L133I, L133V, L133T, L133Q, L133H, L133A, E137N, E137Q, E137H, Y138H, Y138I, E149Q, E149H, E149N, L151I, L151V, L151T, L151Q, L151H, L151A, F154I, F154V, F156I, F156V, L160I, L160V, L160T, L160Q, L160H, L160A, Y163H, Y163I, L164I, L164V, L164T, L164Q, L164H, and L164A.

In another example, a method of increasing protein stability involves the step of introducing one or more amino acid modification that add charged residues to regions of helices A and C such that the polypeptide exhibits increased conformational stability, where the amino acid modifications are chosen from any one or more of L5E, L5D, L5K, L5R, F8E, F8D, F8K, F8R, L9E, L9D, L9K, L9R, S12E, S12D, S12K, S12R, F15E, F15D, F15K, F15R, Q16E, Q16D, Q16K, Q16R, L20E, L20D, L20K, L20R, W22E, W22D, W22K, W22R, Q23E, Q23D, Q23K, Q23R, L24E, L24D, L24K, L24R, G78E, G78D, G78K, G78R, W79E, W79D, W79K, W79R, N80E, N80D, N80K, N80R, T82E, T82D, T82K, T82R, I83E, I83D, I83K, I83R, N86E, N86D, N86K, N86R, L87E, L87D, L87K, L87R, A89E, A89D, A89K, and A89R of a mature IFN-β polypeptide set forth in SEQ ID NO:1.

In an additional example, a method of increasing protein stability involves the step of introducing one or more amino acid modifications that increase polar interactions between helices A and C, such that the polypeptide exhibits increased conformational stability, where the amino acid modifications are chosen from any one or more or M1E, M1D, M1K, M1R, M1N, M1Q, M1S, M1T, L5E, L5D, L5K, L5R, L5N, L5Q, L5S, L5T, L6E, L6D, L6K, L6R, L6N, L6Q, L6S, L6T, L9E, L9D, L9K, L9R, L9N, L9Q, L9S, L9T, Q10E, Q10D, Q10K, Q10R, Q10N, Q10S, Q10T, S13E, S13D, S13K, S13R, S13N, S13Q, S13T, N14E, N14D, N14K, N14R, N14Q, N14S, N14T, Q16E, Q16D, Q16K, Q16R, Q16N, Q16S, Q16T, C17E, C17D, C17K, C17R, C17N, C17Q, C17S, C17T, L20E, L20D, L20K, L20R, L20N, L20Q, L20S, L20T, I83E, I83D, I83K, I83R, I83N, I83Q, I83S, I83T, N86E, N86D, N86K, N86R, N86Q, N86S, N86T, L87E, L87D, L87K, L87R, L87N, L87Q, L87S, L87T, N90E, N90D, N90K, N90R, N90Q, N90S, N90T, V91E, V91D, V91K, V91R, V91N, V91Q, V91S, V91T, Q94E, Q94D, Q94K, Q94R, Q94N, Q94S, Q94T, I95E, I95D, I95K, I95R, I95N, I95Q, I95S, I95T, H97E, H97D, H97K, H97R, H97N, H97Q, H97S, H97T, L98E, L98D, L98K, L98R, L98N, L98Q, L98S, L98T, V101E, V101D, V101K, V101R, V101N, V101Q, V101S, and V101T.

In a further example, a method of increasing protein stability involves the step of introducing one or more amino acid modifications that create disulfide bridges in the polypeptide, such that the polypeptide exhibits increased conformational stability, where the amino acid modifications are chosen from any one or more of M1C, L6C, Q10C, S13C, Q16C, N90C, V91C, Q94C, H97C, L98C, and V101C of a mature IFN-β polypeptide set forth in SEQ ID NO:1. Such disulfide bridges formed are at any two or more of positions C1, C6, C10, C13, C16, C90, C91, C94, C97, C98, C101, and C17, such as but not limited to disulfide bridges formed between C1-C101, C6-C98, C6-C90, C10-C97, C10-C98, C13-C94, C17-C90, and C17-C91.

As another example, a method of increasing protein stability involves the step of introducing one or more amino acid modifications that increase the isoelectric point such that the polypeptide exhibits increased conformational stability, where the amino acid modifications are chosen from any one or more of E43K, E53R, D54K, E61K, E81K, E85K, E103K, E104R, E107R, E109R, and DI 10K of a mature IFN-β polypeptide set forth in SEQ ID NO:1. In some cases, a method also can include increasing protein stability involving the step of introducing one or more amino acid modifications that decrease the isoelectric point such that the polypeptide exhibits increased conformational stability, where the amino acid modifications are chosen from any one or more of R11Q, K45Q, K52Q, K105Q, K108Q, R113Q, K115Q, R124Q, R152Q, R165Q, R11D, K45D, K52D, K105D, K108D, R113E, K115D, R124D, R124E, R152D, and R165D of a mature IFN-β polypeptide set forth in SEQ ID NO:1.

Also provided herein are any of the above methods for increasing protein stability of an IFN-β polypeptide, where the method also includes a further step of increasing protein stability of the polypeptide by introducing one or more additional amino acid modification into the polypeptide that contributes to one or more of glycosylation or PEGylation.

### DETAILED DESCRIPTION

Outline

A. Definitions

B. Interferon-beta (IFN-β)
1. IFN-β Polypeptide and Expression Thereof
2. IFN-β Structure
3. IFN-β Function
4. IFN-β as a Biopharmaceutical

C. Modified IFN-β and Methods of Modification
1. Non-Restricted Rational Mutagenesis One-Dimensional (1D) Scanning
2. Two-Dimensional (2D) Scanning
   a. Identifying in-silico HITs
   b. Identifying Replacing Amino Acids
   c. Construction of Modified Proteins and Biological Assays
3. Three-Dimensional (3D) Scanning
   a. Homology
   b. 3D-Scanning (Structural Homology) Methods
4. Super-LEADs and Additive Directional Mutagenesis (ADM)
5. Multi-Overlapped Primer Extensions

D. Modified IFN-β Polypeptides Exhibiting Increased Protein Stability
1. Protease Resistance
   a. Serine Proteases
   b. Matrix Metalloproteinases
   c. Generation of IFN-β variants by removal of proteolytic sites
   d. Modified IFN-β Polypeptides Exhibiting Increased Protease Resistance
      i. Modified IFN-β Polypeptides Exhibiting Increased Protease Resistance to Gelatinase B
2. Conformational Stability
   a. Addition of charged residues to hydrophobic areas
   b. Increasing polar interactions between helices A and C
   c. Creation of disulfide bridges
   d. Modification of the Isoelectric Point (pI)
      i. Increasing Isoelectric Point (pI)
      ii. Decreasing Isoelectric Point (pI)
3. SuperLEADS
4. Additional Modifications
   a. Immunogenicity
   b. Glycosylation
   c. Other Modifications

E. Production of IFN-β Polypeptides
1. Polypeptide Expression
   a. Prokaryotes
   b. Yeast
   c. Insects and Insect Cells
   d. Mammalian Cells
   e. Plants
2. Purification
3. Fusion Proteins
4. Polypeptide Modification
5. Nucleotide Sequences

F. Assessing modified IFN-β polypeptide activity(ies)
1. Anti-viral assays
2. Anti-proliferative assays
3. Natural Killer Cell Activation
4. Measuring markers of IFN-β activity
5. Non-human animal models

G. Formulation/Packaging/Administration
1 Administration of modified IFN-β polypeptides
   a. Oral administration
2. Administration of nucleic acids encoding modified IFN-β polypeptides (gene therapy)

H. Therapeutic Uses
1. Autoimmune diseases
   a. Multiple sclerosis (MS)
   b. Rheumatoid arthritis (RA)
2. Inflammatory diseases and disorders
   a. Inflammatory bowel diseases (IBD)
      i. Ulcerative colitis
      ii. Crohn's disease
   b. Asthma
   c. Guillain-Barre Syndrome
3. Proliferative disorders
   a. Cancer
   b. Bone homeostasis
4. Viral infections

I. Combination Therapies

J. Articles of Manufacture and Kits

K. EXAMPLES

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it understood that such identifiers can change and particular information on the internet or similar source can come and go, but equivalent information can be found by searching the internet. Reference thereto evidences the availability and public dissemination of such information.

As used herein, an “interferon-β” polypeptide (also referred to herein as interferon-β or IFN-β) refers to any interferon-β polypeptide, including but not limited to, recombinantly produced polypeptide, synthetically produced polypeptide and IFN-β extracted from cells and tissues including, but not limited to, pituitary and placental tissues, and fibroblasts. IFN-β includes related polypeptides from different species including, but not limited to, animals of human and non-human origin. Human IFN-β (hIFN-β) includes IFN-β, allelic variant isoforms, synthetic molecules from nucleic acids, protein isolated from human tissue and cells, and modified forms of any human IFN-β polypeptides.

IFN-β polypeptides exhibit allelic variation and species variation. For example, IFN-β also includes IFN-β from any species, including human and non-human species. Typically, an allelic or species variant of IFN-β differs from a native or wildtype IFN-β by about or at least 40%, 50%, 60%, 70%, 80%, 90%, 91%, 92%, 93%, 94%, 95%, 96%, 97%, 98%, or 99%. Interferon-β polypeptides of non-human origin include, but are not limited to, bovine, ovine, porcine, rat, rabbit, horse, other primates such as chimpanzees and macaques, pig, dog, mice and avian IFN-β polypeptides. Exemplary IFN-β polypeptides of non-human origin are those having amino acid sequences (including signal sequences) such as primates, for example, chimpanzees (Pan troglodytes, SEQ ID NO: 527) and macaques (Macaca fascicularis, SEQ ID NO: 528); pig (Sus scrofa, SEQ ID NO:529); dog (Canis familiaris, SEQ ID NO: 530); horse (Equus caballus, SEQ ID NO: 531); bovine (Bos Taurus, SEQ ID NO: 532); and mice (Mus musculus, SEQ ID NO: 533).

IFN-β also includes synthetic molecules produced from nucleic acid molecules, protein isolated from human and non-human tissue and cells, and modified forms thereof. Human and non-human IFN-β also includes fragments or portions of IFN-β that are of sufficient length or include appropriate regions to retain at least one activity of a full-length mature polypeptide.

As used herein, a “portion or fragment of an IFN-β polypeptide” refers to any portion of a human or non-human IFN-β polypeptide that exhibits one or more activities of the full-length polypeptide. Such activities include, for example, anti-viral or anti-proliferative activities. Activity can be any level of percentage of activity of the polypeptide including but not limited to, 1% of the activity, 2%, 3%, 4%, 5%, 10%, 20%, 30%, 40%, 50%, 60%, 70%, 80%, 90%, 95%, or more of functional activity to the full-length polypeptide.

As used herein, IFN-β-1a refers to an IFN-β polypeptide that is produced in CHO cells into which cDNA encoding IFN-β has been introduced. INF-β-1a is 166 amino acids in length and is identical to mature, native fibroblast-derived human IFN-β, including glycosylation at the asparagine residue on position 80. The amino acid sequence of IFN-β-1a is provided in SEQ ID NO:1. Two commercial forms of IFN-β-1a are AVONEX® (Biogen Inc, CA, USA) and Rebif® (Serono Inc., Geneva, Switzerland). Rebif® IFN-β-1a differs from Avonex® IFN-β-1a in that it is formulated for administration to the skin (i.e., subcutaneously); whereas Avonex® is formulated for intramuscular administration.

As used herein, IFN-β-1b refers to an IFN-β polypeptide that is produced in E. coli that bears a genetically engineered plasmid encoding human IFN-β. The resulting expressed IFN-β-1b product is not glycosylated, is lacking the amino-terminal methionine (Met1), and the cysteine residue at position 17 is mutated to a serine. IFN-β-1b is 165 amino acids in length and does not include the carbohydrate side chains that are found in natural human IFN-β. A commercial form of IFN-β-1b is BETASERON® (Berlex laboratories, Richmond, Calif., USA). The amino acid sequence of IFN-β-1b is provided in SEQ ID NO: 3.

As used herein, “native IFN-β” refers to an interferon-β as produced by an organism in nature. For example, humans produce IFN-β. Exemplary native human IFN-β polypeptide sequences are set forth in SEQ ID NOS: 1 and 2. Other animals, such as mammals, produce native IFN-β, for example, hamster, mouse, cow, monkey, orangutan, baboon, chimpanzee, macaque, gibbon and gorilla. Sequences for other mammalian IFN-β polypeptides are set forth in SEQ ID NOS:527-533.

As used herein, “mature IFN-β” or “mature IFN-β polypeptide” refers to an interferon-β polypeptide that lacks a signal sequence. Typically, a signal sequence is cleaved following secretion of a protein from a cell. Thus, a mature IFN-β polypeptide is typically a secreted protein. For purposes herein, reference to a mature human IFN-β polypeptide is to a native IFN-β polypeptide lacking a signal sequence, such as for example a mature IFN-β polypeptide set forth in SEQ ID NO:1. The amino acid sequence of IFN-β set forth in SEQ ID NO:1 differs from that of the precursor polypeptide set forth in SEQ ID NO:2 in that SEQ ID NO:1 is lacking the signal sequence which includes residues 1-21 of SEQ ID NO:2.

As used herein, an “interferon-α” polypeptide (also referred to herein as interferon-α or IFN-α) refers to any interferon-α polypeptide, including but not limited to, recombinantly produced polypeptide, synthetically produced polypeptide and IFN-α extracted from cells or tissues including, but not limited to, pituitary and placental tissues. There are at least 13 different IFN-α isoforms. IFN-α also includes related polypeptides from different species including, but not limited to animals of human and non-human origin. Human IFN-α (hIFN-α) includes all isoforms of IFN-α, allelic variant isoforms, synthetic molecules from nucleic acids, protein isolated from human tissue and cells, and modified forms thereof. Exemplary precursor and wild-type mature hIFN-α sequences include IFNα-2 isoforms set forth in SEQ ID NOS: 521-526. Human IFN-α also includes fragments of IFN-α that are of sufficient length to be functionally active.

As used herein, “unmodified target protein,” “unmodified protein,” “unmodified polypeptide,” “unmodified cytokine,” “unmodified IFN-β,” or “unmodified interferon-β” or grammatical variations thereof refers to a starting protein that is selected for modification using the methods provided herein. The starting target polypeptide can be the naturally-occurring, wild-type form of a polypeptide. In addition, the starting target polypeptide can have been previously altered or mutated, such that it differs from the native wild type isoform but is nonetheless referred to herein as a starting unmodified target protein relative to the subsequently modified proteins produced herein. Thus, existing proteins known in the art that previously have been modified to have a desired change, such as an increase or decrease or other alteration, in a particular biological activity or property compared to an unmodified reference protein can be selected and used as the starting unmodified target protein. For example, a protein that has been modified from its native form by one or more single amino acid changes and possesses either an increase or decrease in a desired property, such as resistance to proteolysis or reduced immunogenicity (see e.g., US-2005-0054052), can be used in the methods provided herein as the starting unmodified target protein for further modification of either the same or a different property. Exemplary modified IFN-β polypeptides known in the art include any IFN-β polypeptide described in, for example, published U.S. Application Nos. US-2004-0132977 and US-2005-0054052; U.S. Pat. Nos. 6,127,332, 6,531,122, and 4,588,585; and published International Application Nos. WO 2006/020580; WO 2004/087753, WO 2004/031352, WO 2005/003157, WO 00/68387, WO 98/48018, WO 98/03887, and EP 260350.

Existing proteins known in the art that previously have been modified to have a desired alteration, such as an increase or decrease, in a particular biological activity or property compared to an unmodified or reference protein can be selected and used as provided herein for identification of structurally homologous loci on other structurally homologous target proteins. For example, a protein that has been modified by one or more single amino acid changes and possesses either an increase or decrease in a desired property or activity, such as for example resistance to proteolysis, can be utilized with the methods provided herein to identify on structurally homologous target proteins, corresponding structurally homologous loci that can be replaced with suitable replacing amino acids and tested for either an increase or decrease in the desired activity.

Exemplary unmodified human IFN-β polypeptides include, but are not limited to the mature, wild-type IFN-β polypeptide (SEQ ID NO:1) or the wild-type precursor IFN-β polypeptide having a signal peptide (SEQ ID NO: 2) or a commercially available IFN-β polypeptide that is not glycosylated, does not have the amino-terminal methionine and has a mutation of C17S (SEQ ID NO: 3). One of skill in the art recognizes that the referenced positions of SEQ ID NO:1 differs by one amino acid residue when compared to SEQ ID NO: 3, which is a form of IFN-β lacking the amino-terminal methionine (Met1). Thus, the second amino acid residue of SEQ ID NO:1 “corresponds to” the first amino acid residue of SEQ ID NO: 3

As used herein, “in a position or positions corresponding to an amino acid position” or “corresponding to any one or more amino acid positions” or “amino acid modifications corresponding to any one or more” of a protein, refers to amino acid positions that are determined to correspond to one another based on sequence and/or structural alignments with a specified reference protein. For example, in a position corresponding to an amino acid position of a mature IFN-β polypeptide can be determined empirically by aligning the sequence of amino acids set forth in a mature IFN-β polypeptide, such as for example a mature IFN-β polypeptide having a sequence of amino acids set forth in SEQ ID NO:1, with a particular interferon-β polypeptide of interest. Corresponding positions can be determined by such alignment by one of skill in the art using manual alignments or by using the numerous alignment programs available (for example, BLASTP). By aligning the sequences of IFN-β polypeptides, one skilled in the art can identify corresponding residues, using conserved and identical amino acid residues as guides. For example, one of skill in the art recognizes that the referenced positions of SEQ ID NO:1 differ by one amino acid residue when compared to SEQ ID NO: 3, which is a form of IFN-β lacking the amino-terminal methionine (Met1). Thus, the second amino acid residue of SEQ ID NO:1 “corresponds to” the first amino acid residue of SEQ ID NO: 3. In other instances, corresponding regions can be identified. For example, L6 of SEQ ID NO:1 (mature IFN-β) corresponds to L5 of SEQ ID NO:3 (mature IFN-β lacking the amino-terminal methionine and having a mutation of C17S) or L27 of SEQ ID NO:2 (precursor IFN-β with signal peptide). Further, the position C17 in SEQ ID NO:1 corresponds to position S16 in SEQ ID NO:3. One skilled in the art also can employ conserved amino acid residues as guides to find corresponding amino acid residues between and among human and non-human sequences. For example, amino acid residue 27 of SEQ ID NO: 2 is a leucine which is conserved among human and non-human species. Residue L27 of SEQ ID NO: 2 “corresponds to” residue L27 of SEQ ID NO: 527, residue L27 of SEQ ID NO: 528, residue L27 of SEQ ID NO: 529, residue L38 of SEQ ID NO: 530, residue L27 of SEQ ID NO: 531, residue L27 of SEQ ID NO: 532 and residue L27 of SEQ ID NO: 533. Corresponding positions also can be based on structural alignments, for example by using computer simulated alignments of protein structure. Recitation that amino acids of a polypeptide correspond to amino acids in a disclosed sequence refers to amino acids identified upon alignment of the polypeptide with the disclosed sequence to maximize identity or homology (where conserved amino acids are aligned) using a standard alignment algorithm, such as the GAP algorithm. As used herein, “at a position corresponding to” refers to a position of interest (i.e., base number or residue number) in a nucleic acid molecule or protein relative to the position in another reference nucleic acid molecule or protein. The position of interest to the position in another reference protein can be in, for example, a precursor protein, an allelic variant, a heterologous protein, an amino acid sequence from the same protein of another species, etc. Corresponding positions can be determined by comparing and aligning sequences to maximize the number of matching nucleotides or residues, for example, such that identity between the sequences is greater than 95%, greater than 96%, greater than 97%, greater than 98% or greater than 99%. The position of interest is then given the number assigned in the reference nucleic acid molecule.

As used herein, a “variant,” “mutant,” “interferon-β variant,” “mutant interferon-β,” or “modified IFN-β” refers to an interferon-β polypeptide (protein) that differs from a wildtype IFN-β, including allelic variants, of a particular species by about or at least 40%, 50%, 60%, 70%, 80%, 90%, 91%, 92%, 93%, 94%, 95%, 96%, 97%, 98%, or 99% in its primary sequence of amino acids. Typically, a modified IFN-β polypeptide has one or more modifications in primary sequence compared to an unmodified interferon-β. For example, the modified polypeptides provided herein having one or more amino acid modification can have any number of modifications, such as 1, 2, 3, 4, 5, 6, 7, 8, 9, 10, 11, 12, 13, 14, 15, 16, 17, 18, 19, 20 or more amino acid replacements, deletions or insertions. The one or more mutations can be one or more amino acid replacements (substitutions), insertions, deletions and any combination thereof. For example, an amino acid replacement can include replacement of F by V at a position corresponding to amino acid position 70 of mature IFN-β polypeptide set forth in SEQ ID NO:1, also denoted as F70V. For purposes herein, a modified IFN-β polypeptide also is called a LEAD or Super-LEAD (as defined herein).

As used herein, modified IFN-β polypeptides provided herein that exhibit increased protein stability include IFN-β polypeptides modified at any number of residues whereby the targeted activity or property that is modified, such as protease resistance, is modified, and such that at least one activity, typically a therapeutic activity, is retained at a level, so as, for example, to permit formulation of the IFN-β polypeptide at an effective dosage for treatment. In general, the modified IFN-β polypeptides include 1 or 2 modifications, but can include such modifications in addition to modifications that alter other properties. Hence, included are modified IFN-β polypeptides in which a total of 1, 2, 3, 4, 5, 6, 7, 8, 9, 10, 11, 12, 13, 14, 15, 16, 17, 18, 19, 20 or more positions compared to an unmodified IFN-β polypeptide. Modified IFN-β polypeptides include mature forms and precursor forms. Modification for increased protein stability is with reference to the same polypeptide that does not have the particular or corresponding modification. The polypeptide modified can include additional modifications, and modification in that context is with reference to a wildtype human IFN-β polypeptide that includes a sequence of amino acids set forth in SEQ ID NO:1 or SEQ ID NO:3, and also includes modification relative to an allelic or species variants thereof.

As used herein, an IFN-β polypeptide that has 1, 2, 3, 4, 5, 6, 7, 8, 9, 10, 11, 12, 13, 14, 15, 16, 17, 18, 19 or 20 modifications refers to an IFN-β polypeptide that has the number of amino acid modifications in its primary sequence of amino acids with respect to a wild-type or native IFN-β polypeptide. Such modifications can be, for example, in a human IFN-β polypeptide, such that 1, 2, 3, 4, 5, 6, 7, 8, 9, 10, 11, 12, 13, 14, 15, 16, 17, 18, 19 or 20 modifications in a human IFN-β polypeptide refers to the number of modifications in a wild-type mature human IFN-β polypeptide set forth in SEQ ID NO:1 or allelic variant thereof.

As used herein, “primary sequence” refers to the linear sequence of amino acids of a polypeptide.

As used herein, the terms “homology” and “identity” are used interchangeably, but homology for proteins can include conservative amino acid changes. In general to identify corresponding positions the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073).

As use herein, “sequence identity” refers to the number of identical amino acids (or nucleotide bases) in a comparison between a test and a reference polypeptide or polynucleotide. Homologous polypeptides refer to a pre-determined number of identical or homologous amino acid residues. Homology includes conservative amino acid substitutions as well identical residues. Sequence identity can be determined by standard alignment algorithm programs used with default gap penalties established by each supplier. Homologous nucleic acid molecules refer to a pre-determined number of identical or homologous nucleotides. Homology includes substitutions that do not change the encoded amino acid (i.e., “silent substitutions”) as well identical residues. Substantially homologous nucleic acid molecules hybridize typically at moderate stringency or at high stringency all along the length of the nucleic acid or along at least about 70%, 80% or 90% of the full-length nucleic acid molecule of interest. Also contemplated are nucleic acid molecules that contain degenerate codons in place of codons in the hybridizing nucleic acid molecule. (For determination of homology of proteins, conservative amino acids can be aligned as well as identical amino acids; in this case, percentage of identity and percentage homology vary). Whether any two nucleic acid molecules have nucleotide sequences (or any two polypeptides have amino acid sequences) that are at least 80%, 85%, 90%, 95%, 96%, 97%, 98% or 99% “identical” can be determined using known computer algorithms such as the “FASTA” program, using for example, the default parameters as in Pearson et al. Proc. Natl. Acad. Sci. USA 85: 2444 (1988) (other programs include the GCG program package (Devereux, J., et al., Nucleic Acids Research 12(I): 387 (1984)), BLASTP, BLASTN, FASTA (Atschul, S. F., et al., J. Molec. Biol. 215:403 (1990); Guide to Huge Computers, Martin J. Bishop, ed., Academic Press, San Diego (1994), and Carillo et al. SIAM J Applied Math 48: 1073 (1988)). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. J. Mol. Biol. 48: 443 (1970), as revised by Smith and Waterman (Adv. Appl. Math. 2: 482 (1981)). Briefly, a GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and 0 for non identities) and the weighted comparison matrix of Gribskov et al. Nucl. Acids Res. 14: 6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” represents a comparison between a test and a reference polypeptide or polynucleotide. In one non-limiting example, “at least 90% identical to” refers to percent identities from 90 to 100% relative to the reference polypeptides. Identity at a level of 90% or more is indicative of the fact that, assuming for exemplification purposes a test and reference polynucleotide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often without relying on software.

As used herein, it also is understood that the terms “substantially identical” or “similar” varies with the context as understood by those skilled in the relevant art.

As used herein, “a directed evolution method” refers to methods that “adapt” either proteins, including natural proteins, synthetic proteins or protein domains to have changed properties, such as the ability to act in different or existing natural or artificial chemical or biological environments and/or to elicit new functions and/or to increase or decrease a given activity, and/or to modulate a given feature. Exemplary directed evolution methods include, among others, rational directed evolution methods described in U.S. application Ser. Nos. 10/022,249; and U.S. Published Application No. US-2004-0132977-A1.

As used herein, “two dimensional rational mutagenesis scanning (2-D scanning)” refers to the processes provided herein in which two dimensions of a particular protein sequence are scanned: (1) one dimension is to identify specific amino acid residues along the protein sequence to replace with different amino acids, referred to as is-HIT target positions, and (2) the second dimension is the amino acid type selected for replacing the particular is-HIT target, referred to as the replacing amino acid.

As used herein, a “property” of an IFN-β polypeptide refers to any property of an IFN-β polypeptide. Such properties include, but are not limited to, protein stability, resistance to proteolysis, conformational stability, thermal tolerance, and tolerance to pH conditions. Changes in properties can alter an “activity” of the polypeptide.

As used herein, “protein stability” refers to increased protein-half-life under one or more conditions including, but not limited to, exposure to proteases, increased temperature, particular pH conditions and/or exposure to denaturing ingredients. Increased protein stability exhibited by an IFN-β polypeptide can be manifested as increased protease resistance, or increased conformational stability such as increased tolerance to temperature, pH, or tolerance to other denaturing ingredients. Increased protein stability can result in an increase in serum half-life and/or other pharmacokinetic properties in vivo. A modified polypeptide that exhibits increased protein stability in vitro or in vivo is, for example, 1%, 2%, 3%, 4%, 5%, 6%, 7%, 8%, 9%, 10%, . . . 20%, . . . 30%, . . . 40%, . . . 50%, . . . 60%, . . . , 70%, . . . 80%, . . . 90%, 91%, 92%, 93%, 94%, 95%, 96%, 97%, 98%, 99%, 100%, 150%, 200%, 300%, 400%, 500%, 1000% or more stable than an unmodified polypeptide, or a modified polypeptide that exhibits increased protein stability in vitro or in vivo, for example, has an increased half-life increased by an amount that is at least 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times when compared to the half-life of the unmodified IFN-β polypeptide.

The protein stability of a polypeptide can be assessed in vitro or in vivo by any method known to those of skill. For example, it can be assessed in assays that measure protease resistance or conformational stability (i.e. resistance to temperature). Such assays detect change of a predetermined activity, typically over time and/or during exposure to destabilizing conditions. Exemplary assays are provided herein. For example, the resistance of the modified IFN-β polypeptides compared to wild-type IFN-β against enzymatic cleavage by proteases (e.g., α-chymotrypsin, carboxypeptidase, endoproteinase Arg-C, endoproteinase Asp-N, endoproteinase Glu-C, endoproteinase Lys-C, and trypsin) can be empirically tested by treating the polypeptides with proteases over time and then testing the polypeptides for residual functional activity such as for example, anti-viral or anti-proliferative activities.

As used herein, “resistance to proteolysis” refers to any amount of decreased cleavage of polypeptide by a proteolytic agent, such as a protease. This can be achieved by modifying particular amino acid residues that are susceptible to cleavage by a particular protease to render them less susceptible to cleavage compared to cleavage by the same protease under the same conditions. A modified polypeptide that exhibits increased resistance to proteolysis exhibits, for example, 1%, 2%, 3%, 4%, 5%, 6%, 7%, 8%, 9%, 10%, . . . 20%, . . . 30%, . . . 40%, . . . 50%, . . . 60%, . . . , 70%, . . . 80%, . . . 90%, 91%, 92%, 93%, 94%, 95%, 96%, 97%, 98%, 99%, 100%, 200%, 300%, 400%, 500%, or more resistance to proteolysis than an unmodified polypeptide.

As used herein, “conformational stability” refers to any amount of increased tolerance of a polypeptide to denaturation. This an be achieved by modifying particular amino acid residues that are susceptible to denaturation conditions to render them less susceptible to denaturation under the same conditions. Conformational stability can be determined by assessing the resistance or susceptibility of a polypeptide to denaturation conditions, such as resistance to temperature or pH. A modified polypeptide that exhibits increased conformational stability exhibits, for example, 1%, 2%, 3%, 4%, 5%, 6%, 7%, 8%, 9%, 10%, . . . 20%, . . . 30%, . . . 40%, . . . 50%, . . . 60%, . . . , 70%, . . . 80%, . . . 90%, 91%, 92%, 93%, 94%, 95%, 96%, 97%, 98%, 99%, 100%, 200%, 300%, 400%, 500% or more resistance to denaturation than an unmodified polypeptide. As used herein, “denaturation” refers to any noncovalent change in the structure of a protein. This change can alter the secondary, tertiary, or quaternary structure of the polypeptide molecule. Denaturation of a polypeptide can occur by, for example but not limited to, exposure to chaotropic agents such as urea and guanidine hydrochloride, detergents, temperature, pH, and reagents which cleave disulfide bridges such as dithiothreitol or dithioerythritol.

As used herein, “thermal tolerance” refers to any amount of decreased denaturation of a polypeptide after exposure to altered temperatures. A modified polypeptide that exhibits increased thermal tolerance exhibits, for example, 1%, 2%, 3%, 4%, 5%, 6%, 7%, 8%, 9%, 10%, . . . 20%, . . . 30%, . . . 40%, . . . 50%, . . . 60%, . . . , 70%, . . . 80%, . . . 90%, 91%, 92%, 93%, 94%, 95%, 96%, 97%, 98%, 99%, 100%, 200%, 300%, 400%, 500% or more stability at varied temperatures than an unmodified polypeptide. For example, a modified polypeptide can exhibit increased thermal tolerance in vivo when administered to a subject than an unmodified polypeptide.

As used herein, “proteases,” “proteinases” or “peptidases” are interchangeably used to refer to enzymes that catalyze the hydrolysis of covalent peptidic bonds. Proteases include, for example, serine proteases and matrix metalloproteinases. Serine protease or serine endopeptidases constitute a class of peptidases, which are characterized by the presence of a serine residue in the active center of the enzyme. Serine proteases participate in a wide range of functions in the body, including blood clotting, inflammation as well as digestive enzymes in both prokaryotes and eukaryotes. The mechanism of cleavage by “serine proteases,” is based on nucleophilic attack of a targeted peptidic bond by a serine. Cysteine, threonine or water molecules associated with aspartate or metals also can play this role. Aligned side chains of serine, histidine and aspartate form a catalytic triad common to most serine proteases. The active site of serine proteases is shaped as a cleft where the polypeptide substrate binds. Amino acid residues are labeled from N to C termini of a polypeptide substrate (Pi, . . . , P3, P2, P1, P1′, P2′, P3′, . . . , Pj). The respective binding sub-sites are labeled (Si, . . . , S3, S2, S1, S1′, S2′, S3′, . . . , Sj). The cleavage is catalyzed between P1 and P1′.

As used herein, a matrix metalloproteinases (MMP) refers to any of a family of metal-dependent, such as Zn⁺²-dependent, endopeptidases that degrade components of the extracellular matrix (ECM). MMPs include four classes: collagenases, stromelysin, membrane-type metalloproteinases and gelatinases. Proteolytic activities of MMPs and plasminogen activators, and their inhibitors, are important for maintaining the integrity of the ECM. Cell-ECM interactions influence and mediate a wide range of processes including proliferation, differentiation, adhesion and migration of a variety of cell types. MMPs also process a number of cell-surface cytokines, receptors and other soluble proteins and are involved in tissue remodeling processes such as wound healing, pregnancy and angiogenesis. Under physiological conditions in vivo, MMPs are synthesized as inactive precursors (zymogens) and are cleaved to produce an active form. Additionally, the enzymes are specifically regulated by endogenous inhibitors called tissue inhibitors of matrix metalloproteinases (TIMPs).

As used herein, a “functional activity” or “activity” of an interferon-β polypeptide refers to any activity exhibited by an interferon-β polypeptide that can be assessed. Such activities can be tested in vitro and/or in vivo and include, but are not limited to, cell proliferation and/or differentiation activity, anti-inflammatory activity, anti-proliferative activity, anti-viral activity, morphogenetic activity, cellular activations such as activation of NK cells, therapeutic activity, tumor suppressor activity, ontogenetic activity, oncogenetic activity, enzymatic activity, pharmacological activity, cell/tissue tropism and delivery, or induction of IFN-β induced protein or proteins. Activity can be assessed, for example, by measuring cell proliferation in vitro, measuring anti-viral activity in vitro or in vivo, or measuring binding to an interferon-β receptor. The results of such assays correlate with an in vivo activity and hence a biological activity. Assays to determine functionality or activity of modified forms of IFN-β are known to those of skill in the art. Exemplary assays to assess the functional activity of an IFN-β polypeptide are described in Example 5.

As used herein, “retains at least one activity” refers to the activity exhibited by a modified IFN-β polypeptide compared to an unmodified IFN-β polypeptide. Generally, a modified IFN-β polypeptide that retains an activity of an unmodified IFN-β polypeptide either improves or maintains the requisite biological activity (e.g., anti-viral and anti-proliferation activity) of an unmodified IFN-β polypeptide. In some cases, a modified IFN-β polypeptide can retain an activity that is decreased compared to an unmodified IFN-β polypeptide. Activity of a modified polypeptide can be any level of percentage of activity of the unmodified polypeptide, including but not limited to, 1% of the activity, 2%, 3%, 4%, 5%, 10%, 20%, 30%, 40%, 50%, 60%, 70%, 80%, 90%, 100%, 200%, 300%, 400%, 500%, or more of functional activity compared to the unmodified polypeptide.

As used herein, a recitation that a modified IFN-β polypeptide has more anti-viral activity (or other activity) than anti-proliferative activity (or another activity) compared to the unmodified IFN-β polypeptide is comparing the absolute value of the change in each activity compared to the activity of an unmodified or native form.

As used herein, “in silico” refers to research and experiments performed using a computer. In silico methods include, but are not limited to, molecular modeling studies and biomolecular docking experiments.

As used herein, “is-HIT” refers to an in silico identified amino acid position along a target protein sequence that has been identified based on i) the particular protein properties to be evolved, ii) the protein's sequence of amino acids, and/or iii) the known properties of the individual amino acids. These is-HIT loci on the protein sequence are identified without use of experimental biological methods. For example, once the protein feature(s) to be modified is (are) selected, diverse sources of information or previous knowledge (i.e., protein primary, secondary or tertiary structures, literature, patents) are exploited to determine those amino acid positions that are amenable to improved protein fitness by replacement with a different amino acid. This step utilizes protein analysis “in silico.” All possible candidate amino acid positions along a target protein's primary sequence that might be involved in the feature being evolved are referred to herein as “in silico HITs” (“is-HITs”). The collection (library), of all is-HITs identified during this step represents the first dimension (target residue position) of the two-dimensional scanning methods provided herein.

As used herein, “amenable to providing the evolved predetermined property or activity” in the context of identifying is-HITs refers to an amino acid position on a protein that is contemplated, based on in silico analysis, to possess properties or features that when replaced result in the desired property being evolved. The phrase “amenable to providing the evolved predetermined property or activity” in the context of identifying replacement amino acids refers to a particular amino acid type that is contemplated, based on in silico analysis, to possess properties or features that when used to replace the original amino acid in the unmodified starting protein result in the desired property being evolved.

As used herein, “high-throughput screening” (HTS) refers to processes that test a large number of samples, such as samples of test proteins or cells containing nucleic acids encoding the proteins of interest to identify structures of interest or to identify test compounds that interact with the variant proteins or cells containing them. HTS operations are amenable to automation and are typically computerized to handle sample preparation, assay procedures and the subsequent processing of large volumes of data.

As used herein, the term “restricted,” when used in the context of the identification of is-HIT amino acid positions along the protein sequence selected for amino acid replacement and/or the identification of replacing amino acids, means that fewer than all amino acids on the protein-backbone are selected for amino acid replacement and/or fewer than all of the remaining 19 amino acids available to replace the original amino acid present in the unmodified starting protein are selected for replacement. In particular embodiments of the methods provided herein, the is-HIT amino acid positions are restricted such that fewer than all amino acids on the protein-backbone are selected for amino acid replacement. In other embodiments, the replacing amino acids are restricted such that fewer than all of the remaining 19 amino acids available to replace the native amino acid present in the unmodified starting protein are selected as replacing amino acids. In an exemplary embodiment, both of the scans to identify is-HIT amino acid positions and the replacing amino acids are restricted such that fewer than all amino acids on the protein-backbone are selected for amino acid replacement and fewer than all of the remaining 19 amino acids available to replace the native amino acid are selected for replacement.

As used herein, “candidate LEADs” are modified (mutant) proteins that are contemplated as potentially having an alteration in any attribute, chemical, physical or biological property in which such alteration is sought. In the methods herein, candidate LEADs are generally generated by systematically replacing is-HITS loci in a protein or a domain thereof with typically a restricted subset, or all, of the remaining 19 amino acids, such as obtained using PAM analysis. Candidate LEADs can be generated by other methods known to those of skill in the art tested by the high throughput methods herein. Typically, a candidate lead contains one mutation at one is-HIT position. For purposes herein, a candidate LEAD is generated by modification or replacement of is-HITs in an unmodified IFN-β protein. Reference to exemplary amino acid modifications is to amino acid modifications corresponding to any one or more amino acid positions of a mature IFN-β polypeptide. As discussed herein above, one of skill in the art could determine “corresponding amino acid positions” in unmodified forms of IFN-β used to generate a candidate LEAD compared to a mature IFN-β polypeptide, such as for example a mature IFN-β polypeptide having a sequence of amino acids set forth in SEQ ID NO:1. For example, one of skill in the art recognizes that the referenced positions of SEQ ID NO:1 differ by one amino acid residue when compared to SEQ ID NO: 3, which is a form of IFN-β lacking the amino-terminal methionine (Met1). Thus, the second amino acid residue of SEQ ID NO:1 “corresponds to” the first amino acid residue of SEQ ID NO: 3.

As used herein, “LEADs” are “candidate LEADs” whose activity has been demonstrated to be modified or improved for the particular attribute, chemical, physical or property or activity. For purposes herein a “LEAD” typically has activity or exhibits a property with respect to the activity or property of interest that differs by at least about or at least 1%, 5%, 10%, 20%, 30%, 40%, 50%, 60%, 70%, 80%, 90%, 100%, 150%, 200%, 300%, 400%, 500% or more from the unmodified and/or wild type (native) protein. In certain embodiments, the change in activity is at least about 5%, 10%, 15%, 20%, 25%, 30%, 35%, 40%, 45%, 50%, 55%, 60%, 65%, 70%, 75%, 80%, 85%, 90%, 95% or 100%, of the activity of the unmodified target protein. In other embodiments, the change in activity is not more than about 5%, 10%, 15%, 20%, 25%, 30%, 35%, 40%, 45%, 50%, 55%, 60%, 65%, 70%, 75%, 80%, 85%, 90%, 95% or 100% of the activity of the unmodified target protein. In yet other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times greater than the activity of the unmodified target protein. The desired alteration, which can be either an increase or a reduction in activity, depends upon the function or property of interest (e.g., ˜10%, ˜20%, etc.). The LEADs can be further modified by replacement of a plurality (2 or more) of “is-HIT” target positions on the same protein molecule to generate “super-LEADs.”

As used herein, the term “superLEAD” refers to modified polypeptides (or mutant proteins; variants) obtained by combining the single mutations present in two or more of the LEAD molecules in a single polypeptide molecule. Accordingly, in the context of the modified proteins provided herein, the phrase “polypeptides containing or having two or more single amino acid replacements” or “polypeptides containing any one or more modifications” encompasses any combination of two or more of the mutations described herein for a respective protein. For example, the modified polypeptides provided herein having two or more single amino acid replacements can have any combination of 2, 3, 4, 5, 6, 7, 8, 9, 10, 11, 12, 13, 14, 15, 16, 17, 18, 19, 20 or more of the amino acid replacements at the disclosed replacement positions. The collection of super-LEAD mutant molecules is generated, tested and phenotypically characterized one-by-one in addressable arrays. Super-LEAD mutant molecules are molecules containing a variable number and type of LEAD mutation. Those molecules displaying further improved fitness for the particular feature being evolved, are referred to as super-LEADs. Super-LEADs can be generated by other methods known to those of skill in the art and tested by the high throughput methods herein. For purposes herein, a super-LEAD typically has activity with respect to the function of interest that differs from the improved activity of a LEAD by a desired amount, such as at least 10%, 20%, 30%, 40%, 50%, 60%, 70%, 80%, 90%, 100%, 150%, 200%, 300%, 400%, 500% or more from at least one of the LEAD mutants from which it is derived. As with LEADs, the change in the activity for super-LEADs is dependent upon the activity that is being “evolved.” The desired alteration, which can be either an increase or a reduction in activity, depends upon the function or property of interest.

As used herein, the phrase “altered loci” refers to the is-HIT amino acid positions in the LEADs or super-LEADs that are replaced with different replacing amino acids resulting in the desired altered property.

As used herein, an “exposed residue” presents more than 15% of its surface exposed to the solvent.

As used herein, the phrase “structural homology” refers to the degree of coincidence in space between two or more protein backbones. Protein backbones that adopt the same protein structure, fold and show similarity upon three-dimensional structural superposition in space can be considered structurally homologous. Structural homology is not based on sequence homology, but rather on three-dimensional homology. Two amino acids in two different proteins that are homologous based on structural homology between those proteins do not necessarily need to be in sequence-based homologous regions. For example, protein backbones that have a root mean squared (RMS) deviation of less than 3.5, 3.0, 2.5, 2.0, 1.7 or 1.5 angstroms (Å) at a given space position or defined region between each other can be considered to be structurally homologous in that region and are referred to herein as having a “high coincidence” between their backbones. It is contemplated herein that substantially equivalent (e.g., “structurally related”) amino acid positions that are located on two or more different protein sequences that share a certain degree of structural homology have comparable functional tasks; also referred to herein as “structurally homologous loci.” These two amino acids then can be “structurally similar” or “structurally related” with each other, even if their precise primary linear positions on the sequences of amino acids, when these sequences are aligned, do not match with each other. Amino acids that are “structurally related” can be far away from each other in the primary protein sequences, when these sequences are aligned following the rules of classical sequence homology. As used herein, a “structural homolog” is a protein that is generated by structural homology.

As used herein, a “single amino acid replacement” refers to the replacement of one amino acid by another amino acid. The replacement can be by a natural amino acid or non-natural amino acids. When one amino acid is replaced by another amino acid in a protein, the total number of amino acids in the protein is unchanged.

As used herein, the phrase “only one amino acid replacement occurs on each target protein” refers to the modification of a target protein, such that it differs from the unmodified form of the target protein by a single amino acid change. For example, in one embodiment, mutagenesis is performed by the replacement of a single amino acid residue at only one is-HIT target position on the protein backbone (e.g., “one-by-one” in addressable arrays), such that each individual mutant generated is the single product of each single mutagenesis reaction. The single amino acid replacement mutagenesis reactions are repeated for each of the replacing amino acids selected at each of the is-HIT target positions. Thus, a plurality of mutant protein molecules are produced, whereby each mutant protein contains a single amino acid replacement at only one of the is-HIT target positions.

As used herein, the phrase “pseudo-wild type,” in the context of single or multiple amino acid replacements, are those amino acids that, while different from the original (e.g., such as native) amino acid at a given amino acid position, can replace the native one at that position without introducing any measurable change in a particular protein activity. A population (library) of sets of nucleic acid molecules encoding a collection of mutant molecules is generated and phenotypically characterized such that proteins with sequences of amino acids different from the original amino acid, but that still elicit substantially the same level (i.e., at least 10%, 50%, 70%, 90%, 95%, 100%, 200%, 300%, 400%, or 500%, depending upon the protein) and type of desired activity as the original protein are selected.

As used herein, “corresponding structurally-related positions on two or more proteins,” such as IFN-β protein and other cytokines, refer to those amino acid positions determined based upon structural homology to maximize tri-dimensional overlapping between proteins.

As used herein, the phrase “sequence-related proteins” refers to proteins that have at least 50%, at least 60%, at least 70%, at least 80%, at least 90%, or at least 95% amino acid sequence identity or homology with each other.

As used herein, families of non-related proteins or “sequence-non-related proteins” refer to proteins having less than 50%, less than 40%, less than 30%, less than 20% amino acid identity or homology with each other.

As used herein, a composition, such as a pharmaceutical composition “consisting of a modified IFN-β polypeptide” or “consisting essentially of a modified IFN-β polypeptide” means that that composition includes a pharmaceutically acceptable carrier or vehicle and no other active ingredients, particularly any added protease inhibitors. Compositions can contain some endogenous protease inhibitors or other agent in the vehicle, but compositions that consist of or consist essentially of a modified IFN-β polypeptide do not include any added agents, such as added protease inhibitors.

As used herein, “IFN-β-mediated disease or disorder” refers to any disease or disorder in which treatment with IFN-β ameliorates any symptom or manifestation of the disease or disorder. Exemplary IFN-β-mediated diseases and disorders include, but are not limited to, proliferative disorders and inflammatory disorders, such as cancers, including uveal melanoma, colon cancer, liver cancer and metastasis thereof, asthma, inflammatory bowel diseases such as Crohn's disease and ulcerative colitis, Guillain-Barre syndrome, autoimmune diseases such as multiple sclerosis and rheumatoid arthritis, bone disruption diseases such as osteoporosis, and viral infections such as chronic viral hepatitis and myocardial viral infection.

As used herein, a disease or condition responsive to administration of or treatment with IFN-β refers to any disease or condition in which any symptom or manisfestation of the disease or disorder is amelieorated or alleviated following administration of IFN-β.

As used herein, inflammatory bowel diseases refer to chronic disorders of the gastrointestinal tract, especially Crohn's disease or an ulcerative form of colitis, characterized by inflammation of the intestine and resulting in abdominal cramping and persistent diarrhea. Inflammatory bowel diseases are any of several incurable and debilitating diseases of the gastrointestinal tract characterized by inflammation and obstruction of parts of the intestine. Inflammatory bowel disease (IBD) is a group of inflammatory conditions of the large intestine and, in some cases, the small intestine. Principal forms of IBD include: Crohn's disease and ulcerative colitis (UC). A difference between the two is the location and nature of the inflammatory changes in the gut. Crohn's can affect any part of the gastrointestinal tract, from mouth to anus (skip lesions), although a majority of cases start in the terminal ileum. Ulcerative colitis is restricted to the colon and spares the anus. Microscopically, ulcerative colitis is restricted to the mucosa (epithelial lining of the gut), while Crohn's disease affects the whole bowel wall. Crohn's disease and UC present with extra-intestinal manifestations (such as liver problems, arthritis, skin manifestations and eye problems) in different proportions.

As used herein, asthma refers to chronic respiratory disease, often arising from allergies, that is characterized by sudden recurring attacks of labored breathing, chest constriction, and coughing. A chronic inflammatory respiratory disease is characterized by periodic attacks of wheezing, shortness of breath, and a tight feeling in the chest. A cough producing sticky mucus is symptomatic. The symptoms often appear to be caused by the body's reaction to a trigger such as an allergen (commonly pollen, house dust, animal dander), certain drugs, an irritant (such as cigarette smoke or workplace chemicals), exercise, or emotional stress. These triggers can cause the asthmatic's lungs to release chemicals that create inflammation of the bronchial lining, constriction, and bronchial spasms. If the effect on the bronchi becomes severe enough to impede exhalation, carbon dioxide can build up in the lungs and lead to unconsciousness and death.

As used herein, Guillain-Barre syndrome refers to a temporary inflammation of the nerves, causing pain, weakness, and paralysis in the extremities and often progressing to the chest and face. It typically occurs after recovery from a viral infection or, in rare cases, following immunization for influenza. Guillain-Barre Syndrome is a disease of the nervous system due to damage to the myelin sheath around nerves.

As used herein, viral hepatitis refers to any of various forms of hepatitis caused by a virus, including both hepatitis A and hepatitis B. As used herein, hepatitis A refers to an infection of the liver that is caused by an RNA virus, is transmitted by ingestion of infected food and water, and has a shorter incubation and generally milder symptoms than hepatitis B. Hepatitis A also is called infectious hepatitis. Unlike viral hepatitis B and C, hepatitis A virus does not cause chronic persistent liver infection.

As used herein, hepatitis B refers to an acute infection (sometimes fatal) of the liver that is caused by a DNA virus, is transmitted by contaminated blood or blood derivatives in transfusions, by sexual contact with an infected person, or by the use of contaminated needles and instruments. The disease has a long incubation and symptoms that can become severe or chronic, causing serious damage to the liver. Hepatitis B also is called serum hepatitis.

As used herein, an autoimmune disease refers to a disease or condition in which the body attacks itself and the immune system causes the pathogenic features of the disease or condition. Exemplary autoimmune diseases include scleroderma, lupus, Hashimoto's thyroiditis, and rheumatoid arthritis

As used herein, multiple sclerosis refers to a pathogenically heterogeneous chronic inflammatory disease of the central nervous system (CNS). Histological hallmarks of active MS include, for example, infiltration of T cells, macrophages and B cells, degradation of myelin (and to a lesser extent, axons) and reactive changes of astrocytes and microglia. Myelin is the fatty sheath that surrounds and protects nerve fibers and its destruction is called demyelination. Demyelination causes nerve impulses to be slowed and/or halted and produces the symptoms of MS. MS is characterized as an autoimmune disease because the inflammatory changes are due to an autoimmune attack against self myelin components. Two forms of MS are relapsing remitting multiple sclerosis (RRMS) and primary progressive multiple sclerosis (PPMS).

As used herein, an exacerbation, attack, relapse and flare with reference to MS, refer to a sudden worsening of an MS symptom or symptoms, or the appearance of new symptoms, which last at least 24 hours and are separated from a previous exacerbation by at least one month.

As used herein, rheumatoid arthritis refers to a chronic inflammatory disease that affects the synovial tissue in multiple joints, which leads to joint destruction and disability. Activation of T cells is believed to be the causative factor leading to inflammation in RA, which in turn leads to the activation of macrophages and fibroblast-like synoviocytes. Fibroblast-like synoviocytes produce a variety of pro-inflammatory cytokines causing proliferation of synovial tissue associated with destruction of cartilage and bone.

As used herein, cancer refers to the development and growth of abnormal cells in an uncontrolled manner as is commonly understood by those of skill in the art. Cancers include solid tumors and blood born cancers, such as leukemias. Cancers tend to invade surrounding tissues, and spread to distant sites of the body via the blood stream and lymphatic system. Cancer includes any of various malignant neoplasmas characterized by the proliferation of anaplastic cells that tend to invade surrounding tissue and metastasize to new body sites. Cancer includes lung, prostate, bladder, breast, cervical, kidney and ovarian cancers and also lymphomas and leukemias.

As used herein, a tumor refers to an abnormal growth of tissue resulting from uncontrolled, progressive multiplication of cells with no physiological function or to a neoplasm.

As used herein, cancer cells include malignant neoplastic, anaplastic, metastatic, hyperplastic, dysplastic, neoplastic, malignant tumor (solid or blood-borne) cells that display abnormal growth in the body in an uncontrolled manner.

As used herein, neoplasm refers to new and abnormal growth of tissue, which can be cancerous, such as a malignant tumor.

As used herein, neoplastic disease, means a disease brought about by the existence of a neoplasm in the body.

As used herein, metastasis refers to the migration of cancerous cells to other parts of the body.

As used herein, hyperplasia refers to an abnormal increase in the number of cells in an organ or a tissue with consequent enlargement. As used herein, neoplasm and dysplasia refer to abnormal growth of tissues, organs or cells. As used herein, malignant means cancerous or tending to metastasize. As used herein, anaplastic means cells that have become less differentiated.

As used herein, leukemia refers to a cancer of the blood cells. Any of various acute or chronic neoplastic diseases of the bone marrow in which unrestrained proliferation of white blood cells occurs, usually accompanied by anemia, impaired blood clotting, and enlargement of the lymph nodes, liver and spleen. Leukemia occurs when bone marrow cells multiply abnormally cased by mutations in the DNA of stem cells. Bone marrow stem cells, as used herein, refer to undifferentiated stem cells that differentiate into red blood cells and white blood cells. Leukemia is characterized by an excessive production of abnormal white blood cells, overcrowding the bone marrow and spilling into peripheral blood. Various types of leukemias spread to lymph nodes, spleen, liver, the central nervous system and other organs and tissues.

As used herein, lymphoma refers to a malignant tumor that arises in the lymph nodes or other lymphoid tissue.

As used herein, treatment means any manner in which the symptoms of a condition, disorder or disease are ameliorated or otherwise beneficially altered. Treatment also encompasses any pharmaceutical use of the compositions herein.

As used herein, “treating” a subject with a disease or condition means at the subject's symptoms are partially or totally alleviated, or remain static following treatment. Hence treatment encompasses prophylaxis, therapy and/or cure. Prophylaxis refers to prevention of a potential disease and/or a prevention of worsening of symptoms or progression of a disease. Treatment also encompasses any pharmaceutical use of a modified interferon and compositions provided herein.

As used herein, “therapeutically effective amount” or “therapeutically effective dose” refers to an agent, compound, material, or composition containing a compound that is at least sufficient to produce a therapeutic effect.

As used herein, “patient” or “subject” to be treated includes humans and human or non-human animals. Mammals include, primates, such as humans, chimpanzees, gorillas and monkeys; domesticated animals, such as dogs, horses, cats, pigs, goats, cows; and rodents such as mice, rats, hamsters and gerbils.

As used herein, “a naked polypeptide chain” refers to a polypeptide that is not post-translationally-modified or otherwise chemically-modified, but contains only covalently linked amino acids.

As used herein, a polypeptide complex includes polypeptides produced by chemical modification or post-translational modification. Such modifications include, but are not limited to, pegylation, albumination, glycosylation, farnysylation, phosphorylation and other polypeptide modifications known in the art.

As used herein, “output signal” refers to parameters that can be followed over time and, optionally, quantified. For example, when a recombinant protein is introduced into a cell, the cell containing the recombinant protein undergoes a number of changes. Any such change that can be monitored and used to assess the transformation or transfection is an output signal, and the cell is referred to as a reporter cell; the encoding nucleic acid is referred to as a reporter gene; and the construct that includes the encoding nucleic acid is a reporter construct. Output signals include, but are not limited to, enzyme activity, fluorescence, luminescence, amount of product produced and other such signals. Output signals include expression of a gene or gene product, including heterologous genes (transgenes) inserted into the plasmid virus. Output signals are a function of time (“t”) and are related to the amount of protein used in the composition. For higher concentrations of protein, the output signal can be higher or lower. For any particular concentration, the output signal increases as a function of time until a plateau is reached. Output signals also can measure the interaction between cells, expressing heterologous genes and biological agents.

As used herein, the Hill equation is a mathematical model that relates the concentration of a drug (i.e., test compound or substance) to the response measured

$$y = \frac{{{y}_{max} [ D ]}^{x}}{{[ D ]}^{n} + {[ {D}_{50} ]}^{n}}$$

where y is the variable measured (e.g., such as a response signal) yₘₐₓ is the maximal response achievable, [D] is the molar concentration of a drug, [D₅₀] is the concentration that produces a 50% maximal response to the drug, n is the slope parameter, which is 1 if the drug binds to a single site and with no cooperativity between or among sites. A Hill plot is log₁₀ of the ratio of ligand-occupied receptor to free receptor vs log [D] (M). The slope is n, where a slope of greater than 1 indicates cooperativity among binding sites and a slope of less than 1 can indicate heterogeneity of binding. This equation has been employed in methods for assessing interactions in complex biological systems (see, published International PCT application No. WO 01/44809 based on PCT No. PCT/FR00/03503).

As used herein, in the Hill-based analysis (see, e.g., published International PCT application No. WO 01/44809 based on PCT No. PCT/FR00/03503), the parameters, π, κ, τ, ε, η, θ, are as follows:

π is the potency of the biological agent acting on the assay (cell-based) system;

κ is the constant of resistance of the assay system to elicit a response to a biological agent;

ε is the global efficiency of the process or reaction triggered by the biological agent on the assay system;

τ is the apparent titer of the biological agent;

θ is the absolute titer of the biological agent; and

η is the heterogeneity of the biological process or reaction.

In particular, as used herein, the parameters π (potency) or κ (constant of resistance) are used, respectively, to assess the potency of a test agent to produce a response in an assay system and the resistance of the assay system to respond to the agent.

As used herein, ε (efficiency) is the slope at the inflexion point of the Hill curve (or, in general, of any other sigmoidal or linear approximation), to assess the efficiency of the global reaction (the biological agent and the assay system taken together) to elicit the biological or pharmacological response.

As used herein, τ (apparent titer) is used to measure the limiting dilution or the apparent titer of the biological agent.

As used herein, θ (absolute titer) is used to measure the absolute limiting dilution or titer of the biological agent.

As used herein, η (heterogeneity) measures the existence of discontinuous phases along the global reaction, which is reflected by an abrupt change in the value of the Hill coefficient or in the constant of resistance.

As used herein, a population of sets of nucleic acid molecules encoding a collection (library) of mutants refers to a collection of plasmids or other vehicles that carry (encode) the gene variants. Thus, individual plasmids or other individual vehicles carry individual gene variants. Each element (member) of the collection is physically separated from the others in an appropriate addressable array and has been generated as the single product of an independent mutagenesis reaction. When a collection (library) of such proteins is contemplated, it will be so-stated. A library, contains three, four, five, 10, 50, 100, 500, 1000, 10³, 10⁴ or more modified IFN-β polypeptides.

As used herein, a “reporter cell” is the cell that undergoes the change in response to a condition. For example, in response to exposure to a protein or a virus or to a change it its external or internal environment, the reporter cell “reports” (i.e., displays or exhibits the change).

As used herein, “reporter” or “reporter moiety” refers to any moiety that allows for the detection of a molecule of interest, such as a protein expressed by a cell. Reporter moieties include, but are not limited to, fluorescent proteins (e.g., red, blue and green fluorescent proteins), LacZ and other detectable proteins and gene products. For expression in cells, nucleic acids encoding the reporter moiety can be expressed as a fusion protein with a protein of interest or under the control of a promoter of interest.

As used herein, phenotype refers to the physical, physiological or other manifestation of a genotype (a sequence of a gene). In methods herein, phenotypes that result from alteration of a genotype are assessed.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

As used herein, the abbreviations for any protective groups, amino acids and other compounds are, unless indicated otherwise, in accord with their common usage, recognized abbreviations, or the IUPAC-IUB Commission on Biochemical Nomenclature (Biochem. 11: 1726 (1972)). Each naturally occurring L-amino acid is identified by the standard three letter code (or single letter code) or the standard three letter code (or single letter code) with the prefix “L-;” the prefix “D-” indicates that the stereoisomeric form of the amino acid is D.

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243: 3552-3559 (1969), and adopted 37 C.F.R. §§ 1.821-1.822, abbreviations for amino acid residues are shown in Table 1:

TABLE 1
Table of Correspondence

| SYMBOL | | |
|---|---|---|
| 1-Letter | 3-Letter | AMINO ACID |
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | proline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | asparagines |
| B | Asx | Asn and/or Asp |
| C | Cys | Cysteine |
| X | Xaa | Unknown or other |

All amino acid residue sequences represented herein by formulae have a left to right orientation in the conventional direction of amino-terminus to carboxyl-terminus. In addition, the phrase “amino acid residue” includes the amino acids listed in the Table of Correspondence (Table 1) and modified and unusual amino acids, such as those referred to in 37 C.F.R. §§ 1.821-1.822. A dash at the beginning or end of an amino acid residue sequence indicates a peptide bond to a further sequence of one or more amino acid residues, to an amino-terminal group such as NH₂ or to a carboxyl-terminal group such as COOH.

As used herein, “naturally occurring amino acid” refers to any of the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids are known to those of skill in the art, and, include, for example, amino acids or analogs of amino acids other than the 20 naturally-occurring amino acids and include, but are not limited to, the D-isostereomers of amino acids. Exemplary non-natural amino acids are described herein and are known to those of skill in the art. Modified polypeptides include those that contain non-natural amino acids in place of natural amino acids.

As used herein, nucleic acids include DNA, RNA and analogs thereof, including protein nucleic acids (PNA) and mixtures thereof. Nucleic acids can be single- or double-stranded. When referring to probes or primers (optionally labeled with a detectable label, e.g., a fluorescent or a radiolabel), single-stranded molecules are contemplated. Such molecules are typically of a length such that they are statistically unique of low copy number (typically less than 5, generally less than 3) for probing or priming a library. Generally a probe or primer contains at least 10, 20 or 30 contiguous nucleic acid bases of sequence complementary to, or identical to, a gene of interest. Probes and primers can be 5 or more, 10 or more, 20 or more, 30 or more, 50 or more, or 100 or more nucleic acid bases long.

As used herein, heterologous or foreign nucleic acid, such as DNA and RNA, are used interchangeably and refer to DNA or RNA that does not occur naturally as part of the genome in which it is present or is found at a locus or loci in a genome that differs from that in which it occurs in nature. Heterologous nucleic acid includes nucleic acid not endogenous to the cell into which it is introduced, but that has been obtained from another cell or prepared synthetically. Generally, although not necessarily, such nucleic acid encodes RNA and proteins that are not normally produced by the cell in which it is expressed. Heterologous DNA herein encompasses any DNA or RNA that one of skill in the art recognizes or considers as heterologous or foreign to the cell or locus in or at which it is expressed. Heterologous DNA and RNA also can encode RNA or proteins that mediate or alter expression of endogenous DNA by affecting transcription, translation, or other regulatable biochemical processes. Examples of heterologous nucleic acid include, but are not limited to, nucleic acid that encodes traceable marker proteins (e.g., a protein that confers drug resistance), nucleic acid that encodes therapeutically effective substances (e.g., anti-cancer agents), enzymes and hormones, and DNA that encodes other types of proteins (e.g., antibodies). Hence, herein heterologous DNA or foreign DNA, includes a DNA molecule not present in the exact orientation and position as the counterpart DNA molecule found in the genome. It also can refer to a DNA molecule from another organism or species (i.e., exogenous).

As used herein, “isolated with reference to a nucleic acid molecule or polypeptide or other biomolecule” means that the nucleic acid or polypeptide has separated from the genetic environment from which the polypeptide or nucleic acid were obtained. It also can mean altered from the natural state. For example, a polynucleotide or a polypeptide naturally present in a living animal is not “isolated,” but the same polynucleotide or polypeptide separated from the coexisting materials of its natural state is “isolated,” as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been partially or substantially purified from a recombinant host cell or from a native source. For example, a recombinantly produced version of a compound can be substantially purified by the one-step method described in Smith et al., Gene, 67: 31-40 (1988). The terms isolated and purified can be used interchangeably.

Thus, by “isolated” it is meant that the nucleic acid is free of coding sequences of those genes that, in the naturally-occurring genome of the organism (if any), immediately flank the gene encoding the nucleic acid of interest. Isolated DNA can be single-stranded or double-stranded, and can be genomic DNA, cDNA, recombinant hybrid DNA or synthetic DNA. It can be identical to a starting DNA sequence or can differ from such sequence by the deletion, addition, or substitution of one or more nucleotides.

As used herein, “isolated” or “purified” preparations from biological cells or hosts mean cell extracts containing the indicated DNA or protein including a crude extract of the DNA or protein of interest. For example, for a protein, a purified preparation can be obtained using a single preparative or biochemical technique or a series of preparative or biochemical techniques, and the DNA or protein of interest can be present at various degrees of purity in these preparations. The procedures can include, but are not limited to, ammonium sulfate fractionation, gel filtration, ion exchange chromatography, affinity chromatography, density gradient centrifugation and electrophoresis.

As used herein, a preparation of DNA or protein that is “substantially pure” or “isolated” should be understood to mean a preparation free from naturally-occurring materials with which such DNA or protein is normally associated in nature. “Essentially pure” should be understood to mean a highly purified preparation that contains at least 95% of the DNA or protein of interest.

As used herein, a cell extract that contains the DNA molecule or protein of interest refers to a homogenate preparation or cell-free preparation obtained from cells that express the protein or contain the DNA of interest. The term “cell extract” is intended to include culture media, especially spent culture media from which the cells have been removed.

As used herein, a “receptor” refers to a biologically active molecule that specifically binds to (or with) other molecules. The term “receptor protein” can be used to more specifically indicate the proteinaceous nature of a specific receptor.

As used herein, “recombinant” refers to any progeny formed as the result of genetic engineering.

As used herein, a “promoter region” refers to the portion of DNA of a gene that controls transcription of the DNA to which it is operatively linked. The promoter region includes specific sequences of DNA sufficient for RNA polymerase recognition, binding and transcription initiation. This portion of the promoter region is referred to as the “promoter.” In addition, the promoter region includes sequences that modulate this recognition, binding and transcription initiation activity of the RNA polymerase. Promoters, depending upon the nature of the regulation, can be constitutive or regulated by cis acting or trans acting factors.

As used herein, the phrase “operatively-linked” generally means the sequences or segments have been covalently joined into one piece of DNA, whether in single- or double-stranded form, whereby control or regulatory sequences on one segment control or permit expression or replication or other such control of other segments. The two segments are not necessarily contiguous. For gene expression, a DNA sequence and a regulatory sequence(s) are connected in such a way to control or permit gene expression when the appropriate molecular, e.g., transcriptional activator proteins, are bound to the regulatory sequence(s).

As used herein, “production by recombinant means by using recombinant DNA methods” means the use of the well-known methods of molecular biology for expressing proteins encoded by cloned DNA, including cloning expression of genes and methods, such as gene shuffling and phage display with screening for desired specificities.

As used herein, a splice variant refers to a variant produced by differential processing of a primary transcript of genomic DNA that results in more than one type of mRNA.

As used herein, a composition refers to any mixture of two or more products or compounds (e.g., agents, modulators, regulators, etc.). It can be a solution, a suspension, liquid, powder, a paste, aqueous, non-aqueous formulations or any mixtures thereof.

As used herein, “a combination” refers to any association between two or more items or elements.

As used herein, an “article of manufacture” is a product that is made and sold and that includes a container and packaging, and optional instructions for use of the product. For purposes herein, articles of manufacture encompass packaged modified interferon-β polypeptides and/or encoding nucleic acid molecules.

As used herein, a “kit” refers to a combination of a modified interferon-β polypeptide or nucleic acid molecule provided herein and another item for a purpose including, but not limited to, administration, diagnosis, and assessment of a biological activity or property. Kits also include instructions for use.

As used herein, “substantially identical to a product” means sufficiently similar so that the property of interest is sufficiently unchanged so that the substantially identical product can be used in place of the product.

As used herein, vector (or plasmid) refers to discrete elements that are used to introduce heterologous DNA into cells for either expression or replication thereof. Selection and use of such vehicles are well within the skill of the artisan. “Vector” refers to a nucleic acid molecule that transport another nucleic acid to which it has been linked. One type of exemplary vector is an episome, i.e., a nucleic acid capable of extra-chromosomal replication. Exemplary episomal vectors are those capable of autonomous replication and/or expression of nucleic acids to which they are linked; such vectors typically include origins of replication. Vectors also can be designed for integration into host chromosomes. Vectors capable of directing the expression of genes to which they are operatively linked are referred to herein as “expression vectors.” Expression vectors are often in the form of “plasmids,” which refer generally to circular double-stranded DNA loops which, in their vector form are not bound to the chromosome. “Plasmid” and “vector” are used interchangeably as the plasmid is the most commonly used form of vectors. Other such other forms of expression vectors that serve equivalent functions and that become known in the art subsequently hereto.

As used herein, vector also includes “virus vectors” or “viral vectors.” Viral vectors are engineered viruses that are operatively linked to exogenous genes to transfer (as vehicles or shuttles) the exogenous genes into cells.

As used herein, an adenovirus refers to any of a group of DNA-containing viruses that cause conjunctivitis and upper respiratory tract infections in humans. As used herein, naked DNA refers to histone-free DNA that can be used for vaccines and gene therapy. Naked DNA is the genetic material that is passed from cell to cell during a gene transfer processed called transformation. In transformation, purified or naked DNA is taken up by the recipient cell which will give the recipient cell a new characteristic or phenotype.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is homozygous for that gene or allele. When a subject has two different alleles of a gene, the subject is heterozygous for the gene. Alleles of a specific gene can differ from each other in a single nucleotide or several nucleotides, and can include substitutions, deletions and insertions of nucleotides. An allele of a gene also can be a form of a gene containing a mutation.

As used herein, the terms “gene” or “recombinant gene” refer to a nucleic acid molecule having an open reading frame and including at least one exon and, optionally, an intron-encoding sequence. A gene can be either RNA or DNA. Genes can include regions preceding and following the coding region (leader and trailer).

As used herein, “intron” refers to a DNA sequence present in a given gene which is spliced out during mRNA maturation.

As used herein, “nucleotide sequence complementary to the nucleotide sequence encoding the amino acid sequence set forth in SEQ ID NO:” refers to the nucleotide sequence of the complementary strand of a nucleic acid strand encoding an amino acid sequence having the particular SEQ ID NO:. The term “complementary strand” is used herein interchangeably with the term “complement.” The complement of a nucleic acid strand can be the complement of a coding strand or the complement of a non-coding strand. When referring to double-stranded nucleic acids, the complement of a nucleic acid encoding an amino acid sequence having a particular SEQ ID NO: refers to the complementary strand of the strand encoding the amino acid sequence set forth in the particular SEQ ID NO: or to any nucleic acid having the nucleotide sequence of the complementary strand of the particular nucleic acid sequence. When referring to a single-stranded nucleic acid having a nucleotide sequence, the complement of this nucleic acid is a nucleic acid having a nucleotide sequence which is complementary to that of the particular nucleic acid sequence. As used herein, the term “coding sequence” refers to that portion of a gene that encodes a sequence of amino acids present in a protein.

As used herein, the term “sense strand” refers to that strand of a double-stranded nucleic acid molecule that has the sequence of the mRNA that encodes the sequence of amino acids encoded by the double-stranded nucleic acid molecule.

As used herein, the term “anti-sense strand” refers to that strand of a double-stranded nucleic acid molecule that is the complement of the sequence of the mRNA that encodes the sequence of amino acids encoded by the double-stranded nucleic acid molecule.

As used herein, an “array” refers to a collection of elements, such as nucleic acid molecules, containing three or more members. An addressable array is one in which the members of the array are identifiable, typically by position on a solid phase support or by virtue of an identifiable or detectable label, such as by color, fluorescence, electronic signal (i.e., RF, microwave or other frequency that does not substantially alter the interaction of the molecules of interest), bar code or other symbology, chemical or other such label. In certain embodiments, the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface.

As used herein, a “support” (e.g., a matrix support, a matrix, an insoluble support or solid support, etc.) refers to any solid or semisolid or insoluble support to which a molecule of interest (e.g., a biological molecule, organic molecule or biospecific ligand) is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacryl-amide, silicon, rubber, and other materials used as supports for solid phase syntheses, affinity separations and purifications, hybridization reactions, immunoassays and other such applications. The matrix herein can be particulate or can be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads,” are often, but not necessarily, spherical. Such reference, however, does not constrain the geometry of the matrix, which can be any shape, including random shapes, needles, fibers, and elongated. Roughly spherical beads, particularly microspheres that can be used in the liquid phase, also are contemplated. The beads can include additional components, such as magnetic or paramagnetic particles (see, for example, Dynabeads (Dynal, Oslo, Norway)) for separation using magnets as long as the additional components do not interfere with the methods and analyses herein.

As used herein, matrix or support particles refer to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm³ or less and can be order of cubic microns. Such particles are collectively called “beads.”

As used herein, the abbreviations for any protective groups, amino acids and other compounds are, unless indicated otherwise, in accord with their common usage, recognized abbreviations, or the IUPAC-IUB Commission on Biochemical Nomenclature (Biochem., 11: 942-944 (1972)).

### B. INTERFERON-BETA (IFN-β)

Interferons (IFNs) encompass a family of small secreted proteins that can function as extracellular messengers in a variety of biological processes and pathways. IFNs are a family of functionally related cytokines that exhibit anti-viral, anti-proliferative, and immunomodulatory activities. They are divided into three groups: the type I IFNs, the type II IFN (IFN-γ), and a third class called IFN-lambda which contains three isoforms (IL29, IL28A, and IL28B). IFN-β is a member of the type I class of interferons (IFNs) according to its physical and functional properties and its shared receptor. Type I interferons also include subtypes alpha (α), omega (ω), epsilon (ε), and tau (τ). There are at least 13 different alpha isoforms subtypes that exhibit slightly different specificities. Type I interferons are found in many species, including rats, mice, and most other mammals, and also have been identified in birds, reptiles and fish species. Synthesis of interferons is induced in response to chemical or biological agents including viruses and bacteria.

Interferons, including interferon β (IFN-β), are used as therapeutic agents. Treatment with IFN-β is an established therapy. IFN-β is used, for example, as a therapeutic for treatment of diseases such as multiple sclerosis (MS), rheumatoid arthritis and Crohn's disease. However, patients receiving IFN-β are subject to frequent, repeat applications of the drug due to the well-known instability of IFN-β in the blood stream and under storage conditions. Hence, improved IFN-β stability (half-life) in vivo, such as in serum or following oral administration, and/or in in vitro applications can improve its activity and efficiency as a drug. Accordingly, provided herein are modified IFN-β polypeptides that display improved protein stability as assessed by properties such as resistance to proteases and/or increased conformational stability such as due to increased thermal tolerance, thereby possessing increased protein half-life. The modified polypeptides can possess increased stability in the bloodstream or following oral administration (in vivo) and/or under storage conditions (in vitro).

1. IFN-β Polypeptide and Expression Thereof

The gene for IFN-β lacks introns, and encodes a protein possessing 29% sequence identity with human IFN-α and 50% sequence identity to murine IFN-β. The human IFN-β polypeptide has a molecular weight of 22 kDa. The exemplary human IFN-β gene encodes a precursor polypeptide containing 187 amino acids, including a 21 amino acid signal peptide (SEQ ID NO: 2). Mature IFN-β polypeptides can be of variable length typically including polypeptides of 166 amino acids (SEQ ID NO:1), 164 and 165 amino acids in length. Commercial forms of IFN-β include those sold under the trademarks AVONEX®, BETASERON®, and Rebif®. IFN-β-1a (SEQ ID NO:1, Avonex®, Biogen Inc, CA, USA, and Rebif®, Serono Inc., Geneva, Switzerland) is produced in CHO cells into which cDNA encoding IFN-β has been introduced. IFN-β-1a is 166 amino acids in length and is identical to fibroblast-derived human IFN-β, including glycosylation at the asparagine residue on position 80 (Nelissen et al. Brain 126: 1371-1381 (2003)). Rebif® IFN-β-1a differs from Avonex® IFN-β-1a in that it is formulated for administration to the skin (i.e., subcutaneously) rather than intramuscular administration. IFN-β-1b (SEQ ID NO:3, Betaseron®, Berlex laboratories, Richmond, Calif., USA) is produced in E. coli that bears a genetically engineered plasmid encoding human IFN-β. The resulting expressed IFN-β-1b product is not glycosylated, is lacking the amino terminal methionine (Met1), and the cysteine residue at position 17 is mutated to a serine. IFN-β-1b is 165 amino acids in length and does not include the carbohydrate side chains that are found in natural human IFN-β (Nelissen et al. Brain 126: 1371-1381 (2003)).

Human IFN-β and recombinant IFN-β-1a are N-glycosylated at the asparagine residue at position 80. The glycan present on natural IFN-β and recombinant IFN-β-1a is an oligosaccharide chain of the biantennary complex type, containing an α1-6 linked fucose on the peptide proximal N-acetyl-glucosamine (GlcNac) residue and two a 2-3 linked N-acetyl-neuraminic (NANA) on the terminal galactose residues. This glycan possesses a rigid structure and interacts with the side chain of two amino acids (Q23 in helix A and N86 in helix C) via hydrogen bonds.

The hydrophobic area at the vicinity of the glycosylation site is formed by the interface between helices A and C. There are a few polar interactions among the residues of these helices. Thus, this zone is rendered susceptible to denaturation. In the absence of glycosylation, the protein should be destabilized, which could lead to the opening of the interface between helices A and C and, thus, exposure of Cys17. Cys17 becomes reactive and some intermolecular disulfide bridges can be formed; mutation of Cys17 to Ser17 prevents the formation of these disulfide bridges but not the protein aggregation.

The relative in vitro potencies of IFN-β-1a and IFN-β-1b have been compared in functional assays demonstrating that the specific activity of IFN-β-1a is approximately 10-fold greater than the specific activity of IFN-β-1b (Runkel et al., 1998, Pharm. Res. 15: 641-649). The structural basis for these activity differences has been attributed to differences in glycosylation between the two polypeptide forms of IFN-β. The effect of the carbohydrate was largely manifested through its stabilizing role on structure. The stabilizing effect of the carbohydrate was evident in thermal denaturation experiments. For example, non-glycosylated IFN-β, such as IFN-β-1b, possesses an intact secondary structure, but is more sensitive to thermal denaturation (i.e., denaturation at 4-5° C. lower than the glycosylated protein). Lack of glycosylation also was correlated with an increase in aggregation and an increased sensitivity to thermal denaturation. Enzymatic removal of the carbohydrate from IFN-β-1a with PNGase F caused extensive precipitation of the deglycosylated product. Glycosylation also is attributed to the increased susceptibility of IFN-β-1b to proteolysis, such as proteolysis by gelatinase B, compared to IFN-β-1a.

IFN-β is produced by many cell types, including macrophages, dendritic cells, fibroblasts, endothelial cells, and others. Typically, IFN-β is produced in response to infection such as by inflammatory stimuli including cytokines (e.g., IL-1, IL-2, IL-12, TNF, or CSF), or in response to infection by a virus. For example, double-stranded RNA (dsRNA) is one of the primary intracellular signals for IFN-β production. Healthy cells do not normally contain dsRNA, which is often produced during viral replication as it forms the genome of many viruses. Several transcription factors have been identified that regulate the IFN-β gene such as for example IRF-1 and IRF-2 (Harada et al., (1998) Biochime, 80: 641-650).

2. IFN-β Structure

Structurally, IFNs are members of the helical cytokine family, also known as the hematopoietic growth factor family that are characterized by a similar four-helical bundle topology. Other members of this family include, but are not limited to, growth hormone (GH), interleukins (IL), granulocyte colony-stimulating factor (G-CSF), erythropoietin, leptin, and others. The crystal structure of human IFN-β has been determined (Karpusas et al., (1997) PNAS, 94: 11813-11818) IFN-β is a globular protein containing 5 alpha (α) helices. It has a calculated molecular weight of 20 kDa and an apparent molecular weight of 25 kDA due to glycosylation. The secondary structure of the five α helices (A to E) is connected with inter-helical loops (AB, BC, CD, DE). The AB loop (residues F15 to E42 of the mature chain) and the E helix (residues V148 to N158 of the mature chain) of IFN-β constitute the regions interacting with IFNAR2 chain of the receptor. The B helix, (residues Q64 to D73 of the mature chain), C helix (residues Y92 to T100 of the mature chain) and D helix (residues R128 to E137 of the mature chain) of IFN-β constitute the regions interacting with the IFNAR1 chain of the receptor. IFN-β possesses a disulfide bridge between cysteines 31 and 141 of the mature polypeptide. This disulfide bridge links loops AB and DE and the stabilization of the AB loop by this disulfide bridge appears to play an important role in the binding to the receptor. Crystallographic data indicate that IFN-β can be a dimer, which is an artifact of the crystallization process since IFN-β does not dimerize in vivo. The two molecules of the dimer are coupled by a zinc atom coordinated by histidine 121 of one IFN-β molecule and histidines 93 and 97 of a second IFN-β molecule in the dimer. A water molecule completes the tetrahedral coordination of the zinc atom. The AB loop and D helix of one IFN-β molecule of the dimer and A/C helices of the other IFN-β molecule of the dimer make some hydrophobic contacts.

Mutagenesis studies have identified regions on IFN-β that interact with IFNAR1 and IFNAR2 receptor polypeptides that constitute the common receptor for Interferon Type I molecules. The AB loop and the E helix of IFN-β constitute the regions interacting with IFNAR2 chain of the receptor. The B helix, C helix and D helix of IFN-β constitute the regions interacting with the IFNAR1 chain of the receptor. These two regions that interact with the receptor define two continuous zones on IFN-β that correspond to two opposite faces on the cytokine. Each of these regions is characterized by a core of uncharged residues and the presence of peripheral charged residues (mainly positively charged arginine and lysine residues).

This distribution of charged and uncharged residues observed in IFN-β and IFN-α-2a, can contribute to the specificity of action of each interferon. The uncharged residues making up region N86-N90 of IFN-β do not seem to be directly involved in mediating interactions of the cytokine with the receptor. In contrast, the corresponding region in IFN-α-2a, which contains several charged residues (i.e., Q23, N80 and N86 in IFN-β are replaced by charged residues (lysine, aspartic acid and arginine respectively)), plays an important role for the binding. Different interferon polypeptide conformations upon interaction with the receptor can account for the different cellular responses that occur upon the interaction of different interferons with the same cellular receptor.

3. IFN-β Properties and Activities

All type I interferon molecules, including IFN-α and IFN-β, bind to a common, ubiquitously expressed, receptor complex known as IFNAR composed of two chains: IFNAR1 (110 kDa) and IFNAR2 (100 kDa). Both chains are required for signal transduction but make varying contributions to the binding of different interferon species. For example, IFN-β appears to bind to both chains of the receptor. In contrast, some IFN-α species bind to only one chain of the receptor. In the presence of IFN-β, the two chains assemble into a functional receptor complex which initiates signal transduction. Upon assembly of the IFNAR complex, the intracellular domains of IFNAR1 and IFNAR2 associate with two Janus-family tyrosine kinases, JAK1 and Tyk2, which transphosphorylate themselves and phosphorylate the receptor. The phosphorylated IFNAR1 and IFNAR2 bind to signal transducer and activator of transcription (STAT1) and STAT2. Following dimerization of the STAT proteins, they migrate to the nucleus to activate transcription of multiple genes. IFN-α and IFN-β also activate other signaling pathways including the PI 3-kinase/Akt, p38 MAP kinase, and Raf-1/ERK kinase cascades. Other signaling molecules activated following stimulation by type I interferons include Vav and Cbl docking protein.

Binding of type I interferons to the IFNAR receptor leads to the activation of a variety of genes encoding proteins involved in biological processes participating in the maintenance of homeostasis and cellular defense, including anti-viral, anti-proliferative, and immunomodulatory functions. The pleiotropic action of IFN-β is evidenced by the large number of varied and diverse genes induced by IFN-β including cytochromes, cell scaffolding proteins, immunologically active proteins such as complement components, anti-inflammatory cytokines such as IL-10 and TGF-β, and dsRNA adenosine deaminase, among many others. IFN-β also acts to downregulate or inhibit a number of genes including proinflammatory cytokines such as IL-12 and TNF-α. Generally, IFN-β is an anti-inflammatory molecules whose observed effects on a variety of immune cells (e.g., T cells, NK cells, monocytes, macrophages and dendritic cells) include, for example, the following: enhancement of T cell cytotoxity; regulation of antibody production; inhibition of T cell proliferation and migration; downregulation of adhesion molecules; enhanced expression of tumor-associated surface antigens, stimulation of surface molecules such as MHC class I antigens, induction or activation of pro-apoptotic genes and proteins (e.g., tumor necrosis factor-related apoptosis-inducing ligand, caspases, Bak, Bax, and p53), repression of anti-apoptotic genes (e.g., Bcl-2, inhibitor of apoptosis protein), and inhibition of angiogenesis (Pestka et al. Immunological Reviews 202: 8-32 (2004); Holten et al., (2002), Arthritis Research, 4: 346-352).

For example, in response to viral infection, IFN-β is produced and in turn upregulates the expression of a variety of immune genes involved in MHC Class I antigen presentation including the MHC class I molecule, TAP, Lmp2, and Lmp7. Upregulation of these genes increases the presentation of viral peptides by MHC class I molecules in order to facilitate CD8 T cell recognition and destruction of infected cells. IFN-β also can induce the expression of proteins that inhibit protein translation in virally infected cells thus disrupting viral replication. Examples of such proteins include (2′-5′)-oligoadenylate synthetase and dsRNA dependent protein kinase (Biron et al., (1998), Seminars in Immunology, 10: 383-390). Anti-viral effects of IFN-β also is mediated by the direct activation of Natural Killer (NK) cells which selectively kill virus-infected cells.

Type I interferons also regulate immunomodulatory functions of macrophages, dendritic cells, and other immune cells and thereby promote the establishment of an immune response to a variety of pathogens. For example, IFN-α and IFN-β can enhance macrophage antibody-dependent cytotoxicity and modulate cytokine production by macrophages. Type I interferons produced by macrophages also play a role in antimicrobial immunity by acting in an autocrine manner to enhance phagocytosis and the induction of iNOS, the enzyme that produces the antimicrobial compound nitric oxide. Further, type I interferons can be produced by antigen-presenting cells (APCs), such as macrophages and dendritic cells, following infection by viruses or other pathogens. For example, IFN-β is secreted from APCs following stimulation of Toll receptors by a variety of viral or bacterial pattern recognition molecules, such as lipopolysaccharides, CpG DNA, or double stranded RNA. Secreted IFN-β acts on APCs to induce the expression of costimulatory molecules required for activation of T cell responses and antibody production. The immunomodulatory action of IFN-β also is evidenced by the inhibition of mitogen-induced T cell proliferation and T cell responses through downregulation of interleukin-12 and/or upregulation of interleukin-10.

4. IFN-β as a Biopharmaceutical

IFN-β is administered as a therapeutic agent. For example, in humans IFN-β is used as a therapeutic for treatment of Multiple Sclerosis (MS), rheumatoid arthritis (RA), and therapy of tumors such as haemangiomas and Kaposi's sarcoma as an antiangiogenic agent. Treatment with IFN-β is a well-established therapy. IFN-β is administered intramuscularly or subcutaneously. Typically, multiple administrations are used in treatment regimens. The formulations typically are stored in refrigerated (2-8° C.) conditions to ensure retention of activity.

Hence, improved IFN-β stability (half-life) in administered conditions, such as stability in serum or following oral administration and in vitro (e.g., during production, purification and/or storage conditions) improves its utility and efficiency as a drug. Accordingly, provided herein are mutant variants of the IFN-β protein that display improved stability as assessed by resistance to proteases or resistance to denaturation by denaturing agents such as temperature or pH, thereby possessing increased protein half-life. The modified IFN-β proteins that display improved stability possess increased stability in administration conditions such as in the bloodstream, gastrointestinal tract, under low pH conditions (e.g., the stomach), mouth, throat, and/or under storage conditions.

### C. MODIFIED IFN-β AND METHODS OF MODIFICATION

Provided herein are modified IFN-β proteins. The modified IFN-β proteins (also referred to herein as variants) are increased in protein stability compared to unmodified IFN-β. Mutations of amino acid residues in an IFN-β polypeptide provided herein confer increased protein stability by virtue of a change to the primary sequence of the polypeptide. Other modifications that are or are not in the primary sequence of the polypeptide also can be included, such as, but not limited to, the addition of a carbohydrate moiety following glycosylation of the polypeptide, the addition of a polyethylene glycol (PEG) moiety to the polypeptide, etc. Increasing protein stability (for example, the half-life of protein in vivo) can result in a decrease in the frequency of injections needed to maintain a sufficient drug level in serum, thus leading to, for example: i) higher comfort and acceptance by patients, ii) lower doses necessary to achieve comparable biological effects, and iii) as a consequence of (ii), likely attenuation of any secondary effects.

Among the modified IFN-β polypeptides provided are those with altered specific structural features or properties that contribute to IFN-β protein stability (half-life). Increased protein stability of IFN-β can be achieved, for example, by (i) destruction of protease target residues or sequences and/or (ii) by destruction of target residues or sequences contributing to conformational stability that are susceptible to denaturation by temperature, pH, or other denaturation agent. Modification of IFN-β to increase protein stability can be accomplished while keeping an activity unchanged compared to the unmodified or wild-type IFN-β. Any methods known in the art can be used to create modified IFN-β proteins. In the methods described herein, modifications are chosen using the method of 2D- or 3D-scanning mutagenesis (see for example, WO 2004/022747 and WO 2004/022593).

There are several general approaches described for protein-directed evolution based on mutagenesis. Any of these, alone or in combination can be used to modify a polypeptide such as IFN-β to achieve increased conformational stability. Such methods include random mutagenesis, where the amino acids in the starting protein sequence are replaced by all (or a group) of the 20 amino acids either in single or multiple replacements at different amino acid positions are generated on the same molecule, at the same time. Another method, restricted random mutagenesis methods introduces either all of the 20 amino acids or DNA-biased residues. The bias is based on the sequence of the DNA and not on that of the protein, in a stochastic or semi-stochastic manner, respectively, within restricted or predefined regions of the protein, known in advance to be involved in the biological activity being “evolved.” Additionally, as further described herein methods of rational mutagenesis including 1D-scanning, 2D-scanning and 3-D scanning can be used alone or in combination to construct modified IFN-β variants.

1. Non-Restricted Rational Mutagenesis One-Dimensional (1D)-Scanning

Rational mutagenesis, also termed 1-D scanning, is a two-step process and is described in co-pending U.S. application Ser. No. 10/022,249. 1-D scanning can be used to modify IFN-β and, additionally, to identify positions for further modification by other methods such as 2D- and 3D-scanning. Briefly, the first step requires amino acid scanning where all and each of the amino acids in the starting protein sequence, such as IFN-β (SEQ ID NO:1 or 3) are replaced by a third amino acid of reference (e.g., alanine). Only a single amino acid is replaced on each protein molecule at a time. A collection of protein molecules having a single amino acid replacement is generated such that molecules differ from each other by the amino acid position at which the replacement has taken place. Mutant DNA molecules are designed, generated by mutagenesis and cloned individually, such as in addressable arrays, such that they are physically separated from each other and such that each one is the single product of an independent mutagenesis reaction. Mutant protein molecules derived from the collection of mutant nucleic acid molecules also are physically separated from each other, such as by formatting in addressable arrays. Activity assessment on each protein molecule allows for the identification of those amino acid positions that result in a drop in activity when replaced, thus indicating the involvement of that particular amino acid position in the protein's biological activity and/or conformation that leads to fitness of the particular feature being evolved. Those amino acid positions are referred to as HITs.

At the second step, a new collection of molecules is generated such that each molecule differs from each of the others by the amino acid present at the individual HIT positions identified in step 1. All 20 amino acids (19 remaining) are introduced at each of the HIT positions identified in step 1; while each individual molecule contains, in principle, one and only one amino acid replacement. Mutant DNA molecules are designed, generated by mutagenesis and cloned individually, such as in addressable arrays, such that they are physically separated from each other and such that each one is the single product of an independent mutagenesis reaction. Mutant protein molecules derived from the collection of mutant DNA molecules also are physically separated from each other, such as by formatting in addressable arrays. Activity assessment then is individually performed on each individual mutant molecule. The newly generated mutants that lead to a desired alteration (such as an improvement) in a protein activity are referred to as LEADs. This method permits an indirect search for activity alteration, such as improved stability, improved resistance to proteases and/or thermal conditions, and improved interactions between IFN-β and its receptor, based on one rational amino acid replacement and sequence change at a single amino acid position at a time, in search of a new, unpredicted amino acid sequence at some unpredicted regions along a protein to produce a protein that exhibits a desired activity or altered activity, such as better performance than the starting protein.

In this approach, neither the amino acid position nor the replacing amino acid type are restricted. Full length protein scanning is performed during the first step to identify HIT positions, and then all 20 amino acids are tested at each of the HIT positions, to identify LEAD sequences; while, as a starting point, only one amino acid at a time is replaced on each molecule. The selection of the target region (HITs and surrounding amino acids) for the second step is based upon experimental data for activity obtained in the first step. Thus, no prior knowledge of protein structure and/or function is necessary. Using this approach, LEAD sequences have been found on proteins that are located at regions of the protein not previously known to be involved in the particular biological activity being modified; thus emphasizing the power of this approach to discover unpredictable regions (HITs) as targets for fitness improvement.

2. Two-Dimensional (2D) Rational Scanning

The 2-Dimensional rational scanning (or “2-dimensional scanning”) methods for protein rational evolution (see, co-pending U.S. application Ser. Nos. 10/658,355 and 10/658,834 and published International applications WO 2004/022593 and WO 2004/022747) are based on scanning over two dimensions. The first dimension scanned is amino acid position along the protein sequence to identify is-HIT target positions, and the second dimension is the amino acid type selected for replacing a particular is-HIT amino acid position. An advantage of the 2-dimensional scanning methods provided herein is that at least one, and typically both, of the amino acid position scan and/or the replacing amino acid scan can be restricted such that fewer than all amino acids on the protein-backbone are selected for amino acid replacement; and/or fewer than all of the remaining 19 amino acids available to replace an original, such as native, amino acid are selected for replacement.

In particular embodiments, based on i) the particular protein properties to be evolved, ii) the protein's amino acid sequence, and iii) the known properties of the individual amino acids, a number of target positions along the protein sequence are selected, in silico, as “is-HIT target positions.” This number of is-HIT target positions is as large as possible such that all reasonably possible target positions for the particular feature being evolved are included. In particular, embodiments where a restricted number of is-HIT target positions are selected for replacement, the amino acids selected to replace the is-HIT target positions on the particular protein being modified can be either all of the remaining 19 amino acids or, more frequently, a more restricted group having selected amino acids that are contemplated to have the desired effect on protein activity. In another embodiment, so long as a restricted number of replacing amino acids are used, all of the amino acid positions along the protein backbone can be selected as is-HIT target positions for amino acid replacement. Mutagenesis then is performed by the replacement of single amino acid residues at specific is-HIT target positions on the protein backbone (e.g., “one-by-one,” such as in addressable arrays), such that each individual mutant generated is the single product of each single mutagenesis reaction. Mutant DNA molecules are designed, generated by mutagenesis and cloned individually, such as in addressable arrays, such that they are physically separated from each other and that each one is the single product of an independent mutagenesis reaction. Mutant protein molecules derived from the collection of mutant DNA molecules also are physically separated from each other, such as by formatting in addressable arrays. Thus, a plurality of mutant protein molecules are produced. Each mutant protein contains a single amino acid replacement at only one of the is-HIT target positions. Activity assessment is then individually performed on each individual protein mutant molecule, following protein expression and measurement of the appropriate activity. An example of practice of this method is shown in the Examples in which mutant IFN-β molecules are produced.

The newly generated proteins that lead to altered, typically improvement, in a target protein activity are referred to as LEADs. This method relies on an indirect search for protein improvement for a particular property or feature, such as increased resistance to proteolysis, based on a rational amino acid replacement and sequence change at single or, in another embodiment, a limited number of amino acid positions at a time. As a result, modified proteins that have new amino acid sequences at some regions along the protein that perform better (at a particular target activity or other property) than the starting protein are identified and isolated.

2D scanning on IFN-β was used to generate variants improved in protein stability, including improved resistance to proteolysis and improved conformational stability. To effect such modifications, amino acid positions were selected using in silico analysis of IFN-β.

a. Identifying In-Silico HITs

The method for directed evolution includes identifying and selecting (using in silico analysis) specific amino acids and amino acid positions (referred to herein as is-HITs) along the protein sequence that are contemplated to be directly or indirectly involved in the feature being evolved. As noted, the 2-dimensional scanning methods provided include the following two-steps. The first step is an in silico search of a target protein's amino acid sequence to identify all possible amino acid positions that potentially can be targets for the property or feature being evolved. This is effected, for example, by assessing the effect of amino acid residues on the property(ies) to be altered on the protein, using any known standard software. The particulars of the in silico analysis is a function of the property to be modified.

Once identified, these amino acid positions or target sequences are referred to as “is-HITs” (in silico HITs). In silico HITs are defined as those amino acid positions (or target positions) that potentially are involved in the “evolving” feature, such as increased resistance to proteolysis. The discrimination of the is-HITs among all the amino acid positions in a protein sequence can be made based on the amino acid type at each position in addition to the information on the protein secondary or tertiary structure. In silico HITs constitute a collection of mutant molecules such that all possible amino acids, amino acid positions or target sequences potentially involved in the evolving feature are represented. No strong theoretical discrimination among amino acids or amino acid positions is made at this stage. In silico HIT positions are spread over the full length of the protein sequence. Single or a limited number of is-HIT amino acids are replaced at a time on the target protein IFN-β.

A variety of parameters can be analyzed to determine whether or not a particular amino acid on a protein might be involved in the evolving feature, typically a limited number of initial premises (typically no more than 2) are used to determine the in silico HITs. For example, as described herein, to increase the isoelectric point of IFN-β, the first condition is the nature of the amino acids linked to isoelectric point, e.g. negatively charged amino acids. The second premise is typically related to the specific position of those amino acids along the protein structure. For example, some amino acids were not selected because they lie in a region known to participate in IFN-β-receptor interactions.

During the first step of identification of is-HITs according to the methods provided herein, each individual amino acid along the protein sequence is considered individually to assess whether it is a candidate for is-HIT. This search is done one-by-one and the decision on whether the amino acid is considered to be a candidate for a is-HIT is based on (1) the amino acid type itself; (2) the position on the amino acid sequence and protein structure if known; and (3) the predicted interaction between that amino acid and its neighbors in sequence and space.

Using the 3D-scanning methods described herein, once one protein within a family of proteins (e.g., IFN-β within the cytokine family) is modified using the methods provided herein for generating LEAD mutants, is-HITs can be identified on other or all proteins within a particular family by identifying the corresponding amino acid positions therein using structural homology analysis (based upon comparisons of the 3-D structures of the family members with original protein to identify corresponding residues for replacement) as described hereinafter. The is-HITs on family identified in this manner then can be subjected to the next step of identifying replacing amino acids and further assayed to obtain LEADs or super-LEADs as described herein. Similarly, information from 2D-scanning performed on cytokines such as IFN-α, can be used to optimize IFN-β.

Identified Is-HITs provided herein contribute to a number of properties of IFN-β that participate in protein stability such as removal/modification of protease sensitive sites, and modification of sites susceptible to denaturation and conformational stability (i.e. the addition of charges in the hydrophobic region in helices A and C to favor polar interactions with a solvent, increasing intra-molecular polar interactions between helices A and C, creating intra-molecular disulfide bridges, and changing the isoelectric point (pI)), and combinations thereof. Any of the above modifications contribute to protein stability and thereby, to increasing the half-life of an IFN-β polypeptide in vitro, in vivo or ex vivo.

b. Identifying Replacing Amino Acids

Once the is-HITs target positions are selected, the next step is identifying those amino acids that will replace the original, such as native, amino acid at each is-HIT position to alter the activity level for the particular feature being evolved. The set of replacing amino acids to be used to replace the original, such as native, amino acid at each is-HIT position can be different and specific for the particular is-HIT position. The choice of the replacing amino acids takes into account the need to preserve the physicochemical properties such as hydrophobicity, charge and polarity, of essential (e.g., catalytic, binding, etc.) residues. The number of replacing amino acids, of the remaining 19 non-native (or non-original) amino acids, that can be used to replace a particular is-HIT target position ranges from 1 up to about 19, and anywhere in between depending on the properties for the particular modification.

Numerous methods of selecting replacing amino acids (also referred to herein as “replacement amino acids”) are well known in the art. Protein chemists determined that certain amino acid substitutions commonly occur in related proteins from different species. As the protein still functions with these substitutions, the substituted amino acids are compatible with protein structure and function. Often, these substitutions are to a chemically similar amino acid, but other types of changes, although relatively rare, also can occur.

Knowing the types of changes that are most and least common in a large number of proteins can assist with predicting alignments and amino acid substitutions for any set of protein sequences. Amino acid substitution matrices are used for this purpose. A number of matrices are available. A detailed presentation of such matrices can be found in the co-pending U.S. application Ser. Nos. 10/658,355 and 10/658,834 and published International applications WO 2004/022593 and WO 2004/022747. Such matrices also are known and available in the art, for example in the reference listed below.

In amino acid substitution matrices, amino acids are listed across the top of a matrix and down the side, and each matrix position is filled with a score that reflects how often one amino acid would have been paired with the other in an alignment of related protein sequences. The probability of changing amino acid A into amino acid B is assumed to be identical to the reverse probability of changing B into A. This assumption is made because, for any two sequences, the ancestor amino acid in the phylogenetic tree is usually not known. Additionally, the likelihood of replacement should depend on the product of the frequency of occurrence of the two amino acids and on their chemical and physical similarities. A prediction of this model is that amino acid frequencies will not change over evolutionary time (Dayhoff et al., Atlas of Protein Sequence and Structure 5(3): 345-352 (1978)). Several exemplary amino acid substitution matrices, include, but are not limited to, block substitution matrix (BLOSUM) (Henikoff et al., Proc. Natl. Acad. Sci. USA 89: 10915-10919 (1992)), Jones (Jones et al., Comput. Appl. Biosci., 8: 275-282 (1992), Gonnet (Gonnet et al., Science, 256: 1433-1445 (1992)), Fitch (J. Mol. Evol., 16(1):9-16 (1966)), Feng (Feng et al., J. Mol. Evol., 21: 112-125 (1985)), McLachlan (J. Mol. Biol., 61:409-424 (1971)), Grantham (Science 185: 862-864 (1974)), Miyata (J. Mol. Evol. 12: 219-236 (1979)), Rao (J. Pept. Protein Res. 29: 276-281 (1987)), Risler (J. Mol. Biol. 204: 1019-1029 (1988)), Johnson (Johnson et al., J. Mol. Biol. 233: 716-738 (1993)), and percent accepted mutation (PAM) (Dayhoff et al., Atlas of Protein Sequence and Structure, 5(3): 345-352 (1978)).

Dayhoff and coworkers developed a model of protein evolution that resulted in the development of a set of widely used replacement matrices (Dayhoff et al., Atlas of Protein Sequence and Structure, 5(3):345-352 (1978)) termed percent accepted mutation matrices (PAM). In deriving these matrices, each change in the an amino acid at a particular site is assumed to be independent of previous mutational events at that site. Thus, the probability of change of any amino acid A to amino acid B is the same, regardless of the previous changes at that site and also regardless of the position of amino acid A in a protein sequence.

In the Dayhoff approach, replacement rates are derived from alignments of protein sequences that are at least 85% identical; this constraint ensures that the likelihood of a particular mutation being the result of a set of successive mutations is low. Because these changes are observed in closely related proteins, they represent amino acid substitutions that do not significantly change the function of the protein. Hence, they are called “accepted mutations,” as defined as amino acid changes that are accepted by natural selection.

The outcome of the two steps set forth above, which is performed in silico is that: (1) the amino acid positions that will be the target for mutagenesis are identified; these positions are referred to as is-HITs; (2) the replacing amino acids for the original, such as native, amino acids at the is-HITs are identified, to provide a collection of candidate LEAD mutant molecules that are expected to perform different from the native one. These are assayed for a desired modified (or improved or altered) biological activity.

c. Construction of Modified Proteins and Biological Assays

Once is-HITs are selected as set forth above, replacing amino acids are introduced. Mutant proteins typically are prepared using recombinant DNA methods and assessed in appropriate biological assays for the particular biological activity (feature) modified. An exemplary method of preparing the mutant proteins is by mutagenesis of the original, such as native, gene using methods well known in the art. Mutant molecules are generated one-by-one, such as in addressable arrays, such that each individual mutant generated is the single product of each single and independent mutagenesis reaction. Individual mutagenesis reactions are conducted separately, such as in addressable arrays where they are physically separated from each other. Once a population of sets of nucleic acid molecules encoding the respective mutant proteins is prepared, each is separately introduced one-by-one into appropriate cells for the production of the corresponding mutant proteins. This also can be performed, for example, in addressable arrays where each set of nucleic acid molecules encoding a respective mutant protein is introduced into cells confined to a discrete location, such as in a well of a multi-well microtiter plate. Each individual mutant protein is individually phenotypically-characterized and performance is quantitatively assessed using assays appropriate for the feature being modified (i.e., feature being evolved). Again, this step can be performed in addressable arrays. Those mutants displaying a desired increased or decreased performance compared to the original, such as native molecules are identified and designated LEADs. From the beginning of the process of generating the mutant DNA molecules up through the readout and analysis of the performance results, each candidate LEAD mutant is generated, produced and analyzed individually, such as from its own address in an addressable array. The process is amenable to automation.

3. Three-Dimensional (3D) Scanning

An additional method of rational evolution of proteins based on the identification of potential target sites for mutagenesis (is-HITs) is through comparison of patterns of protein backbone folding between structurally related proteins, irrespective of the underlying sequences of the compared proteins. Once the structurally related amino acid positions are identified on the new protein, then suitable amino acid replacement criteria, such as PAM analysis, can be employed to identify candidate LEADs for construction and screening.

For example, analysis of “structural homology” between and among a number of related cytokines can be used to identify on various members of the cytokine family, those amino acid positions and residues that are structurally similar or structurally related. For example, 3D scanning can be used to identify amino acid positions on IFN-β that are structurally similar or structurally related to those found in IFNα-2b mutants that have been modified for improved stability (see, co-pending U.S. application Ser. Nos. 10/658,834 and 10/658,355 and published PCT applications WO2004/022747 and WO2004/022593). This method can be applied to any desired phenotype using any protein, such as a cytokine, as the starting material to which an evolution procedure, such as the rational directed evolution procedure of U.S. application Ser. No. 10/022,249 or the 2-dimensional scanning method described herein. The structurally corresponding residues are then altered on members of the family to produce additional cytokines with similar phenotypic alterations.

a. Homology

Typically, homology between proteins is compared at the level of their amino acid sequences, based on the percent or level of coincidence of individual amino acids, amino acid per amino acid, when sequences are aligned starting from a reference, generally the residue encoded by the start codon. For example, two proteins are “homologous” or bear some degree of homology whenever their respective amino acid sequences show a certain degree of matching upon alignment comparison. Comparative molecular biology is primarily based on this approach. From the degree of homology or coincidence between amino acid sequences, conclusions can be made on the evolutionary distance between or among two or more protein sequences and biological systems.

The concept of “convergent evolution” is applied to describe the phenomena by which phylogenetically-unrelated organisms or biological systems have evolved to share features related to their anatomy, physiology and structure as a response to common forces, constraints, and evolutionary demands from the surrounding environment and living organisms. Alternatively, “divergent evolution,” is applied to describe the phenomena by which strongly phylogenetically related organisms or biological systems have evolved to diverge from identity or similarity as a response to divergent forces, constraints, and evolutionary demands from the surrounding environment and living organisms.

In the typical traditional analysis of homologous proteins there are two conceptual biases corresponding to: i) “convergent evolution,” and ii) “divergent evolution.” Whenever the aligned amino acid sequences of two proteins do not match well with each other, these proteins are considered “not related” or “less related” with each other and have different phylogenetic origins. There is no (or low) homology between these proteins and their respective genes are not homologous (or show little homology). If these two “non-homologous” proteins under study share some common functional features (e.g., interaction with other specific molecules, activity), they are determined to have arisen by “convergent evolution,” i.e., by evolution of their non-homologous amino acid sequences, in such a way that they end up generating functionally “related” structures.

On the other hand, whenever the aligned amino acid sequences of two proteins do match with each other to a certain degree, these proteins are considered to be “related” and to share a common phylogenetic origin. A given degree of homology is assigned between these two proteins and their respective genes likewise share a corresponding degree of homology. During the evolution of their initial highly homologous amino acid sequence, enough changes can be accumulated in such a way that they end up generating “less-related” sequences and less related function. The divergence from perfect matching between these two “homologous” proteins under study comes from “divergent evolution.”

b. 3D-Scanning (Structural Homology) Methods

Structural homology refers to homology between the topology and three-dimensional structure of two proteins. Structural homology is not necessarily related to “convergent evolution” or to “divergent evolution,” nor is it related to the underlying amino acid sequence. Rather, structural homology is likely driven (through natural evolution) by the need of a protein to fit specific conformational demands imposed by its environment. Particular structurally homologous “spots” or “loci” would not be allowed to structurally diverge from the original structure, even when its own underlying sequence does diverge. This structural homology is exploited herein to identify loci for mutation.

Within the amino acid sequence of a protein resides the appropriate biochemical and structural signals to achieve a specific spatial folding in either an independent or a chaperon-assisted manner. Indeed, this specific spatial folding ultimately determines protein traits and activity. Proteins interact with other proteins and molecules in general through their specific topologies and spatial conformations. In principle, these interactions are not based solely on the precise amino acid sequence underlying the involved topology or conformation. If protein traits, activity (behavior and phenotypes) and interactions rely on protein topology and conformation, then evolutionary forces and constraints acting on proteins can be expected to act on topology and conformation. Proteins sharing similar functions will share comparable characteristics in their topology and conformation, despite the underlying amino acid sequences that create those topologies and conformations.

Using the structural similarities and homologies between IFNα-2b and IFN-β, modifications were generated in IFN-β using the 3D-scanning method.

4. Super-LEADs and Additive Directional Mutagenesis (ADM)

IFN-β Modification also can include combining two or more mutations. For example, Additive Directional Mutagenesis (ADM) can be used to assemble on a single mutant protein multiple mutations present on the individual LEAD molecules, so as to generate super-LEAD mutant proteins (see co-pending U.S. application Ser. Nos. 10/658,834 and 10/658,355 and published PCT applications WO 2004/022747 and WO 2004/022593). ADM is a repetitive multi-step process where at each step after the creation of the first LEAD mutant protein a new LEAD mutation is added onto the previous LEAD mutant protein to create successive super-LEAD mutant proteins. ADM is not based on genetic recombination mechanisms, nor on shuffling methodologies; instead it is a simple one-mutation-at-a-time process, repeated as many times as necessary until the total number of desired mutations is introduced on the same molecule. To avoid the exponentially increasing number of all possible combinations that can be generated by putting together on the same molecule a given number of single mutations, a method is provided herein that, although it does not cover all the combinatorial possible space, still captures a big part of the combinatorial potential. “Combinatorial” is used herein in its mathematical meaning (i.e., subsets of a group of elements, containing some of the elements in any possible order) and not in the molecular biological or directed evolution meaning (i.e., generating pools, or mixtures, or collections of molecules by randomly mixing their constitutive elements).

A population of sets of nucleic acid molecules encoding a collection of new super-LEAD mutant molecules is generated, tested and phenotypically characterized one-by-one in addressable arrays. Super-LEAD mutant molecules are such that each molecule contains a variable number and type of LEAD mutations. Those molecules displaying further improved fitness for the particular feature being evolved, are referred to as super-LEADs. Super-LEADs can be generated by other methods known to those of skill in the art and tested by the high throughput methods herein. For purposes herein a super-LEAD typically has activity with respect to the function or biological activity of interest that differs from the improved activity of a LEAD by a desired amount, such as at least 10%, 20%, 30%, 40%, 50%, 60%, 70%, 80%, 90%, 100%, 150%, 200% or more from at least one of the LEAD mutants from which it is derived. In yet other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more greater than at least one of the LEAD molecules from which it is derived. As with LEADs, the change in the activity for super-LEADs is dependent upon the property that is being “evolved.” The desired alteration, which can be either an increase or a reduction in a feature or property, will depend upon the function or property of interest.

In one embodiment, the ADM method employs a number of repetitive steps, such that at each step a new mutation is added on a given molecule. Although numerous different ways are possible for combining each LEAD mutation onto a super-LEAD protein, an exemplary way the new mutations (e.g., mutation 1 (m1), mutation 2 (m2), mutation 3 (m3), mutation 4 (m4), mutation 5 (m5), mutation n (mn)) can be added corresponds to the following diagram:

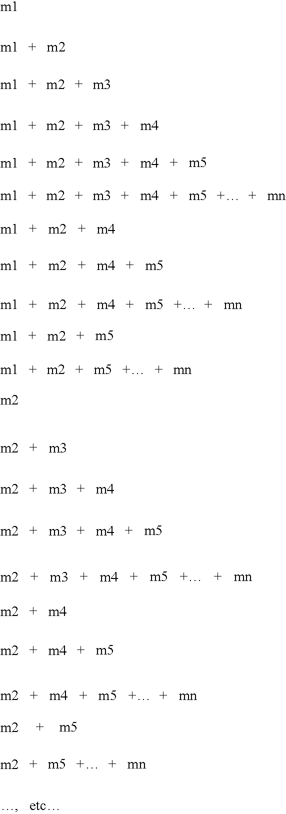

5. Multi-Overlapped Primer Extensions

Another method that can be employed to generate combinations of two or more mutations is using oligonucleotide-mediated mutagenesis referred to as “multi overlapped primer extensions.” This method can be used for the rational combination of mutant LEADs to form super-LEADS. This method allows the simultaneous introduction of several mutations throughout a small protein or protein-region of known sequence. Overlapping oligonucleotides of typically around 70 bases in length (since longer oligonucleotides lead to increased error) are designed from the DNA sequence (gene) encoding the mutant LEAD proteins in such a way that they overlap with each other on a region of typically around 20 bases. Although typically about 70 bases are used to create the overlapping oligonucleotides, the length of additional overlapping oligonucleotides for use can range from about 30 bases up to about 100 bases. Likewise, although typically the overlapping region of the overlapping oligonucleotides is about 20 bases, the length of other overlapping regions for use herein can range from about 5 bases up to about 40 bases. These overlapping oligonucleotides (including or not point mutations) act as both template and primers in a first step of PCR (using a proofreading polymerase, e.g., Pfu DNA polymerase, to avoid unexpected mutations) to create small amounts of full-length gene. The full-length gene resulting from the first PCR is then selectively amplified in a second step of PCR using flanking primers, each one tagged with a restriction site in order to facilitate subsequent cloning. One multi overlapped extension process yields a full-length (multi-mutated) nucleic acid molecule encoding a candidate super-LEAD protein having multiple mutations therein derived from LEAD mutant proteins.

### D. MODIFIED IFN-β POLYPEPTIDES EXHIBITING INCREASED PROTEIN STABILITY

Two approaches were used to increase the protein stability of IFN-β by amino acid replacement or replacements: i) Resistance to proteases by amino acid replacement that leads to higher resistance to proteases by direct destruction of the protease target residue or sequence, while maintaining or improving the requisite biological activity of IFN-β (e.g., anti-viral and anti-proliferation activity), and/or ii) increased conformational stability by amino acid replacement that leads to a decreased susceptibility to denaturation (i.e. by temperature such as at room temperature or at 37° C.), while either improving or maintaining the requisite biological activity (e.g., anti-viral and anti-proliferation activity). An IFN-β polypeptide provided herein exhibiting increased protein stability can lead to an increased half-life of the polypeptide in vivo or in vitro. For example, increased half-life can occur following administration of the polypeptide to a subject, such as a human subject. The increased half-life of the modified IFN-β polypeptide can be increased by an amount that is at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500% or more compared to the half-life of the unmodified IFN-β polypeptide. In some examples, the increased half-life of the modified IFN-β polypeptide can be increased by an amount that is at least 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times when compared to the half-life of the unmodified IFN-β polypeptide. Hence, the modified IFN-β polypeptides provided herein offer IFN-βs with advantages including a decrease in the frequency of injections needed to maintain a sufficient drug level in serum, thus leading to, for example, higher comfort and acceptance by subjects, lower doses necessary to achieve comparable biological effects and attenuation of secondary effects.

Provided herein are modified IFN-β polypeptides containing modifications that alter any one or more of the properties of IFN-β that contribute to increased protein stability (i.e. increased protease resistance, or increased conformational stability that, for example, renders a polypeptide more resistant to denaturation by temperature or pH changes) and any combinations thereof. Generally, modified polypeptides retain one or more activities of an unmodified IFN-β polypeptide. For example, the modified IFN-β polypeptides provided herein exhibit at least one activity that is substantially unchanged (less than 1%, 5% or 10% changed) compared to the unmodified or wild-type IFN-β. In other examples, the activity of a modified IFN-β polypeptide is increased or is decreased compared to an unmodified IFN-β polypeptide. Activity includes, for example, anti-viral, anti-proliferative, activation of Natural Killer cells, or induction of gene or protein markers by IFN-β. Activity can be assessed in vitro or in vivo and can be compared to the unmodified IFN-β polypeptide, such as for example, the mature, wild-type native IFN-β polypeptide (SEQ ID NO:1), the wild-type precursor IFN-β polypeptide (SEQ ID NO: 2), a commercially available mature IFN-β polypeptide, (e.g., Betaseron, SEQ ID No: 3), or any other IFN-β polypeptide known to one of skill in the art that is used as the starting material.

Modified IFN-β polypeptides provided herein include human IFN-β variants. Modified IFN-β polypeptides provided herein can be modified at one or more amino acid position corresponding to amino acid positions of a mature IFN-β polypeptide, for example, a mature IFN-β polypeptide having an amino acid sequence set forth in SEQ ID NO:1. IFN-β polypeptides can be modified compared to a mature or precursor IFN-β polypeptide having an amino acid sequence set forth in SEQ ID NO:1 or 2, respectively. IFN-β polypeptides also can be modified compared to a recombinant form of IFN-β having a sequence of amino acids set forth in SEQ ID NO:3. Modified IFN-β polypeptides provided herein also include variants of IFN-β of non-human origin. For example, modified IFN-β polypeptides can be modified compared to a mammalian IFN-β including, chimpanzee, macaque, pig, dog, horse, cow or mouse, such as set forth in any one of SEQ ID NOS:527-533. Modified IFN-β polypeptides also include polypeptides modified compared to IFN-β hybrids, such as for examples, hybrids of an IFN-β polypeptide sequence with an IFN-α polypeptide sequence, and also synthetic IFN-β sequences constructed from IFN-β sequences known in the art.

Using methods described herein, such as for example the 2D-scanning methodology, one or more target amino acid is-HIT has been identified which can serve to generate candidate LEAD IFN-β polypeptide(s) that exhibit increased protein stability, manifested as increased protease resistance or increased conformational stability as described below, compared to an unmodified IFN-β polypeptide. The following is-HIT positions were identified as targets to increase protein stability of IFN-β: 1, 3, 5, 6, 8, 9, 10, 11, 12, 13, 14, 15, 16, 17, 18, 19, 20, 21, 22, 23, 24, 28, 29, 30, 32, 33, 34, 38, 39, 41, 42, 43, 45, 47, 48, 49, 50, 51, 52, 53, 54, 57, 60, 61, 62, 63, 64, 67, 70, 72, 73, 78, 79, 80, 81, 82, 83, 85, 86, 87, 88, 89, 90, 91, 92, 94, 95, 97, 98, 99, 101, 102, 103, 104, 105, 106, 107, 108, 109, 110, 111, 113, 115, 116, 117, 120, 122, 123, 124, 125, 126, 130, 132, 133, 134, 136, 137, 138, 143, 147, 149, 151, 152, 154, 156, 160, 163, 164, and 165. Amino acid replacement or replacements can correspond to any of the following amino acid positions corresponding to a mature IFN-β polypeptide set forth in SEQ ID NO:1: M1, Y3, L5, L6, F8, L9, Q10, R11, S12, S13, N14, F15, Q16, C17, Q18, K19, L20, L21, W22, Q23, L24, L28, E29, Y30, L32, K33, D34, F38, D39, P41, E42, E43, K45, L47, Q48, Q49, F50, Q51, K52, E53, D54, L57, Y60, E61, M62, L63, Q64, F67, F70, Q72, D73, G78, W79, N80, E81, T82, I83, E85, N86, L87, L88, A89, N90, V91, Y92, Q94, I95, H97, L98, K99, V101, L102, E103, E104, K105, L106, E107, K108, E109, D110, F111, R113, K115, L116, M117, L120, L122, K123, R124, Y125, Y126, L130, Y132, L133, K134, K136, E137, Y138, W143, R147, E149, L151, R152, F154, F156, L160, Y163, L164, and R165. In one example, amino acid modifications can be in an unmodified IFN-β polypeptide, such as for example, an unmodified IFN-β polypeptide having a sequence of amino acids set forth in SEQ ID NO:1 or SEQ ID NO:3.

IFN-β candidate LEAD polypeptides can include amino acid replacement or replacements at any one or more of the is-HIT positions selected using methods described herein or known in the art, such as obtained using PAM analysis. Examples of exemplary amino acid modifications corresponding to amino acid positions of a mature IFN-β polypeptide that can contribute to an increase in protein stability are set forth in Table 2. Other amino acid modifications can include any one or more amino acid modifications set forth in Table 3 and disclosed in published U.S. Application No. US-2004-0132977-A1. In Table 2 and 3 below, the sequence identifier (SEQ ID No.) is in parenthesis next to each substitution.

Typically, modifications include replacement (substitution), addition, deletion, or a combination thereof, of amino acid residues as described herein. Modified IFN-β polypeptides include those with 1, 2, 3, 4, 5, 6, 7, 8, 9, 10, 11, 12, 13, 14, 15, 16, 17, 18, 19, 20, or more modified positions. Generally, the modification results in increased stability without losing at least one activity, such as antiviral activity (i.e. retains at least one activity as defined herein) of an unmodified IFN-β polypeptide. A modified IFN-β exhibiting increased protein stability containing a single amino acid change at an is-HIT position compared to an unmodified IFN-β is called a LEAD and a modified IFN-β containing two or more modifications compared to an unmodified IFN-β is called a Super-LEAD as described in detail herein and below. In one example, a modified IFN-β polypeptide candidate LEAD can contain any single amino acid modification set forth in Table 2, or any combination of amino acid modifications set forth in Table 2. In another example, a modified IFN-β polypeptide can contain two or more amino acid modifications set forth in Table 3. A modified IFN-β polypeptide also can contain a combination of amino acid modifications, such as for example, any one or more amino acid modifications set forth in Table 2 in combination with any one or more amino acid modifications set forth in Table 3.

TABLE 2
IFN-β amino acid modifications that contribute
to increased protein stability

| | | | | |
|---|---|---|---|---|
| Y3H (4) | Y3I (5) | L6I (6) | L6V (7) | L6H (534) |
| L6A (535) | R11D (145) | Q18H (623) | Q18S (624) | Q18T (625) |
| Q18N (626) | K19N (536) | L20I (8) | L20V (9) | L20H (537) |
| L20A (538) | L21I (10) | L21V (11) | L21T (539) | L21Q (540) |
| L21H (541) | L21A (542) | Q23H (627) | Q23S (628) | Q23T (629) |
| Q23N (630) | L24I (12) | L24V (13) | L24T (543) | L24Q (544) |
| L24H (545) | L24A (546) | E29N (547) | K33N (548) | D34N (14) |
| D34Q (15) | D34G (549) | F38I (16) | F38V (17) | D39N (550) |
| P41A (18) | P41S (19) | E42N (551) | E43K (134) | E43Q (20) |
| E43H (21) | E43N (22) | K45D (146) | K45N (552) | Q48H (631) |
| Q48S (632) | Q48T (633) | Q48N (634) | Q49H (635) | Q49S (636) |
| Q49T (637) | Q49N (638) | F50I (23) | F50V (24) | Q51H (639) |
| Q51S (640) | Q51T (641) | Q51N (642) | K52D (147) | K52N (553) |
| E53R (135) | E53Q 25) | E53H (26) | E53N (27) | D54K (136) |
| D54Q (29) | D54N (28) | D54G (554) | L57I (30) | L57V (31) |
| L57T (555) | L57Q (556) | L57H (557) | L57A (558) | Y60H (32) |
| Y60I (33) | E61K (137) | E61Q (34) | E61H (35) | E61N (36) |
| M62I (37) | M62V (38) | M62T (559) | M62Q (560) | M62A (561) |
| L63I (39) | L63V (40) | L63T (562) | L63Q (563) | L63H (564) |
| L63A (565) | Q64H (643) | Q64S (644) | Q64T (645) | Q64N (646) |
| F70I (41) | F70V (42) | Q72H (647) | Q72S (648) | Q72T (649) |
| Q72N (650) | D73N (566) | W79H (43) | W79S (44) | E81K (138) |
| E81N (567) | E85K (139) | E85N (568) | L87I (45) | L87V (46) |
| L87H (569) | L87A (570) | L88I (47) | L88V (48) | L88T (571) |
| L88Q (572) | L88H (573) | L88A (574) | L98I (49) | L98V (50) |
| L98H (575) | L98A (576) | K99N (577) | L102I (51) | L102V (52) |
| L102T (578) | L102Q (579) | L102H (580) | L102A (581) | E103K (140) |
| E103N (582) | E104R (141) | E104N (583) | K105D (148) | K105N (584) |
| L106I (53) | L106V (54) | L106T (585) | L106Q (586) | L106H (587) |
| L106A (588) | E107R (142) | E107N (589) | K108D (149) | K108N (590) |
| E109R (143) | E109N (591) | D110K (144) | D110N (592) | R113E (150) |
| K115D (151) | K115Q (56) | K115N (55) | K115S (593) | K115H (594) |
| M117I (57) | M117V (58) | M117T | M117Q | M117A (598) |
| | | (596) | (597) | |
| L122I (59) | L122V (60) | L122T (599) | L122Q (600) | L122H (601) |
| L122A (602) | K123N (603) | R124D (520) | R124E (519) | Y125H (61) |
| Y125I (62) | Y126H (63) | Y126I (64) | Y132H (65) | Y132I (66) |
| L133I (67) | L133V (68) | L133T (604) | L133Q (605) | L133H (606) |
| L133A (607) | K134N (608) | K136N (609) | E137N (610) | W143H (71) |
| W143S (72) | R147H (73) | R147Q (74) | E149Q (75) | E149H (76) |
| E149N (77) | L151I (78) | L151V (79) | L151T (611) | L151Q (612) |
| L151H (613) | L151A (614) | R152D (152) | F154I (80) | F154V (81) |
| F156I (82) | F156V (83) | L160I (84) | L160V (85) | L160T (615) |
| L160Q (616) | L160H (617) | L160A (618) | L164I (86) | L164V (87) |
| L164T (619) | L164Q (620) | L164H (621) | L164A (622) | R165D (153) |

TABLE 3
Additional IFN-β amino acid modifications that
contribute to increased protein stability

| | | | | |
|---|---|---|---|---|
| M1V (262) | M1I (263) | M1T (264) | M1A (265) | M1Q (261) |
| M1D (322) | M1E (323) | M1K (324) | M1N (325) | M1R (326) |
| M1S (327) | M1C (651) | L5V (266) | L5I (267) | L5T (268) |
| L5Q (269) | L5H (270) | L5A (271) | L5D (328) | L5E (329) |
| L5K (330) | L5R (331) | L5N (332) | L5S (333) | L6D (334) |
| L6E (335) | L6K (336) | L6N (337) | L6Q (338) | L6R (339) |
| L6S (340) | L6T (341) | L6C (652) | F8I (272) | F8V (273) |
| F8D (342) | F8E (343) | F8K (344) | F8R (345) | L9V (274) |
| L9I (275) | L9T (276) | L9Q (277) | L9H (278) | L9A (279) |
| L9D (346) | L9E (347) | L9K (348) | L9N (349) | L9R (350) |
| L9S (351) | Q10D (352) | Q10E (353) | Q10K (354) | Q10N (355) |
| Q10R (356) | Q10S (357) | Q10T (358) | Q10C (653) | R11H (280) |
| R11Q (281) | S12D (359) | S12E (360) | S12K (361) | S12R (362) |
| S13D (363) | S13E (364) | S13K (365) | S13N (366) | S13Q (367) |
| S13R (368) | S13T (369) | S13C (654) | N14D (370) | N14E (371) |
| N14K (372) | N14Q (373) | N14R (374) | N14S (375) | N14T (376) |
| F15I (282) | F15V (283) | F15D (377) | F15E (378) | F15K (379) |
| F15R (380) | Q16D (381) | Q16E (382) | Q16K (383) | Q16N (384) |
| Q16R (385) | Q16S (386) | Q16T (387) | Q16C (655) | C17D (388) |
| C17E (389) | C17K (390) | C17N (391) | C17Q (392) | C17R (393) |
| C17S (394) | C17T (395) | K19Q (284) | K19T (285) | K19S (286) |
| K19H (287) | L20N (396) | L20Q (402) | L20R (398) | L20S (399) |
| L20T (400) | L20D (401) | L20E (397) | L20K (403) | W22S (288) |
| W22H (289) | W22D (404) | W22E (405) | W22K (406) | W22R (407) |
| Q23D (408) | Q23E (409) | Q23K (410) | Q23R (411) | L24D (412) |
| L24E (413) | L24K (414) | L24R (415) | N25H (290) | N25S (291) |
| N25Q (292) | R27H (293) | R27Q (294) | L28V (295) | L28I (296) |
| L28T (297) | L28Q (298) | L28H (299) | L28A (300) | E29Q (301) |
| E29H (302) | Y30H (303) | Y30I (304) | L32V (305) | L32I (306) |
| L32T (307) | L32Q (308) | L32H (309) | L32A (310) | K33Q (311) |
| K33T (312) | K33S (313) | K33H (314) | R35H (315) | R35Q (316) |
| M36V (317) | M36I (318) | M36T (319) | M36Q (320) | M36A (321) |
| D39Q (154) | D39H (155) | D39G (156) | E42Q (157) | E42H (158) |
| K45Q (159) | K45T (160) | K45S (161) | K45H (162) | L47V (163) |
| L47I (164) | L47T (165) | L47Q (166) | L47H (167) | L47A (168) |
| K52Q (169) | K52T (170) | K52S (171) | K52H (172) | F67I (173) |
| F67V (174) | R71H (175) | R71Q (176) | D73Q (177) | D73H (178) |
| D73G (179) | G78D (416) | G78E (417) | G78K (418) | G78R (419) |
| W79D (420) | W79E (421) | W79K (422) | W79R (423) | N80D (424) |
| N80E (425) | N80K (426) | N80R (427) | E81Q (180) | E81H (181) |
| T82D (428) | T82E (429) | T82K (430) | T82R (431) | I83D (432) |
| I83E (433) | I83K (434) | I83R (435) | I83N (436) | I83Q (437) |
| I83S (438) | I83T (439) | E85Q (182) | E85H (183) | N86D (440) |
| N86E (441) | N86K (442) | N86R (443) | N86Q (444) | N86S (445) |
| N86T (446) | L87D (447) | L87E (448) | L87K (449) | L87R (450) |
| L87N (451) | L87Q (452) | L87S (453) | L87T (454) | A89D (455) |
| A89E (456) | A89K (457) | A89R (458) | N90D (459) | N90E (460) |
| N90K (461) | N90Q (462) | N90R (463) | N90S (464) | N90T (465) |
| N90C (129) | V91D (466) | V91E (467) | V91K (468) | V91N (469) |
| V91Q (470) | V91R (471) | V91S (472) | V91T (473) | V91C (131) |
| Y92H (184) | Y92I (185) | Q94D (474) | Q94E (475) | Q94K (476) |
| Q94N (477) | Q94R (478) | Q94S (479) | Q94T (480) | Q94C (656) |
| I95D (481) | I95E (482) | I95K (483) | I95N (484) | I95Q (485) |
| I95R (486) | I95S (487) | I95T (488) | H97D (489) | H97E (490) |
| H97K (491) | H97N (492) | H97Q (493) | H97R (494) | H97S (495) |
| H97T (496) | H97C (657) | L98D (497) | L98E (498) | L98K (499) |
| L98N (500) | L98Q (501) | L98R (502) | L98S (503) | L98T (504) |
| L98C (658) | K99Q (186) | K99T (187) | K99S (188) | K99H (189) |
| V101D (505) | V101E (506) | V101K (507) | V101N (508) | V101Q (509) |
| V101R (510) | V101S (511) | V101T 512) | V101C (659) | E103Q (190) |
| E103H (191) | E104Q (192) | E104H (193) | K105Q (194) | K105T (195) |
| K105S (196) | K105H (197) | E107Q (198) | E107H (199) | K108Q (200) |
| K108T (201) | K108S (202) | K108H (203) | E109H (205) | E109Q (204) |
| D110Q (206) | D110H (207) | D110G (208) | F111I (209) | F111V (210) |
| R113H (211) | R113Q (212) | L116V (213) | L116I (214) | L116T (215) |
| L116Q (216) | L116H (217) | L116A (218) | L120V (219) | L120I (220) |
| L120T (221) | L120Q (222) | L120H (223) | L120A (224) | K123Q (225) |
| K123T (226) | K123S (227) | K123H (228) | R124H (229) | R124Q (230) |
| R128H (231) | R128Q (232) | L130V (233) | L130I (234) | L130T (235) |
| L130Q (236) | L130H (237) | L130A (238) | K134Q (239) | K134T (240) |
| K134S (241) | K134H (242) | K136Q (243) | K136T (244) | K136S (245) |
| K136H (246) | E137Q (247) | E137H (248) | Y138H (253) | Y138I (254) |
| R152H (255) | R152Q (256) | Y155H (257) | Y155I (258) | R159H (259) |
| R159Q (260) | Y163H (249) | Y163I (250) | R165H (251) | R165Q (252) |

Provided herein are modified IFN-β polypeptides exhibiting increased protein stability compared to an unmodified IFN-β polypeptide, where the modified IFN-β polypeptide contains one or more amino acid modifications corresponding to any one or more modification of Y3I, Y3H, L6I, L6V, L6H, L6A, R11D, Q18H, Q18S, Q18T, Q18N, K19N, L20I, L20V, L20H, L20A, L21I, L21V, L21T, L21Q, L21H, L21A, Q23H, Q23S, Q23T, Q23N, L24I, L24V, L24T, L24Q, L24H, L24A, E29N, K33N, D34N, D34Q, D34G, F38I, F38V, D39N, P41A, P41S, E42N, E43K, E43Q, E43H, E43N, K45D, K45N, Q48H, Q48S, Q48T, Q48N, Q49H, Q49S, Q49T, Q49N, F50I, F50V, Q51H, Q51S, Q51T, Q51N, K52D, K52N, E53R, E53Q, E53H, E53N, D54G, L57I, L57V, L57T, L57Q, L57H, L57A, Y60H, Y60I, E61K, E61Q, E61H, E61N, M62I, M62V, M62T, M62Q, M62H, M62A, L63I, L63V, L63T, L63Q, L63H, L63A, Q64H, Q64S, Q64T, Q64N, F70I, F70V, Q72H, Q72S, Q72T, Q72N, D73N, W79H, W79S, E81K, E81N, E85K, E85N, L87I, L87V, L87H, L87A, L88I, L88V, L88T, L88Q, L88H, L88A, L98I, L98V, L98H, L98A, K99N, L102I, L102V, L102T, L102Q, L102H, L102A, E103K, E103N, E104R, E104N, K105D, K105N, L106I, L106V, L106T, L106Q, L106H, L106A, E107R, E107N, K108D, K108N, E109R, E109N, D110K, D110N, R113E, K115D, K115Q, K115N, K115S, K115H, M117I, M117V, M117T, M117Q, M117A, L122I, L122V, L122T, L122Q, L122H, L122A, K123N, R124D, R124E, Y125H, Y125I, Y126H, Y126I, Y132H, Y132I, L133I, L133V, L133T, L133Q, L133H, L133A, K134N, K136N, E137N, W143H, W143S, R147H, R147Q, E149Q, E149H, E149N, L151I, L151V, L151T, L151Q, L151H, L151A, R152D, F154I, F154V, F156I, F156V, L160I, L160V, L160T, L160Q, L160H, L160A, L164I, L164V, L164T, L164Q, L164H, L164A, and R165D of a mature IFN-β polypeptide set forth in SEQ ID NO:1. Generally, the modified IFN-β retains one or more activities of the unmodified IFN-β. In some examples, the modification is in an unmodified IFN-β polypeptide having a sequence of amino acids set forth in SEQ ID NO:1 or SEQ ID NO:3. Also provided herein is a modified IFN-β exhibiting increased protein stability as set forth above, containing a further modification compared to an unmodified IFN-β polypeptide. The further modification can be one or more replacement(s) at an amino acid position corresponding to any of positions 1, 5, 6, 8, 9, 10, 11, 12, 13, 14, 15, 16, 17, 19, 20, 22, 23, 24, 25, 27, 28, 29, 30, 32, 33, 35, 36, 39, 42, 45, 47, 52, 67, 71, 73, 78, 79, 80, 81, 82, 83, 85, 86, 87, 89, 90, 91, 92, 94, 95, 97, 98, 99, 101, 103, 104, 105, 107, 108, 109, 110, 111, 113, 116, 123, 124, 128, 130, 134, 136, 137, 138, 152, 155, 159, 163, and 165. Amino acid replacement or replacements can occur at one or more positions corresponding to amino acid residues positions selected from among M1, L5, L6, F8, L9, Q10, R11, S12, S13, N14, F15, Q16, C17, K19, L20, W22, Q23, L24, N25, R27, L28, E29, Y30, L32, K33, R35, M36, D39, E42, K45, L47, K52, F67, R71, D73, G78, W79, N80, E81, T82, I83, E85, N86, L87, A89, N90, V91, Y92, Q94, I95, H97, L98, K99, V101, E103, E104, K105, E107, K108, E109, D110, F111, R113, L116, K123, R124, R128, L130, K134, K136, E137, Y138, R152, Y155, R159, Y163, and R165 of a mature IFN-β polypeptide set forth in SEQ ID NO:1. For example, the amino acid replacements of the further modification can be any one or more amino acid modifications set forth in Table 3, such as for example, any one or more amino modification corresponding to M1V, M1I, M1T, M1A, M1Q, M1D, M1E, M1K, M1N, M1R, M1S, M1C, L5V, L5I, L5T, L5Q, L5H, L5A, L5D, L5E, L5K, L5R, L5N, L5S, L6D, L6E, L6K, L6N, L6Q, L6R, L6S, L6T, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, R11H, R11Q, S12D, S12E, S12K, S12R, S13D, S13E, S13K, S13N, S13Q, S13R, S13T, S13C, N14D, N14E, N14K, N14Q, N14R, N14S, N14T, F15I, F15V, F15D, F15E, F15K, F15R, Q16D, Q16E, Q16K, Q16N, Q16R, Q16S, Q16T, Q16C, C17D, C17E, C17K, C17N, C17R, C17S, C17T, K19Q, K19T, K19S, K19H, L20N, L20Q, L20R, L20S, L20T, L20D, L20E, L20K, W22S, W22H, W22D, W22E, W22K, W22R, Q23D, Q23E, Q23K, Q23R, L24D, L24E, L24K, L24R, N25H, N25S, N25Q, R27H, R27Q, L28V, L28I, L28T, L28Q, L28H, L28A, E29Q, E29H, Y30H, Y30I, L32V, L32I, L32T, L32Q, L32H, L32A, K33Q, K33T, K33S, K33H, R35H, R35Q, M36V, M36I, M36T, M36Q, M36A, D39Q, D39H, D39G, E42Q, E42H, K45Q, K45T, K45S, K45T, L47V, L47I, L47T, L47Q, L47H, L47A, K52Q, K52T, K52S, K52H, F67I, F67V, R71H, R71Q, D73Q, D73H, D73G, G78D, G78E, G78K, G78R, N80D, N80E, N80K, N80R, E81Q, E81H, T82D, T82E, T82K, T82R, I83D, I83E, I83K, I83R, I83N, 183Q, I83S, I83T, E85Q, E85H, N86D, N86E, N86K, N86R, N86Q, N86S, N86T, L87D, L87E, L87K, L87R, L87N, L87Q, L87S, L87T, A89D, A89E, A89K, A89R, N90D, N90E, N90K, N90Q, N90R, N90S, N90T, N90C, V91D, V91E, V91K, V91N, V91Q, V91R, V91S, V91T, V91C, Y92H, Y92I, Q94D, Q94E, Q94K, Q94N, Q94R, Q94S, Q94T, Q94C, I95D, I95E, I95K, I95N, I95Q, I95R, I95S, I95T, H97D, H97E, H97K, H97N, H97Q, H97R, H97S, H97T, H97C, L98D, L98E, L98K, L98N, L98Q, L98R, L98S, L98T, L98C, K99Q, K99T, K99S, K99H, V101D, V101E, V101K, V101N, V101Q, V101R, V101S, V101T, V101C, E103Q, E103H, E104Q, E104H, K105Q, K105T, K105S, K105H, E107 Q, E107H, K108 Q, K108T, K108S, K108H, E109H, E109Q, D110Q, D110H, D110G, F111I, F111V, R113H, R113Q, L116V, L116I, L116T, L116Q, L116H, L116A, K123Q, K123T, K123S, K123H, R124H, R124Q, R128H, R128Q, L130V, L130I, L130T, L130Q, L130H, L130A, K134Q, K134T, K134S, K134H, K136Q, K136T, K136S, K136H, E137Q, E137H, Y138H, Y138I, R152H, R152Q, Y155H, Y155I, R159H, R159Q, Y163H, Y163I, R165H, and R165Q of a mature IFN-β polypeptide set forth in SEQ ID NO:1.

In one example, provided herein are modified IFN-β polypeptides exhibiting increased protein stability compared to an unmodified IFN-β polypeptide of SEQ ID NO:1 wherein the modified IFN-β polypeptide contains one or more amino acid modifications corresponding to any one or more of Y3I, Q18N, Q18S, K19N, L20I, L20V, K33N, D34N, K33N, P41A, P41S, E42N, E43N, K45D, K45N, Q48H, Q48S, Q48T, Q49H, Q49S, Q49T, F50I, F50V, Q51H, Q51S, Q51T, Q51N, K52D, K52N, E53N, D54G, L57I, Y60I, E61K, E61H, E61N, Q64H, Q64S, Q64T, F70I, F70V, Q72H, Q72S, E85N, L88I, L88V, L98I, L98V, K99N, E103N, E104N, K105D, K105N, L106I, L106V, E107N, E109N, K115D, K115N, K115S, K115H, K123N, Y125I, Y126I, Y132I, K134N, Y136N, R147H, R147Q, E149H, E149N, L151I, and L154V of a mature IFN-β polypeptide set forth in SEQ ID NO:1. Generally, the modified IFN-β retains one or more activities of the unmodified IFN-β. Also provided are modified IFN-β polypeptides exhibiting increased protein stability compared to an unmodified IFN-β polypeptide of SEQ ID NO:3 wherein the modified IFN-β polypeptide contains one or more amino acid modifications corresponding to any of Y3I, Q18S, Q18N, K19N, L20I, L20V, L21I, L21V, K33N, D34N, K33N, P41A, P41S, E42N, E43N, K45D, K45N, Q48H, Q48S, Q48T, Q49H, Q49S, Q49T, F50I, F50V, Q51H, Q51S, Q51T, Q51N, K52D, K52N, E53N, D54G, L57I, Y60I, E61K, E61H, E61N, M62I, M62V, Q64H, Q64S, Q64T, F70I, F70V, Q72H, Q72S, E85N, L88I, L88V, L98I, L98V, K99N, E103N, E104N, K105D, K105N, L106I, L106V, E107N, E109N, K115D, K115N, K115S, K115H, M117I, M117V, L122I, L122V, K123N, Y125I, Y126I, Y132I, K134N, Y136N, R147H, R147Q, E149H, E149N, L151I, L154V, and L160V of a mature IFN-α polypeptide set forth in SEQ ID NO:1. Generally, the modified IFN-β retains one or more activities of the unmodified IFN-β. Additionally, a modified IFN-β that exhibits increased protein stability as set forth above can contain a further modification. The further modification can be one or more replacement(s) at an amino acid position corresponding to any of positions 1, 3, 5, 6, 8, 9, 10, 11, 12, 13, 14, 15, 16, 17, 18, 19, 20, 22, 23, 24, 25, 27, 28, 29, 30, 32, 33, 34, 35, 36, 38, 39, 42, 45, 47, 48, 49, 52, 53, 57, 60, 61, 62, 63, 64, 67, 71, 72, 73, 78, 79, 80, 81, 82, 83, 85, 86, 87, 88, 89, 90, 91, 92, 94, 95, 97, 98, 99, 101, 102, 103, 104, 105, 106, 107, 108, 109, 110, 111, 113, 115, 116, 117, 120, 122, 123, 124, 125, 126, 128, 130, 132, 133, 134, 136, 137, 138, 143, 149, 151, 152, 154, 155, 156, 159, 160, 163, 164, and 165. Amino acid replacement or replacements can occur at one or more of amino acid positions corresponding to any of positions M1, Y3, L5, L6, F8, L9, Q10, R11, S12, S13, N14, F15, Q16, C17, Q18, K19, L20, W22, Q23, L24, N25, R27, L28, E29, Y30, L32, K33, D34, R35, M36, F38, D39, E42, K45, L47, Q48, Q49, K52, E53, L57, Y60, E61, M62, L63, Q64, F67, R71, Q72, D73, G78, W79, N80, E81, T82, I83, E85, N86, L87, L88, A89, N90, V91, Y92, Q94, I95, H97, L98, K99, V101, L102, E103, E104, K105, L106, E107, K108, E109, D110, F111, R113, K115, L116, M117, L120, L122, K123, R124, Y125, Y126, R128, L130, Y132, L133, K134, K136, E137, Y138, W143, E149, L151, R152, F154, Y155, F156, R159, L160, Y163, L164, and R165 of a mature IFN-β polypeptide set forth in SEQ ID NO:1. For example, the amino acid replacements of the further modification can be any one or more modification of M1C, M1D, M1E, M1K, M1N, M1R, M1S, M1V, M1I, M1T, M1A, M1Q, Y3H, L5V, L5I, L5T, L5Q, L5H, L5A, L5D, L5E, L5K, L5R, L5N, L5S, L6I, L6V, L6H, L6A, L6D, L6E, L6K, L6N, L6Q, L6R, L6S, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, R11D, R11H, R11Q, S12D, S12E, S12K, S12R, S13D, S13E, S13K, S13N, S13Q, S13R, S13T, S13C, N14D, N14E, N14K, N14Q, N14R, N14S, N14T, F15D, F15E, F15K, F15R, F15I, F15V, Q16D, Q16E, Q16K, Q16N, Q16R, Q16S, Q16C, Q16T, C17D, C17E, C17K, C17N, C17Q, C17R, C17S, C17T, Q18H, Q18T, K19Q, K19T, K19S, K19H, L20H, L20A, L20N, L20Q, L20R, L20S, L20T, L20D, L20E, L20K, L21I, L21V, L21T, L21Q, L21H, L21A, W22D, W22E, W22K, W22R, W22S, W22H, Q23H, Q23S, Q23T, Q23N, Q23D, Q23E, Q23K, Q23R, L24I, L24V, L24T, L24Q, L24H, L24A, L24D, L24E, L24K, L24R, N25H, N25S, N25Q, R27H, R27Q, L28V, L28I, L28T, L28Q, L28H, L28A, E29N, E29Q, E29H, Y30H, Y30I, L32V, L32I, L32T, L32Q, L32H, L32A, K33Q, K33T, K33S, K33H, D34Q, D34G, R35H, R35Q, M36V, M36I, M36T, M36Q, M36A, F38I, F38V, D39N, D39Q, D39H, D39G, E42Q, E42H, E43K, E43Q, E43H, K45Q, K45T, K45S, K45H, L47V, L47I, L47T, L47Q, L47H, L47A, Q48N, Q49N, K52Q, K52T, K52S, K52H, E53R, E53Q, E53H, L57V, L57T, L57Q, L57H, L57A, Y60H, E61Q, M62I, M62V, M62T, M62Q, M62A, L63V, L63T, L63Q, L63H, L63A, Q64N, F67I, F67V, R71H, R71Q, Q72N, D73N, D73H, D73G, D73Q, G78D, G78E, G78K, G78R, W79H, W79S, W79D, W79E, W79K, W79R, N80D, N80E, N80K, N80R, E81K, E81N, E81Q, E81H, T82D, T82E, T82K, T82R, I83D, I83E, I83K, I83R, I83N, I83Q, I83S, I83T, E85K, E85Q, E85H, N86D, N86E, N86K, N86R, N86Q, N86S, N86T, L87I, L87V, L87H, L87A, L87D, L87E, L87K, L87R, L87N, L87Q, L87S, L87T, L88T, L88Q, L88H, L88A, A89D, A89E, A89K, A89R, N90D, N90E, N90K, N90Q, N90R, N90S, N90T, N90C, V91D, V91E, V91K, V91N, V91Q, V91R, V91S, V91T, V91C, Y92H, Y92I, Q94D, Q94E, Q94K, Q94N, Q94R, Q94S, Q94T, Q94C, I95D, I95E, I95K, I95N, I95Q, I95R, I95S, I95T, H97D, H97E, H97K, H97N, H97Q, H97R, H97S, H97T, H97C, L98H, L98A, L98D, L98E, L98K, L98N, L98Q, L98R, L98S, L98T, L98C, K99Q, K99T, K99S, K99H, V101D, V101E, V101K, V101N, V101Q, V101R, V101S, V101T, V101C, L102I, L102V, L102T, L102Q, L102H, L102A, E103K, E103Q, E103H, E104R, E104Q, E104H, K105Q, K105T, K105S, K105H, L106T, L106Q, L106H, L106A, E107R, E107Q, E107H, K108D, K108N, K108Q, K108T, K108S, K108H, E109R, E109Q, E109H, D110K, D110N, D110Q, D110H, D110G, F111I, F111V, R113E, R113H, R113Q, K115Q, L116V, L116I, L116T, L116Q, L116H, L116A, M117I, M117V, M117T, M117Q, M117Q, M117A, L120V, L120I, L120T, L120Q, L120H, L120A, L122I, L122V, L122T, L122Q, L122H, L122A, K123Q, K123T, K123S, K123H, R124D, R124E, R124H, R124Q, Y125H, Y126H, R128H, R128Q, L130V, L130I, L130T, L130Q, L130H, L130A, Y132H, L133I, L133V, L133T, L133Q, L133H, L133A, K134Q, K134T, K134S, K134H, K136Q, K136T, K136S, K136H, E137N, E137Q, E137H, Y138H, Y138I, W143H, W143S, E149Q, L151V, L151T, L151Q, L151H, L151A, R152D, R152H, R152Q, F154I, Y155H, Y155I, F156I, F156V, R159H, R159Q, L160I, L160V, L160T, L160Q, L160H, L160A, Y163H, Y163I, L164I, L164V, L164T, L164Q, L164H, L164A, R165D, R165Q and R165H. Provided herein are IFN-β candidate LEAD polypeptides exhibiting increased protein stability having a sequence of amino acids set forth in any of SEQ ID NOS: 4-87, 129, 131, 134-512, 519, 520, and 534-659.

1. Protease Resistance

Of interest is a modified IFN-β polypeptide exhibiting increased protein stability manifested as an increased resistance to digestion by proteases. Among modifications of therapeutic proteins are those that increase protection against protease digestion without destroying or eliminating a therapeutic or the therapeutic activity. Such changes are useful for producing longer-lasting therapeutic proteins. The delivery of stable peptide and protein drugs to patients is a major challenge for the pharmaceutical industry. These types of drugs in the human body are constantly eliminated or taken out of circulation by different physiological processes including internalization, glomerular filtration and proteolysis. The latter is often the limiting process affecting the half-life of proteins used as therapeutic agents in per-oral administration and either intravenous or intramuscular injections. Thus, in one aspect, the polypeptides provided herein have been modified to increase resistance to proteolysis, thereby increasing the half-life of the modified polypeptide in vitro (e.g., production, processing, storage, assay, etc.) or in vivo (e.g., serum stability). Thus, the modified polypeptides provided herein are useful as longer-lasting therapeutic proteins.

Proteases, proteinases or peptidases catalyze the hydrolysis of covalent peptidic bonds. Modified IFN-β polypeptides provided herein exhibit increased resistance to proteolysis by proteases, including those that occur, for example, in body fluids and tissues, such as those that include, but are not limited to, saliva, blood, serum, intestinal, stomach, blood, cell lysates, cells and others. These include proteases of all types, such as, for example, serine proteases, cysteine proteases, aspartyl proteases, and matrix metalloproteinases. Modifications include, but are not limited to, amino acid modifications that confer resistance to one or more proteases including, but not limited to, pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, luminal pepsin, microvillar endopeptidase, dipeptidyl peptidase, enteropeptidase, hydrolase, NS3, factor Xa, Granzyme B, thrombin, plasmin, urokinase, tPA and PSA.

Modified IFN-β polypeptides provided herein exhibit increased resistance to proteolysis, particularly by enzymes present in serum, blood, the gut, the mouth and other body fluids. Such increase in resistance is manifested as at least 1%, 2%, 3%, 4%, 5%, 6%, 7%, 8%, 9%, 10%, . . . 20%, . . . 30%, . . . 40%, . . . 50%, . . . 60%, . . . , 70%, . . . 80%, . . . 90%, 91%, 92%, 93%, 94%, 95%, 96%, 97%, 98%, 99%, 100%, 200%, 300%, 400%, 500%, or more resistance to proteolysis compared to the unmodified IFN-β polypeptide. Increasing protein stability to proteases (blood, lysate, intestinal, serum, etc.) is contemplated herein to provide a longer in vitro or in vivo half-life for the particular protein molecule and, thus, a reduction in the frequency of necessary administrations to subjects. Typically, the half-life in vitro or in vivo of the modified IFN-β polypeptides provided herein is increased by an amount selected from at least about or at least 1%, at least 5%, at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500% or more, when compared to the half-life of unmodified or wild-type human IFN-β in either human blood, human serum, in an in vitro preparation or an in vitro mixture containing one or more proteases. In some instances, the half-life of the IFN-β mutants provided herein is increased by an amount, including but not limited to, at least 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more, when compared to the half-life of native IFN-β in either in vivo (human blood, human serum, saliva, digestive fluid, the intestinal tract, etc.) or an in vitro mixture containing one or more proteases.

Typically, the modified IFN-β polypeptides provided herein exhibit at least one activity that is substantially unchanged (less than 1%, 5% or 10% changed) compared to the unmodified or wild-type IFN-β. In some examples, the activity is increased compared to the unmodified IFN-β. In other examples, the activity is decreased compared to the unmodified IFN-β polypeptide. Activity includes, for example, anti-viral or anti-proliferative activity, and can be compared to the unmodified polypeptide, such as for example, the mature, wild-type native IFN-β polypeptide (SEQ ID NO:1), the wild-type precursor IFN-β polypeptide (SEQ ID NO: 2), a commercially available IFN-β polypeptide, (e.g., Betaseron, SEQ ID No: 3), or any other IFN-β polypeptide used as the starting material. In one example, activity of modified IFN-β is assessed in an assay measuring the capacity of the modified IFN-β to modulate cell proliferation or anti-viral activity when added to the appropriate cells. Prior to the measurement of the activity, IFN-β molecules can be challenged with proteases (e.g., blood, intestinal, etc.) under one or more in vitro conditions mimicking administered conditions, such as administration in serum, blood, saliva, or digestive tract (in vitro assays), and/or administed to a subject such as a mouse or human (in vivo assays) during different incubation or post-injection times. The activity measured, corresponds then to the residual activity following exposure to proteolytic mixtures.

a. Serine Proteases

Serine proteases participate in a range of functions in the body, including blood clotting, inflammation as well as digestive enzymes in both prokaryotes and eukaryotes. Serine proteases are sequence specific. While cascades of protease activations control blood clotting and complement, other proteases are involved in signaling pathways, enzyme activation and degradative functions in different cellular or extracellular compartments.

Serine proteases include, but are not limited, to chymotrypsin, trypsin, elastase, NS3, factor Xa, Granzyme B, thrombin, trypsin, plasmin, urokinase, tPA and PSA. Chymotrypsin, trypsin and elastase are synthesized by the pancreatic acinar cells, secreted in the small intestine and are responsible for catalyzing the hydrolysis of peptide bonds. All three of these enzymes are similar in structure, as shown through their X-ray structures. Each of these digestive serine proteases targets different regions of the polypeptide chain, based upon the amino acid residues and side chains surrounding the site of cleavage. The active site of serine proteases is shaped as a cleft where the polypeptide substrate binds. Amino acid residues are labeled from N to C term of the polypeptide substrate (Pi, . . . , P3, P2, P1, P1′, P2′, P3′, . . . , Pj) and their respective binding sub-sites (Si, . . . , S3, S2, S1, S1′, S2′, S3′, . . . , Sj). The cleavage is catalyzed between P1 and P1′. Chymotrypsin is responsible for cleaving peptide bonds flanked with bulky hydrophobic amino acid residues. Particular residues include phenylalanine, tryptophan and tyrosine, which fit into a snug hydrophobic pocket. Trypsin is responsible for cleaving peptide bonds flanked with positively charged amino acid residues. Instead of having the hydrophobic pocket of the chymotrypsin, there exists an aspartic acid residue at the back of the pocket. This can then interact with positively charged residues such as arginine and lysine. Elastase is responsible for cleaving peptide bonds flanked with small neutral amino acid residues, such as alanine, glycine and valine. The pocket that is in trypsin and chymotrypsin is now lined with valine and threonine, rendering it a mere depression, which can accommodate these smaller amino acid residues. Serine proteases are ubiquitous in prokaryotes and eukaryotes and serve important and diverse biological functions such as hemostasis, fibrinolysis, complement formation and the digestion of dietary proteins.

b. Matrix Metalloproteinases

Matrix metalloproteinases (MMPs) constitute a family of Zn⁺²- and calcium-dependent endopeptidases that degrade components of the extracellular matrix (ECM). MMPs also can process a number of cell-surface cytokines, receptors and other soluble proteins. They are involved in normal tissue remodeling processes such as wound healing, pregnancy and angiogenesis. Under physiological conditions, MMPs are synthesized as inactive precursors (zymogens) and are processed to their active form. Additionally, the enzymes are specifically regulated by endogenous inhibitors called tissue inhibitors of matrix metalloproteinases (TIMPs). The proteolytic activity of MMPs acts as an effector mechanism of tissue remodeling in physiologic and pathologic conditions, and as modulators of inflammation. The excess synthesis and production of these proteins lead to accelerated degradation of the ECM which is associated with a variety of diseases and conditions such as, for example, bone homeostasis, arthritis, cancer, multiple sclerosis and rheumatoid arthritis. In the context of neuroinflammatory diseases, MMPs have been implicated in processes such as (a) blood-brain barrier (BBB) and blood-nerve barrier opening, (b) invasion of neural tissue by blood-derived immune cells, (c) shedding of cytokines and cytokine receptors, and (d) direct cellular damage in diseases of the peripheral and central nervous system (Leppert et al. Brain Res. Rev. 36(2-3): 249-57 (2001); Borkakoti et al. Prog. Biophys. Mol. Biol. 70(1): 73-94 (1998)).

Members of the MMP family include collagenases, gelatinases, stromelysins, matrilysin, and membrane-bound MMPs. Most MMPs are secreted in the inactive proenzyme form. The secreted proenzyme MMPs can be activated by several proinflammatory agents such as oxidants, proteinases including elastase, plasmin, and trypsin, and other MMPs. In tissues, physiological MMP activators include tissue or plasma proteinases or opportunistic bacterial proteinases. For example, the plasminogen activator/plasmin system, including ubiquitous plasminogen by urokinase (u-Pa) and tissue-type plasminogen activator (t-Pa), is an important activator of pro-MMP in pathological situations. MMP activity can be inhibited by tissue inhibitors of metalloproteinases (TIMPs), by serine proteinase inhibitors (serpins), and by nonspecific proteinase inhibitors, such as α2-macroglobulin. TIMPs inhibit the MMP activity through noncovalent binding of the active zinc-binding sites of MMPs.

Gelatinase B (MMP-9; type IV collagenase) belongs to a sub-family of MMPs that plays an important role in tissue remodeling in normal and pathological inflammatory processes and is the terminal member of the protease cascade which leads to matrix degradation. It cleaves gelatins and other substrates, such as IFN-β, and is involved in matrix remodeling during embryogenesis, tissue remodeling and development. Gelatinase B is secreted by a variety of leukocytes including neutrophils, macrophages, lymphocytes, and eosinophils. Generally, the expression of gelatinase B is regulated, however, neutrophils store gelatinase B in secretory granules for rapid release. The expression of gelatinase B in cells can be induced by a variety of inflammatory stimuli including interleukin-1β, tumor necrosis factor-α, lymphotoxin, interferon beta, and lipopolysaccharides (LPS), and by other agents stimulating cell migration. For example, gelatinase B is up-regulated in pathological states such as invasion of cancer cells and when leukocytes are released from the bone marrow and migrate toward an inflammatory event. After stimulation by inflammatory cytokines, or upon delivery of bi-directional activation signals following integrin-mediated cell-cell or cell-ECM contact, gelatinase B also can be secreted by lymphocytes and stromal cells.

Gelatinase B, like other gelatinases and MMPs, is secreted in a latent inactive form and is converted to an active species by other proteolytic enzymes, including other MMPs. For example, activated gelatinase A can activate progelatinase B in a process that is inhibited by TIMP-1 and TIMP-2 (Fridman et al, Cancer Research, 55:2548-2555 (1995). Progelatinase B also can be converted to an active form via an interacting protease cascade involving plasmin and stromelysin-1 (MMP-3). Plasmin, generated by the endogenous plasminogen activator (uPA), is not an efficient activator of progelatinase B. Plasmin is able to generate active stromelysin-1 from an inactive proform and the activated stromelysin-1 is itself a potent activator of gelatinase B (Hahn-Dantona et al., Ann NY Acad. Sci, 878:372-387 (1999). Latent gelatinase B also can be activated by other proteases including cathepsin G, kallikrein, and trypsin or by incubation with p-aminophenylmercuric acetate (APMA).

Gelatinase B cleaves a variety of substrates including collagen type II, human myelin basis protein, insulin, and interferon beta. The substrate recognition specificity of gelatinase B has been studied using a phage display library of random hexamers (Kridel et al., (2001), J. Biol. Chem. 276:20572-20578) and has been empirically assessed on a variety of substrates (Descamps, F J et al., (2003) FASEB, 17(8):887-9; Van Den Steen et al., (2002) FASEB, 16: 379-389; and Nelissen et al. (2003) Brain 126: 1371-1381). Gelatinase B typically has a preference for hydrophobic residues at the P1′ position (the position before which cleavage occurs), such as for example Leucine (L). Other amino acid residues that have been recognized as preferentially cleaved by gelatinase B include Phenylalanine (F), Glutamic Acid (E), Tyrosine (Y), and Glutamine (Q). In some cases, protein glycosylation can affect gelatinase B cleavage. For example, proteolysis is more pronounced with IFN-β-1b than with IFN-β.

Cleavage of substrates by gelatinase B can have a regulatory function. For example, cleavage of the neutrophil chemokine IL-8 by truncation of the amino terminal six amino acids potentiates its activity. In addition, gelatinase B cleavage of endothelin-1 facilitates activation of neutrophils and promotes leukocyte-endothelial cell adhesion and subsequent neutrophil trafficking into inflamed tissues. In contrast, cleavage of substrates by gelatinase B can mediate pathological conditions. For example, evidence suggests that cleavage of substrates produces immunodominant peptides which contribute to the generation of autoimmune diseases, such as multiple sclerosis and rheumatoid arthritis. Cleavage of IFN-β by gelatinase B kills its anti-viral activity, and by killing the activity of IFN-β, gelatinase B counteracts the anti-viral and immunotherapeutic effects of the cytokine.

Excessive production of gelatinase B is linked to tissue damage and degenerative inflammatory disorders (St-Pierre et al. Curr. Drug Targets Inflamm. Allergy 2(3): 206-215 (2003); Opdenakker, G. Verh. K. Acad. Geneeskd. Belg. 59(6): 489-514 (1997)). Proteinases, such as gelatinase B, have been implicated in chronic inflammation and autoimmunity due to cleavage of extracellular structural proteins and generation of proteolytic fragments. The expression of gelatinase B has been associated with a variety of infections, autoimmune diseases and inflammatory diseases, including, for example, multiple sclerosis and rheumatoid arthritis, cancer, bone homeostasis, and inflammatory bowel diseases (Opdenakker and Van Damme, Immunol. Today 15: 103-107 (1994); Opdenakker et al. Trends Immunol. 22: 571-579 (2001); Van den Steen et al., FASEB J. 16: 379-389 (2002)). Gelatinase B is increased in the serum and cerebral spinal fluid (CSF) of multiple sclerosis patients and is disease promoting (Gijbels et al., J. Neuroimmunol. 41: 29-34 (1992)). In multiple sclerosis, gelatinase B contributes to the destruction of the blood-brain barrier (Mun-Bryce and Rosenberg, Am. J. Physiol. 274: R1203-R1211 (1998); Lukes et al., Mol. Neurobiol. 19: 267-284 (1999)), and further regulates the inflammatory response by activating or destroying chemokines and cytokines (Schonbeck et al., J. Immunol. 161: 3340-3346 (1998); Van den Steen et al. Blood 96: 2673-2681 (2000)), by assisting the in vivo migration of leukocytes to sites of inflammation under the influence of chemotactic gradients (D'Haese et al., J. Interferon Cytokine Res. 20: 667-674 (2000)).

c. Generation of IFN-β Variants by Removal of Proteolytic Sites

In an example of generating variants exhibiting increased stability by removal of proteolytic sites, IFN-β was modified. The first step in the design of IFN-β mutants resistant to proteolysis includes identifying sites vulnerable to proteolysis along the protein sequence. Based on a list of selected blood, intestinal or any other type of proteases considered (see e.g., Table 4), an exemplary list of amino acids or sequence of amino acids in IFN-β that can be targeted by those proteases was first determined in silico. The protease targets (amino acids or sequence of amino acids) are named in silico HITs (is-HITs). Since protease mixtures in the body are quite complex in composition, it can be expected that the majority of the residues in a given protein sequence can be targeted for proteolysis.

The second step in the design of IFN-β mutants that are resistant to proteolysis includes identifying the appropriate replacing amino acids such that the replacement of the natural amino acid in IFN-β at each is-HIT produces a protein that (i) becomes resistant to proteolysis; and (ii) elicits a level of activity at least comparable to the unmodified or wildtype IFN-β polypeptide. The choice of the replacing amino acids must consider the broad specificity of certain proteases and the need to preserve the physiochemical properties, such as for example hydrophobicity, charge and polarity, of essential (e.g., catalytic, binding, etc.) residues in IFN-β,

Point Accepted Mutation (PAM; Dayhoff et al., 1978) can be used as part of the 2D scanning approach. PAM values, originally developed to produce alignments between protein sequences, are available in the form of probability matrices that reflect an evolutionary distance between amino acids. Conservative substitutions of a residue in a reference sequence are those substitutions that are physically and functionally similar to the corresponding reference residues, i.e., that have a similar size, shape, electric charge, and/or chemical properties, including the ability to form covalent or hydrogen bonds and other such interactions. Conservative substitutions show the highest scores fitting with the PAM matrix criteria in the form of accepted point mutations. The PAM250 matrix is used in the frame of 2D-scanning to identify candidate replacing amino acids for the is-HITs in order to generate conservative mutations without affecting protein function. Typically, at least the two amino acids with the highest values in PAM250 matrix corresponding to conservative substitutions or accepted point mutations were chosen for replacement at each is-HIT. In most cases, the replacement of amino acids by cysteine residues is explicitly avoided since this change potentially leads to the formation of intermolecular disulfide bonds.

Briefly, using the algorithm PROTEOL (on-line at infobiogen.fr and at bioinfo.hku.hk/services/analyseq/cgi-bin/proteol_in.pl), a list of residues along the IFN-β protein of 166 amino acids (SEQ ID NO:1), which can be recognized as substrate for proteases (blood, intestinal, etc.) was established. The algorithm generates a proteolytic digestion map based on a list of proteases, the proteolytic specificity of the proteases and the polypeptide amino acid sequence that is entered. Table 4 provides a non-limiting list of exemplary proteases for which an is-HIT target amino acid can be identified depending on the known substrate specificity of the protease. Modification of IFN-β to confer resistance to other proteases including serine, cysteine, metalloproteases, and aspartyl proteases also is contemplated, based on the known substrate specificity of the protease. For example, modifications to confer resistance of IFN-β to any one or more proteases of pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, luminal pepsin, microvillar endopeptidase, dipeptidyl peptidase, enteropeptidase, hydrolase, NS3, factor Xa, Granzyme B, thrombin, plasmin, urokinase, tPA and PSA also is contemplated.

TABLE 4

| | Amino Acid | Protease or chemical |
|---|---|---|
| Abbreviation | Position | Treatment |
| AspN | D | Endoproteinase Asp-N |
| Chymo | (F, W, Y, M, L)˜P | Chymotrypsin |
| Clos | R | Clostripain |
| CnBr | M | Cyanogen Bromide |
| IBzO | W | IodosoBenzoate |
| Myxo | K | Myxobacter |
| NH₂OH | N G | Hydroxylamine |
| pH2.5 | D P | pH 2.5 |
| ProEn | P | Proline Endopeptidase |
| Staph | E | Staphylococcal Protease |
| Tryp | (K, R)˜P | Trypsin |
| TrypK | K˜P | Trypsin (Arg blocked) |
| TrypR | R˜P | Trypsin (Lys blocked) |

Table 4 shows the in silico identification of exemplary amino acid positions that are targets for proteolysis using selected proteases and chemical treatment.

d. Modified IFN-β Polypeptides Exhibiting Increased Protease Resistance

Using the methods described herein, is-HIT positions can be identified that, upon modification, result in a polypeptide that exhibits increased protein stability as manifested by increased resistance to proteases compared to an unmodified IFN-β polypeptide. Generally, the modified IFN-β polypeptide retains one or more activities, such as anti-viral activity, of the unmodified IFN-β. In one example, modifications are in an unmodified IFN-β having a sequence of amino acids set forth in SEQ ID NO:1 or 3. Using the methods described herein, the following is-HIT positions were identified to eliminate protease sensitive sites and increase protein stability of IFN-β: 3, 18, 21, 34, 38, 41, 43, 48, 49, 50, 51, 53, 54, 57, 60, 61, 62, 63, 64, 70, 72, 88, 102, 106, 115, 117, 122, 125, 126, 132, 133, 143, 147, 149, 151, 154, 156, 160 and 164. Amino acid replacement or replacements can be at any one or more position corresponding to any of the following positions: Y3, Q18, L21, D34, F38, P41, E43, Q48, Q49, F50, Q51, E53, D54, L57, Y60, E61, M62, L63, Q64, F70, Q72, L88, L102, L106, K115, M117, L122, Y125, Y126, Y132, L133, W143, R147, E149, L151, F154, F156, L160 and L164 of a mature IFN-β polypeptide set forth in SEQ ID NO:1. In a particular embodiment, the amino acid replacement or replacements rendering the modified polypeptide more resistant to proteolysis is (are) replacing Y by H or I; replacing L by I, V, H, A, T, or Q; replacing M by I, T, Q, H, A or V; replacing F by I or V; replacing D by G, N or Q; replacing E by Q, H or N; replacing P by A or S; replacing W by H or S; replacing K by Q, S, H or N; replacing Q by H, S, T or N; and replacing R by H or Q. Table 5 provides non-limiting examples of amino acid replacements, corresponding to amino acid positions of a mature IFN-β polypeptide, that increase resistance to proteolysis and, thereby, protein stability. Other non-limiting amino acid modifications that exhibit increased resistance to proteolysis compared to an unmodified IFN-β polypeptide include any one or more amino acid modifications set forth in Table 6 and disclosed in published U.S. Application No. US-2004-0132977-A1. In Tables 5 and 6 below, the sequence identifier (SEQ ID No.) is in parenthesis next to each substitution.

TABLE 5
IFN-β Mutations to Increase Resistance to Proteolysis

| | | | | |
|---|---|---|---|---|
| Y3H (4) | Y3I (5) | L6I (6) | L6V (7) | L20I (8) |
| L20V (9) | L21I (10) | L21V (11) | L24I (12) | L24V (13) |
| D34N (14) | D34Q (15) | F38I (16) | F38V (17) | P41A (18) |
| P41S (19) | E43Q (20 | E43H (21) | E43N (22) | F50I (23) |
| F50V (24) | E53Q (25) | E53H (26) | E53N (27) | D54N (28) |
| D54Q (29) | L57I (30 | L57V (31) | Y60H (32) | Y60I (33) |
| E61Q (34) | E61H (35) | E61N (36) | M62I (37) | M62V (38) |
| L63I (39) | L63V (40) | F70I (41) | F70V (42) | W79H (43) |
| W79S (44) | L87I (45) | L87V (46) | L88I (47) | L88V (48) |
| L98I (49) | L98V (50) | L102I (51) | L102V (52) | L106I (53) |
| L106V (54) | K115N (55) | K115Q (56) | M117I (57) | M117V (58) |
| L122I (59) | L122V (60) | Y125H (61) | Y125I (62) | Y126H (63) |
| Y126I (64) | Y132H (65) | Y132I (66) | L133I (67) | L133V (68) |
| W143H (71) | W143S (72) | R147H (73) | R147Q (74) | E149Q (75) |
| E149H (76) | E149N (77) | L151I (78) | L151V (79) | F154I (80) |
| F154V (81) | F156I (82) | F156V (83) | L160I (84) | L160V (85) |
| L164I (86) | L164V (87) | L6H (534) | L6A (535) | L20H (537) |
| L20A (538) | L21T (539) | L21Q (540) | L21H (541) | L21A (542) |
| L24T (543) | L24Q (544) | L24H (545) | L24A (546) | D34G (549) |
| D54G (554) | L57T (555) | L57Q (556) | L57H (557) | L57A (558) |
| M62T (559) | M62Q (560) | M62A (561) | L63T (562) | L63Q (563) |
| L63H (564) | L63A (565) | L87H (569) | L87A (570) | L88T (571) |
| L88Q (572) | L88H (573) | L88A (574) | L98H (575) | L98A (576) |
| L102T (578) | L102Q (579) | L102H (580) | L102A (581) | L106T (585) |
| L106Q (586) | L106H (587) | L106A (588) | K115S (593) | K115H (594) |
| M117T | M117Q | M117A (598) | L122T (599) | L122Q (600) |
| (596) | (597) | | | |
| L122H (601) | L122A (602) | L133T (604) | L133Q (605) | L133H (606) |
| L133A (607) | L151T (611) | L151Q (612) | L151H (613) | L151A (614) |
| L160T (615) | L160Q (616) | L160H (617) | L160A (618) | L164T (619) |
| L164Q (620) | L164H (621) | L164A (622) | K19N (536) | E29N (547) |
| K33N (548) | D39N (550) | E42N (551) | K45N (552) | K52N (553) |
| D73N (566) | E81N (567) | E85N (568) | K99N (577) | E103N (582) |
| E104N (583) | K105N (584) | E107N (589) | K108N (590) | E109N (591) |
| D110N (592) | K123N (603) | K134N (608) | K136N (609) | E137N (610) |
| Q18H (623) | Q18S (624) | Q18T (625) | Q18N (626) | Q23H (627) |
| Q23S (628) | Q23T (629) | Q23N (630) | Q48H (631) | Q48S (632) |
| Q48T (633) | Q48N (634) | Q49H (635) | Q49S (636) | Q49T (637) |
| Q49N (638) | Q51H (639) | Q51S (640) | Q51T (641) | Q51N (642) |
| Q64H (643) | Q64S (644) | Q64T (645) | Q64N (646) | Q72H (647) |
| Q72S (648) | Q72T (649) | Q72N (650) | | |

TABLE 6
Additional IFN-β Mutations to Increase Resistance to Proteolysis

| | | | | |
|---|---|---|---|---|
| M1V (262) | M1I (263) | M1T (264) | M1A (265) | M1Q (261) |
| M1D (322) | M1E (323) | M1K (324) | M1N (325) | M1R (326) |
| M1S (327) | M1C (651) | L5V (266) | L5I (267) | L5T (268) |
| L5Q (269) | L5H (270) | L5A (271) | L5D (328) | L5E (329) |
| L5K (330) | L5R (331) | L5N (332) | L5S (333) | L6D (334) |
| L6E (335) | L6K (336) | L6N (337) | L6Q (338) | L6R (339) |
| L6S (340) | L6T (341) | L6C (652) | F8I (272) | F8V (273) |
| F8D (342) | F8E (343) | F8K (344) | F8R (345) | L9V (274) |
| L9I (275) | L9T (276) | L9Q (277) | L9H (278) | L9A (279) |
| L9D (346) | L9E (347) | L9K (348) | L9N (349) | L9R (350) |
| L9S (351) | Q10D (352) | Q10E (353) | Q10K (354) | Q10N (355) |
| Q10R (356) | Q10S (357) | Q10T (358) | Q10C (653) | R11H (280) |
| R11Q (281) | S12D (359) | S12E (360) | S12K (361) | S12R (362) |
| S13D (363) | S13E (364) | S13K (365) | S13N (366) | S13Q (367) |
| S13R (368) | S13T (369) | S13C (654) | N14D (370) | N14E (371) |
| N14K (372) | N14Q (373) | N14R (374) | N14S (375) | N14T (376) |
| F15I (282) | F15V (283) | F15D (377) | F15E (378) | F15K (379) |
| F15R (380) | Q16D (381) | Q16E (382) | Q16K (383) | Q16N (384) |
| Q16R (385) | Q16S (386) | Q16T (387) | Q16C (655) | C17D (388) |
| C17E (389) | C17K (390) | C17N (391) | C17Q (392) | C17R (393) |
| C17S (394) | C17T (395) | K19Q (284) | K19T (285) | K19S (286) |
| K19H (287) | L20N (396) | L20Q (402) | L20R (398) | L20S (399) |
| L20T (400) | L20D (401) | L20E (397) | L20K (403) | W22S (288) |
| W22H (289) | W22D (404) | W22E (405) | W22K (406) | W22R (407) |
| Q23D (408) | Q23E (409) | Q23K (410) | Q23R (411) | L24D (412) |
| L24E (413) | L24K (414) | L24R (415) | N25H (290) | N25S (291) |
| N25Q (292) | R27H (293) | R27Q (294) | L28V (295) | L28I (296) |
| L28T (297) | L28Q (298) | L28H (299) | L28A (300) | E29Q (301) |
| E29H (302) | Y30H (303) | Y30I (304) | L32V (305) | L32I (306) |
| L32T (307) | L32Q (308) | L32H (309) | L32A (310) | K33Q (311) |
| K33T (312) | K33S (313) | K33H (314) | R35H (315) | R35Q (316) |
| M36V (317) | M36I (318) | M36T (319) | M36Q (320) | M36A (321) |
| D39Q (154) | D39H (155) | D39G (156) | E42Q (157) | E42H (158) |
| K45Q (159) | K45T (160) | K45S (161) | K45H (162) | L47V (163) |
| L47I (164) | L47T (165) | L47Q (166) | L47H (167) | L47A (168) |
| K52Q (169) | K52T (170) | K52S (171) | K52H (172) | F67I (173) |
| F67V (174) | R71H (175) | R71Q (176) | D73Q (177) | D73H (178) |
| D73G (179) | G78D (416) | G78E (417) | G78K (418) | G78R (419) |
| W79D (420) | W79E (421) | W79K (422) | W79R (423) | N80D (424) |
| N80E (425) | N80K (426) | N80R (427) | E81Q (180) | E81H (181) |
| T82D (428) | T82E (429) | T82K (430) | T82R (431) | I83D (432) |
| I83E (433) | I83K (434) | I83R (435) | I83N (436) | I83Q (437) |
| I83S (438) | I83T (439) | E85Q (182) | E85H (183) | N86D (440) |
| N86E (441) | N86K (442) | N86R (443) | N86Q (444) | N86S (445) |
| N86T (446) | L87D (447) | L87E (448) | L87K (449) | L87R (450) |
| L87N (451) | L87Q (452) | L87S (453) | L87T (454) | A89D (455) |
| A89E (456) | A89K (457) | A89R (458) | N90D (459) | N90E (460) |
| N90K (461) | N90Q (462) | N90R (463) | N90S (464) | N90T (465) |
| N90C (129) | V91D (466) | V91E (467) | V91K (468) | V91N (469) |
| V91Q (470) | V91R (471) | V91S (472) | V91T (473) | V91C (131) |
| Y92H (184) | Y92I (185) | Q94D (474) | Q94E (475) | Q94K (476) |
| Q94N (477) | Q94R (478) | Q94S (479) | Q94T (480) | Q94C (656) |
| I95D (481) | I95E (482) | I95K (483) | I95N (484) | I95Q (485) |
| I95R (486) | I95S (487) | I95T (488) | H97D (489) | H97E (490) |
| H97K (491) | H97N (492) | H97Q (493) | H97R (494) | H97S (495) |
| H97T (496) | H97C (657) | L98D (497) | L98E (498) | L98K (499) |
| L98N (500) | L98Q (501) | L98R (502) | L98S (503) | L98T (504) |
| L98C (658) | K99Q (186) | K99T (187) | K99S (188) | K99H (189) |
| V101D (505) | V101E (506) | V101K (507) | V101N (508) | V101Q (509) |
| V101R (510) | V101S (511) | V101T 512) | V101C (659) | E103Q (190) |
| E103H (191) | E104Q (192) | E104H (193) | K105Q (194) | K105T (195) |
| K105S (196) | K105H (197) | E107Q (198) | E107H (199) | K108Q (200) |
| K108T (201) | K108S (202) | K108H (203) | E109H (205) | E109Q (204) |
| D110Q (206) | D110H (207) | D110G (208) | F111I (209) | F111V (210) |
| R113H (211) | R113Q (212) | L116V (213) | L116I (214) | L116T (215) |
| L116Q (216) | L116H (217) | L116A (218) | L120V (219) | L120I (220) |
| L120T (221) | L120Q (222) | L120H (223) | L120A (224) | K123Q (225) |
| K123T (226) | K123S (227) | K123H (228) | R124H (229) | R124Q (230) |
| R128H (231) | R128Q (232) | L130V (233) | L130I (234) | L130T (235) |
| L130Q (236) | L130H (237) | L130A (238) | K134Q (239) | K134T (240) |
| K134S (241) | K134H (242) | K136Q (243) | K136T (244) | K136S (245) |
| K136H (246) | E137Q (247) | E137H (248) | Y138H (253) | Y138I (254) |
| R152H (255) | R152Q (256) | Y155H (257) | Y155I (258) | R159H (259) |
| R159Q (260) | Y163H (249) | Y163I (250) | R165H (251) | R165Q (252) |

A modified IFN-β polypeptide provided herein that exhibits increased protease resistance can contain one or more amino acid modifications corresponding to any one or more modifications of Y3H, Y3I, L6I, L6V, L6H, L6A, Q18H, Q18S, Q18T, Q18N, K19N, L20I, L20V, L20H, L20A, L21I, L21V, L21T, L21Q, L21H, L21A, Q23H, Q23S, Q23T, Q23N, L24I, L24V, L24T, L24Q, L24H, L24A, E29N, K33N, D34N, D34Q, D34G, F38I, F38V, D39N, P41A, P41S, E42N, E43Q, E43H, E43N, K45N, Q48H, Q48S, Q48T, Q48N, Q49H, Q49S, Q49T, Q49N, F50I, F50V, Q51H, Q51S, Q51T, Q51N, K52N, E53Q, E53H, E53N, D54N, D54Q, D54G, L57I, L57V, L57T, L57Q, L57H, L57A, Y60H, Y60I, E61Q, E61H, E61N, M62I, M62V, M62T, M62Q, M62A, L63I, L63V, L63T, L63Q, L63H, L63A, Q64H, Q64S, Q64T, Q64N, F70I, F70V, Q72H, Q72S, Q72T, Q72N, D73N, W79H, W79S, E81N, E85N, L87I, L87V, L87H, L87A, L88I, L88V, L88T, L88Q, L88H, L88A, L98I, L98V, L98H, L98A, K99N, L102I, L102V, L102T, L102Q, L102H, L102A, E103N, E104N, K105N, L106I, L106V, L106T, L106Q, L106H, L106A, E107N, K108N, E109N, D110N, K115N, K115Q, K115S, K115H, M117I, M117V, M117T, M117Q, M117A, L122I, L122V, L122T, L122Q, L122H, L122A, K123N, Y125H, Y125I, Y126H, Y126I, Y132H, Y132I, L133I, L133V, L133T, L133Q, L133H, L133A, K134N, K136N, E137N, W143H, W143S, R147H, R147Q, E149Q, E149H, E149N, L151I, L151V, L151T, L151Q, L151H, L151A, F154I, F154V, F156I, F156V, L160I, L160V, L160T, L160Q, L160H, L160A, L164I, L164V, L164T, L164Q, L164H, and L164A of a mature IFN-β polypeptide set forth in SEQ ID NO:1. In some examples, the modifications are in an unmodified IFN-β polypeptide, such as an IFN-β having a sequence of amino acids set forth in SEQ ID NO:1 or SEQ ID NO:3. Exemplary modified IFN-β LEAD candidate polypeptides are set forth in any one of SEQ ID NOS: 4-68, 71-87, 534, 535, 536-594, and 596-650. Additionally, a modified IFN-β as set forth above can contain a further modification compared to an unmodified IFN-β polypeptide. Generally, the resulting modified IFN-β polypeptide retains one or more activities of the unmodified IFN-β. The further modification can be one or more amino acid replacement(s) at an amino acid position corresponding to any of positions 1, 5, 6, 8, 9, 10, 11, 12, 13, 14, 15, 16, 17, 19, 20, 22, 23, 24, 25, 27, 28, 29, 30, 32, 33, 35, 36, 39, 42, 45, 47, 52, 67, 71, 73, 78, 79, 80, 81, 82, 83, 85, 86, 87, 89, 90, 91, 92, 94, 95, 97, 98, 99, 101, 103, 104, 105, 107, 108, 109, 110, 111, 113, 116, 123, 124, 128, 130, 134, 136, 137, 138, 152, 155, 159, 163, and 165. Amino acid replacement or replacements can occur at one or more of amino acid residues corresponding to any of positions M1, L5, L6, F8, L9, Q10, R11, S12, S13, N14, F15, Q16, C17, K19, L20, W22, Q23, L24, N25, R27, L28, E29, Y30, L32, K33, R35, M36, D39, E42, K45, L47, K52, F67, R71, D73, G78, W79, N80, E81, T82, I83, E85, N86, L87, A89, N90, V91, Y92, Q94, I95, H97, L98, K99, V101, E103, E104, K105, E107, K108, E109, D110, F111, R113, L116, K123, R124, R128, L130, K134, K136, E137, Y138, R152, Y155, R159, Y163, and R165 of a mature IFN-β polypeptide set forth in SEQ ID NO:1. For example, the amino acid replacements of the further modification can include a modification, set forth in Table 6 above, such as for example, any one or more amino acid modifications corresponding to any of M1V, M1I, M1T, M1A, M1Q, M1D, M1E, M1K, M1N, M1R, M1S, M1C, L5V, L5I, L5T, L5Q, L5H, L5A, L5D, L5E, L5K, L5R, L5N, L5S, L6D, L6E, L6K, L6N, L6Q, L6R, L6S, L6T, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, R11H, R11Q, S12D, S12E, S12K, S12R, S13D, S13E, S13K, S13N, S13Q, S13R, S13T, S13C, N14D, N14E, N14K, N14Q, N14R, N14S, N14T, F15I, F15V, F15D, F15E, F15K, F15R, Q16D, Q16E, Q16K, Q16N, Q16R, Q16S, Q16T, Q16C, C17D, C17E, C17K, C17N, C17R, C17S, C17T, K19Q, K19T, K19S, K19H, L20N, L20Q, L20R, L20S, L20T, L20D, L20E, L20K, W22S, W22H, W22D, W22E, W22K, W22R, Q23D, Q23E, Q23K, Q23R, L24D, L24E, L24K, L24R, N25H, N25S, N25Q, R27H, R27Q, L28V, L28I, L28T, L28Q, L28H, L28A, E29Q, E29H, Y30H, Y30I, L32V, L32I, L32T, L32Q, L32H, L32A, K33Q, K33T, K33S, K33H, R35H, R35Q, M36V, M36I, M36T, M36Q, M36A, D39Q, D39H, D39G, E42Q, E42H, K45Q, K45T, K45S, K45T, L47V, L47I, L47T, L47Q, L47H, L47A, K52Q, K52T, K52S, K52H, F67I, F67V, R71H, R71Q, D73Q, D73H, D73G, G78D, G78E, G78K, G78R, N80D, N80E, N80K, N80R, E81Q, E81H, T82D, T82E, T82K, T82R, I83D, I83E, I83K, I83R, I83N, I83Q, I83S, I83T, E85 Q, E85H, N86D, N86E, N86K, N86R, N86Q, N86S, N86T, L87D, L87E, L87K, L87R, L87N, L87Q, L87S, L87T, A89D, A89E, A89K, A89R, N90D, N90E, N90K, N90Q, N90R, N90S, N90T, N90C, V91D, V91E, V91K, V91N, V91Q, V91R, V91S, V91T, V91C, Y92H, Y92I, Q94D, Q94E, Q94K, Q94N, Q94R, Q94S, Q94T, Q94C, I95D, I95E, I95K, I95N, I95Q, I95R, I95S, I95T, H97D, H97E, H97K, H97N, H97Q, H97R, H97S, H97T, H97C, L98D, L98E, L98K, L98N, L98Q, L98R, L98S, L98T, L98C, K99Q, K99T, K99S, K99H, V101D, V101E, V101K, V101N, V101Q, V101R, V101S, V101T, V101C, E103Q, E103H, E104Q, E104H, K105Q, K105T, K105S, K105H, E107Q, E107H, K108 Q, K108T, K108S, K108H, E109H, E109Q, D110Q, D110H, D110G, F111I, F111V, R113H, R113Q, L116V, L116I, L116T, L116Q, L116H, L116A, K123Q, K123T, K123S, K123H, R124H, R124Q, R128H, R128Q, L130V, L130I, L130T, L130Q, L130H, L130A, K134Q, K134T, K134S, K134H, K136Q, K136T, K136S, K136H, E137Q, E137H, Y138H, Y138I, R152H, R152Q, Y155H, Y155I, R159H, R159Q, Y163H, Y163I, R165H, and R165Q of a mature IFN-β polypeptide set forth in SEQ ID NO:1.

In one example, exemplary modified IFN-β polypeptides provided herein exhibiting increased protease resistance can contain one or more amino acid modifications compared to an unmodified IFN-β polypeptide of SEQ ID NO:1, such as for example, any one or more amino acid modifications corresponding to Y3I, K19N, Q18S, Q18N, L20I, L20V, K33N, D34N, P41A, P41S, E42N, E43N, K45N, Q48H, Q48S, Q48T, Q49H, Q49S, Q49T, F50I, F50V, Q51H, Q51S, Q51T, Q51N, K52N, E53N, D54N, D54G, L57I, Y60I, E61H, E61N, L63I, Q64H, Q64S, Q64T, F70I, F70V, Q72H, Q72S, E85N, L88I, L88V, L98I, L98V, K99N, E103N, E104N, K105N, L106I, L106V, E107N, E109N, K115N, K115S, K115H, K115N, K123N, Y125I, Y126I, Y132I, K134N, K136N, R147H, R147Q, E149H, E149N, L151I, and F154V of a mature IFN-β polypeptide set forth in SEQ ID NO:1. Exemplary modified IFN-β candidate LEAD polypeptides are set forth in any one of SEQ ID NOS: 5, 8, 9, 14, 18, 19, 22-24, 27, 28, 30, 33, 35, 36, 39, 41, 42, 47-50, 53-55, 62, 64, 66, 73, 74, 76-78, 81, 536, 548, 551-554, 568, 577, 582-584, 589, 591, 593, 594, 603, 608, 609, 624, 626, 631-633, 635-637, 639-645, 647, and 648. In another example, an IFN-β polypeptide provided herein exhibiting increased protease resistance can contain one or more amino acid modifications compared to an unmodified IFN-β polypeptide having a sequence of amino acids set forth in SEQ ID NO:3, such as for example, any one or more amino acid modifications corresponding to Y3I, K19N, Q18S, Q18N, L20I, L20V, L21I, L21V, K33N, D34N, P41A, P41S, E42N, E43N, K45N, Q48H, Q48S, Q48T, Q49H, Q49S, Q49T, F50I, F50V, Q51H, Q51S, Q51T, Q51N, K52N, E53N, D54N, D54G, L57I, Y60I, E61H, E61N, M62I, M62V, L63I, Q64H, Q64S, Q64T, F70I, F70V, Q72H, Q72S, E85N, L88I, L88V, L98I, L98V, K99N, E103N, E104N, K105N, L106I, L106V, E107N, E109N, K115N, K115S, K115H, K115N, M117I, M117V, L122I, L122V, K123N, Y125I, Y126I, Y132I, K134N, K136N, R147H, R147Q, E149H, E149N, L151I, F154V and L160V of a mature IFN-α polypeptide set forth in SEQ ID NO:1. Additionally, a modified IFN-β containing any one or more modification as set forth above can contain a further modification. Generally, the resulting modified polypeptide exhibits increased protease resistance and retains one or more activities of the unmodified IFN-β. The further modification can be one or more replacement(s) at an amino acid position corresponding to any of positions 1, 3, 5, 6, 8, 9, 10, 11, 12, 13, 14, 15, 16, 17, 18, 19, 20, 21, 22, 23, 24, 25, 27, 28, 29, 30, 32, 33, 34, 35, 36, 38, 39, 42, 43, 45, 47, 48, 49, 52, 53, 54, 57, 60, 61, 62, 63, 64, 67, 71, 72, 73, 78, 79, 80, 81, 82, 83, 85, 86, 87, 88, 89, 90, 91, 92, 94, 95, 97, 98, 99, 101, 102, 103, 104, 105, 106, 107, 108, 109, 110, 111, 113, 115, 116, 117, 122, 123, 124, 125, 126, 128, 130, 132, 133, 134, 136, 137, 138, 143, 149, 151, 152, 155, 156, 159, 160, 163, 164, and 165. Amino acid replacement or replacements can occur at one or more of amino acid positions corresponding to any of positions M1, Y3, L5, L6, F8, L9, Q10, R11, S12, S13, N14, F15, Q16, C17, Q18, K19, L20, L21, W22, Q23, L24, N25, R27, L28, E29, Y30, L32, K33, D34, R35, M36, F38, D39, E42, E43, K45, L47, Q48, Q49, K52, E53, D54, L57, Y60, E61, M62, L63, Q64, F67, R71, Q72, D73, G78, W79, N80, E81, T82, I83, E85, N86, L87, L88, A89, N90, V91, Y92, Q94, I95, H97, L98, K99, V101, L102, E103, E104, K105, L106, E107, K108, E109, D110, F111, R113, K115, L116, M117, L122, K123, R124, Y125, Y126, R128, L130, Y132, L133, K134, K136, E137, Y138, W143, E149, L151, R152, Y155, F156, R159, L160, Y163, L164 and R165 of a mature IFN-β polypeptide set forth in SEQ ID NO:1. For example, the amino acid replacements of the further modification can correspond to any one or more modification of M1V, M1I, M1T, M1A, M1Q, M1D, M1E, M1K, M1N, M1R, M1S, M1C, Y3H, L5V, L5I, L5T, L5Q, L5H, L5A, L5D, L5E, L5K, L5R, L5N, L5S, L6H, L6A, L6I, L6V, L6D, L6E, L6K, L6N, L6Q, L6R, L6S, L6T, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, R11H, R11Q, S12D, S12E, S12K, S12R, S13D, S13E, S13K, S13N, S13Q, S13R, S13T, S13C, N14D, N14E, N14K, N14Q, N14R, N14S, N14T, F15I, F15V, F15D, F15E, F15K, F15R, Q16D, Q16E, Q16K, Q16N, Q16R, Q16S, Q16T, Q16C, C17D, C17E, C17K, C17N, C17R, C17S, C17T, Q18H, Q18T, K19Q, K19T, K19S, K19H, L20H, L20A, L20N, L20Q, L20R, L20S, L20T, L20D, L20E, L20K, L21I, L21V, L21T, L21Q, L21H, L21A, W22S, W22H, W22D, W22E, W22K, W22R, Q23H, Q23S, Q23T, Q23N, Q23D, Q23E, Q23K, Q23R, L24T, L24Q, L24H, L24I, L24V, L24D, L24E, L24K, L24R, N25H, N25S, N25Q, R27H, R27Q, L28V, L28I, L28T, L28Q, L28H, L28A, E29N, E29Q, E29H, Y30H, Y30I, L32V, L32I, L32T, L32Q, L32H, L32A, K33Q, K33T, K33S, K33H, D34Q, D34G, R35H, R35Q, M36V, M36I, M36T, M36Q, M36A, F38I, F38V, D39N, D39Q, D39H, D39G, E42Q, E42H, E43Q, E43H, K45Q, K45T, K45S, K45T, L47V, L47I, L47T, L47Q, L47H, L47A, Q48N, Q49N, K52Q, K52T, K52S, K52H, E53Q, E53H, D54Q, L57T, L57Q, L57H, L57A, L57V, Y60H, E61Q, M62I, M62V, M62T, M62Q, M62A, L63T, L63Q, L63H, L63A, L63V, Q64N, F67I, F67V, R71H, R71Q, Q72T, Q72N, D73N, D73Q, D73H, D73G, G78D, G78E, G78K, G78R, W79H, W79S, N80D, N80E, N80K, N80R, E81N, E81Q, E81H, T82D, T82E, T82K, T82R, I83D, I83E, I83K, I83R, I83N, I83Q, I83S, I83T, E85 Q, E85H, N86D, N86E, N86K, N86R, N86Q, N86S, N86T, L87H, L87A, L88T, L88Q, L88H, L88A, L87I, L87V, L87D, L87E, L87K, L87R, L87N, L87Q, L87S, L87T, A89D, A89E, A89K, A89R, N90D, N90E, N90K, N90Q, N90R, N90S, N90T, N90C, V91D, V91E, V91K, V91N, V91Q, V91R, V91S, V91T, V91C, Y92H, Y92I, Q94D, Q94E, Q94K, Q94N, Q94R, Q94S, Q94T, Q94C, I95D, I95E, I95K, I95N, I95Q, I95R, I95S, I95T, H97D, H97E, H97K, H97N, H97Q, H97R, H97S, H97T, H97C, L98H, L98A, L98D, L98E, L98K, L98N, L98Q, L98R, L98S, L98T, L98C, K99Q, K99T, K99S, K99H, V101D, V101E, V101K, V101N, V101Q, V101R, V101S, V101T, V101C, L102T, L102Q, L102H, L102A, L102I, L102V, E103Q, E103H, E104Q, E104H, K105Q, K105T, K105S, K105H, L106T, L106Q, L106H, L106A, E107Q, E107H, K108N, K108Q, K108T, K108S, K108H, E109H, E109Q, D110N, D110Q, D110H, D110G, F111I, F111V, R113H, R113Q, K115Q, L116V, L116I, L116T, L116Q, L116H, L116A, M117I, M117V, M117T, M117Q, M117A, L122I, L122V, L122T, L122Q, L122H, L122A, K123Q, K123T, K123S, K123H, R124H, R124Q, Y125H, Y126H, R128H, R128Q, L130V, L130I, L130T, L130Q, L130H, L130A, L133T, L133Q, L133H, L133A, Y132I, K134Q, K134T, K134S, K134H, K136Q, K136T, K136S, K136H, E137N, E137Q, E137H, Y138H, Y138I, W143H, W143S, E149Q, L151T, L151Q, L151H, L151A, L151V, R152H, R152Q, F154V, F154I, Y155H, Y155I, F156I, F156V, R159H, R159Q, L160V, L160T, L160Q, L160H, L160A, L160I, Y163H, Y163I, L164T, L164Q, L164H, L164A, L164I, L164V, R165H, and R165Q of a mature IFN-β polypeptide set forth in SEQ ID NO:1.

In another example, an IFN-β polypeptide provided herein exhibiting increased protease resistance can contain any two or more amino acid modifications set forth above compared to an unmodified IFN-β polypeptide, such as for example compared to an unmodified IFN-β polypeptide set forth in SEQ ID NO:1 or 3. For example, an IFN-β polypeptide can contain two or more amino acid modifications at is-HIT positions set forth in Table 5 above, two or more amino acid modifications at is-HIT positions set forth in Table 6 above, or any combination thereof, such as one or more amino acid modifications set forth in Table 5 and one or more amino acid modifications set forth in Table 6. Generally, the resulting IFN-β polypeptide exhibits increased protease resistance and retains one more activities of an unmodified IFN-β polypeptide. Non-limiting examples of IFN-β SuperLead polypeptides containing two or more amino acid modifications and exhibiting increased resistance to proteolysis are described in Example 7 and can include amino acid replacements at amino acid residues corresponding to L5E/Q10D, L5E/K108S, L6Q/L47I, L5D/K108S, L5N/L6E, L5Q/K108S, L5N/Q10D, L6Q/M36I, L5D/N86Q, L5N/K108S, L5D/L6Q, L6E/Q10D, L5Q/N86Q, L6E/K108S, L5D/L47I, L6Q/K108S, L5N/L6Q, L6E/N86Q, L5Q/L6Q, L5D/M36I, L5N/L47I, L6Q/N86Q of a mature IFN-β polypeptide set forth in SEQ ID NO:1. Exemplary modified IFN-β SuperLead polypeptides containing two or more amino acid modifications and exhibiting increased protease resistance have a sequence of amino acids set forth in any one of SEQ ID NOS: 89, 92, 96-98, 102, 104-109, 111, 114, 116, 117, and 120-125.

i. Modified IFN-β Polypeptides Exhibiting Increased Protease Resistance to Gelatinase B

Exemplary of modified IFN-β polypeptides exhibiting increased protease resistance are IFN-β polypeptides exhibiting increased resistance to gelatinase B. Non-limiting modifications in an IFN-β polypeptide that confer increased resistance to gelatinase B can be rationally determined based on the known substrate specificity of gelatinase B (see e.g. Descamps, F J et al., (2003) FASEB, 17(8):887-9). For example, based on the cleavage of gelatinase B substrates, the following amino acids were identified as target amino acids: Phenylalanine (F), Leucine (L), Glutamic Acid (E), Tyrosine (Y), and Glutamine (Q). The following is-HIT positions were identified to eliminate gelatinase B sensitive sites and increase protein stability of IFN-β: 3, 5, 6, 8, 9, 10, 15, 16, 18, 20, 21, 23, 24, 28, 29, 30, 32, 38, 42, 43, 47, 48, 49, 50, 51, 53, 57, 60, 61, 63, 64, 67, 70, 72, 81, 85, 87, 88, 92, 94, 98, 102, 103, 104, 106, 107, 109, 111, 116, 120, 125, 126, 130, 132, 133, 137, 138, 149, 151, 154, 156, 160, 163, and 164, corresponding to amino acid positions in a mature IFN-β polypeptide set forth in SEQ ID NO:1. Amino acid modifications can be at any one or more positions corresponding to any of the following positions: Y3, L5, L6, F8, L9, Q10, F15, Q16, Q18, L20, L21, Q23, L24, L28, E29, Y30, L32, F38, E42, E43, L47, Q48, Q49, F50, Q51, E53, L57, Y60, E61, L63, Q64, F67, F70, Q72, E81, E85, L87, L88, Y92, Q94, L98, L102, E103, E104, L106, E107, E109, F111, L116, L120, Y125, Y126, L130, Y132, L133, E137, Y138, E149, L151, F154, F156, L160, Y163, and L164 of a mature IFN-β polypeptide set forth in SEQ ID NO:1. Candidate leads can be obtained by replacement or replacements of amino acids at is-HIT positions such as, but not limited to, amino acid modifications as described for candidate LEADs in Table 5 and in Table 6. Candidate leads can be empirically tested to determine those that confer resistance to gelatinase B, as is described in Example 8 for some non-limiting IFN-β polypeptides. Table 7 provides non-limiting examples of amino acid modifications that increase resistance to proteolysis by gelatinase B and, thereby, protein stability. Generally, a resulting modified IFN-β polypeptide retains one or more activities of a mature or unmodified IFN-β polypeptide. In Table 7 below, the sequence identifier (SEQ ID No.) is in parenthesis next to each substitution.

TABLE 7
IFN-β Mutations to Increase Resistance to Proteolysis by Gelatinase B

| | | | | |
|---|---|---|---|---|
| Y3H (4) | Y3I (5) | L5V (266) | L5I (267) | L5T (268) |
| L5Q (269) | L5H (270) | L5A (271) | L5D (328) | L5E (329) |
| L5K (330) | L5R (331) | L5N (332) | L5S (333) | L6I (6) |
| L6V (7) | L6H (534) | L6A (535) | L6D (334) | L6E (335) |
| L6K (336) | L6N (337) | L6Q (338) | L6R (339) | L6S (340) |
| L6T (341) | L6C (652) | F8I (272) | F8V (273) | F8D (342) |
| F8E (343) | F8K (344) | F8R (345) | L9V (274) | L9I (275) |
| L9T (276) | L9Q (277) | L9H (278) | L9A (279) | L9D (346) |
| L9E (347) | L9K (348) | L9N (349) | L9R (350) | L9S (351) |
| Q10D (352) | Q10B (353) | Q10K (354) | Q10N (355) | Q10R (356) |
| Q10S (357) | Q10T (358) | Q10C (653) | F15I (282) | F15V (283) |
| F15D (377) | F15E (378) | F15K (379) | F15R (380) | Q16D (381) |
| Q16E (382) | Q16K (383) | Q16N (384) | Q16R (385) | Q16S (386) |
| Q16T (387) | Q16C (655) | Q18H (623) | Q18S (624) | Q18T (625) |
| Q18N (626) | L20I (8) | L20V (9) | L20H (537) | L20A (538) |
| L20N (396) | L20Q (402) | L20R (398) | L20S (399) | L20T (400) |
| L20D (401) | L20E (397) | L20K (403) | L21I (10) | L21V (11) |
| L21T (539) | L21Q (540) | L21H (541) | L21A (542) | Q23H (627) |
| Q23S (628) | Q23T (629) | Q23N (630) | Q23D (408) | Q23E (409) |
| Q23K (410) | Q23R (411) | L28V (295) | L28I (296) | L28T (297) |
| L28Q (298) | L28H (299) | L28A (300) | E29N (547) | E29Q (301) |
| E29H (302) | Y30H (303) | Y30I (304) | L32V (305) | L32I (306) |
| L32T (307) | L32Q (308) | L32H (309) | L32A (310) | F38I (16) |
| F38V (17) | E42N (551) | E42Q (157) | E42H (158) | E43Q (20) |
| E43H (21) | E43N (22) | L47V (163) | L47I (164) | L47T (165) |
| L47Q (166) | L47H (167) | L47A (168) | Q48H (631) | Q48S (632) |
| Q48T (633) | Q48N (634) | Q49H (635) | Q49S (636) | Q49T (637) |
| Q49N (638) | F501 (23) | F50V (24) | Q51H (639) | Q51S (640) |
| Q51T (641) | Q51N (642) | E53Q (25) | E53H (26) | E53N (27) |
| L57I (30) | L57V (31) | L57T (555) | L57Q (556) | L57H (557) |
| L57A (558) | Y60H (32) | Y60I (33) | E61Q (34) | E61H (35) |
| E61N (36) | L63I (39) | L63V (40) | L63T (562) | L63Q (563) |
| L63H (564) | L63A (565) | Q64H (643) | Q64S (644) | Q64T (645) |
| Q64N (646) | F67I (173) | F67V (174) | F70I (41) | F70V (42) |
| Q72H (647) | Q72S (648) | Q72T (649) | Q72N (650) | E81N (567) |
| E81Q (180) | E81H (181) | E85N (568) | E85Q (182) | E85H (183) |
| L87I (45) | L87V (46) | L87H (569) | L87A (570) | L87D (447) |
| L87E (448) | L87K (449) | L87R (450) | L87N (451) | L87Q (452) |
| L87S (453) | L87T (454) | L88I (47) | L88V (48) | L88T (571) |
| L88Q (572) | L88H (573) | L88A (574) | Y92H (184) | Y92I (185) |
| Q94D (474) | Q94E (475) | Q94K (476) | Q94N (477) | Q94R (478) |
| Q94S (479) | Q94T (480) | Q94C (656) | L98I (49) | L98V (50) |
| L98H (575) | L98A (576) | L98D (497) | L98E (498) | L98K (499) |
| L98N (500) | L98Q (501) | L98R (502) | L98S (503) | L98T (504) |
| L98C (658) | L102I (51) | L102V (52) | L102T (578) | L102Q (579) |
| L102H (580) | L102A (581) | E103N (582) | E103Q (190) | E103H (191) |
| E104N (583) | E104Q (192) | E104H (193) | L106I (53) | L106V (54) |
| L106T (585) | L106Q (586) | L106H (587) | L106A (588) | E107N (589) |
| E107Q (198) | E107H (199) | E109N (591) | E109H (205) | E109Q (204) |
| F111I (209) | F111V (210) | L116V (213) | L116I (214) | L116T (215) |
| L116Q (216) | L116H (217) | L116A (218) | L120V (219) | L120I (220) |
| L120T (221) | L120Q (222) | L120H (223) | L120A (224) | Y125H (61) |
| Y125I (62) | Y126H (63) | Y126I (64) | L130V (233) | L130I (234) |
| L130T (235) | L130Q (236) | L130H (237) | L130A (238) | Y132H (65) |
| Y132I (66) | L133I (67) | L133V (68) | L133T (604) | L133Q (605) |
| L133H (606) | L133A (607) | E137N (610) | E137Q (247) | E137H (248) |
| Y138H (69) | Y138I (70) | E149Q (75) | E149H (76) | E149N (77) |
| L151I (78) | L151V (79) | L151T (611) | L151Q (612) | L151H (613) |
| L151A (614) | F154I (80) | F154V (81) | F156I (82) | F156V (83) |
| L160I (84) | L160V (85) | L160T (615) | L160Q (616) | L160H (617) |
| L160A (618) | Y163H (249) | Y163I (250) | L164I (86) | L164V (87) |
| L164T (619) | L164Q (620) | L164H (621) | L164A (622) | |

A modified IFN-β polypeptide provided herein that exhibits increased protease resistance to gelatinase B can contain one or more amino acid modifications corresponding to modifications selected from any of Y3H, Y3I, L5V, L5I, L5T, L5Q, L5H, L5A, L5D, L5E, L5K, L5R, L5N, L5S, L6I, L6V, L6H, L6A, L6D, L6E, L6K, L6N, L6Q, L6R, L6S, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, F15I, F15V, F15D, F15E, F15K, F15R, Q16D, Q16E, Q16K, Q16N, Q16R, Q16S, Q16T, Q16C, Q18H, Q18S, Q18T, Q18N, L20I, L20V, L20H, L20A, L20N, L20Q, L20R, L20S, L20T, L20D, L20E, L20K, L21I, L21V, L21T, L21Q, L21H, L21A, Q23H, Q23S, Q23T, Q23N, Q23D, Q23E, Q23K, Q23R, L28V, L28I, L28T, L28Q, L28H, L28A, E29N, E29Q, E29H, Y30H, Y30I, L32V, L32I, L32T, L32Q, L32H, L32A, F38I, F38V, E42N, E42Q, E42H, E43Q, E43H, E43N, L47V, L47I, L47T, L47Q, L47H, L47A, Q48H, Q48S, Q48T, Q48N, Q49H, Q49S, Q49T, Q49N, F50I, F50V, Q51H, Q51S, Q51T, Q51N, E53Q, E53H, E53N, L57I, L57V, L57T, L57Q, L57H, L57A, Y60H, Y60I, E61Q, E61H, E61N, L63I, L63V, L63T, L63Q, L63H, L63A, Q64H, Q64S, Q64T, Q64N, F67I, F67V, F70I, F70V, Q72H, Q72S, Q72T, Q72N, E81N, E81Q, E81H, E85N, E85Q, E85H, L87I, L87V, L87H, L87A, L87D, L87E, L87, L87R, L87N, L87Q, L87S, L87T, L88I, L88V, L88T, L88Q, L88H, L88A, Y92H, Y92I, Q94D, Q94E, Q94K, Q94N, Q94R, Q94S, Q94T, Q94C, L98I, L98V, L98H, L98A, L98D, L98E, L98K, L98N, L98Q, L98R, L98S, L98T, L98C, L102I, L102V, L102T, L102Q, L102H, L102A, E103N, E103Q, E103H, E104N, E104Q, E104H, L106I, L106V, L106T, L106Q, L106H, L106A, E107N, E107Q, E107H, E109N, E109H, E109Q, F111I, F111V, L116V, L116I, L116T, L116Q, L116H, L116A. L116V, L116I, L116T, L116Q, L116H, L116A, Y125H, Y125I, Y126H, Y126I, L130V, L130I, L130T, L130Q, L130H, L130A, Y132H, Y132I, L133I, L133V, L133T, L133Q, L133H, L133A, E137N, E137Q, E137H, Y138H, Y138I, E149Q, E149H, E149N, L151Q, L151V, L151T, L151Q, L151H, L151A, F154I, F154V, F156I, F156V, L160I, L160V, L160T, L160Q, L160H, L160A, Y163H, Y163I, L164I, L164V, L164T, L164Q, L164H, and L164A of a mature IFN-β polypeptide set forth in SEQ ID NO:1. In some examples, the modifications are in an IFN-β polypeptide having a sequence of amino acids set forth in SEQ ID NO:1 or SEQ ID NO:3. Exemplary modified IFN-β candidate LEAD polypeptides are set forth in any one of SEQ ID NOS: 4-11, 16, 17, 20-27, 30-36, 39-42, 45-54, 61-70, 75-87, 157, 158, 163-168, 173, 174, 180-185, 190-193, 198, 199, 204, 205, 209, 210, 213-224, 233-238, 247-250, 266-279, 282, 283, 295-310, 328-358, 377-387, 396-403, 408-411, 447-454, 474-479, 497-504, 540-542, 547, 551, 555-558, 562-576, 578-583, 585-589, 591, 604-607, 610-614, 616-650, 652, 653, 655, 656, and 658.

In one example, modified IFN-β polypeptides exhibit increased protease resistance to gelatinase B compared to an unmodified IFN-β polypeptide of SEQ ID NO:1 and contain one or more amino acid modifications corresponding to modification of any of Y3I, Q18S, Q18N, L20I, L20V, E42N, E43N, Q48H, Q48S, Q48T, Q49H, Q49S, Q49T, F50I, F50V, Q51H, Q51S, Q51T, Q51N, E53N, L57I, Y60I, E61H, E61N, L63I, Q64H, Q64S, Q64T, F70I, F70V, Q72H, Q72S, E85N, L88I, L88V, L98I, L98V, E103N, E104N, L106I, L106V, E107N, E109N, Y125I, Y126I, Y132I, E149H, E149N, L151I, and F154V of a mature IFN-β polypeptide set forth in SEQ ID NO:1. Exemplary modified IFN-β candidate LEAD polypeptides are set forth in any one of SEQ ID NOS: 5, 8, 9, 22-24, 27, 30, 33, 35, 36, 39, 41, 42, 47-50, 53, 54, 62, 64, 66, 76-78, 81, 551, 568, 582, 583, 589, 591, 624, 626, 631-633, 635-637, 639-645, 647, and 648. In another example, an IFN-β polypeptide exhibits increased resistance to gelatinase B compared to an unmodified IFN-β polypeptide of SEQ ID NO:3 and contains one or more amino acid modifications corresponding to any one or more modification of any of Y3I, Q18S, Q18N, L20I, L20V, L21I, L21V, E42N, E43N, Q48H, Q48S, Q48T, Q49H, Q49S, Q49T, F50I, F50V, Q51H, Q51S, Q51T, Q51N, E53N, L57I, Y60I, E61H, E61N, L63I, Q64H, Q64S, Q64T, F70I, F70V, Q72H, Q72S, E85N, L88I, L88V, L98I, L98V, E103N, E104N, L106I, L106V, E107N, E109N, Y125I, Y126I, Y132I, E149H, E149N, L151I, F154V, L160I, and L160V of a mature IFN-β polypeptide set forth in SEQ ID NO:1. Additionally, a modified IFN-β containing any one or more modification as set forth above can contain a further modification. Generally, the resulting polypeptide exhibits increased resistance to gelatinase B and retains one or more activities of the unmodified IFN-β. The further modification can be one or more replacement(s) at an amino acid position corresponding to any of positions 3, 5, 6, 8, 9, 10, 15, 16, 18, 20, 21, 23, 28, 29, 30, 32, 38, 42, 43, 47, 48, 49, 53, 57, 60, 61, 63, 64, 67, 72, 81, 85, 87, 88, 92, 94, 98, 102, 103, 104, 106, 107, 109, 111, 116, 120, 125, 126, 130, 132, 133, 137, 138, 149, 151, 154, 156, 160, 163, and 164 of a mature IFN-β polypeptide set forth in SEQ ID NO:1. Amino acid modifications can occur at one or more of amino acid positions corresponding to any of positions Y3, L5, L6, F8, L9, Q10, F15, Q16, Q18, L20, L21, W22, Q23, L28, E29, Y30, L32, F38, E42, E43, L47, Q48, Q49, E53, L57, Y60, E61, L63, Q64, F67, Q72, E81, E85, L87, L88, Y92, Q94, L98, L102, E103, E104, L106, E107, E109, F111, L1116, L120, Y125, Y126, L130, Y132, L133, E137, Y138, E149, L151, F154, F156, L160, Y163, and L164 of a mature IFN-β polypeptide set forth in SEQ ID NO:1. For example, the further amino acid modification can be any one or more modification corresponding to modification of any of Y3H, L5V, L5I, L5T, L5Q, L5H, L5A, L5D, L5E, L5K, L5R, L5N, L5S, L6I, L6V, L6H, L6A, L6D, L6E, L6K, L6N, L6Q, L6R, L6S, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, F15I, F15V, F15D, F15E, F15K, F15R, Q16D, Q16E, Q16K, Q16N, Q16R, Q16S, Q16T, Q16C, Q18H, Q18T, L20H, L20A, L20N, L20Q, L20R, L20S, L20T, L20D, L20E, L20K, L21I, L21V, L21T, L21Q, L21H, L21A, Q23H, Q23S, Q23T, Q23N, Q23D, Q23E, Q23K, Q23R, L28V, L28I, L28T, L28Q, L28H, L28A, E29N, E29Q, E29H, Y30H, Y30I, L32V, L32I, L32T, L32Q, L32H, L32A, F38I, F38V, E42Q, E42H, E43Q, E43H, L47V, L47I, L47T, L47Q, L47H, L47A, Q48N, Q49N, E53Q, E53H, L57V, L57T, L57Q, L57H, L57A, Y60H, E61Q, L63V, L63T, L63Q, L63H, L63A, Q64N, F67I, F67V, Q72T, Q72N, E81N, E81Q, E81H, E85Q, E85H, L87I, L87V, L87H, L87A, L87D, L87E, L87, L87R, L87N, L87Q, L87S, L87T, L88T, L88Q, L88H, L88A, Y92H, Y92I, Q94D, Q94E, Q94K, Q94N, Q94R, Q94S, Q94T, Q94C, L98H, L98A, L98D, L98E, L98K, L98N, L98Q, L98R, L98S, L98T, L98C, L102I, L102V, L102T, L102Q, L102H, L102A, E103Q, E103H, E104Q, E104H, L106T, L106Q, L106H, L106A, E107Q, E107H, E109H, E109Q, F111I, F111V, L116V, L116I, L116T, L116Q, L116H, L116A. L116V, L116I, L116T, L116Q, L116H, L116A, Y125H, Y126H, L130V, L130I, L130T, L130Q, L130H, L130A, Y132H, L133I, L133V, L133T, L133Q, L133H, L133A, E137N, E137Q, E137H, Y138H, Y138I, E149Q, L151V, L151T, L151Q, L151H, L151A, F154I, F156I, F156V, L160I, L160V, L160T, L160Q, L160H, L160A, Y163H, Y163I, L164I, L164V, L164T, L164Q, L164H, and L164A of a mature IFN-β polypeptide set forth in SEQ ID NO:1.

In another example, an IFN-β polypeptide provided herein exhibiting increased protease resistance to gelatinase B can contain any two or more amino acid modifications. In one example, the two or more amino acid modifications can by any two or more modifications set forth in Table 7 above. The modifications can be in an unmodified human IFN-β polypeptide set forth in SEQ ID NO:1 or 3. For example, an IFN-β polypeptide can contain two or more amino acid modifications corresponding to any two or more modifications of any of Y3H, Y3I, L5V, L5I, L5T, L5Q, L5H, L5A, L5D, L5E, L5K, L5R, L5N, L5S, L6I, L6V, L6H, L6A, L6D, L6E, L6K, L6N, L6Q, L6R, L6S, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, F15I, F15V, F15D, F15E, F15K, F15R, Q16D, Q16E, Q16K, Q16N, Q16R, Q16S, Q16T, Q16C, Q18H, Q18S, Q18T, Q18N, L20I, L20V, L20H, L20A, L20N, L20Q, L20R, L20S, L20T, L20D, L20E, L20K, L21I, L21V, L21T, L21Q, L21H, L21A, Q23H, Q23S, Q23T, Q23N, Q23D, Q23E, Q23K, Q23R, L28V, L28I, L28T, L28Q, L28H, L28A, E29N, E29Q, E29H, Y30H, Y30I, L32V, L32I, L32T, L32Q, L32H, L32A, F38I, F38V, E42N, E42Q, E42H, E43Q, E43H, E43N, L47V, L47I, L47T, L47Q, L47H, L47A, Q48H, Q48S, Q48T, Q48N, Q49H, Q49S, Q49T, Q49N, F50I, F50V, Q51H, Q51S, Q51T, Q51N, E53Q, E53H, E53N, L57I, L57V, L57T, L57Q, L57H, L57A, Y60H, Y60I, E61Q, E61H, E61N, L63I, L63V, L63T, L63Q, L63H, L63A, Q64H, Q64S, Q64T, Q64N, F67I, F67V, F70I, F70V, Q72H, Q72S, Q72T, Q72N, E81N, E81Q, E81H, E85N, E85Q, E85H, L87I, L87V, L87H, L87A, L87D, L87E, L87, L87R, L87N, L87Q, L87S, L87T, L88I, L88V, L88T, L88Q, L88H, L88A, Y92H, Y92I, Q94D, Q94E, Q94K, Q94N, Q94R, Q94S, Q94T, Q94C, L98I, L98V, L98H, L98A, L98D, L98E, L98K, L98N, L98Q, L98R, L98S, L98T, L98C, L102I, L102V, L102T, L102Q, L102H, L102A, E103N, E103Q, E103H, E104N, E104Q, E104H, L106I, L106V, L106T, L106Q, L106H, L106A, E107N, E107Q, E107H, E109N, E109H, E109Q, F111I, F111V, L116V, L116I, L116T, L116Q, L116H, L116A. L116V, L116I, L116T, L116Q, L116H, L116A, Y125H, Y125I, Y126H, Y126I, L130V, L130I, L130T, L130Q, L130H, L130A, Y132H, Y132I, L133I, L133V, L133T, L133Q, L133H, L133A, E137N, E137Q, E137H, Y138H, Y138I, E149Q, E149H, E149N, L151I, L151V, L151T, L151Q, L151H, L151A, F154I, F154V, F156I, F156V, L160I, L160V, L160T, L160Q, L160H, L160A, Y163H, Y163I, L164I, L164V, L164T, L164Q, L164H, and L164A of a mature IFN-β polypeptide set forth in SEQ ID NO:1. Generally, the resulting IFN-β polypeptide exhibits increased protease resistance to gelatinase B and retains one more activities of an unmodified IFN-β polypeptide. In an additional example, an IFN-β polypeptide provided herein can contain any one or more modification set forth in Table 7 above and a further modification or modifications. In one example, the further modification can be one or more replacement(s) at an amino acid position corresponding to any of positions 1, 11, 12, 13, 14, 17, 19, 22, 24, 25, 27, 33, 34, 35, 36, 39, 41, 45, 50, 51, 52, 54, 62, 70, 71, 73, 78, 79, 80, 82, 83, 86, 89, 90, 91, 95, 97, 99, 101, 105, 108, 110, 113, 115, 117, 122, 123, 124, 128, 134, 136, 143, 147, 152, 155, 159, and 165 of a mature IFN-β polypeptide set forth in SEQ ID NO:1. Amino acid modifications can occur at one or more of amino acid positions corresponding to any of positions M1, R11, S12, S13, N14, C17, K19, W22, L24, N25, R27, K33, D34, R35, M36, D39, P41, K45, F50, Q51, K52, D54, M62, F70, R71, D73, G78, W79, N80, T82, I83, N86, A89, N90, V91, I95, H97, K99, V101, K105, K108, D110, R113, K115, M117, L122, K123, R124, R128, K134, K136, W143, R147, R152, Y155, R159, and R165 of a mature IFN-β polypeptide set forth in SEQ ID NO:1. For example, the further amino acid modification can correspond to any one or more of M1V, M1I, M1T, M1A, M1Q, M1D, M1E, M1K, M1N, M1R, M1S, M1C, R11D, R11H, R11Q, S12D, S12E, S12K, S12R, S13D, S13E, S13K, S13N, S13Q, S13R, S13T, S13C, N14D, N14E, N14K, N14Q, N14R, N14S, N14T, C17D, C17E, C17K, C17N, C17Q, C17R, C17S, C17T, K19N, K19Q, K19T, K19S, K19H, W22S, W22H, W22D, W22E, W22K, W22R, L24I, L24V, L24T, L24Q, L24H, L24A, L24D, L24E, L24K, L24R, N25H, N25S, N25Q, R27H, R27Q, K33N, K33Q, K33T, K33S, K33H, D34N, D34Q, D34G, R35H, R35Q, M36V, M36I, M36T, M36Q, M36A, D39N, D39Q, D39H, D39G, P41A, P41S, K45D, K45N, K45Q, K45T, K45S, K45H, F50I, F50V, Q51H, Q51S, Q51T, Q51N, K52D, K52N, K52Q, K52T, K52S, K52H, D54K, D54Q, D54N, D54G, M62I, M62V, M62T, M62Q, M62A, F70I, F70V, R71H, R71Q, D73N, D73Q, D73H, D73G, G78D, G78E, G78K, G78R, W79H, W79S, W79D, W79E, W79K, W79R, N80D, N80E, N80K, N80R, T82D, T82E, T82K, T82R, I83D, I83E, I83K, I83R, I83Q, I83S, I83T, N86D, N86E, N86K, N86R, N86Q, N86S, N86T, A89D, A89E, A89K, A89R, N90D, N90E, N90K, N90Q, N90R, N90S, N90T, N90C, V91D, V91E, V91K, V91N, V91Q, V91R, V91S, V91T, V91C, I95D, I95E, I95K, I95N, I95Q, I95R, I95S, I95T, H97D, H97E, H97K, H97N, H97Q, H97R, H97S, H97T, H97C, K99N, K99Q, K99T, K99S, K99H, V101D, V101E, V101K, V101N, V101Q, V10R, V101S, V101T, V101C, K105D, K105N, K105Q, K105T, K105S, K105H, K108D, K108N, K108Q, K108T, K108S, K108H, D110K, D110N, D110Q, D110H, D110G, R113E, R113H, R113Q, K115D, K115Q, K115N, K115S, K115H, M117I, M117V, M117T, M117Q, M117A, L122I, L122V, L122T, L122Q, L122H, L122A, K123N, K123Q, K123T, K123S, K123H, R124D, R124E, R124H, R124Q, R128H, R128Q, K134N, K134Q, K134T, K134S, K134H, K136N, K136Q, K136T, K136S, K136H, W143H, W143S, R147H, R147Q, R152D, R152H, R152Q, Y155H, Y1551, R159H, R159Q, R165D, R165H, and R165Q of a mature IFN-β polypeptide set forth in SEQ ID NO:1. Non-limiting examples of IFN-β SuperLeads containing two or more amino acid modifications and exhibiting increased resistance to proteolysis by gelatinase B are described in Example 8 and include amino acid replacements at amino acid residues corresponding to L6E/K108S, L5Q/K108S, L5E/K108S, L5N/Q10D, and L5N/K108S of a mature IFN-β polypeptide. Exemplary modified IFN-β SuperLead polypeptides are set forth in any one of SEQ ID NOS: 92, 102, 104, 107, and 112.

2. Conformational Stability

Also provided herein is a modified IFN-β polypeptide exhibiting increased protein stability manifested as increased conformational stability. Generally, the modification results in a polypeptide either improving or maintaining the requisite biological activity (e.g., anti-viral or anti-proliferation activity) of the unmodified IFN-β. Among modifications of interest for therapeutic proteins are those that increase the stability of IFN-β by minimizing denaturation from increased temperatures (i.e. thermal stability) and/or changes in pH. For example, increased conformational stability can be assayed by measuring resistance to temperature such as is described in Example 9. Increased conformational stability can increase the half-life of the protein for production, storage and for therapeutic use. Examples of modifications that contribute to the conformational stability of an IFN-β polypeptide include the addition of charges to a hydrophobic area (e.g., regions in helices A and C) to favor polar interactions with a solvent, increasing intra-molecular polar interactions between helices A and C, creating intra-molecular disulfide bridges, and changing the isoelectric point (pI). Variants with such modifications can be more stable to production conditions, allowing greater flexibility in the use of methods and conditions used to produce and purify the protein. Variants also can be more stable in storage, and thus therapeutic compositions can be prepared in advance and stored under a variety of conditions, making them more accessible therapies. Variants can be more stable for therapeutic use, for example, by increasing half-life (i.e., half-life in vitro, half-life in vivo, at room temperature or at 37° C.) after administration and/or by exhibiting greater stability in a variety of formulations and administration regimes.

The two-dimensional scanning process for protein evolution can be used to design and generate highly stable, longer lasting proteins, or proteins having a longer half-life. The method includes: i) identifying some or all possible target sites (i.e. is-HIT(s)) on the protein sequence that can participate in conformational stability such as, for example, sites that participate in the interaction and creation of the hydrophobic region of helices A and C and/or in the creation of disulfide bonds; ii) identifying appropriate replacing amino acids, specific for each is-HIT, such that upon replacement of one or more of the original, such as native, amino acids at that specific is-HIT, they can be expected to increase the is-HIT's stability while, at the same time, maintaining or improving the requisite biological activity and specificity of the protein (candidate LEADs); and/or iii) systematically introducing the specific replacing amino acids (candidate LEADs) at every specific is-HIT target position to generate a collection containing the corresponding mutant candidate LEAD molecules. Mutants are generated, produced and phenotypically characterized one-by-one in addressable assays, such as for example resistance to temperature, such that each mutant molecule contains, initially, an amino acid modification at only one is-HIT site. In particular embodiments, such as in subsequent rounds, mutant molecules also can be generated that contain multiple is-HIT sites that have been replaced by candidate LEAD amino acids (i.e. super-LEADs).

Using the methods described herein, the following is-HIT positions were identified to increase conformational stability of IFN-β: 1, 5, 6, 8, 9, 10, 11, 12, 13, 14, 15, 16, 17, 20, 22, 23, 24, 43, 45, 52, 53, 54, 61, 78, 79, 80, 81, 82, 83, 85, 86, 87, 89, 90, 91, 94, 95, 97, 98, 101, 103, 104, 105, 107, 108, 109, 110, 113, 115, 124, 152, and 165, corresponding to amino acid positions in a mature IFN-β polypeptide set forth in SEQ ID NO:1. As described in detail below, modification of any one or more of the above positions can contribute to increased conformational stability due to the addition of charges to a hydrophobic area (e.g., regions in helices A and C) to favor polar interactions with a solvent, increasing intra-molecular polar interactions between helices A and C, creating intra-molecular disulfide bridges, and changing the isoelectric point (pI). Exemplary amino acids modifications at is-HIT positions include any one or more amino acid modification corresponding to modifications of any of M1E, M1D, M1K, M1R, M1N, M1Q, M1S, M1T, M1C, L5E, L5D, L5K, L5R, L5N, L5Q, L5S, L5T, L6C, F8E, F8D, F8K, F8R, L9E, L9D, L9K, L9R, L9N, L9Q, L9S, L9T, Q10C, Q10E, Q10D, Q10K, Q10R, Q10N, Q10S, Q10T, R11Q, R11D, S12E, S12D, S12K, S12R, S13E, S13D, S13K, S13R, S13N, S13Q, S13T, S13C, N14E, N14D, N14K, N14R, N14Q, N14S, N14T, F15E, F15D, F15K, F15R, Q16E, Q16D, Q16K, Q16R, Q16N, Q16S, Q16T, Q16C, C17E, C17D, C17K, C17R, C17N, C17Q, C17S, C17T, L20E, L20D, L20K, L20R, L20N, L20Q, L20S, L20T, W22E, W22D, W22K, W22R, Q23E, Q23D, Q23K, Q23R, L24E, L24D, L24K, L24R, E43K, K45Q, K45D, K52Q, K52D, E53R, D54K, E61K, G78E, G78D, G78K, G78R, W79E, W79D, W79K, W79R, N80E, N80D, N80K, N80R, E81K, T82E, T82D, T82K, T82R, I83E, I83D, I83K, I83R, I83N, I83Q, I83S, I83T, E85K, N86E, N86D, N86K, N86R, N86Q, N86S, N86T, L87E, L87D, L87K, L87R, L87N, L87Q, L87S, L87T, A89E, A89D, A89K, A89R, N90E, N90D, N90K, N90R, N90Q, N90S, N90T, N90C, V91E, V91D, V91K, V91R, V91N, V91Q, V91S, V91T, V91C, Q94E, Q94D, Q94K, Q94R, Q94N, Q94S, Q94T, Q94C, I95E, I95D, I95K, I95R, I95N, I95Q, I95S, I95T, H97E, H97D, H97K, H97R, H97N, H97Q, H97S, H97T, H97C, L98E, L98D, L98K, L98R, L98N, L98Q, L98S, L98T, L98C, V101C, V101E, V101D, V101K, V101R, V101N, V101Q, V101S, V101T, E103K, E104R, K105Q, K105D, E107R, K108Q, K108D, E109R, D110K, R113Q, R113E, K115Q, K115D, R124Q, R124D, R124E, R152Q, R152D, R165Q, and R165D of a mature IFN-β polypeptide set forth in SEQ ID NO:1. In one example, modifications can be in an unmodified IFN-β polypeptide having a sequence of amino acids set forth in SEQ ID NO:1 or 3.

Modified IFN-β polypeptides provided herein exhibit increased resistance to denaturation, and thereby increased conformational stability. Such increase in resistance is manifested as at least 1%, 2%, 3%, 4%, 5%, 6%, 7%, 8%, 9%, 10%, . . . 20%, . . . 30%, . . . 40%, . . . 50%, . . . 60%, . . . , 70%, . . . 80%, . . . 90%, 91%, 92%, 93%, 94%, 95%, 96%, 97%, 98%, 99%, 100%, 200%, 300%, 400%, 500%, or more resistant to denaturation compared to the unmodified IFN-β polypeptide. In some examples, denaturation can be assessed as tolerance to temperature (temperature stability). Typically, the half-life in vitro or in vivo (protein stability) of the IFN-β mutants provided herein is increased by an amount selected from at least 5%, at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500% or more, when compared to the half-life of unmodified or wild-type human IFN-β exposed to particular denaturing conditions, such as thermal conditions (e.g., incubation at room temperature, about 25° C.; or body temperature, about 37° C.).

Generally, the modified IFN-β polypeptides provided herein exhibit at least one activity that is substantially unchanged (less than 1%, 5% or 10% changed) compared to the unmodified or wild-type IFN-β. In some examples, the modified IFN-β polypeptide exhibits a decrease in an activity. In other examples, a modified IFN-β polypeptide exhibits an increase in an activity. Activity includes, for example, anti-viral or anti-proliferative activity, and can be compared to an unmodified IFN-β polypeptide, such as for example the mature, wild-type IFN-β polypeptide (SEQ ID NO:1), the wild-type precursor IFN-β polypeptide (SEQ ID NO: 2), a commercially available IFN-β polypeptide, (e.g., Betaseron, SEQ ID No: 3), or any other IFN-β polypeptide used as the starting material.

a. Addition of Charged Residues to Hydrophobic Areas

Regions of helices A and C of IFN-β form a hydrophobic interface and are protected from exposure to solvent due to glycosylation of the folded polypeptide. There is an N-glycosylation site at position N80. The glycan present on the protein is an oligosaccharide chain of the biantennary complex type, containing an α1-6 linked fucose on the peptide proximal N-acetyl-glucosamine (GlcNac) residue and two α2-3 linked N-acetyl-neuraminic (NANA) on the terminal galactose residues. This glycan possesses a rigid structure and interacts with two side chains of IFN-β via hydrogen bonds (Q23 in helix A and N86 in helix C). Changes in the hydrophobic region of helices A and C can be made to favor polar interactions with the solvent, thereby stabilizing the protein conformation. Additionally, the hydrophobic region of helices A and C also plays a role in the specificity of action of IFN-β. Changes in this region can alter IFN-β activity, for example, by modifying IFN-β activity towards IFN-α activity. Modified polypeptides can be assessed for increased stability, for example, by increased thermal tolerance, such as described herein. In addition, assays that discriminate between IFN-α and IFN-β can be used to assess whether the mutation also affects IFN-β specificity. Any assays known in the art to assess IFN-α and IFN-β activity can be employed.

Using the methods described herein, the following is-HIT positions were identified to increase conformational stability due to the addition of charges to the hydrophobic regions of helices A and C of IFN-β: 5, 8, 9, 12, 15, 16, 20, 22, 23, 24, 78, 79, 80, 82, 83, 86, 87 and 89, corresponding to positions of a mature IFN-β polypeptide set forth in SEQ ID NO:1. Amino acid modifications can occur at one or more of amino acid positions corresponding to any of positions L5, F8, L9, S12, F15, Q16, L20, W22, Q23, L24, G78, W79, N80, T82, I83, N86, L87, and A89 of a mature IFN-β polypeptide set forth in SEQ ID NO:1. Modification of one or more amino acid residues thereof can add charges to the hydrophobic regions of helices A and C. Exemplary amino acids chosen to introduce an additional charge into the region of helices A and C include glutamic acid (E), aspartic acid (D), lysine (K) and arginine (R). Table 8 provides non-limiting examples of amino acid modifications that increase conformational stability compared to an unmodified IFN-β polypeptide by adding charges to the hydrophobic regions of helices A and C. Generally, a resulting modified polypeptide retains one or more activities of an unmodified IFN-β polypeptide. In one example, modifications can be an unmodified IFN-β polypeptide having a sequence of amino acids set forth in SEQ ID NO:1 or SEQ ID NO:3. In Table 8 below, the sequence identifier (SEQ ID NO:) is in parenthesis next to each substitution.

TABLE 8
Modifications to Add Charged Residues to Increase
Polar Interactions With Solvent

| | | | |
|---|---|---|---|
| L5E (329) | Q16E (382) | G78E (417) | N86E (441) |
| L5D (328) | Q16D (381) | G78D (416) | N86D (440) |
| L5K (330) | Q16K (383) | G78K (418) | N86K (442) |
| L5R (331) | Q16R (385) | G78R (419) | N86R (443) |
| F8E (343) | L20E (397) | W79E (421) | L87E (448) |
| F8D (342) | L20D (401) | W79D (420) | L87D (447) |
| F8K (344) | L20K (403) | W79K (422) | L87K (449) |
| F8R (345) | L20R (398) | W79R (423) | L87R (450) |
| L9E (347) | W22E (405) | N80E (425) | A89E (456) |
| L9D (346) | W22D (404) | N80D (424) | A89D (455) |
| L9K (348) | W22K (406) | N80K (426) | A89K (457) |
| L9R (350) | W22R (407) | N80R (427) | A89R (458) |
| S12E (360) | Q23E (409) | T82E (429) | |
| S12D (359) | Q23D (408) | T82D (428) | |
| S12K (361) | Q23K (410) | T82K (430) | |
| S12R (362) | Q23R (411) | T82R (431) | |
| F15E (378) | L24E (413) | I83E (433) | |
| F15D (377) | L24D (412) | I83D (432) | |
| F15K (379) | L24K (414) | I83K (434) | |
| F15R (380) | L24R (415) | I83R (435) | |

Modified IFN-β polypeptides provided herein that exhibit increased conformational stability due to the presence of charges in the hydrophobic region between helices A and C can contain one or more amino acid modification corresponding to any one or more modification of L5E, L5D, L5K, L5R, F8E, F8D, F8K, F8R, L9E, L9D, L9K, L9R, S12E, S12D, S12K, S12R, F15E, F15D, F15K, F15R, Q16E, Q16D, Q16K, Q16R, L20E, L20D, L20K, L20R, W22E, W22D, W22K, W22R, Q23E, Q23D, Q23K, Q23R, L24E, L24D, L24K, L24R, G78E, G78D, G78K, G78R, W79E, W79D, W79K, W79R, N80E, N80D, N80K, N80R, T82E, T82D, T82K, T82R, I83E, I83D, I83K, I83R, N86E, N86D, N86K, N86R, L87E, L87D, L87K, L87R, A89E, A89D, A89K, and A89R of a mature IFN-β polypeptide set forth in SEQ ID NO:1. In some examples, the modifications can be in an unmodified IFN-β polypeptide having a sequence of amino acids set forth in SEQ ID NO:1 or SEQ ID NO:3. Exemplary modified IFN-β candidate LEAD polypeptides are set forth in any one of SEQ ID NOS: 329-331, 342-348, 350, 359-362, 377-383, 385 397-398, 401, 403-435, 440-443, 447-450, and 455-458.

In another example, an IFN-β polypeptide provided herein exhibiting increased conformational stability due to the presence of charges in the hydrophobic region between helices A and C can contain any two or more amino acid modifications. In one example, the two or more amino acid modifications can be any two or more modifications set forth in Table 8 above. The amino acid modifications can be in an unmodified IFN-β polypeptide, such as for example an unmodified IFN-β polypeptide set forth in SEQ ID NO:1 or 3. For example, an IFN-β polypeptide can contain two or more amino acid modifications corresponding to any two or more modifications of L5E, L5D, L5K, L5R, F8E, F8D, F8K, F8R, L9E, L9D, L9K, L9R, S12E, S12D, S12K, S12R, F15E, F15D, F15K, F15R, Q16E, Q16D, Q16K, Q16R, L20E, L20D, L20K, L20R, W22E, W22D, W22K, W22R, Q23E, Q23D, Q23K, Q23R, L24E, L24D, L24K, L24R, G78E, G78D, G78K, G78R, W79E, W79D, W79K, W79R, N80E, N80D, N80K, N80R, T82E, T82D, T82K, T82R, I83E, I83D, I83K, I83R, N86E, N86D, N86K, N86R, L87E, L87D, L87K, L87R, A89E, A89D, A89K, and A89R of a mature IFN-β polypeptide set forth in SEQ ID NO: 1. Generally, the resulting IFN-β polypeptide exhibits increased conformational stability and retains one more activities of an unmodified IFN-β polypeptide.

b. Increasing Polar Interactions Between Helices A and C

The conformational stability of an IFN-β polypeptide also can be achieved by changes in helices A and C that increase polar interactions between the helices. As described above, the hydrophobic area at the vicinity of the glycosylation site is formed by the interface between helices A and C. There are few polar interactions between the residues of these helices. This renders this region susceptible to denaturation. For example, in the absence of glycosylation, the protein could be destabilized and this could lead to the opening of the interface between helices A and C and thus expose a cysteine residue at position 17 that can become reactive and contribute to the formation of intermolecular disulfide bonds and possibly protein aggregation. Increasing polar interactions between helices A and C can increase the conformational stability and, thereby, the stability and half-life of IFN-β.

Using the methods described herein, the following is-HIT positions were identified to increase conformational stability by increasing polar interactions between helices A and C of IFN-β: 1, 5, 6, 9, 10, 13, 14, 16, 17, 20, 83, 86, 87, 90, 91, 94, 95, 97, 98, and 101, corresponding to amino acid positions of a mature IFN-β polypeptide set forth in SEQ ID NO:1. Amino acid modifications can occur at one or more of amino acid positions corresponding to any of positions M1, L5, L6, L9, Q10, S13, N14, Q16, C17, L20, I83, N86, L87, N90, V91, Q94, I95, H97, L98, and V101 of a mature IFN-β polypeptide set forth in SEQ ID NO:1. Modification of one or more amino acid residues thereof can contribute to polar interactions between helices A and C. Exemplary amino acids chosen to increase polar interactions between the region of helices A and C include glutamic acid (E), aspartic acid (D), lysine (L), arginine (R), asparagine (N), glutamine (Q), serine (S), and threonine (T). Table 9 provides non-limiting examples of amino acid modifications that contribute to increased conformational stability of an IFN-β polypeptide by increasing polar interactions between helices A and C and, thereby, protein stability. Generally, a resulting modified polypeptide retains one or more activities of the unmodified IFN-β polypeptide. In Table 9 below, the sequence identifier (SEQ ID NO:) is in parenthesis next to each substitution.

TABLE 9
Modifications to Increase Polar Interactions Between Helices A and C

| | | | | | |
|---|---|---|---|---|---|
| M1E (323) | L9K (348) | N14T (376) | I83K (434) | N90S (464) | H97D (489) |
| M1D (322) | L9R (350) | Q16E (382) | I83R (435) | N90T (465) | H97K (491) |
| M1K (324) | L9N (349) | Q16D (381) | I83N (436) | V91E (467) | H97R (494) |
| M1R (326) | L9Q (277) | Q16K (383) | I83Q (437) | V91D (466) | H97N (492) |
| M1N (325) | L9S (351) | Q16R (385) | I83S (438) | V91K (468) | H97Q (493) |
| M1Q (261) | L9T (276) | Q16N (384) | I83T (439) | V91R (471) | H97S (495) |
| M1S (327) | Q10E (353) | Q16S (386) | N86E (441) | V91N (469) | H97T (496) |
| M1T (264) | Q10D (352) | Q16T (387) | N86D (440) | V91Q (470) | L98E (498) |
| L5E (329) | Q10K (354) | C17E (389) | N86K (442) | V91S (472) | L98D (497) |
| L5D (328) | Q10R (356) | C17D (388) | N86R (443) | V91T (473) | L98K (499) |
| L5K (330) | Q10N (355) | C17K (390) | N86Q (444) | Q94E (475) | L98R (502) |
| L5R (331) | Q10S (357) | C17R (393) | N86S (445) | Q94D (474) | L98N (500) |
| L5N (332) | Q10T (358) | C17N (391) | N86T (446) | Q94K (476) | L98Q (501) |
| L5Q (269) | S13E (364) | C17Q (392) | L87E (448) | Q94R (478) | L98S (503) |
| L5S (333) | S13D (363) | C17S (394) | L87D (447) | Q94N (477) | L98T (504) |
| L5T (268) | S13K (365) | C17T (395) | L87K (449) | Q94S (479) | V101E (506) |
| L6E (335) | S13R (368) | L20E (397) | L87R (450) | Q94T (480) | V101D (505) |
| L6D (334) | S13N (366) | L20D (401) | L87N (451) | I95E (482) | V101K (507) |
| L6K (336) | S13Q (367) | L20K (403) | L87Q (452) | I95D (481) | V101R (510) |
| L6R (339) | S13T (369) | L20R (398) | L87S (453) | I95K (483) | V101N (508) |
| L6N (337) | N14E (371) | L20N (396) | L87t (454) | I95R (486) | V101Q (509) |
| L6Q (338) | N14D (370) | L20Q (402) | N90E (460) | I95N (484) | V101S (511) |
| L6S (340) | N14K 372) | L20S (399) | N90D (459) | I95Q (485) | V101T (512) |
| L6T (341) | N14R (374) | L20T (400) | N90K (461) | I95S (487) | |
| L9E (347) | N14Q (373) | I83E (433) | N90R (463) | I95T (488) | |
| L9D (346) | N14S (375) | I83D (432) | N90Q (462) | H97E (490) | |

Modified IFN-β polypeptide provided herein that exhibit increased conformational stability due to increased polar interactions between helices A and C can contain one or more amino acid modifications corresponding to any one or more modification of M1E, M1D, M1K, M1R, M1N, M1Q, M1S, M1T, L5E, L5D, L5K, L5R, L5N, L5Q, L5S, L5T, L6E, L6D, L6K, L6R, L6N, L6Q, L6S, L6T, L9E, L9D, L9K, L9R, L9N, L9Q, L9S, L9T, Q10E, Q10D, Q10K, Q10R, Q10N, Q10S, Q10T, S13E, S13D, S13K, S13R, S13N, S13Q, S13T, N14E, N14D, N14K, N14R, N14Q, N14S, N14T, Q16E, Q16D, Q16K, Q16R, Q16N, Q16S, Q16T, C17E, C17D, C17K, C17R, C17N, C17Q, C17S, C17T, L20E, L20D, L20K, L20R, L20N, L20Q, L20S, L20T, I83E, I83D, I83K, I83R, I83N, I83Q, I83S, I83T, N86E, N86D, N86K, N86R, N86Q, N86S, N86T, L87E, L87D, L87K, L87R, L87N, L87Q, L87S, L87T, N90E, N90D, N90K, N90R, N90Q, N90S, N90T, V91E, V91D, V91K, V91R, V91N, V91Q, V91S, V91T, Q94E, Q94D, Q94K, Q94R, Q94N, Q94S, Q94T, I95E, I95D, I95K, I95R, I95N, I95Q, I95S, I95T, H97E, H97D, H97K, H97R, H97N, H97Q, H97S, H97T, L98E, L98D, L98K, L98R, L98N, L98Q, L98S, L98T, V101E, V101D, V101K, V101R, V101N, V101Q, V101S, and V101T of a mature IFN-β polypeptide set forth in SEQ ID NO:1. In some examples, the modifications can be in an unmodified IFN-β polypeptide having a sequence of amino acids set forth in SEQ ID NO:1 or SEQ ID NO:3. Exemplary modified IFN-β candidate LEAD polypeptides are set forth in any one of SEQ ID NOS: 264, 268, 269, 276, 277, 322-341, 346-358, 363-376, 381-403, 432-454, and 459-512.

In another example, an IFN-β polypeptide provided herein exhibiting increased conformational stability due to increased polar interactions between helices A and C can contain any two or more amino acid modifications. The two or more amino acid modifications can by any two or more modifications set forth in Table 9 above. The two or more modifications can be in an unmodified IFN-β polypeptide, such as but not limited to, an unmodified IFN-β polypeptide set forth in SEQ ID NO:1 or 3. For example, an IFN-β polypeptide can contain two or more amino acid modifications corresponding to any two or more modifications of M1E, M1D, M1K, M1R, M1N, M1Q, M1S, M1T, L5E, L5D, L5K, L5R, L5N, L5Q, L5S, L5T, L6E, L6D, L6K, L6R, L6N, L6Q, L6S, L6T, L9E, L9D, L9K, L9R, L9N, L9Q, L9S, L9T, Q10E, Q10D, Q10K, Q10R, Q10N, Q10S, Q10T, S13E, S13D, S13K, S13R, S13N, S13Q, S13T, N14E, N14D, N14K, N14R, N14Q, N14S, N14T, Q16E, Q16D, Q16K, Q16R, Q16N, Q16S, Q16T, C17E, C17D, C17K, C17R, C17N, C17Q, C17S, C17T, L20E, L20D, L20K, L20R, L20N, L20Q, L20S, L20T, I83E, I83D, I83K, I83R, I83N, I83Q, I83S, I83T, N86E, N86D, N86K, N86R, N86Q, N86S, N86T, L87E, L87D, L87K, L87R, L87N, L87Q, L87S, L87T, N90E, N90D, N90K, N90R, N90Q, N90S, N90T, V91E, V91D, V91K, V91R, V91N, V91Q, V91S, V91T, Q94E, Q94D, Q94K, Q94R, Q94N, Q94S, Q94T, I195E, I95D, I95K, I95R, I95N, I95Q, I95S, I95T, H97E, H97D, H97K, H97R, H97N, H97Q, H97S, H97T, L98E, L98D, L98K, L98R, L98N, L98Q, L98S, L98T, V101E, V101D, V101K, V101R, V101N, V101Q, V101S, and V101T of a mature IFN-α polypeptide set forth in SEQ ID NO:1. Generally, the resulting IFN-β polypeptide exhibits increased conformational stability and retains one more activities of an unmodified IFN-β polypeptide.

c. Creation of Disulfide Bridges

Among modifications of interest for therapeutic proteins are those that increase conformational stability of IFN-β by minimizing denaturation and thus increasing the half-life of the protein for production, storage and for therapeutic use. One such type of modification is the introduction of intra-molecular disulfide bridges. For example, the risk of denaturation of an IFN-β polypeptide can be reduced by creating disulfide bridges between helices A and C. Introduction of disulfide bridges can minimize protein denaturation, such as denaturation from increased temperatures, and/or changes in pH.

Using the methods described herein, the following is-HIT positions were identified to increase conformational stability by introducing disulfide bridges in IFN-β: 1, 6, 10, 13, 16, 90, 91, 94, 97, 98, and 101, corresponding to amino acid positions of a mature IFN-β polypeptide set forth in SEQ ID NO:1. Amino acid modifications can occur at one or more amino acid positions corresponding to any of positions M1, L6, Q10, S13, Q16, N90, V91, Q94, H97, L98, and V101 of a mature IFN-β polypeptide set forth in SEQ ID NO:1. Modification of one or more amino acid residues thereof to a cysteine (C) can contribute to the formation of a disulfide bridge in an IFN-β polypeptide. Table 10 provides non-limiting examples of amino acid modifications that increase conformational stability by contributing to the formation of disulfide bridges between helices A and C and, thereby, protein stability of a modified IFN-β polypeptide. Generally, a resulting IFN-β polypeptide retains one or more activities of the unmodified IFN-β polypeptide. In one example, a modified IFN-β polypeptide containing one modification as set forth above can exhibit conformational stability by the formation of a disulfide bridge with a cysteine occurring in the native human IFN-β. For example, position C17 in a native human IFN-β, such as an IFN-β polypeptide having a sequence of amino acids set forth in SEQ ID NO:1, can contribute to the formation of a disulfide bridge. In another example, a modified IFN-β polypeptide containing two modifications as set forth above can exhibit conformational stability by the formation of a disulfide bridge between the modified cysteine residues. In Table 10 below, the sequence identifier (SEQ ID No.) is in parenthesis next to each substitution.

TABLE 10
Modification to Create Disulfide Bridges

| | |
|---|---|
| M1C (651) | V91C (131) |
| L6C (652) | Q94C (656) |
| Q10C (653) | H97C (657) |
| S13C (654) | L98C (658) |
| Q16C (655) | V101C (659) |
| N90C (129) | |

Modified IFN-β polypeptides provided herein that exhibit increased conformational stability due to the creation of intra-molecular disulfide bridges between helices A and C can contain one or more amino acid modifications corresponding to any one or more modifications of M1C, L6C, Q10C, S13C, Q16C, N90C, V91C, Q94C, H97C, L98C, and V101C of a mature IFN-β polypeptide set forth in SEQ ID NO:1. Generally, the resulting IFN-β polypeptide exhibits increased conformational stability and retains one or more activities of an unmodified IFN-β polypeptide. In one example, an unmodified IFN-β polypeptide has a sequence of amino acids set forth in SEQ ID NO:1 or 3. Exemplary modified IFN-β candidate LEAD polypeptides are set forth in any one of SEQ ID NOS: 129, 131, 651-659. In some examples, a modified IFN-β polypeptide contains two amino acid modifications such as any two modifications corresponding to any modifications of M1C, L6C, Q10C, S13C, Q16C, N90C, V91C, Q94C, H97C, L98C, and V101C of a mature IFN-α polypeptide set forth in SEQ ID NO:1. Exemplary IFN-β candidate LEAD polypeptides, containing one or more modifications set forth above, that contribute to the formation of an intra-molecular disulfide bridge are set forth in Table 11. In Table 11 below, the sequence identifier (SEQ ID No.) is in parenthesis next to each exemplary IFN-β LEAD polypeptide. Provided herein are modified IFN-β candidate LEAD polypeptides exhibiting increased conformational stability due to the formation of disulfide bridges having a sequence of amino acids set forth in any of SEQ ID NOS: 126-133.

TABLE 11
Exemplary IFN-β LEAD Polypeptides for the
Formation of Disulfide Bridges

| | |
|---|---|
| M1C-V101C (126) | Q10C-H97C (130) |
| Q16C-N90C (127) | C17C-V91C (131) |
| L6C-L98C (128) | Q10C-L98C (132) |
| C17C-N90C (129) | S13C-Q94C (133) |

d. Modification of the Isoelectric Point (pI)

Protein-protein interactions can affect protein stability. For example, protein multimerization can stabilize a protein by allowing protein surfaces (1) to interact, (2) to form a stable structure and/or (3) to be protected from interaction with solvent and other molecules. Protein-protein interactions, however, can contribute to aggregation. For example, upon denaturation of a protein, hydrophobic regions that are normally shielded from exposure to solvent can become exposed. As a result, the denatured protein aggregates by interaction of hydrophobic regions to protect exposure to solvent. Multimers formed in aggregation tend to be less structured and less stable. In some cases, protein aggregates are targeted for degradation. Thus, reducing aggregation is one method for increasing protein stability and protein half-life. The charged state of a protein can affect the ability of the protein to multimerize and/or aggregate. The isoelectric point (pI) is a measure of the charged state of a protein relative to pH such that at the pH of the isoelectric point, the protein is no longer charged and is neutral. The solubility of a protein is lowest at its isoelectric point. Thus, aggregation of a protein often occurs when the pH is close to the isoelectric point of a protein. For example, there are approximately 40 charged amino acids in IFN-β and the isoelectric point of IFN-β is 8.93. Aggregation of IFN-β has been observed at pH 6-7, but IFN-β is observed to have increased stability at pH 4. This increased stability could be related to surface charge of the protein at low pH. This is because a protein exposed to a pH that is lowered far below its pI will lose its negative charge and will have a predominantly positive charge. The like charges will repel each other and prevent the protein from aggregating as readily.

Modifications of interest contemplated herein are modifications that alter the isoelectric point of IFN-β, thereby contributing to the conformational stability of an IFN-β polypeptide at a more neutral pH of 6-7 by reducing protein aggregation, yet retaining one or more activities of an unmodified IFN-β polypeptide. In one example, the pI can be increased above its native pI of 8.93, such that, for example, the pI can be increased to greater than 9.0, 9.2, 9.4, 9.6, 9.8, 10.0, 10.2, 10.4, 10.6, 10.8, 11.0, 11.2, 11.4, 11.6, 11.8, 12.0. For example, the pI can be increased such that the same charged state observed at pH 4 is observed at a more neutral pH of 6 (i.e. pI 11) so that aggregation is minimized. In another example, the pI can be decreased such that at a neutral pH the protein exhibits a surface charge that prevents aggregation. In selecting is-HIT positions, positions situated in regions interacting with the receptor, IFNAR-1 (amino acid residues 64-73, 92-100 and 128-137 of SEQ ID NO:1) and IFNAR-2 (amino acid residues 15-42 and 148-158 of SEQ ID NO:1) were excluded as is-HITs to maintain IFN-β activity.

i. Increasing Isoelectric Point (pI)

Provided herein are modified IFN-β polypeptides exhibiting increased conformational stability due to an increased isoelectric point compared to an unmodified IFN-β polypeptide. In one example, modified IFN-β polypeptides provided herein contain one or more modifications that cause an increase in the pI of the modified IFN-β polypeptide by about 0.3 or 0.3 compared to an unmodified IFN-β polypeptide. Using the methods described herein, the following is-HIT positions were identified that contribute to increasing the isoelectric point of IFN-β by about 0.3 or 0.3: 43, 53, 54, 61, 81, 85, 103, 104, 107, 109, and 110, corresponding to amino acid positions of a mature IFN-β polypeptide set forth in SEQ ID NO:1. Amino acid modifications can occur at one or more of amino acid positions corresponding to any of positions E43, E53, D54, E61, E81, E85, E103, E104, E107, E109 and D110 of a mature IFN-β polypeptide set forth in SEQ ID NO:1. In some examples, modifications are in an unmodified IFN-β polypeptide having sequence of amino acids set forth in SEQ ID NO:1 or SEQ ID NO:3. Modification of one or more amino acid residues of a Glutamic Acid (E) or an Aspartic Acid (D) to a Lysine (K) or an Arginine (R) can contribute to an increase in the pI of an IFN-β polypeptide. Table 12 provides non-limiting examples of amino acid modifications that increase conformational stability of an IFN-β polypeptide by contributing to an increase in the isoelectric point and, thereby, protein stability. Generally, a resulting polypeptide retains one or more activities of the unmodified IFN-β polypeptide. In Table 12 below, the sequence identifier (SEQ ID No.) is in parenthesis next to each substitution.

TABLE 12
Modification to increase the pI about 0.3 or 0.3

| | |
|---|---|
| E43K (134) | E103K (140) |
| E53R (135) | E104R (141) |
| D54K (136) | E107R (142) |
| E61K (137) | E109R (143) |
| E81K (138) | D110K (144) |
| E85K (139) | |

Modified IFN-β polypeptides provided herein that exhibit increased conformational stability due to an increased isoelectric point can contain one or more amino acid modifications corresponding to any one or more modification of E43K, E53R, D54K, E61K, E81K, E85K, E103K, E104R, E107R, E109R, and D110K of a mature IFN-β polypeptide set forth in SEQ ID NO:1. Modifications can be in an unmodified IFN-β polypeptide having a sequence of amino acids set forth in SEQ ID NO:1 or SEQ ID NO:3. Exemplary modified IFN-β candidate LEAD polypeptides are set forth in any one of SEQ ID NOS: 134-144.

In one example, a modified IFN-β polypeptide that exhibits increased conformational stability due to an increased isoelectric point can contain one or more amino acid modifications corresponding to any one or both of D54K and E61K of a mature IFN-β polypeptide set forth in SEQ ID NO:1. Modifications can be an unmodified IFN-β polypeptide having a sequence of amino acids set forth in SEQ ID NO:1 or SEQ ID NO:3. Additionally, a modified IFN-β as set forth above can contain a further modification. The further modification can be any one or more amino acid modifications corresponding to any one or more modification of E43K, E53R, E81K, E85K, E103K, E104R, E104R, E107R, E109R, and D110K of a mature IFN-β polypeptide set forth in SEQ ID NO:1. In another example, a modified IFN-β polypeptide that exhibits increased conformational stability due to an increased isoelectric point can contain two or more amino acid modifications corresponding to any two or more modifications of E43K, E53R, D54K, E61K, E81K, E85K, E103K, E104R, E107R, E109R, and D110K of a mature IFN-β polypeptide set forth in SEQ ID NO:1. Generally, the resulting polypeptide exhibits increased conformational stability and retains one or more activities of the unmodified IFN-β.

ii. Decreasing Isoelectric Point (pI)

Provided herein are modified IFN-β polypeptides exhibiting increased conformational stability compared to an unmodified IFN-β polypeptide, due to a decreased isoelectric point. The pI of IFN-β can be decreased by replacing positively charged amino acids with non-charged amino acids. The pI of IFN-β also can be decreased by replacing positively charged amino acids with negatively charged amino acids. For example, arginine and lysine residues can be replaced with either aspartic acid or glutamic acid residues. In one non-limiting example, the pI of a modified IFN-β polypeptide is decreased such that the pI is less than the native IFN-β pI (i.e., 8.93). For example, the pI of a modified IFN-β polypeptide can be decreased to less than 8.9, 8.7, 8.5, 8.3, 8.1, 7.9, 7.7, 7.5, 7.3, 7.1, 6.9, 6.7, 6.5, 6.3, 6.1 or 5.9. In one example, modified IFN-β polypeptides provided herein can contain one or more modification that cause a decrease in the pI of the modified IFN-β polypeptide by about 0.55 or 0.5 compared to an unmodified IFN-β polypeptide.

Using the methods described herein, the following is-HIT positions were identified that contribute to decreasing the isoelectric point of IFN-β: 11, 45, 52, 105, 108, 113, 115, 124, 152, and 165, corresponding to amino acid positions of a mature IFN-β polypeptide set forth in SEQ ID NO:1. Amino acid modifications can occur at one or more of amino acid positions corresponding to any of positions R11, K45, K52, K105, K108, R113, K115, R124, R152, and R165 of a mature IFN-β polypeptide set forth in SEQ ID NO:1. In one example, modification of one or more amino acid residues of a Lysine (K) or an Arginine (R) to a Glutamine (Q) can contribute to a decrease in the pI of an IFN-β polypeptide by about 0.55 or 0.55. Table 13 provides non-limiting examples of amino acid modifications that increase conformational stability by contributing to a decrease in the isoelectric point by about 0.55 or 0.55 and, thereby, protein stability. In another example, modification of one or more amino acid residues of a Lysine (K) or an Arginine (R) to a Glutamic Acid (E) or an Aspartic Acid (D) can contribute to a decrease in the pI of an IFN-β polypeptide by about 0.2 or 0.2. Table 14 provides non-limiting examples of amino acid replacements that increase conformational stability by contributing to a decrease in the isoelectric point by about 0.2 or 0.2 and, thereby, protein stability. Generally, a resulting polypeptide retains one or more activities of the unmodified IFN-β polypeptide. In Tables 13 and 14 below, the sequence identifier (SEQ ID No.) is in parenthesis next to each substitution.

TABLE 13
Modification to decrease the pI about 0.55 or 0.55

| | |
|---|---|
| R11Q (281) | K115Q (56) |
| K45Q (159) | R124Q (230) |
| K52Q (169) | R152Q (256) |
| K105Q (194) | R165Q (252) |
| K108Q (200) | |
| R113Q (212) | |

TABLE 14
Modification to decrease the pI about 0.2 or 0.2

| | |
|---|---|
| R11D (145) | K115D (151) |
| K45D (146) | R124D (520) |
| K52D (147) | R124E (519) |
| K105D (148) | R152D (152) |
| K108D (149) | R165D (153) |
| R113E (150) | |

Modified IFN-β polypeptides provided herein that exhibit increased conformational stability due to a decreased isoelectric point can contain one or more amino acid modifications corresponding to any one or more modification of R11Q, R11D, K45Q, K45D, K52Q, K52D, K105Q, K105D, K108Q, K108D, R113Q, R113E, K115Q, K115D, R124Q, R124D, R124E, R152Q, R152D, R165Q, and R165D of a mature IFN-β polypeptide set forth in SEQ ID NO:1. In one example, modifications are in an unmodified IFN-β polypeptide having a sequence of amino acids set forth in SEQ ID NO:1 or SEQ ID NO:3. Exemplary modified IFN-β candidate LEAD polypeptides are set forth in any one of SEQ ID NOS: 56, 145-153, 159, 169, 194, 200, 212, 230, 252, 256, 281, 519, and 520.

In one example, a modified IFN-β polypeptide that exhibits increased conformational stability due to a decreased isoelectric point can contain one or more amino acid modifications compared to an unmodified IFN-β polypeptide corresponding to any one or more modifications of K45D, K52D, K105D, and K115D of a mature IFN-β polypeptide set forth in SEQ ID NO:1. Modifications can be in an unmodified IFN-β polypeptide having a sequence of amino acids set forth in SEQ ID NO:1 or SEQ ID NO:3. Additionally, a modified IFN-β as set forth above can contain a further modification. The further modification can be any one or more amino acid modifications corresponding to any one or more modification of R11Q, R11D, K45Q, K52Q, K105Q, K108Q, K108D, R113Q, R113E, K115Q, R124Q, R124D, R124E, R152Q, R152D, R165Q, and R165D of a mature IFN-β polypeptide set forth in SEQ ID NO:1. In another example, a modified IFN-β polypeptide that exhibits increased conformational stability due to a decreased isoelectric point can contain two or more amino acid modifications corresponding to any two or more modifications of R11Q, R11D, K45Q, K45D, K52Q, K52D, K105Q, K105D, K108Q, K108D, R113Q, R113E, K115Q, K115D, R124Q, R124D, R124E, R152Q, R152D, R165Q, and R165D of a mature IFN-β polypeptide set forth in SEQ ID NO:1. The unmodified IFN-β polypeptide can be an IFN-β polypeptide having a sequence of amino acids set forth in SEQ ID NO:1 or SEQ ID NO:3. Generally, any resulting polypeptide retains one or more activities of the unmodified IFN-β.

3. SuperLeads

IFN-β superLEAD mutant polypeptides are a combination of single amino acid mutations present in two or more of the respective IFN-β LEAD mutant polypeptides as set forth above. Thus, IFN-β superLEAD mutant polypeptides have two or more of the single amino acid mutations derived from two or more of the respective IFN-β LEAD mutant polypeptides. As described above and in detail below, modified IFN-β polypeptides provided herein exhibit increased protein stability manifested as an increased resistance to proteolysis or as an increased conformational stability. Typically, IFN-β LEAD mutant polypeptides created are those whose performance has been optimized with respect to the unmodified polypeptide by modification of a single amino acid replacement at one is-HIT position. IFN-β SuperLead polypeptides are created such that the polypeptide contains two or more IFN-β LEAD modifications, each at a different is-HIT position. In one example, an IFN-β polypeptide exhibiting improved protein stability can contain two or more modifications that are manifested as increased resistance to proteolysis. In another example, an IFN-β polypeptide exhibiting improved protein stability can contain two or more modifications that are manifested as increased conformational stability. Additionally, an IFN-β polypeptide that exhibits increased protein stability can contain any combination of modifications, such as one or more modifications manifested as increased resistance to proteases and one or more modifications manifested as increased conformational stability. Once the LEAD mutant polypeptides have been identified using, for example, 2D-scanning methods, superLEADs can be generated by combining two or more individual LEAD mutant mutations using methods well known in the art, such as recombination, mutagenesis and DNA shuffling, and by methods such as additive directional mutagenesis and multi-overlapped primer extensions.

Exemplary modified IFN-β Super-LEAD polypeptides exhibiting increased protease stability can include IFN-β molecules containing two or more amino acid modifications compared to an unmodified IFN-β polypeptide. In some examples, an IFN-β polypeptide can contain 2, 3, 4, 5, 6, 7, 8, 9, 10, 11, 12, 13, 14, 15, 16, 17, 18, 19, 20, or more modified positions. Generally, the resulting IFN-β polypeptide exhibits increased protein stability and retains at least one activity of an unmodified IFN-β polypeptide. A modified IFN-β polypeptide can include any two or more amino acid modifications set forth in Table 2 above. For example, the modified IFN-β polypeptide can contain two or more amino acid modifications corresponding to any two or more modifications of Y3I, Y3H, L6I, L6V, L6H, L6A, R11D, Q18H, Q18S, Q18T, Q18N, K19N, L20I, L20V, L20H, L20A, L21I, L21V, L21T, L21Q, L21H, L21A, Q23H, Q23S, Q23T, Q23N, L24I, L24V, L24T, L24Q, L24H, L24A, E29N, K33N, D34N, D34Q, D34G, F38I, F38V, D39N, P41A, P41S, E42N, E43K, E43Q, E43H, E43N, K45D, K45N, Q48H, Q48S, Q48T, Q48N, Q49H, Q49S, Q49T, Q49N, F50I, F50V, Q51H, Q51S, Q51T, Q51N, K52D, K52N, E53R, E53Q, E53H, E53N, D54G, L57I, L57V, L57T, L57Q, L57H, L57A, Y60H, Y60I, E61K, E61Q, E61H, E61N, M62I, M62V, M62T, M62Q, M62H, M62A, L63I, L63V, L63T, L63Q, L63H, L63A, Q64H, Q64S, Q64T, Q64N, F70I, F70V, Q72H, Q72S, Q72T, Q72N, D73N, W79H, W79S, E81K, E81N, E85K, E85N, L87I, L87V, L87H, L87A, L88I, L88V, L88T, L88Q, L88H, L88A, L98I, L98V, L98H, L98A, K99N, L102I, L102V, L102T, L102Q, L102H, L102A, E103K, E103N, E104R, E104N, K105D, K105N, L106I, L106V, L106T, L106Q, L106H, L106A, E107R, E107N, K108D, K108N, E109R, E109N, D110K, D110N, R113E, K115D, K115Q, K115N, K115S, K115H, M117I, M117V, M117T, M117Q, M117A, L122I, L122V, L122T, L122Q, L122H, L122A, K123N, R124D, R124E, Y125H, Y125I, Y126H, Y126I, Y132H, Y132I, L133I, L133V, L133T, L133Q, L133H, L133A, K134N, K136N, E137N, W143H, W143S, R147H, R147Q, E149Q, E149H, E149N, L151I, L151V, L151T, L151Q, L151H, L151A, R152D, F154I, F154V, F156I, F156V, L160I, L160V, L160T, L160Q, L160H, L160A, L164I, L164V, L164T, L164Q, L164H, L164A, and R165D of a mature IFN-β polypeptide set forth in SEQ ID NO:1. In another example, an IFN-β polypeptide can contain any two or more amino acid modifications set forth in Table 3 above, such as any two or more amino acid modifications corresponding to any two or more modifications of M1V, M1I, M1T, M1A, M1Q, M1D, M1E, M1K, M1N, M1R, M1S, M1C, L5V, L5I, L5T, L5Q, L5H, L5A, L5D, L5E, L5K, L5R, L5N, L5S, L6D, L6E, L6K, L6N, L6Q, L6R, L6S, L6T, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, R11H, R11Q, S12D, S12E, S12K, S12R, S13D, S13E, S13K, S13N, S13Q, S13R, S13T, S13C, N14D, N14E, N14K, N14Q, N14R, N14S, N14T, F15I, F15V, F15D, F15E, F15K, F15R, Q16D, Q16E, Q16K, Q16N, Q16R, Q16S, Q16T, Q16C, C17D, C17E, C17K, C17N, C17R, C17S, C17T, K19Q, K19T, K19S, K19H, L20N, L20Q, L20R, L20S, L20T, L20D, L20E, L20K, W22S, W22H, W22D, W22E, W22K, W22R, Q23D, Q23E, Q23K, Q23R, L24D, L24E, L24K, L24R, N25H, N25S, N25Q, R27H, R27Q, L28V, L28I, L28T, L28Q, L28H, L28A, E29Q, E29H, Y30H, Y30I, L32V, L32I, L32T, L32Q, L32H, L32A, K33Q, K33T, K33S, K33H, R35H, R35Q, M36V, M36I, M36T, M36Q, M36A, D39Q, D39H, D39G, E42Q, E42H, K45Q, K45T, K45S, K45T, L47V, L47I, L47T, L47Q, L47H, L47A, K52Q, K52T, K52S, K52H, F67I, F67V, R71H, R71Q, D73Q, D73H, D73G, G78D, G78E, G78K, G78R, N80D, N80E, N80K, N80R, E81Q, E81H, T82D, T82E, T82K, T82R, I83D, I83E, I83K, I83R, I83N, I83Q, I83S, I83T, E85Q, E85H, N86D, N86E, N86K, N86R, N86Q, N86S, N86T, L87D, L87E, L87K, L87R, L87N, L87Q, L87S, L87T, A89D, A89E, A89K, A89R, N90D, N90E, N90K, N90Q, N90R, N90S, N90T, N90C, V91D, V91E, V91K, V91N, V91Q, V91R, V91S, V91T, V91C, Y92H, Y92I, Q94D, Q94E, Q94K, Q94N, Q94R, Q94S, Q94T, Q94C, I95D, I95E, I95K, I95N, I95Q, I95R, I95S, I95T, H97D, H97E, H97K, H97N, H97Q, H97R, H97S, H97T, H97C, L98D, L98E, L98K, L98N, L98Q, L98R, L98S, L98T, L98C, K99Q, K99T, K99S, K99H, V101D, V101E, V101K, V101N, V101Q, V101R, V101S, V101T, V101C, E103Q, E103H, E104Q, E104H, K105Q, K105T, K105S, K105H, E107 Q, E107H, K108Q, K108T, K108S, K108H, E109H, E109Q, D110Q, D110H, D110G, F111I, F111V, R113H, R113Q, L116V, L116I, L116T, L116Q, L116H, L116A, K123Q, K123T, K123S, K123H, R124H, R124Q, R128H, R128Q, L130V, L130I, L130T, L130Q, L130H, L130A, K134Q, K134T, K134S, K134H, K136Q, K136T, K136S, K136H, E137Q, E137H, Y138H, Y138I, R152H, R152Q, Y155H, Y155I, R159H, R159Q, Y163H, Y163I, R165H, and R165Q of a mature IFN-β polypeptide set forth in SEQ ID NO:1. In some examples, the modifications are in an unmodified IFN-β polypeptide having a sequence of amino acids set forth in SEQ ID NO:1 or 3. Also contemplated herein are any combination of IFN-β modifications set forth in Table 2 and Table 3. For example, an IFN-β polypeptide can contain one or more amino acid modifications compared to an unmodified IFN-β polypeptide set forth in Table 2 and/or set forth in Table 3.

In one non-limiting example, an IFN-β polypeptide can contain a modification at a position corresponding to L5 of a mature IFN-β polypeptide set forth in SEQ ID NO:1, and can contain a further amino acid modifications, such as any modification set forth in Table 2 or 3. In another example, an IFN-β polypeptide can contain a modification at a position corresponding to L6 of a mature IFN-β polypeptide set forth in SEQ ID NO:1, and can contain one or more further amino acid modification set forth in Table 2 or 3. Exemplary SuperLead IFN-β polypeptides are set forth in Table 15 with the sequence identifier (SEQ ID No.) in parenthesis next to each substitution. Resulting SuperLEADs can be tested for one or more parameters to assess protein stability (e.g., increased resistance to proteases and/or increased thermal tolerance using any of the assays described herein), such as is described in Examples 7-9. Provided herein are modified IFN-β Super-LEAD polypeptides containing two or more amino acid modifications and exhibiting increased protein stability having a sequence of amino acids set forth in any of SEQ ID NOS: 88-125.

TABLE 15
Exemplary amino acid modifications in IFN-β SuperLeads
that exhibit increased protein stability

| | | | | |
|---|---|---|---|---|
| L5D/L6E (88) | L5E/Q10D (89) | L5Q/M36I (90) | L6E/L47I (91) | L5E/K108S (92) |
| L5E/L6E (93) | L5D/Q10D (94) | L5N/M36I (95) | L6Q/L47I (96) | L5D/K108S (97) |
| L5N/L6E (98) | L5Q/Q10D (99) | L6E/M36I (100) | L5E/N86Q (101) | L5Q/K108S (102) |
| L5Q/L6E (103) | L5N/Q10D (104) | L6Q/M36I (105) | L5D/N86Q (106) | L5N/K108S (107) |
| L5D/L6Q (108) | L6E/Q10D (109) | L5E/L47I (110) | L5Q/N86Q (111) | L5E/L6Q (113) |
| L6Q/Q10D (114) | L5D/L47I (115) | L6Q/K108S (117) | L5N/L6Q (118) | L5E/M36I (119) |
| L5Q/L47I (120) | L6E/N86Q (121) | L5Q/L6Q (122) | L5D/M36I (123) | L5N/L47I (124) |
| L6Q/N86Q (125) | L6E/K108S (112) | L5N/N86Q (116) | | |

4. Other Modifications

In addition to any one or more amino acid modifications provided herein, a modified IFN-β polypeptide also can contain one or more other modifications, including those known to those of skill in the art, such as PEGylation, hyperglycosylation, deimmunization and others (see e.g. published U.S. Application Nos. US-2005-0054052; U.S. Pat. Nos. 6,127,332, 6,531,122, and 4,588,585; and published International Application Nos. WO 2004/087753, WO 2004/031352, WO 2005/003157, WO 2006/020580, WO 00/68387, WO 98/48018, WO 98/03887, and EP 260350). Generally, the modification results in increased stability without losing at least one activity, such as antiviral activity. (i.e. retains at least one activity as defined herein) of an unmodified IFN-β polypeptide. For example, other further modifications in an IFN-β polypeptide include one or more additional amino acid modification and/or one or more chemical modifications. Such modifications include, but are not limited to, those that alter the immunogenicity, glycosylation, activity, or any other known property of an IFN-β polypeptide. In another example, chemical modifications include post-translational modifications of a protein, such as for example, glycosylation by a carbohydrate moiety; acylation; methylation; phosphorylation; sulfation; prenylation; Vitamin C-dependent modifications such as for example, proline and lysine hydroxylations and carboxy terminal amidation; Vitamin K-dependent modifications such as for example, carboxylation of glutamic acid residues (i.e. gla residue); and incorporation of selenium to form a selenocysteine. Other protein modifications of an IFN-β polypeptide include PEGylation. In addition, protein modifications also can include modification to facilitate the detection, purification, and assay development of a polypeptide, such as for example, modification of a polypeptide with a Sulfo-NHS-LC-biotin for covalent attachment to a primary amine on a protein, or other similar modification for florescent, non-isotopic, or radioactive labels. Exemplary further modifications in an IFN-β polypeptide are described below. Modified polypeptides that are conjugates and/or labeled also are provided. For example, provided herein are modified polypeptides that are conjugated to a PEG moiety or contain a carbohydrate moiety covalently linked to one or more glycosylation site on the polypeptide.

a. Immunogenicity

There are many instances where the efficacy of a therapeutic protein is limited by an unwanted immune reaction to the therapeutic protein. An immune response to a therapeutic protein, such as IFN-β, proceeds via the MHC class II peptide presentation pathway. Here, exogenous proteins are engulfed and processed for presentation in association with MHC class II molecules of the DR, DQ, or DP type. MHC class II molecules are expressed by professional antigen presenting cells (APCs), such as macrophages and dendritic cells, amongst others. Engagement of a MHC class II peptide complex by a cognate T-cell receptor on the surface of the T cell, together with the cross binding of certain other co-receptors, such as the CD4 molecule, can induce an activated state within the T cell. Activation leads to the release of cytokines, further activating other lymphocytes such as B cells to produce antibodies or activating T killer cells as a full cellular immune response.

The ability of a peptide (T cell epitope) to bind a given MHC class II molecule for presentation on the surface of an APC is dependent on a number of factors, most notably its primary sequence. This will influence both its propensity for proteolytic cleavage and also its affinity for binding within the peptide binding cleft of the MHC class II molecule. The MHC class II/peptide complex on the APC surface presents a binding face to a particular T cell receptor (TCR) able to recognize determinants provided both by exposed residues of the peptide and the MHC class II molecule.

The identification of potential T cell epitopes can be carried out according to methods known in the art (see e.g., WO 98/59244; WO 98/52976; WO 00/34317; and US 2005/0054052) and can be used to identify the binding propensity of IFN-β peptides to an MHC class II molecule.

Further modifications to a modified IFN-β provided herein can include modifications of at least one amino acid residue resulting in a substantial reduction in activity of or elimination of one or more potential T cell epitopes from the protein, i.e. deimmunization of the polypeptide. One or more amino acid modification at particular positions within any of the potential MHC class II ligands can result in a deimmunized IFN-β polypeptide with a reduced immunogenic potential when administered as a therapeutic to a host, such as for example, a human host.

Exemplary amino acid positions for modification of a T cell epitope, and thereby a deimmunized IFN-β polypeptide with a reduced immunogenic potential, include positions 3, 6, 8, 9, 10, 11, 12, 13, 14, 15, 16, 17, 18, 19, 20, 21, 22, 23, 24, 25, 26, 28, 29, 30, 31, 32, 33, 34, 35, 36, 37, 38, 39, 40, 42, 43, 44, 45, 46, 47, 48, 50, 51, 52, 53, 54, 55, 56, 57, 58, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 95, 98, 100, 101, 102, 103, 106, 104, 105, 106, 107, 108, 109, 110, 111, 112, 114, 116, 117, 118, 119, 120, 122, 123, 124, 125, 126, 127, 128, 129, 130, 132, 133, 134, 135, 136, 137, 138, 139, 140, 141, 143, 145, 146, 148, 150, 151, 152, 153, 154, 155, 156, 157, 158, 159, 160, 161, 162, and 164, corresponding to positions of a mature IFN-β polypeptide set forth in SEQ ID NO:1. Amino acid modifications can be at one or more position corresponding to any of the following positions: Y3, L6, F8, L9, Q10, R11, S12, S13, N14, F15, Q16, C17, Q18, K19, L20, L21, W22, Q23, L24, N25, G26, L28, E29, Y30, C31, L32, K33, D34, R35, M36, N37, F38, D39, I40, E42, E43, I44, K45, Q46, L47, Q48, F50, Q51, K52, E53, D54, A55, A56, L57, T58, Y60, E61, M62, L63, Q64, N65, I66, F67, A68, I69, F70, R71, Q72, D73, S74, S75, S76, T77, G78, W79, N80, E81, T82, I83, V84, E85, N86, L87, L88, A89, N90, V91, Y92, I95, L98, T100, V101, L102, E103, E104, K105, L106, E107, K108, E109, D110, F111, T112, G114, L116, M117, S118, S119, L120, L122, K123, R124, Y125, Y126, G127, R128, I129, L130, Y132, L133, K134, A135, K136, E137, Y138, S139, H140, C141, W143, I145, V146, V148, I150, L151, R152, N153, F154, Y155, F156, I157, N158, R159, L160, T161, G162, and L164 of a mature IFN-β polypeptide set forth in SEQ ID NO:1. Exemplary amino acid modifications that can contribute to reduced immunogenicity of an IFN-β polypeptide include any one or more amino acid modifications corresponding to any one or more modification of Y3A, Y3C, Y3D, Y3E, Y3G, Y3H, Y3K, Y3N, Y3P, Y3Q, Y3R, Y3S, Y3T, L6N, L6P, L6Q, L6R, L6S, L6T, L6A, L6C, L6D, L6E, L6F, L6G, L6H, L6I, L6K, L6V, L6W, L6Y, F8A, F8C, F8D, F8E, F8G, F8H, F8K, F8N, F8P, F8Q, F8R, F8S, F8T, L9A, L9C, L9D, L9E, L9G, L9H, L9K, L9N, L9N, L9P, L9Q, L9R, L9S, L9T, L9F, L9I, L9M, L9V, L9W, L9Y, Q10A, Q10C, Q10G, Q10I, Q10P, R11A, R11C, R11G, R11P, S12P, S12T, S13A, S13C, S13G, S13P, N14D, N14H, N14P, F15A, F15C, F15D, F15E, F16G, F15H, F15K, F15N, F15P, F15Q, F15R, F15S, F15T, F15M, F15W, F15Y, Q16A, Q16C, Q16G, Q16P, C17D, C17E, C17H, C17K, C17N, C17P, C17Q, C17R, C17S, C17T, Q18H, Q18P, Q18T, K19A, K19C, K19G, L20A, L20C, L20D, L20E, L20G, L20H, L20K, L20N, L20P, L20Q, L20R, L20S, L20T, L20W, L20Y, L21A, L21C, L21D, L21E, L21G, L21H, L21K, L21N, L21P, L21Q, L21R, L21S, L21T, L21M, L21W, L21Y, W22A W22C, W22D, W22E, W22G, W22H, W22K, W22N, W22P, W22Q, W22R, W22S, W22T, Q23H, Q23P, Q23T, L24A, L24C, L24D, L24E, L24G, L24H, L24K, L24N, L24P, L24Q, L24R, L24S, L24T, L24F, L24I, L24M, L24V, L24W, L24Y, N25A, N25C, N25G, N25P, G26H, G26T, L28A, L28C, L28D, L28E, L28G, L28H, L28K, L28N, L28N, L28P, L28Q, L28R, L28S, L28T, L28F, L28I, L28M, L28V, L28W, L28Y E29A, E29C, E29G, E29H, E29P, E29W, Y30A, Y30C, Y30D, Y30E, Y30G, Y30H, Y30K, Y30N, Y30P, Y30Q, Y30R, Y30S Y30T, Y30M, C31D, C31E, C31H, C31K, C31N, C31P, C31Q, C31R, C31S, C31T, L32A, L32C, L32D, L32E, L32G, L32H, L32K, L32N, L32P, L32Q, L32R, L32S, L32F, L32I, L32M, L32V, L32W, L32Y, K33A, K33C, K33G, K33H, K33P, K33T, D34A, D34C, D34G, D34P, D34T, R35A, R35C, R35H, R35P, R35T, M36A, M36C, M36D, M36E, M36G, M36H, M36K, M36N, M36P, M36Q, M36R, M36S, M36T, M36F, M36I, M36V, M36V, V36W, M36Y, N37A, N37C, N37G, N37H, N37P, N37W, F38A, F38C, F38D, F38E, F38G, F38H, F38K, F38N, F38P, F38Q, F38R, F38S, F38T, F38I, F38M, F38V, F38W, F38Y, D39A, D39C, D39G, D39P, I40A, I40C, I40D, I40E, I40G, I40H, I40K, I40N, I40P, I40Q, I40R, I40S, I40T, E42A, E42C, E42G, E42P, E43H, E43P, I44A, I44C, I44D, I44E, I44G, I44H, I44K, I44N, I44P, I44Q, I44R, I44S, I44T, I44M, I44W, K45A, K45C, K45G, K45P, Q46P, Q46T, L47A, L47C, L47D, L47E, L47G, L47H, L47K, L47N, L47P, L47Q, L47R, L47S, L47T, L47M, L47W, L47Y, Q48A, Q48C, Q48G, Q48P, F50A, F50C, F50D, F50E, F50G, F50H, F50K, F50N, F50P, F50Q, F50R, F50S, F50T, F50M, F50W, Q51A, Q51C, Q51G, Q51P, K52A, K52C, K52G, K52H, K52P, K52T, E53H, E53P, E53T, D54A, D54C, D54G, D54P, A55C, A55D, A55E, A55G, A55H, A55K, A55N, A55P, A55Q, A55R, A55S, A55T, A56D, A56E, A56G, A56H, A56K, A56N, A56P, A56Q, A56R, A56S, A56T, L57A, L57C, L57D, L57E, L57G, L57H, L57K, L57N, L57P, L57Q, L57R, L57S, L57T, L57M, L57V, L57W, L57Y, T58A, T58C, T58G, T58P, Y60A, Y60D, Y60E, Y60E, Y60G, Y60H, Y60K, Y60N, Y60P, Y60Q, Y60R, Y60S, Y60T, E61A, E61C, E61G, E61P, M62A, M62C, M62D, M62E, M62G, M62H, M62K, M62N, M62P, M62Q, M62R, M62S, M62T, M62W, M62Y, L63A, L63C, L63D, L63E, L63G, L63H, L63K, L63N, L63P, L63R, L63S, L63T, L63F, L63M, L63V, L63W, L63Y, Q64A, Q64C, Q64G, Q64P, N65H, N65P, N65T, I66A, I66C, I66D, I66E, I66G, I66H, I66K, I66N, I66P, I66Q, I66R, I66S, I66T, I66M, I66W, I66Y, F67A, F67C, F67D, F67E, F67G, F67H, F67K, F67N, F67P, F67Q, F67R, F67S, F67T, F67M, F67W, M67Y, A68D, A68E, A68F, A68H, A68K, A68N, A68P, A68Q, A68R, A68S, A68T, I69A, I69C, I69D, I69E, I69G, I69H, I69K, I169N, I69P, I69Q, I69R, I69S, I69T, I69M, F70A, F70C, F70D, F70E, F70G, F70H, F70K, F70N, F70P, F70Q, F70R, F70S, F70T, F70M, F70W, R71A, R71C, R71G, R71P, Q72A, Q72C, Q72G, Q72P, Q72T, D73A, D73C, D73G, D73P, D73T, S74A, S74C, S74G, S74P, S74T, S75P, S75T, S76A, S76C, S76G, S76P, T77A, T77C, T77G, T77P, G78D, G78E, G78H, G78K, G78N, G78P, G78Q, G78R, G78S, G78T, W79A, W79C, W79D, W79E, W79G, W79H, W79K, W79N, W79P, W79Q, W79R, W79S, W79T, N80A, N80C, N80G, N80P, E81A, E81C, E81G, E81P, T82P, I83A, I83C, I83D, I83E, I83G, I83H, I83K, I83N, I83P, I83Q, I83R, I83S, I83T, V84A, V84C, V84D, V84E, V84K, V84N, V84P, V84Q, V84R, V84S, V84T, B84L, V84M, V84W, V84Y, E85P, E85T, N86A, N86C, N86G, N86P, L87A, L87C, L87D, L87E, L87G, L87H, L87K, L87N, L87P, L87Q, L87R, L87S, L87T, L87F, L87I, L87M, L87V, L87W, L87Y, L88A, L88C, L88D, L88E, L88G, L88H, L88K, L88N, L88P, L88Q, L88R, L88R, L88S, L88T, A89H, A89P, N90T, V91A, V91C, V91D, V91E, V91G, V91H, V91K, V91N, V91P, V91Q, V91R, V91S, V91T, Y92A, Y92C, Y92D, Y92E, Y92G, Y92H, Y92K, Y92N, Y92P, Y92Q, Y92R, Y92S, Y92T, I95A, I95C, I95D, I95E, I95G, I95H, I95K, I95N, I95P, I95Q, I95R, I95S, I95T, L98A, L98C, L98D, L98E, L98G, L98H, L98K, L98N, L98P, L98Q, L98R, L98S, L98T, T100H, V101A, V101C, V101D, V101E, V101G, V101H, V101K, V101N, V101P, V101Q, V101R, V101S, V101T, L102A, L102C, L102D, L102E, L102G, L102H, L102K, L102N, L102P, L102R, L102S, L102T, L102I, L102M, L102V, L102W, L102Y, E103A, E103C, E102G, E103P, E103T, E104P, K105H, K105Q, K105S, K105T, L106A, L106C, L106D, L106E, L106G, L106H, L106K, L106N, L106P, L106Q, L106R, L106S, L106T, L106M, L106W, L106Y, E107H, K108H, K108N, K108Q, K108S, K108T, E109P, E109T, D110H, D110Q, D110S, D110T, F111A, F111C, F111D, F111E, F111G, F111H, F111K, F111N, F111P, F111Q, F111R, F111S, F111T, F111M, F111W, F111Y, T112A, T112C, T112G, T112P, G114H, G114K, G114N, G114P, G114Q, G114S, G114T, L116A, L116C, L116D, L116E, L116E, L116G, L116H, L116K, L116N, L116P, L116Q, L116R, L116S, L116T, L116F, L116I, L116M, L116V, L116W, L116Y, M117A, M117C, M117D, M117E, M117G, M117H, M117K, M117N, M117P, M117Q, M117R, M117S, M117T, S118A, S118C, S118G, S118P, S119P, S119T, L120A, L120C, L120D, L120E, L120G, L120H, L120K, L120N, L120P, L120Q, L120R, L120S, L120T, L120M, L120W, L120Y, L122A, L122C, L122D, L122E, L122G, L122H, L122K, L122N, L122P, L122Q, L122R, L122S, L122T, L122W, L122Y, K123A, K123C, K123G, K123P, K123T, R124A, R124C, R124G, R124P, R124T, Y125A, Y125C, Y125D, Y125E, Y125G, Y125H, Y125K, Y125N, Y125P, Y125Q, Y125R, Y125S, Y125T, Y126A, Y126C, Y126D, Y126E, Y126G, Y126H, Y126K, Y126N, Y126P, Y126Q, Y126R, Y126S, Y126T, G127P, R128H, R128P, R128T, I129A, I129C, I129D, I129E, I129G, I129H, I129K, I129N, I129P, I129Q, I129R, I129S, I129T, I129W, I129Y, L130A, L130C, L130D, L130E, L130G, L130H, L130K, L130N, L130P, L130R, L130S, L130T, L130W, L130Y, Y132A, Y132C, Y132D, Y132E, Y132G, Y132H, Y132K, Y132K, Y132N, Y132P, Y132Q, Y132R, Y132S, Y132T, L133A, L133C, L133D, L133E, L133G, L133H, L133K, L133N, L133P, L133Q, L133R, L133S, L133T, L133I, L133M, L133V, L133W, L133Y, K134A, K134C, K134G, K134H, K134P, A135C, A135G, A135H, A135K, A135N, A135P, A135Q, A135R, A135S, A135T, K136P, K136T, E137A, E137C, E137G, E137P, E137T, Y138A, Y138C, Y138D, Y138E, Y138G, Y138H, Y138K, Y138N, Y138P, Y138Q, Y138R, Y138S, Y138T, S139P, S139T, H140A, H140C, H140G, H140P, C141D, C141E, C141H, C141K, C141N, C141P, C141Q, C141R, C141S, C141T, W143A, W143C, W143D, W143E, W143G, W143H, W143K, W143N, W143P, W143Q, W143R, W143W, W143T, I145A, I145C, I145D, I145E, I145G, I145H, I145K, I145N, I145P, I145Q, I145R, I145S, I145T, I145W, V146A, V146C, V146D, V146E, V146G, V146H, V146K, V146N, V146P, V146Q, V146R, V146S, V146T, V148A, V148C, V148D, V148E, V148G, V148H, V148K, V148N, V148P, V148Q, V148R, V148S, V148T, V148I, V148L, V148W, V148Y, I150A, I150C, I150D, I150E, I150G, I150H, I150K, I150N, I150P, I150Q, I150R, I150S, I150T, L151A, L151C, L151D, L151E, L151G, L151H, L151K, L151N, L151P, L151Q, L151R, L151S, L151T, L151F, L151M, L151V, L151W, L151Y, R152A, R152C, R152G, R152P, R152W, R152Y, N153A, N153C, N153G, N153P, N153T, F154A, F154C, F154D, F154E, F154G, F154H, F154K, F154N, F154P, F154Q, F154R, F154S, F154T, F154M, Y155A, Y155C, Y155D, Y155E, Y155G, Y155H, Y155K, Y155N, Y155P, Y155Q, Y155R, Y155S, Y155T, F156A, F156D, F156E, F156G, F156H, F156K, F156N, F156P, F156Q, F156R, F156S, F156T, F156I, F156M, F156W, F156Y, I157T, N158A, N158C, N158F, N158G, N158I, N158L, N158M, N158P, N158V, N158W, N158Y, R159D, R159F, R159H, R159I, R159K, R159N, R159P, R159Q, R159S, R159T, R159V, R159W, R159Y, L160D, L160E, L160F, L160G, L160H, L160I, L160K, L160N, L160P, L160Q, L160R, L160S, L160T, L160Y, T161D, T161E, T161F, T161H, T161I, T161L, T161N, T161P, T161Q, T161S, T161V, T161W, T161Y, G162D, G162E, G162F, G162H, G162I, G162K, G162N, G162P, G162Q, G162R, G162S, G162T, G162V, G162W, G162Y, L164A, L164C, L164D, L164E, L164F, L164G, L164H, L164I, L164K, L164M, L164N, L164N, L164P, L164Q, L164R, L164S, L164T, L164V, L164W, and L164Y of a mature IFN-β polypeptide set forth in SEQ ID NO:1.

b. Glycosylation

Many proteins with therapeutic potential include one or more glycosylation sites, e.g., amino acid sequences that are glycosylated by a eukaryotic cell. There have been various reports of attempts to increase the degree of glycosylation of therapeutic proteins in order to achieve 1) reduced immunogenicity; 2) less frequent administration of the protein; 3) increased protein stability such as increased serum half-life; and 4) reduction in adverse side effects such as inflammation. The glycosylation site(s) provides a site for attachment of a carbohydrate moiety on the subject polypeptide, such that when the subject polypeptide is produced in a eukaryotic cell capable of glycosylation, the subject polypeptide is glycosylated. The further glycosylation of an IFN-β polypeptide confers one or more advantages including increased serum half-life; reduced immunogenicity; increased functional in vivo half-life; reduced degradation by gastrointestinal tract conditions such as gastrointestinal tract proteases; and increased rate of absorption by gut epithelial cells. An increased rate of absorption by gut epithelial cells and reduced degradation by gastrointestinal tract conditions is important for enteral (e.g. oral) formulations of an IFN-β polypeptide.

Glycosylation of proteins results in the formation of glycoproteins due to the covalent attachment of oligosaccharides to a polypeptide. The carbohydrate modifications found in glycoproteins are linked to the protein component through either O-glycosidic or N-glycosidic bonds. The predominant carbohydrate attachment in glycoproteins of mammalian cells is via N-glycosidic linkage. The N-glycosidic linkage is through the amide group of asparagines. The site of carbohydrate attachment to N-linked glycoproteins is found within a consensus sequence of amino acids, N—X—S/T, where X is any amino acid except proline. In N-linked glycosylation, the carbohydrate directly attached to the protein is GlcNAc. Since glycosylation is known to be highly host cell-dependent, the sugar chains associated with N-linked glycosylation of a protein can differ (Kagawa et al., (1988) JBC 263:17508-17515). The O-glycosidic linkage is to the hydroxyl of serine, threonine or hydroxyllysine. In Ser- and Thr-type O-linked glycoproteins, the carbohydrate directly attached to the protein is GalNAc. A number of O-linked glycosylation sites are known in the art and have been reported in the literature, see e.g. Ten Hagen et al. (1999) J. Biol. Chem., 274:27867-74; Hanisch et al. (2001) Glycobiology, 11:731-740; and Ten Hagen et al., (2003) Glycobiology, 13:1R-16R.

Modified IFN-β polypeptides provided herein can further be glycosylated (i.e. hyperglycosylated) compared to an unmodified IFN-β polypeptide due to 1) a carbohydrate moiety covalently linked to at least one non-native glycosylation site not found in the unmodified IFN-β protein or 2) a carbohydrate moiety covalently linked to at least one native glycosylating site found but not glycosylated in the unmodified IFN-β protein. A hyperglycosylated IFN-β polypeptide can include O-linked glycosylation, N-linked glycosylation, and/or a combination thereof. Addition of glycosylation sites to variant IFN-β molecules can be accomplished by, for example, the incorporation of one or more serine or threonine residues to the native sequence or modified IFN-β polypeptide (for O-linked glycosylation sites) or by incorporation of a canonical N-linked glycosylation site, including but not limited to, N—X—Y, where X is any amino acid except for proline and Y is typically serine, threonine, or cysteine. In some examples, a hyperglycosylated IFN-β polypeptide can include 1, 2, 3, 4, or 5 carbohydrate moieties, each linked to different glycosylation sites. The glycosylation site can be a native glycosylation site. In other examples, the hyperglycosylated polypeptide can be glycosylated at a single non-native glycosylation site. In still other examples, the hyperglycosylated polypeptide can be glycosylated at more than one non-native glycosylation site, e.g., the hyperglycosylated IFN-β polypeptide can be glycosylated at 2, 3, or 4 non-native glycosylation sites.

In some instances, a hyperglycosylated IFN-β polypeptide is glycosylated at a native glycosylation site. For example, IFN-β, such as for example human IFN-β having an amino acid sequence set forth in SEQ ID NO:1, contains a single N-linked glycosylation site at residue N80 (Hosoi et al., (1988) J Interferon Res, 8: 375-84). The IFN-β polypeptide can be glycosylated at a single native glycosylation site, or at more than one native glycosylation site, e.g., at 2, 3, or 4 native glycosylation sites. A hyperglycosylated IFN-β polypeptide also can be glycosylated at both a native glycosylation site and a non-native glycosylation site.

Modified IFN-β polypeptide provided herein can have at least one additional carbohydrate moiety not found in the unmodified IFN-β polypeptide when each is synthesized in a eukaryotic cell that is capable of N- and/or O-linked protein glycosylation. Thus, e.g., compared to an unmodified IFN-β polypeptide, a hyperglycosylated modified IFN-β polypeptide can have at least 1, at least 2, at least 3, at least 4, or more, additional carbohydrate moieties. For example, where an unmodified IFN-β polypeptide has one covalently linked carbohydrate moiety, a hyperglycosylated IFN-β polypeptide can have 2, 3, 4, or more covalently linked carbohydrate moieties. In some examples, a hyperglycosylated IFN-β polypeptide of a modified IFN-β polypeptide provided herein, lacks a carbohydrate moiety covalently linked to a non-native glycosylation site, and has instead at least 1, at least 2, at least 3, or at least 4, or more additional carbohydrate moieties attached to native glycosylation sites. In other examples, a hyperglycosylated IFN-β polypeptide lacks a carbohydrate moiety covalently linked to a native glycosylation site, and has instead at least 2, at least 3, or at least 4, or more carbohydrate moieties attached to non-native glycosylation sites.

Whether a subject IFN-β polypeptide has N-linked and/or O-linked glycosylation is readily determined using standard techniques, see e.g., “Techniques in Glycobiology” R. Townsend and A. Hotchkiss, eds. (1997) Marcel Dekker; and “Glycoanalysis Protocols (Methods in Molecular Biology, Vol. 76)” E. Hounsell, ed. (1998) Humana Press. The change in electrophoretic mobility of a protein before and after treatment with chemical or enzymatic deglycosylation (e.g., using endoglycosidases and/or exoglycosidases) is routinely used to determine the glycosylation status of a protein. Enzymatic deglycosylation can be carried out using any of a variety of enzymes, including, but not limited to, peptide-N4-(N-acetyl-β-D-glycosaminyl) asparagine amidase (PNGase F); endoglycosidase F1, endoglycosidase F2, endoglycosidase F3, α(2→3,6,8,9) neuraminidase, and the like. For example, sodium docecyl sulfate-polyacrylamide gel electrophoresis (SDS-PAGE) analysis of the protein, either pre-treated with PNGase F or untreated with PNGaseF, is conducted. A marked decrease in band width and change in migration position after treatment with PNGaseF is considered diagnostic of N-linked glycosylation. The carbohydrate content of a glycosylated protein also can be detected using lectin analysis of protein blots (e.g., proteins separated by SDS-PAGE and transferred to a support, such as a nylon membrane). Lectins, carbohydrate binding proteins from various plant tissues, have both high affinity and narrow specificity for a wide range of defined sugar epitopes found on glycoprotein glycans (Cummings (1994) Methods in Enzymol. 230:66-86). Lectins can be detectably labeled (either directly or indirectly), allowing detection of binding of lectins to carbohydrates on glycosylated proteins. For example, when conjugated with biotin or digoxigenin, a lectin bound to a glycosylated protein can be easily identified on membrane blots through a reaction utilizing avidin or anti-digoxigenin antibodies conjugated with an enzyme such as alkaline phosphatase, β-galactosidase, luciferase, or horse radish peroxidase, to yield a detectable product. Screening with a panel of lectins with well-defined specificity provides considerable information about a glycoprotein's carbohydrate complement.

Exemplary amino acid positions contemplated herein for modification of a glycosylation site, for attachment of a carbohydrate moiety, include positions corresponding to positions 74, 109, and 111 of a mature IFN-β polypeptide set forth in SEQ ID NO:1. Amino acid replacement or replacements can correspond to any of the following positions: S74, E109, and F11 of mature IFN-β. In a particular embodiment, the amino acid replacement or replacements contributing to hyperglycosylation of modified IFN-β polypeptides is (are) replacement of amino acids by asparagines (N) or threonine (T). Thus, provided herein are modified IFN-β polypeptides containing a further modification corresponding to any one or more of S74N, S74T, E109N, E109T, F111N, and F11T of a mature IFN-β polypeptide set forth in SEQ ID NO:1

In one example, an exemplary hyperglycosylation modification in an IFN-β polypeptide, such as a modified IFN-β polypeptide provided herein, is (a) an amino acid modification corresponding to S74N of a mature IFN-β polypeptide; and (b) a carbohydrate moiety covalently attached to the R-group of the asparagine (N) residue.

In some examples, a hyperglycosylation modification in a modified IFN-β polypeptide provided herein can be (a) amino acid modifications corresponding to S74N and E109N of a mature IFN-β polypeptide; and (b) a carbohydrate moiety covalently attached to the R-group of each of the asparagine (N) residues. In an additional example, a hyperglycosylation modification in a modified IFN-β polypeptide provided herein can be (a) an amino acid modification corresponding to S74N, E109N, and F111T of a mature IFN-β polypeptide; and (b) a carbohydrate moiety covalently attached to the R-group of each of the asparagine (N) residues.

Additional hyperglycosylation modifications in an IFN-β polypeptide include (a) an amino acid modification corresponding to E109N of a mature IFN-β polypeptide; and (b) a carbohydrate moiety covalently attached to the R-group of the asparagine residue. In additional examples, a hyperglycosylation modification in a modified IFN-β polypeptide provided herein can be (a) an amino acid modification corresponding to E109N and F111T of a mature IFN-β polypeptide; and (b) a carbohydrate moiety covalently attached to the R-group of the asparagine residue.

Hyperglycosylation modifications in an IFN-β polypeptide also can include (a) an amino acid modification corresponding to E109T of a mature IFN-β polypeptide; and (b) a carbohydrate moiety covalently attached to the R-group of the threonine residue. In some examples, amino acid modifications in a modified IFN-β polypeptide provided herein can include amino acid modifications corresponding to S74N and E109T of a mature IFN-β; and (b) a carbohydrate moiety covalently attached to the R-group of the asparagine and threonine residues.

c. Additional Modifications

Additional modifications of polypeptides provided herein include chemical derivatization of polypeptides, including but not limited to, acetylation and carboxylation; changes in amino acid sequence that make the protein susceptible to PEGylation or other modification. A modified IFN-β polypeptide provided herein can be modified with one or more polyethylene glycol moieties (PEGylated). In some instances, a modified IFN-β polypeptide provided herein can contain one or more non-naturally occurring pegylation sites that are engineered to provide PEG-derivatized polypeptides with reduced serum clearance. Also contemplated are sequences that have phosphorylated amino acid residues, e.g. phosphotyrosine, phosphoserine, or phosphothreonine.

Other suitable additional modifications of a modified IFN-β polypeptide provided herein are polypeptides that have been modified using ordinary chemical techniques so as to improve their resistance to proteolytic degradation, to optimize solubility properties, or to render them more suitable as a therapeutic agent. For example, the backbone of the peptide can be cyclized to enhance stability (see e.g., Friedler et al. (2000) J. Biol. Chem. 275:23783-23789). Analogs can be used that include residues other than naturally occurring L-amino acids, e.g., D-amino acids or non-naturally occurring synthetic amino acids. The protein can be pegylated to enhance stability.

### E. PRODUCTION OF IFN-β POLYPEPTIDES

1. Polypeptide Expression

IFN-β polypeptides can be produced by any methods known in the art for protein production, including the introduction of nucleic acid molecules encoding IFN-β into a host cell, host animal and expression from nucleic acid molecules encoding IFN-β in vitro. Expression hosts include E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification. Components of a therapeutic complex need not all be expressed in the same host.

Expression in eukaryotic hosts can include expression in yeasts such as Saccharomyces cerevisae and Picchia Pastoria, insect cells such as Drosophila cells and lepidopteran cells, plants and plant cells such as tobacco, corn, rice, algae and lemna. Eukaryotic cells for expression also include mammalian cells lines such as Chinese hamster ovary (CHO) cells. Eukaryotic expression hosts also include production in transgenic animals, for example, including production in milk and eggs.

Many expression vectors are available for the expression of IFN-β. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

a. Prokaryotes

Prokaryotes, especially E. coli, provide a system for producing large amounts of IFN-β (see for example, Platis et al. Protein Exp. Purif. 31(2):222-30 (2003); and Khalizzadeh et al. J. Ind. Microbiol. Biotechnol. 31(2): 63-69 (2004)). Transformation of E. coli is simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λP_{L} promoter.

IFN-β can be expressed in the cytoplasmic environment of E. coli. The cytoplasm is a reducing environment and for some molecules, this can result in the formation of insoluble inclusion bodies. Reducing agents such as dithiothreotol and β-mercaptoethanol and denaturants, such as guanidine-HCl and urea, can be used to resolubilize the proteins. An alternative approach is the expression of IFN-β in the periplasmic space of bacteria which provides an oxidizing environment and chaperonin-like and disulfide isomerases which lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Mutations also can be used to increase solubility of expressed proteins. Typically, bacteria produce aglycosylated proteins. Thus, if proteins require glycosylation for function, glycosylation can be added in vitro after purification from host cells.

b. Yeast

Yeasts such as Saccharomyces cerevisae, Schizosaccharomyces pombe, Yarrowia lipolytica, Kluyveromyces lactis and Pichia pastoris are useful expression hosts for IFN-β (see for example, Skoko et al. Biotechnol. Appl. Biochem. 38(Pt3): 257-65 (2003)). Yeast can be transformed with episomal replicating vectors or by stable chromosomal integration by homologous recombination. Typically, inducible promoters are used to regulate gene expression. Example of such promoters include GAL1, GAL7 and GAL5 and metallothionein promoters such as CUP1. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed therapeutic components and complexes as they exit the secretion pathway. Yeast also is capable of glycosylation at Asn-X-Ser/Thr motifs.

c. Insects and Insect Cells

Insects and insect cells, particularly using baculovirus expression, are useful for expressing interferons including IFN-β (see, for example, Muneta et al. J. Vet. Med. Sci. 65(2): 219-23 (2003)). Insect cells and insect larvae, including expression in the haemolymph, express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the bombyx mori nuclear polyhedrosis virus (BmNPV) and an insect cell line such as Sf9 derived from Spodoptera frugiperda, Pseudaletia unipuncta (A7S) and Danaus plexippus (DpN1). For high level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express components of the therapeutic complexes and the complexes. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to those from genes such as elastase I, insulin, immunoglobulin, mouse mammary tumor virus, albumin, alpha-fetoprotein, alpha 1-antitrypsin, beta-globin, myelin basic protein, myosin light chain-2, and gonadotropic releasing hormone gene control. Selectable markers can be used to select for and maintain cells with the expression construct. Examples of selectable marker genes include, but are not limited to hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, Hela, MT2, mouse NSO (non-secreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. One such example is the serum-free EBNA-1 cell line (Pham et al., Biotechnol. Bioeng. 84: 332-42 (2003)).

e. Plants

Transgenic plant cells and plants can be used for the expression of IFN-β. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline syntase promoter, the ribose bisphosphate carboxylase promoter and the ubiquitin and UBQ3 promoters. Selectable markers such as hygromycin, phosphmannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce proteins (see for example, Mayfield et al. PNAS 100: 438-442 (2003)). Because plants have different glycosylation patterns than mammalian cells, this can influence the choice to produce IFN-β in these hosts.

2. Purification

Method for purification of IFN-β polypeptides from host cells will depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary the proteins can be extracted and further purified using standard methods in the art.

IFN-β can be purified using standard protein purification techniques known in the art including but not limited to, SDS-PAGE, size fraction and size exclusion chromatography, ammonium sulfate precipitation and ionic exchange chromatography. Affinity purification techniques also can be utilized to improve the efficiency and purity of the preparations. For example, antibodies, receptors and other molecules that bind IFN-β can be used in affinity purification. Expression constructs also can be engineered to add an affinity tag to a protein such as a myc epitope, GST fusion or His₆ and affinity purified with myc antibody, glutathione resin and Ni-resin, respectively. Purity can be assessed by any method known in the art including gel electrophoresis and staining and spectrophotometric techniques.

3. Fusion Proteins

Fusion proteins containing a targeting agent and a modified IFN-β protein also are provided. Pharmaceutical compositions containing such fusion proteins formulated for administration by a suitable route are provided. Fusion proteins are formed by linking in any order the modified IFN-β and an agent, such as an antibody or fragment thereof, growth factor, receptor, ligand and other such agent for directing the mutant protein to a targeted cell or tissue. Linkage can be effected directly or indirectly via a linker. The fusion proteins can be produced recombinantly or chemically by chemical linkage, such as via heterobifunctional agents or thiol linkages or other such linkages. The fusion proteins can contain additional components, such as E. coli maltose binding protein (MBP) that aid in uptake of the protein by cells (see, International PCT application No. WO 01/32711).

4. Polypeptide Modification

Modified IFN-β polypeptides can be prepared as naked polypeptide chains or as a complex. For some applications, it can be desirable to prepare modified IFN-β in a “naked” form without post-translational or other chemical modifications. Naked polypeptide chains can be prepared in suitable hosts that do not post-translationally modify IFN-β. Polypeptides also can be prepared in in vitro systems and using chemical polypeptide synthesis. For other applications, particular modifications can be desired including pegylation, albumination, glycosylation, phosphorylation or other known modifications. Such modifications can be made in vitro or for example, by producing the modified IFN-β is a suitable host that produces such modifications.

5. Nucleotide Sequences

Nucleic acid molecules encoding modified IFN-β proteins, provided herein, or the fusion protein operably-linked to a promoter, such as an inducible promoter for expression in mammalian cells also are provided. Such promoters include, but are not limited to, CMV and SV40 promoters; adenovirus promoters, such as the E2 gene promoter, which is responsive to the HPV E7 oncoprotein; a PV promoter, such as the PBV p89 promoter that is responsive to the PV E2 protein; and other promoters that are activated by the HIV or PV or oncogenes.

Modified IFN-β proteins provided herein, also can be delivered to the cells in gene transfer vectors. The transfer vectors also can encode additional other therapeutic agent(s) for treatment of the disease or disorder, such cancer or HIV infection, for which the modified IFN-β is administered. Transfer vectors encoding modified IFN-β can be used systemically, by administering the nucleic acid to a subject. For example, the transfer vector can be a viral vector, such as an adenovirus vector. Vectors encoding IFN-β also can be incorporated into stem cells and such stem cells administered to a subject such as by transplanting or engrafting the stem cells at sites for therapy. For example, mesenchymal stem cells (MSCs) can be engineered to express a modified IFN-β and such MSCs engrafted at a tumor site for therapy.

### F. ASSESSING MODIFIED IFN-β POLYPEPTIDE ACTIVITY(IES)

IFN-β activity can be assessed in vitro and/or in vivo. In one example, IFN-β variants can be assessed in comparison to unmodified and/or wild-type IFN-β. In other examples, a modified IFN-β polypeptide can be assessed for biological activity following in vitro or in vivo exposure to protein stability-altering conditions (i.e. exposure to proteases, or denaturing agents such as temperature or pH). In vitro assays include any laboratory assay known to one of skill in the art, such as for example, cell-based assays including proliferation assays, protein assays, and molecular biology assays. In vivo assays include IFN-β assays in animal models as well as administration to humans. In some cases, activity of IFN-β in vivo can be determined by assessing blood, serum, or other bodily fluid for assay determinants. Examples of assays to assess biological activity can be found in Fellous et al. (1982) Proc. Natl. Acad. Sci. USA 79:3082-3086; Czerniecki et al. (1984) J. Virol. 49(2):490-496; Mark et al. (1984) Proc. Natl. Acad. Sci. USA 81:5662-5666; Branca et al. (981) Nature 277:221-223; Williams et al. (1979) Nature 282:582-586; Herberman et al. (1979) Nature 277:221-223; Anderson et al. (1982) J. Biol. Chem. 257(19):11301-11304; or as described herein.

1. Anti-Viral Assays

Anti-viral activity of IFN-β can be determined by assessing the ability of IFN-β to protect cells from virus-induced cytopathic effects. Such a viral resistance assay can assay protection of cells including but not limited to Hela cells, A549 cells, a human “Wish” cell line, monkey VERO cells, or others. Typically, viruses that are routinely used to induce cytopathic affects include EMC (mouse encephalomyocarditis) virus or VSV virus. In one example, serial dilutions of IFN-β can be added to plated HeLa cells. After 24 hours of growth, a 1/1000 EMC virus dilution solution can be placed in each well, except for a cell only control row. After 48 hours of incubation, treated, infected cells can be stained with a cellular viability dye, such as for example Trypan Blue, to determine the proportion of intact cells. The cell-bound dye can be extracted and the absorbance of the dye can be measured such as by using an absorbance reader or an ELISA plate reader. The anti-viral activity of IFN-β can be depicted as the concentration of IFN-β needed for 50% protection of the cells against EMC virus-induced cytopathic effects (i.e., EC₅₀ average).

In some examples, the unmodified, wild-type IFN-β or modified IFN-β can be exposed to conditions that affect protein stability, such as for example, exposure to proteases, temperature or pH. The exposure can occur in vitro or in vivo. For example, a modified IFN-β polypeptide can be preincubated with a protease cocktail for increasing amounts of time, followed by quenching of the protease activity such as with EDTA. The protease-treated IFN-β can then be tested for its anti-viral activity in the assay described above to determine if it exhibits residual biological activity following protease treatment. In another example, the pharmacokinetics of a modified or unmodified IFN-β polypeptide can be assessed to determine if in vivo conditions affect the biological activity of an IFN-β polypeptide. In vivo conditions that can affect protein stability include temperature (i.e. such as at 37° C.), pH changes, exposure to proteases, etc. For example, unmodified wild-type IFN-β or modified IFN-β polypeptides can be administered by injection (intravenous, subcutaneous, oral, etc. . . . ) of an animal or human. Blood samples can be drawn over time. Serial dilutions of the collected plasma can be added to HeLa cells to assess for protection of cytopathic effects following infection with ECMV.

2. Cell Proliferation Assays

Anti-proliferative activity of IFN-β can be determined by assessing the capacity of wildtype or modified IFN-β to inhibit proliferation of Daudi cells (ATCC) using cell proliferation assays known to one of skill in the art. For example, serial dilutions of modified or unmodified IFN-β can be added to plated Daudi cells. After 72 hours of growth, the proliferation of the cells can be assessed such as for example by any standard method to assess proliferation, e.g., tritium incorporation, trypan blue staining, Cell titer 96® Aqueous one solution reagent (Promega), or others. The EC₅₀ value can be determined as the concentration of IFN-β necessary to give one-half the maximum response (see e.g., Examples 5 and 9).

3. Natural Killer Cell Activation

Activation of Natural Killer (NK) cell can be assessed following incubation with unmodified or modified IFN-β. Lymphocytes, such as human peripheral blood lymphocytes isolated on a Ficoll/Hypaque gradient, can be treated with an IFN-β. Following incubation, such as overnight, the lymphocytes can be mixed at a 50:1 ratio with target cells (i.e. Daudi cells labeled with chromium). Killing of the target cells can be assessed by measuring the chromium in the supernatant using a γ-counter. Percent release or killing can be calculated as a ratio of the measured radioactivity (cpm) in an IFN-β test sample compared to total radioactivity.

4. Measuring Markers of IFN-β Activity

IFN-β activity can be assessed by measuring specific IFN-β-induced protein markers that have been demonstrated to peak after in vitro stimulation of cells or in vivo injection of IFN-β (e.g., Bertolotto et al., (2004) J of Neurology Neurosurgery and Psychiatry, 75:1294-1299). Examples of protein markers induced by IFN-β include, but are not limited to, myxovirus resistance protein A (MxA), 2′-5′ oligoadenylate synthetase (OAS), and β2-microglobulin. Measurement of a protein marker from a cell extract, conditioned medium, blood, serum, or other source can be determined by Western Blot, ELISA, or other similar assays known to one of skill in the art. In some examples, the measurement of a specific transcript offers a better measure of biological activity since mRNA has a shorter half-life than protein. For example, PCR methods such as quantitative PCR or real-time PCR (RT-PCR) can be used to measure the induction of markers by IFN-β. In one example, MxA mRNA can be measured using quantitative-competitive PCR. Such an assay allows the determination and analysis of fluctuations of MxA expression over time, such as during treatment with IFN-β or a modified IFN-β. For example, blood samples can be obtained from animals or human patients who received treatment with a modified IFN-β to obtain a population of peripheral blood mononuclear cells (PBMCs). Using standard recombinant DNA techniques, total RNA can be extracted from the cells, cDNA prepared, and a qc-PCR reaction set up with two competitor cDNA fragments (e.g., co-MxA and co-glyceraldehyde phosphate dehydrogenase (GAPDH)). The amplified PCR products can be resolved and visualized following separation by agarose gel electrophoresis. The ratios between competitors and target cDNA can be evaluated as ratios between band values, taking as ratio=1 an amount of starting targets (MxA or GAPDH) equal to the amount of each competitor. The MxA mRNA levels can be normalized using GAPDH as a housekeeping gene to avoid differences due to possible RNA degradation/contamination or different reverse transcription efficiency.

5. Non-Human Animal Models

Non-human animal models are useful tools to assess activity and stability of IFN-β variants. For example, non-human animals can be used as models for a disease or condition. Non-human animals can be injected with disease and/or phenotype-inducing substances and then IFN-β variants administered to monitor the effects on disease progression. Genetic models also are useful. Animals such as mice can be generated which mimic a disease or condition by the overexpression, underexpression or knock-out of one or more genes. Such animals can be generated by transgenic animal production techniques well-known in the art or using naturally-occurring or induced mutant strains. Examples of useful non-human animal models of diseases associated with IFN-β activities include, but are not limited to, collagen-induced arthritis (CIA) mouse model of rheumatoid arthritis (Van Holten et al., (2004) Arthritis Research & Therapy, 6:239-249), experimental autoimmune encephalomyelitis (EAE) animal model of multiple sclerosis (Schmidt et al., (2001), J Neurosci Res. 65:59-67), animal models of cancer and angiogenesis such as malignant mesothelioma (Odaka et al., (2001) Experimental Therapeutics, 61:6201-6212), neuroblastoma (Streck et al., (2005), Cancer Lett. 228:163-70), and human xenografts tumors in ex vivo and in vivo models (Qin et al., (2001) Mol Ther, 4:356-64).

Animal models can further be used to monitor protein stability, half-life and clearance of IFN-β variants. Such assays can be useful for comparing IFN-β variants and for calculating doses and dose regimens for further non-human animal and human trials. For example, a modified IFN-β can be injected into the tail vein of mice. Blood samples can be taken at time-points after injection (such as minutes, hours and days afterwards) and then the level of the IFN-β variant in bodily samples including, but not limited to serum or plasma can be monitored at specific time-points, for example, by ELISA or radioimmunoassay.

### G. FORMULATION/PACKAGING/ADMINISTRATION

Pharmaceutical compositions containing a modified cytokine produced herein, including IFN-β variant (modified) polypeptides, modified IFN-β fusion proteins or encoding nucleic acid molecules, can be formulated in any conventional manner by mixing a selected amount of the polypeptide with one or more physiologically acceptable carriers or excipients. Selection of the carrier or excipient is within the skill of the administering profession and can depend upon a number of parameters. These include, for example, the mode of administration (i.e., systemic, oral, nasal, pulmonary, local, topical or any other mode) and disorder treated. The pharmaceutical compositions provided herein can be formulated for single dosage (direct) administration or for dilution or other modification. The concentrations of the compounds in the formulations are effective for delivery of an amount, upon administration, that is effective for the intended treatment. Typically, the compositions are formulated for single dosage administration. To formulate a composition, the weight fraction of a compound or mixture thereof is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

1. Administration of Modified IFN-β Polypeptides

The polypeptides can be formulated as the sole pharmaceutically active ingredient in the composition or can be combined with other active ingredients. The polypeptides can be targeted for delivery, such as by conjugation to a targeting agent, such as an antibody. Liposomal suspensions, including tissue-targeted liposomes, also can be suitable as pharmaceutically acceptable carriers. These can be prepared according to methods known to those skilled in the art. For example, liposome formulations can be prepared as described in U.S. Pat. No. 4,522,811. Liposomal delivery also can include slow release formulations, including pharmaceutical matrices such as collagen gels and liposomes modified with fibronectin (see, for example, Weiner et al. (1985) J Pharm Sci. 74(9): 922-5).

The active compound is included in the pharmaceutically acceptable carrier in an amount sufficient to exert a therapeutically useful effect in the absence of undesirable side effects on the subject treated. The therapeutically effective concentration can be determined empirically by testing the compounds in known in vitro and in vivo systems, such as the assays provided herein. The active compounds can be administered by any appropriate route, for example, orally, nasally, pulmonary, parenterally, intravenously, intradermally, intramuscularly, subcutaneously, or topically, in liquid, semi-liquid or solid form and are formulated in a manner suitable for each route of administration.

The modified IFN-β and physiologically acceptable salts and solvates can be formulated for administration by injection. For administration by inhalation, the modified IFN-β can be delivered in the form of a liquid or powder. In the case of a liquid, the modified polypeptides can be injected from a syringe or an auto-injector. In the case of a powder, the modified polypeptides can be reconstituted with a pharmaceutically acceptable excipient, such as pharmaceutically-acceptable saline, prior to administration. Administration can be by a medical professional or self-administration.

The modified IFN-β and physiologically acceptable salts and solvates can be formulated for administration by inhalation (either through the mouth or the nose), oral, transdermal, pulmonary, parenteral or rectal administration. For administration by inhalation, the modified IFN-β can be delivered in the form of an aerosol spray presentation from pressurized packs or a nebulizer with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of e.g., gelatin for use in an inhaler or insufflator, can be formulated containing a powder mix of a therapeutic compound and a suitable powder base such as lactose or starch.

For pulmonary administration to the lungs, the modified IFN-β can be delivered in the form of an aerosol spray presentation from a nebulizer, turbonebulizer, or microprocessor-controlled metered dose oral inhaler with the use of a suitable propellant. Generally, particle size is small, such as in the range of 0.5 to 5 microns. In the case of a pharmaceutical composition formulated for pulmonary administration, detergent surfactants are not typically used. Pulmonary drug delivery is a promising non-invasive method of systemic administration. The lungs represent an attractive route for drug delivery, mainly due to the high surface area for absorption, thin alveolar epithelium, extensive vascularization, lack of hepatic first-pass metabolism, and relatively low metabolic activity.

The modified IFN-β polypeptides can be formulated as a depot preparation. Such long-acting formulations can be administered by implantation (for example, subcutaneously or intramuscularly) or by intramuscular injection. Thus, for example, the therapeutic compounds can be formulated with suitable polymeric or hydrophobic materials (for example as an emulsion in an acceptable oil) or ion exchange resins, or as sparingly soluble derivatives, for example, as a sparingly soluble salt.

The modified IFN-β can be formulated for parenteral administration by injection (e.g., by bolus injection or continuous infusion). Formulations for injection can be presented in unit dosage form (e.g., in ampoules or in multi-dose containers) with an added preservative. The compositions can take such forms as suspensions, solutions or emulsions in oily or aqueous vehicles and can contain formulatory agents such as suspending, stabilizing and/or dispersing agents. Alternatively, the active ingredient can be in powder-lyophilized form for constitution with a suitable vehicle, e.g., sterile pyrogen-free water, before use.

Preparations for oral administration can be formulated for controlled release of the active compound. For buccal administration the compositions can take the form of tablets or lozenges formulated in conventional manner.

The pharmaceutical compositions can be formulated for local or topical application, such as for topical application to the skin (transdermal) and mucous membranes, such as in the eye, in the form of gels, creams, and lotions and for application to the eye or for intracisternal or intraspinal application. Such solutions, particularly those intended for ophthalmic use, can be formulated as 0.01%-10% isotonic solutions and pH about 5-7 with appropriate salts. The compounds can be formulated as aerosols for topical application, such as by inhalation (see, for example, U.S. Pat. Nos. 4,044,126, 4,414,209 and 4,364,923, which describe aerosols for delivery of a steroid useful for treatment inflammatory diseases, particularly asthma).

The concentration of active compound in the drug composition depends on absorption, inactivation and excretion rates of the active compound, the dosage schedule, and amount administered as well as other factors known to those of skill in the art. As described further herein, dosages can be determined empirically using dosages known in the art for administration of unmodified interferon-β, and comparisons of properties and activities (e.g., stability and biological activity) of the modified IFN-β compared to the unmodified and/or native IFN-β.

The compositions, if desired, can be presented in a package, in a kit or dispenser device, that can contain one or more unit dosage forms containing the active ingredient. The package, for example, contains metal or plastic foil, such as a blister pack. The pack or dispenser device can be accompanied by instructions for administration. The compositions containing the active agents can be packaged as articles of manufacture containing packaging material, an agent provided herein, and a label that indicates the disorder for which the agent is provided.

a. Oral Administration

Among the modified IFN-β polypeptides provided herein are IFN-βs modified to increase protein stability to conditions amendable to oral delivery. Oral delivery can include administration to the mouth and/or gastrointestinal tract. Such modifications can include increased protein-half life under one or more conditions such as exposure to saliva, exposure to proteases in the gastrointestinal tract, exposure to increased temperature, and exposure to particular pH conditions, such as the low pH of the stomach and/or pH conditions in the intestine. For example, modifications can include resistance to one or more proteases including pepsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, trypsin, luminal pepsin, microvillar endopeptidase, dipeptidyl peptidase, enteropeptidase, hydrolase, NS3, elastase, factor Xa, Granzyme B, thrombin, plasmin, urokinase, tPA and PSA. Modifications also can include increasing overall stability to potentially denaturing or conformation-altering conditions such as tolerance to temperature, and tolerance to mixing and aeration (e.g., chewing).

IFN-β polypeptides modified for suitability to oral delivery can be prepared using any of the methods described herein. For example, 2D- and 3D-scanning mutagenesis methods for protein rational evolution (see, co-pending U.S. application Ser. No. 10/658,355 and U.S. Published Application No. US-2004-0132977-A1 and published International applications WO 2004022593 and WO 2004022747) can be used to prepare modified cytokines. Modification of IFN-β polypeptides for suitability for oral delivery can include removal of proteolytic digestion sites in a cytokine and/or increasing the overall stability of the cytokine structure. Such modified IFN-β polypeptides exhibit increased protein half-life compared to an unmodified and/or wild-type native IFN-β polypeptide in one or more conditions for oral delivery. For example, a modified IFN-β polypeptide can have increased protein half-life and/or bioavailability in the mouth, throat (e.g., through the mucosal lining), the gastrointestinal tract or systemically.

The half-life in vitro or in vivo (protein stability) of the modified IFN-β polypeptides provided herein can be increased by an amount selected from at least about or at least 1%, at least 5%, 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500% or more, when compared to the half-life of an unmodified or wild-type IFN-β exposed to one or more conditions (i.e. proteases, pH, temperature) for oral delivery. In other embodiments, the half-life in vitro or in vivo (protein stability) of the modified cytokines provided herein is increased by an amount selected from at least 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more, when compared to the half-life of an unmodified or wild-type IFN-β exposed to one or more conditions for oral delivery (i.e. proteases, pH, temperature).

In one example, half-life in vitro or in vivo (protein stability) of the modified IFN-β cytokine is assessed by increased half-life in the presence of one or more proteases. Proteases include, but are not limited to, proteases in blood, serum, the gastrointestinal tract, and the stomach. For example, proteases include, but are not limited to, pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, luminal pepsin, microvillar endopeptidase, dipeptidyl peptidase, enteropeptidase, hydrolase, NS3, factor Xa, Granzyme B, thrombin, plasmin, urokinase, tPA and PSA. Exemplary modified IFN-β polypeptides provided herein include IFN-β polypeptides modified by mutation of gelatinase B substrate recognition sites for decreased proteolysis by gelatinase B. To assess protease resistance, modified IFN-β polypeptides can be mixed with one or more proteases and then assayed for biological activity and/or protein structure after a suitable reaction time. In one embodiment, the modified polypeptide is at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% more resistant to proteolysis.

Assessment of half-life also can include exposure to increased temperature, such as the body temperature of a subject; exposure to gastric juices and/or simulated gastric juices; exposure to particular pH conditions and/or a combination of two or more conditions. Following exposure to one or more conditions, biological activity and/or assessment of protein structure can be used to assess the half-life of the modified cytokine in comparison to an appropriate control (i.e., an unmodified and/or wild-type cytokine protein), such as is described herein.

The modified IFN-β polypeptides can be formulated for oral administration or delivery. Oral administration include tablets, capsules, liquids and other suitable vehicle for oral administration. The capsules or tablets can be formulated with an enteric coating to render them more gastro-resistant than in the absence thereof. Preparation of pharmaceutical compositions containing a modified IFN-β for oral delivery can include formulating modified cytokines with oral formulations known in the art and described herein. The compositions as formulated do not require addition of protease inhibitors and/or other ingredients that for stabilization of unmodified and wild-type cytokines upon exposure of proteases, pH and other conditions of oral delivery. For example, such compositions do not require addition of proteases, such as actinonin or epiactinonin and derivatives thereof; Bowman-Birk inhibitor and conjugates thereof; aprotinin and camostat. In other examples, the preparations for oral administration can be formulated with the use of protease inhibitors.

The IFN-β polypeptides can be formulated for mucosal delivery, including oral-mucosal delivery. Mucosal delivery, in which the composition is contacted with the mucosa, such as oral mucosa and delivered to the bloodstream substantially by-passing the gastrointestinal tract, is distinct from oral delivery in which the composition passes through the gastrointestinal tract.

Additionally, because modified IFN-β polypeptides provided herein exhibit increased protein stability, there is more flexibility in the administration of pharmaceutical compositions than their unmodified counterparts. For example, orally ingested IFN-β polypeptides are administered in the morning before eating (i.e., before digestive enzymes are activated). The modified IFN-β polypeptides herein exhibit protease resistance to digestive enzymes and can be administered any other time during the day and under conditions when digestive enzymes are present and active.

For oral administration, the pharmaceutical compositions can take the form of, for example, tablets and/or capsules, which prepared by conventional means with pharmaceutically acceptable excipients such as binding agents (e.g., pre-gelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The active ingredient present in the capsule can be in, for example, liquid or lyophilized form. The tablets or capsules can be coated by methods well known in the art. Tablets and capsules can be coated, for example, with an enteric coating. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol or fractionated vegetable oils); and preservatives (e.g., methyl or propyl-p hydroxybenzoates or sorbic acid). The preparations also can contain buffer salts, flavoring, coloring and/or sweetening agents as appropriate.

The compositions for oral administration can be formulated, for example, as gastro-resistant capsules or tablets. Such gastro-resistant capsules are modified release capsules that are intended to resist the gastric fluid and to release their active ingredient or ingredients in the intestinal fluid. They are prepared by providing hard or soft capsules with a gastro-resistant shell (enteric capsules) or by filling capsules with granules or with particles covered with a gastro-resistant coating.

The enteric coating is typically, although not necessarily, a polymeric material. Enteric coating materials contain bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating to achieve delivery of the active ingredient to the lower gastrointestinal tract. The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers, such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, met acrylic acid, methyl acrylate, ammonium methylacrylate, ethyl acrylate, methyl methacrylate and/or ethyl methacrylate (e.g., those copolymers sold under the trade name EUDRAGIT); vinyl polymers and copolymers, such as polyvinyl pyrrolidone (PVP), polyvinyl acetate, polyvinyl acetate phthalate, vinyl acetate crotonic acid copolymer, and ethylene-vinyl acetate copolymers; and shellac (purified lac). Combinations of different coating materials also can be used to coat a single capsule. Exemplary of such gastro-resistant capsules are hard gelatin capsules (sold by Torpac or Capsugel) size 9, coated with cellulose acetate phthalate (CAP) at 12% in acetone.

The enteric coating provides for controlled release of the active agent, such that drug release can be accomplished at some generally predictable location in the lower intestinal tract below the point at which drug release would occur without the enteric coating. The enteric coating also prevents exposure of the hydrophilic therapeutic agent and carrier to the epithelial and mucosal tissue of the buccal cavity, pharynx, esophagus, and stomach, and to the enzymes associated with these tissues. The enteric coating therefore helps to protect the active agent and a patient's internal tissue from any adverse event prior to drug release at the desired site of delivery. Furthermore, the coated capsules can permit optimization of drug absorption, active agent protection, and safety. Multiple enteric coatings targeted to release the active agent at various regions in the lower gastrointestinal tract would enable even more effective and sustained improved delivery throughout the lower gastrointestinal tract.

The coating can contain a plasticizer to prevent the formation of pores and cracks that would permit the penetration of the gastric fluids. Suitable plasticizers include, but are not limited to, triethyl citrate (CITROFLEX 2), triacetin (glyceryl triacetate), acetyl triethyl citrate (CITROFLEC A2), CARBOWAX 400 (polyethylene glycol 400), diethyl phthalate, tributyl citrate, acetylated monoglycerides, glycerol, fatty acid esters, propylene glycol, and dibutyl phthalate. In particular, a coating containing an anionic carboxylic acrylic polymer will typically contain less than about 50% by weight, generally less than about 30% by weight, and typically, about 10% to about 25% by weight, based on the total weight of the coating, of a plasticizer, particularly dibutyl phthalate, polyethylene glycol, triethyl citrate and triacetin. The coating also can contain other coating excipients, such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The coating can be applied to the capsule or tablet using conventional coating methods and equipment. For example, an enteric coating is applied to a capsule using a coating pan, an airless spray technique, fluidized bed coating equipment, or the like. Detailed information concerning materials, equipment and processes for preparing coated dosage forms can be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6^{th} Edition (Media, Pa.: Williams & Wilkins, 1995). The coating thickness, as noted above, must be sufficient to ensure that the oral dosage form remains intact until the desired site of topical delivery in the lower intestinal tract is reached.

Preparations for oral administration can be formulated to give controlled or sustained release or for release after passage through the stomach or in the small intestine of the active compound. For oral administration the compositions can take the form of tablets, capsules, liquids, lozenges and other forms suitable for oral administration Formulations suitable for oral administration include lozenges and other formulations that deliver the pharmaceutical composition to the mucosa of the mouth, throat and/or gastrointestinal tract. Lozenges can be formulated with suitable ingredients including excipients for example, anhydrous crystalline maltose and magnesium stearate. As noted, modified cytokines herein exhibit resistance to blood or intestinal proteases and can exhibit increased half-life in the gastrointestinal tract. Thus, preparations of oral administration can be suitably formulated without additional protease inhibitors or other protective compounds, such as a Bowman-Birk inhibitor, a conjugated Bowman-Birk inhibitor, aprotinin and camostat. Preparations for oral administration also can include a modified cytokine resistance to proteolysis formulated with one or more additional ingredients that also confer proteases resistance, or stability in other conditions such as particular pH conditions.

2. Administration of Nucleic Acids Encoding Modified IFN-β Polypeptides (Gene Therapy)

Also provided are compositions of nucleic acid molecules encoding the IFN-β polypeptides and expression vectors encoding them that are suitable for gene therapy. Rather than deliver the protein, nucleic acid can be administered in vivo under, such as systemically or by other route, or ex vivo, such as by removal of cells, including lymphocytes, introduction of the nucleic acid therein, and reintroduction into the host or a compatible recipient.

IFN-β polypeptides can be delivered to cells and tissues by expression of nucleic acid molecules. IFN-β polypeptides can be administered as nucleic acid molecules encoding IFN-β polypeptides, including ex vivo techniques and direct in vivo expression. Nucleic acids can be delivered to cells and tissues by any method known to those of skill in the art. The isolated nucleic acid can be incorporated into vectors for further manipulation.

Methods for administering IFN-β polypeptides by expression of encoding nucleic acid molecules include administration of recombinant vectors. The vector can be designed to remain episomal, such as by inclusion of an origin of replication or can be designed to integrate into a chromosome in the cell. IFN-β polypeptides also can be used in ex vivo gene expression therapy using non-viral vectors. For example, cells can be engineered to express an IFN-β polypeptide, such as by integrating an IFN-β polypeptide encoding-nucleic acid into a genomic location, either operatively linked to regulatory sequences or such that it is placed operatively linked to regulatory sequences in a genomic location. Such cells then can be administered locally or systemically to a subject, such as a patient in need of treatment.

Viral vectors, include, for example adenoviruses, herpes viruses, retroviruses and others designed for gene therapy can be employed. The vectors can remain episomal or can integrate into chromosomes of the treated subject. An IFN-β polypeptide can be expressed by a virus, which is administered to a subject in need of treatment. Virus vectors suitable for gene therapy include adenovirus, adeno-associated virus, retroviruses, lentiviruses and others noted above. For example, adenovirus expression technology is well-known in the art and adenovirus production and administration methods also are well known. Adenovirus serotypes are available, for example, from the American Type Culture Collection (ATCC, Rockville, Md.). Adenovirus can be used ex vivo, for example, cells are isolated from a patient in need of treatment, and transduced with an IFN-β polypeptide-expressing adenovirus vector. After a suitable culturing period, the transduced cells are administered to a subject, locally and/or systemically. Alternatively, IFN-β polypeptide-expressing adenovirus particles are isolated and formulated in a pharmaceutically-acceptable carrier for delivery of a therapeutically effective amount to prevent, treat or ameliorate a disease or condition of a subject. Typically, adenovirus particles are delivered at a dose ranging from 1 particle to 1014 particles per kilogram subject weight, generally between 106 or 108 particles to 1012 particles per kilogram subject weight. In some situations it is desirable to provide a nucleic acid source with an agent that targets cells, such as an antibody specific for a cell surface membrane protein or a target cell, or a ligand for a receptor on a target cell. Polynucleotides and expression vectors provided herein can be made by any suitable method. Further provided are nucleic acid vectors containing nucleic acid molecules as described above. Exemplary nucleic acid molecules have a sequence of nucleotides that encodes the polypeptide as set forth in any of SEQ ID NOS: 4-512, 519, 520, 534-659 or a biologically active fragment thereof. Further provided are nucleic acid vectors containing nucleic acid molecules as described above and cells containing these vectors.

The nucleic acid molecules can be introduced into artificial chromosomes and other non-viral vectors. Artificial chromosomes, such as ACES (see, Lindenbaum et al. Nucleic Acids Res. 2004 Dec. 7; 32(21):e172) can be engineered to encode and express the isoform. Briefly, mammalian artificial chromosomes (MACs) provide a means to introduce large payloads of genetic information into the cell in an autonomously replicating, non-integrating format. Unique among MACs, the mammalian satellite DNA-based Artificial Chromosome Expression (ACE) can be reproducibly generated de novo in cell lines of different species and readily purified from the host cells' chromosomes. Purified mammalian ACEs can then be re-introduced into a variety of recipient cell lines where they have been stably maintained for extended periods in the absence of selective pressure using an ACE System. Using this approach, specific loading of one or two gene targets has been achieved in LMTK(−) and CHO cells.

Another method for introducing nucleic acids encoding the modified IFN-β polypeptides is a two-step gene replacement technique in yeast, starting with a complete adenovirus genome (Ad2; Ketner et al. (1994) Proc. Natl. Acad. Sci. USA 91: 6186-6190) cloned in a Yeast Artificial Chromosome (YAC) and a plasmid containing adenovirus sequences to target a specific region in the YAC clone, an expression cassette for the gene of interest and a positive and negative selectable marker. YACs are of particular interest because they permit incorporation of larger genes. This approach can be used for construction of adenovirus-based vectors bearing nucleic acids encoding any of the described modified IFN-β polypeptides for gene transfer to mammalian cells or whole animals.

The nucleic acids can be encapsulated in a vehicle, such as a liposome, or introduced into a cell, such as a bacterial cell, particularly an attenuated bacterium or introduced into a viral vector. For example, when liposomes are employed, proteins that bind to a cell surface membrane protein associated with endocytosis can be used for targeting and/or to facilitate uptake, e.g., capsid proteins or fragments thereof tropic for a particular cell type, antibodies for proteins which undergo internalization in cycling, and proteins that target intracellular localization and enhance intracellular half-life.

For ex vivo and in vivo methods, nucleic acid molecules encoding the IFN-β polypeptide is introduced into cells that are from a suitable donor or the subject to be treated. Cells into which a nucleic acid can be introduced for purposes of therapy include, for example, any desired, available cell type appropriate for the disease or condition to be treated, including but not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., such as stem cells obtained from bone marrow, umbilical cord blood, peripheral blood, fetal liver, and other sources thereof.

For ex vivo treatment, cells from a donor compatible with the subject to be treated or the subject to be treated cells are removed, the nucleic acid is introduced into these isolated cells and the modified cells are administered to the subject. Treatment includes direct administration, such as, for example, encapsulated within porous membranes, which are implanted into the patient (see, e.g., U.S. Pat. Nos. 4,892,538 and 5,283,187). Techniques suitable for the transfer of nucleic acid into mammalian cells in vitro include the use of liposomes and cationic lipids (e.g., DOTMA, DOPE and DC-Chol) electroporation, microinjection, cell fusion, DEAE-dextran, and calcium phosphate precipitation methods. Methods of DNA delivery can be used to express IFN-β polypeptides in vivo. Such methods include liposome delivery of nucleic acids and naked DNA delivery, including local and systemic delivery such as using electroporation, ultrasound and calcium-phosphate delivery. Other techniques include microinjection, cell fusion, chromosome-mediated gene transfer, microcell-mediated gene transfer and spheroplast fusion.

In vivo expression of an IFN-β polypeptide can be linked to expression of additional molecules. For example, expression of an IFN-β polypeptide can be linked with expression of a cytotoxic product such as in an engineered virus or expressed in a cytotoxic virus. Such viruses can be targeted to a particular cell type that is a target for a therapeutic effect. The expressed IFN-β polypeptide can be used to enhance the cytotoxicity of the virus.

In vivo expression of a IFN-β polypeptide can include operatively linking an IFN-β polypeptide encoding nucleic acid molecule to specific regulatory sequences such as a cell-specific or tissue-specific promoter. IFN-β polypeptides also can be expressed from vectors that specifically infect and/or replicate in target cell types and/or tissues. Inducible promoters can be used to selectively regulate IFN-β polypeptide expression.

Nucleic acid molecules, as naked nucleic acids or in vectors, artificial chromosomes, liposomes and other vehicles can be administered to the subject by systemic administration, topical, local and other routes of administration. When systemic and in vivo, the nucleic acid molecule or vehicle containing the nucleic acid molecule can be targeted to a cell.

Administration also can be direct, such as by administration of a vector or cells that typically targets a cell or tissue. For example, tumor cells and proliferating cells can be targeted cells for in vivo expression of IFN-β polypeptides. Cells used for in vivo expression of an IFN-β polypeptide also include cells autologous to the patient. Such cells can be removed from a patient, nucleic acids for expression of an IFN-β polypeptide introduced, and then administered to a patient such as by injection or engraftment.

### H. THERAPEUTIC USES

The modified IFN-β polypeptides and nucleic acid molecules provided herein can be used for treatment of any condition for which unmodified IFN-β is employed. This section provides exemplary uses of and administration methods. These described therapies are exemplary and do not limit the applications of IFN-β.

The modified IFN-β polypeptides provided herein can be used in various therapeutic as well as diagnostic methods in which IFN-β is employed. Such methods include, but are not limited to, methods of treatment of physiological and medical conditions described and listed below. Modified IFN-β polypeptides provided herein can exhibit improvement of in vivo activities and therapeutic effects compared to wild-type IFN-β, including lower dosage to achieve the same effect, a more sustained therapeutic effect and other improvements in administration and treatment.

In particular, modified IFN-β polypeptides, are intended for use in therapeutic methods in which IFN-β has been used for treatment. Such methods include, but are not limited to, methods of treatment of infectious diseases, allergies, microbial diseases, pregnancy related diseases, bacterial diseases, heart diseases, viral diseases, histological diseases, genetic diseases, blood related diseases, fungal diseases, adrenal diseases, cancers, liver diseases, autoimmune diseases, growth disorders, diabetes, neurodegenerative diseases, including multiple sclerosis, Parkinson's disease and Alzheimer's disease.

Treatment of diseases and conditions with IFN-β variants can be effected by any suitable route of administration using suitable formulations as described herein including, but not limited to, subcutaneous injection, oral and transdermal administration. If necessary, a particular dosage and duration and treatment protocol can be empirically determined or extrapolated. For example, exemplary doses of recombinant and native IFN-β polypeptides can be used as a starting point to determine appropriate dosages. IFN-β variants that are more stable and have an increased half-life in vivo, can be effective at reduced dosage amounts and or frequencies. Dosages provided herein for treatments and therapies with IFN-β and recombinant forms are exemplary dosages. Such exemplary dosages, however, can provide guidance in selecting dosing regimes for IFN-β variants. Since the variants provided herein exhibit increased stability, dosages and administration regimens can differ from those for the unmodified IFN-β polypeptides. Particular dosages and regimens can be empirically determined.

Dosage levels can be determined based on a variety of factors, such as body weight of the individual, general health, age, the activity of the specific compound employed, sex, diet, time of administration, rate of excretion, drug combination, the severity and course of the disease, and the patient's disposition to the disease and the judgment of the treating physician. The amount of active ingredient that can be combined with the carrier materials to produce a single dosage form will vary depending upon the host treated and the particular mode of administration. Recombinant IFN-β polypeptides are administered using the following dosages and regimens:

(1) Rebif® (IFN-β-1a) is administered by subcutaneous injection three times per week; it is administered at 8.8 mcg for weeks 1-2, at 22 mcg for weeks 3-4 and at 44 mcg for week 5 and beyond. Peak effectiveness occurs approximately 14 hours post-injection and the recombinant polypeptide has a half-life of about 69 hours.

(2) Avonex® (IFN-β-1a) is administered once-weekly by intramuscular injection at a dose of 30 mcg. Dosage does not change over time. Peak effectiveness occurs 3-15 hours post-injection and the recombinant polypeptide has a half-life of about 10 hours.

(3) Betaseron® (IFN-β-1b) is administered every other day by subcutaneous injection at a dose of 250 mcg. Dosage does not change over time. Peak effectiveness occurs 1-8 hours post-injection and the recombinant polypeptide has a half-life of about 8 minutes to 4.3 hours.

The modified IFN-β polypeptides provided herein exhibit increased protein stability and improved half-life when administered to a subject having a disease or condition that is treated by administration of IFN-β. Of particular interest are those modified IFN-β polypeptides that are resistant to the matrix metalloproteinase, gelatinase B (MMP-9). Thus, modified IFN-β can be used to deliver longer lasting, more stable disease therapies, such as for example as a therapeutic for treating Multiple Sclerosis (MS). Examples of therapeutic improvements using modified IFN-β polypeptides include, for example, but are not limited to, lower dosages, fewer and/or less frequent administrations, decreased side effects and increased therapeutic effects. Modified IFN-β polypeptides can be tested for therapeutic effectiveness, for example by using known disease models as described elsewhere herein. Progression of disease symptoms and phenotypes can be monitored to assess the effects of the modified IFN-β. As comparisons, placebo-treated animals and animals treated with unmodified IFN-β can be used as controls. Thus the modified IFN-β polypeptides provided herein can be administered at lower dosages and/or less frequently than unmodified or wild-type IFN-β or recombinant forms of IFN-β including Rebif®, Avonex® or Betaseron® while retaining one or more therapeutic activities and/or having one or fewer/decreased side effects.

Upon improvement of a patient's condition, a maintenance dose of a compound or compositions can be administered, if necessary; and the dosage, the dosage form, or frequency of administration, or a combination thereof can be modified. In some cases, a subject can require intermittent treatment on a long-term basis upon any recurrence of disease symptoms.

1. Autoimmune Diseases

A number of autoimmune diseases are targets for IFN-β therapy. Exemplary autoimmune diseases include, for example, multiple sclerosis and rheumatoid arthritis. The modified IFN-β proteins herein, and nucleic acids encoding modified IFN-βs can be used in therapies for autoimmune diseases. The modified IFN-βs herein provide increased protein stability and improved half-life. Thus, modified IFN-β can be used to deliver longer lasting, more stable therapies. Examples of therapeutic improvements using modified IFN-βs include, for example, but are not limited to, lower dosages, fewer and/or less frequent administrations, decreased side effects and increased therapeutic effects.

a. Multiple Sclerosis (MS)

Multiple sclerosis (MS) is a pathogenically heterogeneous chronic inflammatory disease of the central nervous system (CNS) and is one of the most common neurological diseases of young adults in Europe and North America. Approximately one (1) million people world-wide are afflicted by MS. Histological hallmarks of active MS include, for example, infiltration of T cells, macrophages and B cells, degradation of myelin (and to a lesser extent, axons) and reactive changes of astrocytes and microglia. Myelin is the fatty sheath that surrounds and protects nerve fibers and its destruction is called demyelination. Demyelination causes nerve impulses to be slowed and/or halted and produces the symptoms of MS (see, e.g., 2005 National MS Society Information Handbook). MS can be considered an autoimmune disease because inflammatory changes result from an attack against self myelin components. An influx of mononuclear cells occurs through a disrupted BBB into an immune-privileged CNS and the secretion of a variety of inflammatory cytokines and chemokines from glial cells leads to loss of myelin, disruption of oligodendrocyte integrity and axonal loss (Al-Omaishi et al., J. Leukocyte Biology, 65(4): 444-452 (1999). Four types of MS lesions have been proposed: macrophage-mediated demyelination (type I), antibody-mediated demyelination (type II), distal oligodendrogliopathy (type III), and demyelination secondary to oligodendrogliopathy (type IV). There is some speculation that the inflammatory reaction is primarily directed against an unknown infectious agent or that the inflammatory changes are secondary to a primary degenerative process. Studies of MS and MS lesions rely on animal models of inflammatory demyelination, such as experimental autoimmune encephalomyelitis (EAE). EAE can be induced in many species, such as mice, by active immunization with myelin antigens, such as MBP, and non-myelin antigens, such as proteolipid protein and myelin oligodendrocyte glycoprotein (MOG) (Hohlfeld and Wekerle, PNAS 101(Supp 2): 14599-14606 (2004)).

Matrix metalloproteinases (MMPs) facilitate T cell migration into the CNS, disrupt the blood-brain-barrier (BBB) and play a role in myelin break-down. MMPs are increased in brain tissue, cerebral spinal fluid (CSF) and blood of MS patients and function as effector molecules in several steps of MS pathogenesis (Gilli et al. Brain 127(Pt. 2): 259-268 (2004)). Gelatinase B (MMP-9) is capable of destroying the BBB and is capable of cleaving myelin basic protein (MBP) into immunodominant and encephalitogenic fragments (Nelissen et al. Brain 126: 1371-1381 (2003)). Increases in gelatinase B serum levels correlate with disease activity in relapsing remitting multiple sclerosis (RRMS). Additionally, gelatinase B can cleave IFN-β, thereby reducing the anti-viral and immunotherapeutic activities of the cytokine. An imbalance between gelatinase B and expression of its endogenous inhibitor, tissue inhibitor of metalloproteinase-1 (TIMP-1), is a feature in MS. Gelatinase B plays a functional role and is a therapeutic target in multiple sclerosis (Gilli et al. Brain 127(Pt. 2): 259-268 (2004); Lee et al. Brain 122: 191-197 (1999)). Inhibition of gelatinase B with inhibitors has been shown to suppress the development or reverse ongoing clinical EAE in a dose-dependent manner (Gijbels et al. J. Clin. Invest. 94: 2177-2182 (1994).

Administration of IFN-γ to MS patients has been shown to exacerbate the disease and induce major histocompatibility (MHC) class II expression in the central nervous system, where it is absent under normal physiological conditions. Presentation of antigens in the CNS through IFN-γ-induced class II expression leads to activation of autoreactive T cells, a primary event in pathogenesis of MS. IFN-β administration, on the other hand, has been shown to reduce exacerbations and actively diminish disease progression. IFN-β significantly down-regulates IFN-γ-induced FcγRI surface expression in peripheral blood monocytes. Down-regulation of FcγRI surface expression correlates with diminished cellular signaling through FcγRI, thereby decreasing release of proinflammatory cytokines (Van Weyenbergh et al. J. Immunol. 161: 1568-1574 (1998)).

IFN-β administration is an established treatment for RRMS and can act by inhibiting T-cell migration in vitro, down-regulating MMP expression, delaying clinical relapse and delaying progression of disability caused by the disease. Additionally, IFN-β has been shown to suppress IFN-γ production and inflammatory activity, and in MS, to help limit damage caused by inflammation and coordinate the immune system. IFN-β treatment also can result in a decrease in the severity of inflammation and de-myelination in the central nervous system, phenotypes often associated with MS. IFN-β can be administered for example, by injection, to treat MS. One of the molecular mechanisms by which IFN-β exerts therapeutic benefits is by reducing gelatinase B expression and increasing it endogenous inhibitor, TIMP-1. Primary progressive multiple sclerosis (PPMS) differs from RRMS in demographic and immunological aspect and MRI criteria. In PPMS patients, administration of IFN-β-1b decreases serum levels of gelatinase B and, thus, has therapeutic potential for treatment of PPMS (Yushchenko et al. J. Neurol. 250(10): 1224-1228 (2003)).

The modified IFN-β polypeptides provided herein, and nucleic acids encoding modified IFN-βs provided herein can be used in therapies for MS. The modified IFN-βs herein provide increased protein stability and improved half-life when administered to a subject having multiple sclerosis. Of particular interest are those modified IFN-β polypeptides that are resistant to gelatinase B. Thus, modified IFN-β can be used to deliver longer lasting, more stable MS therapies. Examples of therapeutic improvements using modified IFN-βs include, for example, but are not limited to, lower dosages, fewer and/or less frequent administrations, decreased side effects and increased therapeutic effects. Modified IFN-βs can be tested for therapeutic effectiveness, for example by using experimental autoimmune encephalomyelitis (EAE) mice, or any other known disease model for MS. Progression of disease symptoms and phenotypes is monitored to assess the effects of the modified IFN-β. As comparisons, placebo-treated animals and animals treated with unmodified IFN-β can be used as controls.

b. Rheumatoid Arthritis (RA)

Rheumatoid arthritis (RA) is a chronic inflammatory disease that affects the synovial tissue in multiple joints, which leads to joint destruction and disability. Activation of T cells is believed to be the causative factor leading to inflammation in RA, which in turn leads to the activation of macrophages and fibroblast-like synoviocytes. Fibroblast-like synoviocytes produce a variety of pro-inflammatory cytokines causing proliferation of synovial tissue associated with destruction of cartilage and bone. Tissue destruction in RA is closely related to the production of matrix metalloproteinases and other proteinases, which are able to degrade collagen and proteoglycans. Macrophages, which are found in the synovial lining layer, amplify stimulatory signals and tissue destruction. In the case of RA, macrophages are activated and mediate inflammation by the production of cytokines such as TNF-α, IL-1β, IL-6, IL-12, IL-15, IL-18, PDGF and TGF, which in turn activate fibroblast-like synoviocytes. It is apparent that there is an imbalance between pro-inflammatory and anti-inflammatory molecules in RA joints. Increased levels of MMP have been found in cartilage from RA patients and enzyme activity was found to correlate with lesion severity. Also, synovial fluid from RA patients contained greater levels of MMP than controls (Van Holten et al. Arthritis Research 4(6): 346-352 (2002)). One of the therapeutic targets in RA is MMPs to prevent destruction of cartilage and bone. Damage to bone, cartilage, tendons and ligaments is largely mediated by proteinases (e.g., metalloproteinases) involved in the breakdown of the extracellular matrix (ECM). Elevated levels of three matrix metalloproteinases have been have been observed in patients suffering from RA and correlate with disease progression: collagenase (MMP-1), stromelysin (MMP-3) and gelatinase B (MMP-9) (Grillet et al. Br. J. Rheum. 36: 744-747 (1997)). Inhibition of MMP activity has been demonstrated in models of MS and other inflammatory models, where MMPs are of pathological importance, such as rheumatoid arthritis (RA).

Natural MMP inhibitors exist and are produced locally by chondrocytes and fibroblast-like synoviocytes, and are termed tissue inhibitors of metalloproteinases (TIMPs). An imbalance of MMPs and TIMPs contributes to joint destruction. Several chemotherapeutic agents, antibiotics and synthetic peptides can inhibit MMP activity. MMP inhibitors are not being used extensively in practice, however, minocycline is an antibiotic that inhibits MMP activity and has been shown to be elevated in RA trials compared to control groups (Van Holten et al. Arthritis Research 4(6): 346-352 (2002)).

Administration of IFN-β to RA patients has been shown to cause a statistically significant reduction in the mean immunohistologic scores for CD3⁺ T cells and the expression of MMP-1, TIMP-1, CD38⁺ plasma cells, IL-6 and IL-1-β in synovial tissue. Thus, IFN-β therapy has immunomodulating effects on rheumatoid synovium and can help to diminish both joint inflammation and destruction (Smeets et al. Arthritis Rheum. 43(2): 270-4 (2000); van Holten et al. Arthritis Research 4(6): 346-253 (2002)). IFN-β-treated animals in a collagen-induced arthritis model displayed significantly less cartilage and bone destruction than controls, a result that correlated with a decreased number of positive cells of two gene products required for ostoclastogenesis, receptor activation of NF-κB ligand and c-Fos.

Administration of IFN-β also has been shown to be effective in alleviating overall symptoms of collagen-induced arthritis (CIA; Tak et al. Rheumatology 38(4): 362-9 (1999)) and juvenile rheumatoid arthritis (van Holten et al. Arthritis Research 4(6): 346-253 (2002)).

The modified IFN-β polypeptides provided herein, and nucleic acids encoding modified IFN-βs provided herein can be used in therapies for RA and CIA. The modified IFN-βs herein provide increase protein stability and improved half-life. Thus, modified IFN-β can be used to deliver longer lasting, more stable RA therapies. Examples of therapeutic improvements using modified IFN-βs include, for example, but are not limited to, lower dosages, fewer and/or less frequent administrations, decreased side effects and increased therapeutic effects. Modified IFN-βs can be tested for therapeutic effectiveness, for example by using animal models. For example, collagen-induced arthritis mice (DBA/1), or any other known disease model for RA or CIA, is treated with a modified IFN-β. Progression of disease symptoms and phenotypes is monitored to assess the effects of the modified IFN-β. As comparisons, placebo treated animals and animals treated with unmodified IFN-β can be used as controls.

2. Inflammatory Diseases and Disorders

A number of inflammatory diseases and disorders are targets for IFN-β therapy. Exemplary diseases and disorders include, for example, inflammatory bowel diseases such as ulcerative colitis and Crohn's disease, asthma and Guillain-Barre syndrome. The modified IFN-β proteins herein, and nucleic acids encoding modified IFN-βs can be used in therapies for inflammatory diseases and disorders. The modified IFN-βs herein provide increased protein stability and improved half-life. Thus, modified IFN-β can be used to deliver longer lasting, more stable therapies. Examples of therapeutic improvements using modified IFN-βs include, for example, but are not limited to, lower dosages, fewer and/or less frequent administrations, decreased side effects and increased therapeutic effects.

a. Inflammatory Bowel Disease (IBD)

Inflammatory bowel diseases are chronic disorders of the gastrointestinal tract characterized by inflammation of the intestine (increase in number and activity of inflammatory cells in the gut mucosa), obstruction of parts of the intestine and resulting in abdominal cramping and persistent diarrhea. Inflammatory bowel diseases are generally incurable and debilitating. IBDs can affect both the large intestine and the small intestine. The main forms of IBD are: Crohn's disease and ulcerative colitis (UC). A difference between the two is the location and nature of the inflammatory changes in the gut.

Overproduction of Th1 cytokines, such as IL-12 and IFN-γ, leads to initiation of intestinal inflammation and is associated with inadequate secretion of the counter-regulatory and anti-inflammatory cytokines. IFN-γ, has shown to be present in increased amounts in the gut wall of patients with IBD. IFN-γ can act synergistically with other pro-inflammatory cytokines such as TNF-α, to modulate the epithelial barrier function and facilitate the development of chronic inflammatory infiltrates.

Production of chemoattractant factors by intestinal epithelial cells can contribute to mucosal infiltration by inflammatory cells. Specifically, secretion of IL-8 has been shown to be a chemoattractant for neutrophils (Warhurst et al. Gut 42: 208-213 (1998)). Presence of inflammatory cells and their secreted products are associated with tissue damage and ulceration that are hallmarks of IBDs. Additionally, the gut epithelium can initiate leukocyte recruitment into the mucosa by synthesizing and secreting chemokines, such as IL-8. IL-8 levels have been shown, in vivo, to be elevated in IBD patients.

Animal models for IBD include those in which animals IBD spontaneously occurs, animals that have been treated with agents that promote intestinal inflammation, rodents that have been genetically manipulated through gene targeting or the introduction of transgenes and immunodeficient animals into which cell populations that mediate intestinal inflammation have been transferred. Examples of animal IBD models are provided in Table 16.

TABLE 16
Animal IBD Models

| | |
|---|---|
| Spontaneously-occurring | C3H/HeJBir mouse strain |
| | SAMP1/Yit strain mice |
| Treatment with mucosal-injuring | Trinitrobenzene sulfonic acid (TNBS) |
| agents | enemas |
| | Dextran sulfate sodium (DDS) |
| | administration |
| Alteration of cytokine function | IL-10 knockout mouse |
| | IL-2 knockout mouse |
| | TNF ΔARE mice |
| | STAT-4 transgenic mice |
| Alteration of T cell function | T-cell receptor α knockout mouse |
| | T-cell receptor β knockout mouse |
| Impairment of epithelial barrier | Mutated multidrug-resistant gene mice |
| function | Intestinal trefoil factor knockout mouse |

Anti-IL-12 and anti-IFN-γ administration have been shown to have some effect in the treatment of IBDs, albeit with varying effects. IFN-β has been shown to suppress IFN-γ production and early events in the IFN-γ signaling pathway. IFN-β has also been shown to suppress inflammatory activity, increase expression of the anti-inflammatory cytokine IL-10, enhance T suppressor and natural killer cell activity, limit damage caused by inflammation and to coordinate the immune system. IFN-β acts by inhibiting T-cell migration, down-regulating MMP expression, delaying clinical relapse and delaying progression of disability caused by the disease. IFN-β treatment also can result in a decrease in the severity of inflammation in the large and small intestines, a phenotype often associated with inflammatory bowel diseases. IFN-β can be administered for example, by injection, to treat inflammatory bowel diseases. One of the molecular mechanisms by which IFN-β exerts therapeutic benefits is by reducing gelatinase B expression and increasing its endogenous inhibitor, TIMP-1.

i. Ulcerative Colitis

Ulcerative colitis is a largely superficial inflammation of the mucosa that leads to early ulcer formation and is a condition in which inflammatory responses and morphologic changes are limited to the colon. Ulcerative colitis usually begins in the rectal and sigmoid areas and progresses upward continuously. Inflammation is primarily limited to the mucosa and includes continuous involvement of variable severity with ulceration, edema and hemorrhage along the length of the colon. Histologically, polymorphonuclear leukocytes and mononuclear cells, goblet cell depletion, distortion of the mucosal glands and crypt abscesses cause acute and chronic inflammation of the mucosa (Hendrickson et al. Clin. Microbiol. Rev. 1591: 79-94 (2002)).

Type I interferons, such as IFN-β, have been shown to have promising clinical effects in Th2-dominated diseases, such as ulcerative colitis (Tilg and Kaser, Expert Opin. Biol. Ther. 4(4): 469-481 (2004)). IFN-β has been shown to inhibit the production of IFN-γ and TNF, and to antagonize early events in the IFN-γ signaling pathway. Additionally, IFN-β has been shown to increase expression of the anti-inflammatory cytokine, IL-10 and enhance T suppressor and natural killer cell activity (Nikolaus et al. Gut 52: 1286-1290 (2003)).

The modified IFN-β proteins herein, and nucleic acids encoding modified IFN-βs can be used in treatment of ulcerative colitis. The modified IFN-βs herein provide increased protein stability and improved half-life. Thus, modified IFN-β can be used to deliver longer lasting, more stable therapies. Examples of therapeutic improvements using modified IFN-βs include, for example, but are not limited to, lower dosages, fewer and/or less frequent administrations, decreased side effects and increased therapeutic effects. Modified IFN-βs can be tested for therapeutic effectiveness for airway responsiveness in ulcerative colitis models. IFN-β also can be administered to animal models as well as subjects such as in clinical trials to assess in vivo effectiveness in comparison to placebo controls and/or controls using unmodified IFN-β.

ii. Crohn's Disease

Crohn's disease manifests primarily as a transmural inflammation involving the full thickness of the bowel wall that frequently leads to bowel obstruction, fistulas and abscess formation. Treatments for Crohn's disease include anti-inflammatory agents, such as aminosalicylates or steroids, and immunosuppressive agents, such as 6-mercaptopurine, each of which suppresses symptoms rather than cures the disease.

Crohn's disease can affect any part of the gastrointestinal tract, from mouth to anus (skip lesions), although a majority of the cases start in the terminal ileum and ascending colon. The disease is discontinuous with areas of inflammation alternating normal areas. Activated T cells and macrophages accumulate in dense areas, and in some cases, are organized into typical granulomas. Interactions between T lymphocytes and macrophages and their secreted products, are the major contributors to development of Crohn's disease, and the pathogenic findings appear to result from interactions of environmental and genetic factors (Ann. Intern. Med. 128: 848-856 (1998)).

T cells from patients having Crohn's disease produced increased levels of IFN-γ. It has been shown that IFN-β suppresses IFN-γ activity, and therefore, could be administered as a therapeutic agent for Crohn's disease.

The modified IFN-β proteins herein, and nucleic acids encoding modified IFN-βs can be used in treatment of Crohn's disease. The modified IFN-βs herein provide increase protein stability and improved half-life. Thus, modified IFN-β can be used to deliver longer lasting, more stable therapies. Examples of therapeutic improvements using modified IFN-βs include, for example, but are not limited to, lower dosages, fewer and/or less frequent administrations, decreased side effects and increased therapeutic effects. Modified IFN-βs can be tested for therapeutic effectiveness for airway responsiveness in Crohn's disease models. IFN-β also can be administered to animal models as well as subjects such as in clinical trials to assess in vivo effectiveness in comparison to placebo controls and/or controls using unmodified IFN-β.

b. Asthma

Asthma is a chronic respiratory disease, often arising from allergies, that is characterized by sudden recurring attacks of labored breathing, chest constriction, and coughing. A chronic inflammatory respiratory disease characterized by periodic attacks of wheezing, shortness of breath, and a tight feeling in the chest. A cough producing sticky mucus is symptomatic. The symptoms often appear to be caused by the body's reaction to a trigger such as an allergen (commonly pollen, house dust, animal dander), certain drugs, an irritant (such as cigarette smoke or workplace chemicals), exercise, or emotional stress. These triggers can cause the asthmatic's lungs to release chemicals that create inflammation of the bronchial lining, constriction, and bronchial spasms. If the effect on the bronchi becomes severe enough to impede exhalation, carbon dioxide can build up in the lungs and lead to unconsciousness and death.

Type I interferons, such as IFN-β, have been shown to have promising clinical effects in Th2 dominated diseases, such as allergic asthma (Tilg and Kaser, Expert Opin. Biol. Ther. 4(4): 469-481 (2004)). Specifically, oral administration of IFN-β inhibited the late asthmatic response by suppressing the increase of respiratory resistance (Satoh et al. J. Interferon Cytokine Res 19(8): 887-894 (1999)).

The modified IFN-β proteins herein, and nucleic acids encoding modified IFN-βs can be used in treatment of asthma. The modified IFN-βs herein provide increased protein stability and improved half-life. Thus, modified IFN-β can be used to deliver longer lasting, more stable therapies. Examples of therapeutic improvements using modified IFN-βs include, for example, but are not limited to, lower dosages, fewer and/or less frequent administrations, decreased side effects and increased therapeutic effects. Modified IFN-βs can be tested for therapeutic effectiveness for airway responsiveness in asthmatic animal models. IFN-β also can be administered to animal models as well as subjects such as in clinical trials to assess in vivo effectiveness in comparison to placebo controls and/or controls using unmodified IFN-β.

c. Guillain-Barre Syndrome

Guillain-Barre syndrome (also sometimes called infectious polyneuritis or Landry's paralysis) is a condition characterized by temporary inflammation of the nerves, causing pain, weakness, and paralysis in the extremities and often progressing to the chest and face. It typically occurs after recovery from a viral infection or, in rare cases, following immunization for influenza. Guillain-Barre syndrome is a disease of the nervous system due to damage to the myelin sheath around nerves. The myelin sheath acts as an insulator the same as rubber or plastic around electrical wires. Guillain-Barre syndrome is the most frequently acquired nerve disease and, in many cases, it follows shortly after a virus infection. It also is rarely associated with immunizations, surgery, and childbirth. Symptoms of Guillain-Barre Syndrome include weakness, typically beginning in the legs and progressing upward; weakness is accompanied by decreased feeling (paresthesia). In severe cases, breathing can be affected enough to require a ventilator and, rarely, the heart can be affected. Guillain-Barre syndrome has been associated with increased circulating levels of gelatinase B (MMP-9).

Experimental autoimmune neuritis (EAN) is a well-known animal model of the human Guillain-Barre syndrome (GBS) and can be used to investigate autoimmune inflammation of the peripheral nervous system. Recombinant rat IFN-β (rrIFN-β) prevented clinical signs of EAN, and when treatment began after onset of symptoms, rrIFN-β ameliorated EAN. Additionally, both B- and T-cell responses towards peripheral myelin were suppressed by rrIFN-β as indicated by a strong decrease in the numbers of infiltrating CD4(+) T cells, macrophages, and other inflammatory cells as well as a significant reduction in MHC class II antigen expression and monocyte chemotactic protein-1 (MCP-1) production. Thus, suppression of EAN by IFN-β is associated with a decrease in the migration of inflammatory cells into peripheral nervous tissue Zou et al. (J. Neurosci. Res. 56(2): 123-30 (1999)). IFN-β modulates motility of activated normal lymphocytes across both human brain microvascular endothelial cells and extracellular matrix proteins. This inhibitor action is generally associated with decreased production of gelatinase B (MMP-9). IFN-β has been shown to induce a dose-dependent inhibition of lymphocyte adhesion to recombinant VCAM-1 and recombinant ICAM-1. Inhibition of the adhesion phase of leukodiapedesis is an important event for recovery of Guillain-Barre syndrome (Creange et al. Neurology. 57(9): 1704-6 (2001)).

The modified IFN-β proteins herein, and nucleic acids encoding modified IFN-βs can be used in treatment of Guillain-Barre syndrome. The modified IFN-βs herein provide increase protein stability and improved half-life. Thus, modified IFN-β can be used to deliver longer lasting, more stable therapies. Examples of therapeutic improvements using modified IFN-βs include, for example, but are not limited to, lower dosages, fewer and/or less frequent administrations, decreased side effects and increased therapeutic effects. Modified IFN-βs can be tested for therapeutic effectiveness in experimental autoimmune neuritis animal models. IFN-β also can be administered to animal models as well as subjects such as in clinical trials to assess in vivo effectiveness in comparison to placebo controls and/or controls using unmodified IFN-β.

3. Proliferative Disorders

A number of proliferative diseases and disorders are targets for IFN-β therapy. Exemplary diseases and disorders include, for example, cancers and bone disorders. The modified IFN-β proteins herein, and nucleic acids encoding modified IFN-βs can be used in therapies for proliferative diseases and disorders. The modified IFN-βs herein provide increased protein stability and improved half-life. Thus, modified IFN-β can be used to deliver longer lasting, more stable therapies. Examples of therapeutic improvements using modified IFN-βs include, for example, but are not limited to, lower dosages, fewer and/or less frequent administrations, decreased side effects and increased therapeutic effects.

a. Cancer

IFN-β therapy can be used to treat a wide variety of cancers, including but not limited to, melanomas, such as uveal melanomas and their metastasis, colon and liver cancers and metastatic tumors, including, metastasis of cancers to colon, lungs and liver, and carcinomas. Treatments include systemic and localized administration of IFN-β. For example, IFN-β can be administered alone or in combination with, prior to, or subsequent to other cancer treating agents such as chemotherapeutic compounds. Cancer treatments include reduction of metastasis as well as treatment at tumor sites. Modes of administration include, but are not limited to, IFN-β protein injection, stem cell engraftment at tumor sites, administration of an adenovirus vector encoding an IFN-β systemically and/or at the tumor site. For eyes diseases such as melanoma, IFN-β can be administered by intraocular administration of protein and or nucleic acids (e.g. transfer vectors such as adenovirus and naked DNA). IFN-β also can be expressed in stem cells and stem cell engrafted at the tumor site used for targeted therapy.

The modified IFN-β proteins herein, and nucleic acids encoding modified IFN-βs can be used in cancer therapies. The modified IFN-βs herein provide increase protein stability and improved half-life. Thus, modified IFN-β can be used to deliver longer lasting, more stable cancer therapies. Examples of therapeutic improvements using modified IFN-βs include, for example, but are not limited to, lower dosages, fewer and/or less frequent administrations, decreased side effects and increased therapeutic effects.

Modified IFN-βs can be tested for therapeutic effectiveness using animal models for cancers. In one non-limiting example, an animal model such as a BALB/c mouse model for colon cancer is injected with a modified IFN-β preparation following tumor implantation into the model. Tumor size and metastasis is monitored over time compared to control animals not injected with IFN-β and/or animals injected with unmodified IFN-β. Additional immune responses such as induction of cell death and stimulation of natural killer cells can be monitored.

b. Bone Homeostasis

Osteoclasts are cells of monocyte/macrophage origin that erode bone matrix and regulation of their differentiation is central to the understanding of the pathogenesis and treatment of bone diseases such as osteoporosis. IFN-β has a role in bone homeostasis and has chondroprotective properties (Van Holten et al. Arthritis Research 4(6): 346-352 (2002)). Mice deficient in IFN-β signaling exhibit severe osteopenia (loss of bone mass) accompanied by enhanced osteoclastogenesis (Takayanagi et al. Nature 416(6882): 744-749 (2002)). Briefly, bone-resorbing osteoclasts and bone-forming osteoblasts are essential to maintaining a balance between bone resorption and formation. When the balance is disrupted in favor of osteoclasts, bone destruction occurs, such as observed in rheumatoid arthritis. Receptor activator of nuclear factor-κB ligand (RANKL) promotes the formation of osteoclasts, whereas osteoprotegerin inhibits osteoclast formation. The relative number of osteoclasts depends on the levels of RANKL versus osteoprotegerin. Additionally, osteoclasts control their own differentiation through a negative feedback mechanism. RANKL induces expression of IFN-β in osteoclast precursor cells via the transcription factor c-Fos. The cells then release IFN-β, which binds to and activates IFNAR1 and IFNAR2 on the precursors, thereby causing a decrease in c-Fos levels. Lack of c-Fos results in inhibition of osteoclast differentiation. Thus, IFN-β is central to bone homeostasis by inhibiting bone destruction.

The modified IFN-β proteins herein, and nucleic acids encoding modified IFN-βs can be used in treatment of diseases or disorders in which bone destruction occurs, including, but not limited to osteoporosis and osteopenia, as well as bone destruction in arthritis. The modified IFN-βs herein provide increased protein stability and improved half-life. Thus, modified IFN-β can be used to deliver longer lasting, more stable therapies. Examples of therapeutic improvements using modified IFN-βs include, for example, but are not limited to, lower dosages, fewer and/or less frequent administrations, decreased side effects and increased therapeutic effects. Modified IFN-βs can be tested for therapeutic effectiveness in collagen-induced arthritis animal models. IFN-β also can be administered to animal models as well as subjects such as in clinical trials to assess in vivo effectiveness in comparison to placebo controls and/or controls using unmodified IFN-β.

4. Viral Infections

IFN-β can be used in the treatment of viral infections. For example, IFN-β therapies are used for the treatment of chronic viral hepatitis, such as hepatitis A and hepatitis B. Additionally, IFN-β can be used in the treatment of myocardial viral infection, including myocardial enteroviral persistence and myocardial adenovirus persistence. Treatment effects include reduction and/or elimination of the viral genomes as well as improved left ventricular function. IFN-β also can be used in the treatment of severe acute respiratory syndrome (SARS).

The modified IFN-β proteins herein, and nucleic acids encoding modified IFN-βs can be used in treatment of viral infections. The modified IFN-βs herein provide increased protein stability and improved half-life. Thus, modified IFN-β can be used to deliver longer lasting, more stable therapies. Examples of therapeutic improvements using modified IFN-βs include, for example, but are not limited to, lower dosages, fewer and/or less frequent administrations, decreased side effects and increased therapeutic effects. Modified IFN-βs can be tested for therapeutic effectiveness against viral infection in in vitro cell systems as well as in animal models. For example, viral replication can be measured in an in vitro cell culture system by infecting the cells with virus, and expressing IFN-β in such cells or administering IFN-β to the cells. Inhibition of viral replication can be monitored in comparison to controls using unmodified IFN-β. IFN-β also can be administered to animal models as well as subjects such as in clinical trials to assess in vivo effectiveness in comparison to placebo controls and/or controls using unmodified IFN-β.

### I. COMBINATION THERAPIES

In addition to the therapeutic uses described above, the modified IFN-β proteins, and nucleic acid molecules encoding modified IFN-β proteins can be administered in combination with other therapies including other biologics and small molecule compounds. For example, in viral therapies, such as treatment for hepatitis, modified IFN-β can be administered with additional anti-viral compounds, for example, flavin adenine dinucleotide. In another example, IFN-β can be used in the treatment of cancers with other anti-cancer treatments such as chemotherapeutic compounds, including 5-fluorouracil, cisplatin and doxorubicin. In another example, IFN-β can be used in the treatment of multiple sclerosis with additional compounds such as copolymer 1.

The modified IFN-β polypeptides also, optionally, can be administered with other cytokines such as for example, G-CSF and GM-CSF and others, including cytokines that have been modified for increased stability.

### J. ARTICLES OF MANUFACTURE AND KITS

Pharmaceutical compounds of modified IFN-β polypeptides or nucleic acids encoding modified IFN-β polypeptides, or a derivative or a biologically active portion thereof can be packaged as articles of manufacture containing packaging material, a pharmaceutical composition which is effective for treating an IFN-β-mediated disease or disorder, and a label that indicates that modified IFN-β polypeptide or nucleic acid molecule is to be used for treating a IFN-β-mediated disease or disorder.

The articles of manufacture provided herein contain packaging materials. Packaging materials for use in packaging pharmaceutical products are well known to those of skill in the art. See, for example, U.S. Pat. Nos. 5,323,907 and 5,052,558 each of which is incorporated herein in its entirety. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any IFN-β-mediated disease or disorder.

Modified IFN-β polypeptides and nucleic acid molecules also can be provided as kits. Kits can include a pharmaceutical composition described herein and an item for administration. For example a modified IFN-β can be supplied with a device for administration, such as a syringe, an inhaler, a dosage cup, a dropper, or an applicator. The kit can, optionally, include instructions for application including dosages, dosing regimens and instructions for modes of administration. Kits also can include a pharmaceutical composition described herein and an item for diagnosis. For example, such kits can include an item for measuring the concentration, amount or activity of IFN-β or an IFN-β regulated system of a subject.

### K. EXAMPLES

The following examples are included for illustrative purposes only and are not intended to limit the scope of the invention(s).

### Example 1

### Cloning of IFN-β into pNAUT-a Mammalian Expression Vector

The cDNA encoding IFN-β was cloned into a mammalian expression vector, prior to the generation of the selected mutations. A collection of pre-designed, targeted mutants was then generated such that each individual mutant was created and processed individually, physically separated from each other and in addressable arrays.

The mammalian expression vector was designed by first engineering the SSV9 CMV 0.3 pA vector (SSV9, also called psub201, is a clone containing the entire adeno-associated virus (AAV) genome inserted into the PvuII site of plasmid pEMBL, see e.g., Du et al., (1996) Gene Ther 3:254-261; Samulski et al. (1987) J Virol 61:3096-3101; U.S. Pat. No. 5,753,500) as follows:

Prior to the introduction of a new EcoRI restriction site by Quickchange® mutagenesis (Stratagene), the pSSV9 CMV 0.3 pA was cut by PvuII and re-ligated to get rid of the ITR (AAV inverted terminal repeat) functions. The oligonucleotides primers were:

EcoRI forward primer:

(SEQ ID NO: 513)

5′-GCCTGTATGATTTATTGGATGTTGGAATTCCCTGATGCGGTATTTTC
TCCTTACG-3′
and
EcoRI reverse primer:

(SEQ ID NO: 514)

5′-CGTAAGGAGAAAATACCGCATCAGGGAATTCCAACATCCAATAAATC
ATACAGGC-3′.
The construct sequence was confirmed by using the
following oligonucleotides:
Seq ClaI forward primer:

(SEQ ID NO: 515)

5′-CTGATTATCAACCGGGGTACATATGATTGACATGC-3′
and
Seq XmnI reverse primer:

(SEQ ID NO: 516)

5′-TACGGGATAATACCGCGCCACATAGCAGAAC-3′.

Following digestion with XmnI and ClaI, the XmnI-ClaI fragment containing the newly introduced EcoRI site was cloned into pSSV9 CMV 0.3 pA to replace the corresponding wild-type fragment and produce the construct pSSV9-2EcoRI.

The IFN β-cDNA was obtained from the pLG104R (containing human IFN-β1 gene, ATCC # 31902) construct. The sequence of the IFN β-cDNA was confirmed by sequencing using the primers below:

| | | |
|---|---|---|
| Seq forward primer: | 5′-CCTGATGAAGGAGGACTC-3′ | (SEQ ID NO: 517) |
| Seq reverse primer: | 5′-CCAAGCAGCAGATGAGTC-3′. | (SEQ ID NO: 518) |

The verified IFN β-encoding cDNA first was cloned into an E. coli vector (pTOPO-TA, Invitrogen). After checking of the cDNA sequence by automatic DNA sequencing, restriction of the vector at HindIII and XbaI restriction sites yielded a HindIII-XbaI fragment containing the IFN-β cDNA which was sub-cloned into the corresponding sites of pSSV9-2EcoRI, leading to the construct pAAV-EcoRI-IFN-beta (pNB-AAV-IFN-beta). Finally, following digestion with PvuII, the PvuII digested fragment of plasmid pNB-AAV-IFN-beta was sub-cloned into the PvuII site of pUC18 leading the final construct pUC-CMV-IFN-beta pA called pNAUT-IFN-beta (SEQ ID NO:660).

### Example 2

### Design of IFN-β Variants by 2D-Scanning and 3D-Scanning

2D- and 3D-scanning technology, described herein and also described in published Application No. US-2004-0132977-A1 and U.S. application Ser. No. 10/658,355, was used to design and obtain IFN-β mutants with improved resistance to proteolysis and/or improved conformational stability, such as improved thermal stability. Is-HITs were identified based upon (1) protein property to be evolved (i.e., resistance to proteolysis or conformational stability); (2) amino acid sequence; and (3) properties of individual amino acids.

A. LEADS Created for Higher Resistance to Proteolysis

Variants were designed using 2D-scanning or 3D-scanning. Positions selected (is-HITs) on IFN-β (SEQ ID NO:1 or 3) were (numbering corresponds to amino acid positions in the mature protein of SEQ ID NO:1): Y3, L6, Q18, K19, L20, Q23, E29, L31, L24, K33, D34, F38, D39, P41, E42, E43, K45, Q48, Q49, F50, Q51, K52, E53, D54, L57, Y60, E61, M62, L63, Q64, F70, Q72, D73, W79, E81, E85, L87, L88, L98, K99, L102, E103, E104, K105, L106, E107, K108, E109, D110, K115, M117, L122, K123, Y125, Y126, Y132, L133, K134, K136, E137, Y138, W143, R147, E149, L151, F154, F156, L160, and L164. The native amino acids at each of the is-HIT positions listed and shown above was replaced by residues as listed in Table 17.

TABLE 17

| Amino acid at is-HIT | Replacing amino acids |
|---|---|
| Y | H, I |
| L | I, V, H, A, T, Q |
| D | N, Q, G |
| F | I, V |
| P | A, S |
| E | Q, H, N |
| M | I, V, T, Q, H, A |
| W | H, S |
| K | N, Q, S, H |
| R | H, Q |
| Q | H, S, T, N |

The variants generated were as follows Y3H, Y3I, L6I, L6V, L6H, L6A, L20I, L20V, L20H, L20A, L21I, L21V, L21T, L21Q, L21H, L21A, L24I, L24V, L24T, L24Q, L24H, L24A, D34N, D34Q, D34G, F38I, F38V, P41A, P41S, E43Q, E43H, E43N, F50I, F50V, E53Q, E53H, E53N, D54N, D54Q, D54G, L57I, L57V, L57T, L57Q, L57H, L57A, Y60H, Y60I, E61Q, E61H, E61N, M62I, M62V, M62T, M62Q, M62A, L63I, L63V, L63T, L63Q, L63H, L63A, F70I, F70V, W79H, W79S, L87I, L87V, L87H, L87A, L88I, L88V, L88T, L88Q, L88H, L88A, L98I, L98V, L98H, L98A, L102I, L102V, L102T, L102Q, L102H, L102A, L106I, L106V, L106T, L106Q, L106H, L106A, K115N, K115Q, K115S, K115H, K115N, M117I, M117V, M117T, M117Q, M117A, L122I, L122V, L122T, L122Q, L122H, L122A, Y125H, Y125I, Y126H, Y126I, Y132H, Y132I, L133I, L133V, L133T, L133Q, L133H, L133A, W143H, W143S, R147H, R147Q, E149Q, E149H, E149N, L151I, L151V, L151T, L151Q, L151H, L151A, F154I, F154V, F156I, F156V, L160I, L160V, L160T, L160Q, L160H, L160A, L164I, L164V, L164T, L164Q, L164H, L164A, K19N, E29N, K33N, D39N, E42N, K45N, K52N, D73N, E81N, E85N, K99N, E103N, E104N, K105N, E107N, K108N, E109N, D110N, K123N, K134N, K136N, E137N, Q18H, Q18S, Q18T, Q18N, Q23H, Q23S, Q23T, Q23N, Q48H, Q48S, Q48T, Q48N, Q49H, Q49S, Q49T, Q49N, Q51H, Q51S, Q51T, Q51N, Q64H, Q64S, Q64T, Q64N, Q72H, Q72S, Q72T, and Q72N. See SEQ ID NOS: 4-68, 71-87, 534, 535, 536-594, and 596-650. The variants were tested for biologic activity as described in Example 5 and for resistance to proteolysis as described in Example 7.

### LEADS Created for Higher Resistance to Proteolysis by Gelatinase B

Variants were designed using 2D scanning. Amino acids Phenylalanine (F), Leucine (L), Glutamic Acid (E), Tyrosine (Y), and Glutamine (Q) were chosen as target amino acids for replacement to increase proteolysis resistance of an IFN-β polypeptide by gelatinase B. Positions selected (is-HITs) on IFN-β (SEQ ID NO:1 or 3) were (numbering corresponds to amino acid positions in the mature protein of SEQ ID NO:1): Y3, L5, L6, F8, L9, Q10, F15, Q16, Q18, L20, L21, Q23, L24, L28, E29, Y30, L32, F38, E42, E43, L47, Q48, Q49, F50, Q51, E53, L57, Y60, E61, L63, Q64, F67, F70, Q72, E81, E85, L87, L88, Y92, Q94, L98, L102, E103, E104, L106, E107, E109, F111, L116, L120, Y125, Y126, L130, Y132, L133, E137, Y138, E149, L151, F154, F156, L160, Y163, and L164. The variants generated were as follows: Y3H, Y3I, L5V, L5I, L5T, L5Q, L5H, L5A, L5D, L5E, L5K, L5R, L5N, L5S, L6I, L6V, L6H, L6A, L6D, L6E, L6K, L6N, L6Q, L6R, L6S, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, F15I, F15V, F15D, F15E, F15K, F15R, Q16D, Q16E, Q16K, Q16N, Q16R, Q16S, Q16T, Q16C, Q18H, Q18S, Q18T, Q18N, L20I, L20V, L20H, L20A, L20N, L20Q, L20R, L20S, L20T, L20D, L20E, L20K, L21I, L21V, L21T, L21Q, L21H, L21A, Q23H, Q23S, Q23T, Q23N, Q23D, Q23E, Q23K, Q23R, L28V, L28I, L28T, L28Q, L28H, L28A, E29N, E29Q, E29H, Y30H, Y30I, L32V, L32I, L32T, L32Q, L32H, L32A, F38I, F38V, E42N, E42Q, E42H, E43Q, E43H, E43N, L47V, L47I, L47T, L47Q, L47H, L47A, Q48H, Q48S, Q48T, Q48N, Q49H, Q49S, Q49T, Q49N, F50I, F50V, Q51H, Q51S, Q51T, Q51N, E53Q, E53H, E53N, L57I, L57V, L57T, L57Q, L57H, L57A, Y60H, Y60I, E61Q, E61H, E61N, L63I, L63V, L63T, L63Q, L63H, L63A, Q64H, Q64S, Q64T, Q64N, F67I, F67V, F70I, F70V, Q72H, Q72S, Q72T, Q72N, E81N, E81Q, E81H, E85N, E85Q, E85H, L87I, L87V, L87H, L87A, L87D, L87E, L87, L87R, L87N, L87Q, L87S, L87T, L88I, L88V, L88T, L88Q, L88H, L88A, Y92H, Y92I, Q94D, Q94E, Q94K, Q94N, Q94R, Q94S, Q94T, Q94C, L98I, L98V, L98H, L98A, L98D, L98E, L98K, L98N, L98Q, L98R, L98S, L98T, L98C, L102I, L102V, L102T, L102Q, L102H, L102A, E103N, E103Q, E103H, E104N, E104Q, E104H, L106I, L106V, L106T, L106Q, L106H, L106A, E107N, E107Q, E107H, E109N, E109H, E109Q, F111I, F111V, L116V, L116I, L116T, L116Q, L116H, L116A. L116V, L116I, L116T, L116Q, L116H, L116A, Y125H, Y125I, Y126H, Y126I, L130V, L130I, L130T, L130Q, L130H, L130A, Y132H, Y132I, L133I, L133V, L133T, L133Q, L133H, L133A, E137N, E137Q, E137H, Y138H, Y138I, E149Q, E149H, E149N, L151I, L151V, L151T, L151Q, L151H, L151A, F154I, F154V, F156I, F156V, L160I, L160V, L160T, L160Q, L160H, L160A, Y163H, Y163I, L164I, L164V, L164T, L164Q, L164H, and L164A. See SEQ ID NOS: 4-11, 16, 17, 20-27, 30-36, 39-42, 45-54, 61-70, 75-87, 157, 158, 163-168, 173, 174, 180-185, 190-193, 198, 199, 204, 205, 209, 210, 213-224, 233-238, 247-250, 266-279, 282, 283, 295-310, 328-358, 377-387, 396-403, 408-411, 447-454, 474-479, 497-504, 540-542, 547, 551, 555-558, 562-576, 578-583, 585-589, 591, 604-607, 610-614, 616-650, 652, 653, 655, 656, and 658. The variants were tested for biologic activity as described in Example 5 and for resistance to proteolysis by gelatinase B as described in Example 8.

B. LEADS Created to Stabilize IFN-β by Increasing Polar Interactions

Using the 2D scanning methods described above, charges were introduced into the hydrophobic areas of the IFN-β protein to favor polar interactions with the solvent. Positions containing amino acid residues with a side-chain oriented toward the solvent were selected for replacement with amino acids E, D, K, and R. The variants generated were as follows L5E, L5D, L5K, L5R, F8E, F8D, F8K, F8R, L9E, L9D, L9K, S12E, S12D, S12K S12R, F15E, F15D, F15K, F15R, Q16E, Q16D, Q16K, Q16R, L20E, L20D, L20K, L20R, W22E, W22D, W22K, Q23E, Q23D, Q23K, Q23R, L24E, L24D, L24K, L24R, G78E, G78D, G78K, G78R, W79E, W79D, W79K, W79R, N80E, N80D, N80K, T82E, T82D, T82K, T82R, I83E, I83D, I83K, I83R, N86E, N86D, N86K, N86R, L87E, L87D, L87K, L87R, A89E, A89D, A89K, and A89R. See SEQ ID NOS: 329-331, 342-348, 350, 359-362, 377-383, 385 397-398, 401, 403-435, 440-443, 447-450, and 455-458. Activity was assessed as described in Example 5 and conformational stability was assessed by resistance to temperature as is described in Example 9.

C. LEADS Created to Stabilize IFN-β by Increasing Polar Interactions Between Helices A and C

Mutations were made using the 2D scanning methods described above to increase polar interactions between helices A and C. Selected is-HIT positions were replaced with a selection of amino acids from E, D, K, R, N, Q, S and T. The variants generated were as follows M1E, M1D, M1K, M1R, M1N, M1Q, M1S, M1T, L5E, L5D, L5K, L5R, L5N, L5Q, L5S, L5T, L6E, L6D, L6K, L6R, L6N, L6Q, L6S, L6T, L9E, L9D, L9K, L9R, L9N, L9Q, L9S, L9T, Q10E, Q10D, Q10K, Q10R, Q10N, Q10S, Q10T, S13E, S13D, S13K, S13R, S13N, S13Q, S13T, N14E, N14D, N14K, N14R, N14Q, N14S, N14T, Q16E, Q16D, Q16K, Q16R, Q16N, Q16S, Q16T, C17E, C17D, C17K, C17R, C17N, C17Q, C17S, C17T, L20E, L20D, L20K, L20R, L20N, L20Q, L20S, L20T, I83E, I83D, I83K, I83R, I83N, I83Q, I83S, I83T, N86E, N86D, N86K, N86R, N86Q, N86S, N86T, L87E, L87D, L87K, L87R, L87N, L87Q, L87S, L87T, N90E, N90D, N90K, N90R, N90Q, N90S, N90T, V91E, V91D, V91K, V91R, V91N, V91Q, V91S, V91T, Q94E, Q94D, Q94K, Q94R, Q94N, Q94S, Q94T, I95E, I95D, I95K, I95R, I95N, I95Q, I95S, I95T, H97E, H97D, H97K, H97R, H97N, H97Q, H97S, H97T, L98E, L98D, L98K, L98R, L98N, L98Q, L98S, L98T, V101E, V101D, V101K, V101R, V101N, V101Q, V101S, and V101T. See SEQ ID NOS: 264, 268, 2669, 276, 277, 322-341, 346-358, 363-376, 381-403, 432-454, and 459-512. Activity was assessed as described in Example 5 and conformational stability was assessed by resistance to temperature as is described in Example 9.

D. LEADS Created to Stabilize IFN-B by Forming Disulfide Bridges Between Helices A and C

Mutations were made using the 2D scanning methods described above to create new intra-molecular disulfide bridges. The bridges impose conformational constraints and minimize denaturation by temperature or changes in pH during the production, storage or injection of the protein. Amino acids at selected is-HIT positions M1, L6, S10, S13, Q16, N90, V91, Q94, L98, and V101 were replaced by a cysteine (C). Combinations of one or more is-HIT position with the corresponding amino acid modification to a cysteine created a disulfide bridge. Each new bridge is based on two mutations, except bridges #7 and #8 which utilize the cysteine residue at amino acid position 17. The disulfide bridges are formed by the following pairs of variants: bridge #1 is M1C-V101C; bridge #2 is L6C-L98C; bridge #3 is Q10C-H97C; bridge #4 is Q10C-L98C; bridge #5 is S13C-Q94C; bridge #6 is Q16C-N90C; bridge #7 is N90C-C17; and bridge #8 is V91C-C17. See SEQ ID NOS: 126-133. Activity was assessed as described in Example 5 and conformational stability was assessed by resistance to temperature as is described in Example 9.

E. LEADS Created to Stabilize IFN-β by Altering the Isoelectric Point

The isoelectric point (pI) is the pH at which a protein has net charge of zero. IFN-β is a basic protein with a pI of 8.93 and 40 charged amino acids. It has been observed that IFN-β aggregates at pH 6-7, but is stable at pH 4 and that these properties are related to the surface charge. Thus, two strategies were performed to increase the stability of IFN-β: 1) increasing and 2) decreasing the isoelectric point of IFN-β.

Mutations at selected is-HIT positions E43, E53, D54, E61, E81, E85, E103, E104, E107, E109, and D110 by replacing E or D with K or R to add positive charges to the protein resulting in an increased pI by about 0.3 or 0.3. The variants were as follows: E43K, E53R, D54K, E61K, E81K, E85K, E103K, E104R, E107R, E109R, and D110K. See SEQ ID NOS: 134-144. Activity was assessed as described in Example 5 and conformational stability was assessed by resistance to temperature as is described in Example 9.

Mutations at selected is-HIT positions R11, K45, K52, K105, K108, R113, K115, R124, R152, and R165 by replacing K or R with Q decreased the pI by about 0.55 or 0.55. The variants were as follows: R11Q, K45Q, K52Q, K105Q, K108Q, R113Q, K115Q, R124Q, R152Q, and R165Q. See SEQ ID NOS: 56, 159, 169, 194, 200, 212, 230, 252, 256, and 281. Activity was assessed as described in Example 5 and conformational stability was assessed by resistance to temperature as is described in Example 9.

Mutations at selected is-HIT positions R11, K45, K52, K105, K108, R113, K115, R124, R152, and R165 by replacing basic amino acids K or R with acidic amino acids D or E decreased the pI by about 0.2 or 0.2. The variants were as follows: R11D, K45D, K52D, K105D, K108D, R113E, K115D, R124D, R124E, R152D, and R165D. See SEQ ID NOS: 145-153, 519, and 520. Activity was assessed as described in Example 5 and conformational stability was assessed by resistance to temperature as is described in Example 9.

### Example 3

### Mutagenesis

Mutagenesis was performed by replacing single amino acid residues at specific is-HIT target positions one-by-one. Once replacing amino acids were identified, they were systematically introduced to replace the is-HIT loci in the protein and thus, candidate LEADs were produced. Using standard recombinant DNA methods, mutagenesis reactions were performed with the Quickchange kit (Invitrogen) using pNAUT-IFNβ (SEQ ID NO:660) as the template and the presence of the mutation was verified by sequencing. Each mutant generated was the single product of an individual mutagenesis reaction. Substituted amino acids were compatible with protein structure and function. Mutant proteins were assessed in appropriate biological assays (see Example 5) and for the particular property modified (see Examples 7-9).

### Example 4

### Production and Normalization of Native and Modified IFN-β in Mammalian Cells

IFN-β was produced in Chinese Hamster Ovarian (CHO) cells (obtained from ATCC), using Dulbecco's modified Eagle's medium supplemented with glucose (4.5 g/L; Gibco-BRL) and fetal bovine serum (5%, Hyclone). Production of either native or mutant IFN-β was performed by transient transfection. Cells were transiently transfected as follows: 0.6×10⁵ cells were seeded into 6-well plates and grown for 24 h before transfection. Cells, at about 70% confluence, were supplemented with 1.0 pg of plasmid (from the library of pNAUT-IFN-β mutants, see above) by lipofectamine plus reagent (Invitrogen). After gently shaking, cells were incubated for 24 h with 1 ml of culture medium supplemented with 1% serum. IFN-β was obtained from culture supernatants 24 h after transfection and stored in aliquots at −80° C. until use.

Normalization of IFN-β concentration from culture supernatants was performed by ELISA. IFN-β concentrations from wild-type, and mutant samples were estimated by using an international reference standard provided by the National Institute for Biological Standard and Controls (NIBSC, United Kingdom). Preparations of IFN-β produced from transfected cells were screened following sequential biological activity assays as described below.

### Example 5

### Analysis of the Activity of the Modified IFN-β Polypeptides

Two activities were measured directly on IFN-β samples: anti-viral and anti-proliferation activities. Dose (concentration)-response (activity) experiments for anti-viral or anti-proliferation activity allowed for the calculation of the “potency” of anti-viral and anti-proliferation activities, respectively. Anti-viral and anti-proliferation activities also were measured after incubation with proteolytic samples such as specific proteases, mixtures of selected proteases, human serum or human blood as described below in Example 7. Assessment of activity following incubation with proteolytic samples allowed for the determination of the residual (anti-viral or anti-proliferation) activity and the respective kinetics of half-life upon exposure to proteases.

a. Assessment of Anti-Viral Activity

Anti-viral activity can be measured by cytopathic effects (CPE). Anti-viral activity of IFN-β was determined by the capacity of the cytokine to protect HeLa cells against EMC (mouse encephalomyocarditis) virus-induced cytopathic effects. The day before, HeLa cells (2×10⁵ cells/ml) were seeded in flat-bottomed 96-well plates containing 100 μl/well of Dulbecco's MEM-Glutamax-sodium pyruvate medium supplemented with 5% SVF and 0.2% of gentamicin. Cells were grown at 37° C. in an atmosphere of 5% CO₂ for 24 hours.

Two-fold serial dilutions of IFN-β samples were made with MEM complete media into 96-deep well plates with the final concentration ranging from 1600 to 0.6 pg/ml of IFN-β polypeptide. The medium was aspirated from each well and 100 μl of IFN-β dilutions were added to HeLa cells. Each IFN β sample dilution was assessed in triplicate. The two last rows of the plates were filled with 100 μl of medium without IFN-β dilution samples in order to serve as controls for cells with and without virus.

After 24 hours of growth, a 1/1000 EMC virus dilution solution was placed in each well, except for the cell control row. Plates were returned to the CO₂ incubator for 48 hours. Then, the medium was aspirated and the cells were stained for 1 hour with 100 μl of Trypan Blue staining solution to determine the proportion of intact cells. Plates were washed in a distilled water bath. The cell-bound dye was extracted using 100 μl of ethylene-glycol mono-ethyl-ether (Sigma). The absorbance of the dye was measured using an ELISA plate reader (Spectramax). The anti-viral activity of IFN-β samples (expressed as EC₅₀ average, pg/ml) was determined as the concentration of IFN-β needed for 50% protection of the cells against EMC virus-induced cytopathic effects.

b. Assessment of Anti-Proliferation Activity

Anti-proliferative activity of IFN-β was determined by assessing the capacity of wildtype or modified IFN-β to inhibit proliferation of Daudi cells. Daudi cells (ATCC, 1×10⁴ cells) were seeded in flat-bottomed 96-well plates containing 50 μl/well of RPMI-1640 medium supplemented with 10% SVF, 1× glutamine and 1 mL of gentamicin. No cells were added to the last row (“H” row) of the flat-bottomed 96-well plates in order to evaluate background absorbance of culture medium.

At the same time, two-fold serial dilutions of IFN-β samples were made with RPMI 1640 complete media into 96-deep-well plates with final concentration ranging from 6000 to 2.9 pg/ml. IFN-β dilutions (50 μl) were added to each well containing 50 μl of Daudi cells for a total volume in each well of 100 μl. Each IFN-β sample dilution was assessed in triplicate. Each well of the “G” row of the plates was filled with 50 μl of RPMI 1640 complete media as a positive control. The plates were incubated for 72 hours at 37° C. in a humidified, 5% CO₂ atmosphere.

After 72 hours of growth, 20 μl of Cell titer 96® Aqueous one solution reagent (Promega) was added to each well and incubated for 1.5 hours at 37° C. in an atmosphere of 5% CO₂. To measure the amount of colored soluble formazan produced by cellular reduction of the tetrazolium compound (3-(4,5-dimethylthiazol-2-yl)-5-(3-carboxymethoxyphenyl)-2-(4-sulfophenyl)-2H-tetrazolium, inner salt; MTS) in the Cell titer 96® Aqueous one solution reagent, the absorbance of the dye was measured using an ELISA plate reader (Spectramax) at 490 nm.

The corrected absorbances (“H” row background value subtracted) obtained at 490 nm were plotted versus concentration of IFN-β. The EC₅₀ value was calculated by determining the X-axis value corresponding to one-half the difference between the maximum and minimum absorbance values. (EC₅₀=the concentration of IFN-β necessary to give one-half the maximum response, see Example 6).

### Example 6

### Evaluation of IFN-β Variants

Various biological activities (see Example 5) and properties (see Examples 7-9), including protease resistance and potency of each individual mutant, were analyzed using a mathematical model and algorithm (NautScan; Fr. Patent No. 9915884; see, also published International PCT application No. WO 01/44809 based on PCT No. PCT/FR00/03503; and described above).

Data was processed using a Hill equation-based model that uses key feature indicators of the performance of each individual mutant. Briefly, the Hill equation is a mathematical model that relates the concentration of a drug (i.e., test compound or substance) to the response measured.

$$y = \frac{{{y}_{max} [ D ]}^{x}}{{[ D ]}^{n} + {[ {D}_{50} ]}^{n}}$$

y is the variable measured, such as a response, signal, yₘₐₓ is the maximal response achievable, [D] is the molar concentration of a drug (e.g., the IFN-β or modified IFN-β), [D₅₀] is the concentration that produces a 50% maximal response to the drug, n is the slope parameter, which is 1 if the drug binds to a single site and with no cooperativity between or among sites. A Hill plot is log₁₀ of the ratio of ligand-occupied receptor to free receptor vs. log [D] (M). The slope is n, where a slope of greater than 1 indicates cooperativity among binding sites, and a slope of less than 1 can indicate heterogeneity of binding. This equation has been employed in methods for assessing interactions in complex biological systems, the parameters, π, κ, τ, ε, η, θ, are as follows:

π is the potency of the biological agent acting on the assay (cell-based) system;

κ is the constant of resistance of the assay system to elicit a response to a biological agent;

ε is the slope at the inflexion point of the Hill curve (or, in general, of any other sigmoidal or linear approximation), to assess the efficiency of the global reaction (the biological agent and the assay system taken together) to elicit the biological or pharmacological response.

τ is used to measure the limiting dilution or the apparent titer of the biological agent.

θ is used to measure the absolute limiting dilution or titer of the biological agent.

η is the heterogeneity of the biological process or reaction. η measures the existence of discontinuous phases along the global reaction, which is reflected by an abrupt change in the value of the Hill coefficient or in the constant of resistance.

Modified IFN-β polypeptides were ranked based on the values of their individual performance, as assessed by EC₅₀. The biological specific activity (i.e., activity per unit of protein mass; units/mg protein) was determined for each mutant using serial dilutions of the mutant in a cell-based proliferation assay or anti-viral assay (see Example 5). Twelve dilutions were made for each curve and each dilution was assayed in triplicate. Using the data from the serial dilution assays, the concentration needed to achieve 50% activity (EC₅₀) was obtained. Experimental points were fitted to a sigmoidal curve using Gnuplot 5.0 (software for drawing data curves; available online at gnuplot.info) integrated to the NEMO (Newly evolved Molecules) software (Nautilus Biotech) as follows. The equation used for the sigmoidal curve fitting was:
Sig(x)=base+pmax*(x exp. nu)/X exp. nu)+kappa
where base, pmax, nu and kappa are parameters for each curve as follows: base=0.1, pmax=1, kappa=5000, nu=2.5 and n between 5 and 150. Gnuplot iterates fitting “n” times until it finds the best curve that fits the experimental data while minimizing the sum of the squares of the distance between each experimental point and the theoretical point on the fitting curve. Once the fitting curve for each mutant was obtained, the corresponding EC₅₀ and specific activity are calculated from the curves. Those on the top of the ranking list were selected as LEADs.

Exemplary IFN-β polypeptides tested for their activity included native wildtype IFN-β and exemplary candidate SuperLEADs such as set forth in Table 18 below. Table 18 below depicts the anti-viral activity (EC₅₀ average, pg/ml) and specific activity (average, IU/mg) of exemplary non-limiting IFN-β Super-LEAD polypeptides compared to wildtype IFN-β.

TABLE 18
Biological activity of IFN-β SuperLEADS

| | | EC₅₀ | | |
|---|---|---|---|---|
| NEMO | | Average | Specific Activity | Rate: |
| code | Mutation | (pg/mL) | Average (IU/mg) | Mutant/WT |
| 524 | L5D/L6E | 18.8 | 7.49 × 10⁸ | 2.9 |
| 525 | L5E/Q10D | 26.2 | 1.23 × 10⁹ | 4.7 |
| 526 | L5Q/M36I | 13.5 | 8.89 × 10⁸ | 3.4 |
| 527 | L6E/L47I | nd | nd | nd |
| 528 | L5E/K108S | 248.4 | 4.03 × 10⁷ | 0.2 |
| 529 | L5E/L6E | 32.8 | 2.14 × 10⁹ | 8.2 |
| 530 | L5D/Q10D | 4.50 | 1.12 × 10⁹ | 4.3 |
| 531 | L5N/M36I | 48.4 | 2.12 × 10⁸ | 0.8 |
| 532 | L6Q/L47I | 4.10 | 2.42 × 10⁹ | 9.3 |
| 533 | L5D/K108S | 15.2 | 1.31 × 10⁹ | 5.0 |
| 534 | L5N/L6E | 7.90 | 1.38 × 10⁹ | 5.3 |
| 535 | L5Q/Q10D | 21.8 | 4.58 × 10⁸ | 1.8 |
| 536 | L6E/M36I | 38.3 | 3.69 × 10⁸ | 1.4 |
| 537 | L5E/N86Q | 47.5 | 5.89 × 10⁸ | 2.3 |
| 538 | L5Q/K108S | 9.60 | 1.06 × 10⁹ | 4.1 |
| 539 | L5Q/L6E | 4.10 | 2.45 × 10⁹ | 9.4 |
| 540 | L5N/Q10D | 7.70 | 1.31 × 10⁹ | 5.0 |
| 541 | L6Q/M36I | 13.1 | 8.13 × 10⁸ | 3.1 |
| 542 | L5D/N86Q | 32.2 | 5.20 × 10⁸ | 2.0 |
| 543 | L5N/K108S | 66.0 | 3.31 × 10⁸ | 1.3 |
| 544 | L5D/L6Q | 8.90 | 1.15 × 10⁹ | 4.4 |
| 545 | L6E/Q10D | 5.80 | 1.74 × 10⁹ | 6.7 |
| 546 | L5E/L47I | nd | nd | nd |
| 547 | L5Q/N86Q | 26.9 | 3.72 × 10⁸ | 1.4 |
| 548 | L6E/K108S | 24.7 | 5.48 × 10⁸ | 2.1 |
| 549 | L5E/L6Q | 39.2 | 6.22 × 10⁸ | 2.4 |
| 550 | L6Q/Q10D | 13.9 | 7.18 × 10⁸ | 2.8 |
| 551 | L5D/L47I | 10.7 | 2.36 × 10⁹ | 9.1 |
| 552 | L5N/N86Q | 21.4 | 6.75 × 10⁸ | 2.6 |
| 553 | L6Q/K108S | 79.0 | 5.08 × 10⁸ | 1.9 |
| 554 | L5N/L6Q | 44.3 | 3.84 × 10⁸ | 1.5 |
| 555 | L5E/M36I | 5.90 | 1.71 × 10⁹ | 6.6 |
| 556 | L5Q/L47I | nd | nd | nd |
| 557 | L6E/N86Q | 25.3 | 4.00 × 10⁸ | 1.5 |
| 558 | L5Q/L6Q | 7.70 | 1.30 × 10⁹ | 5.0 |
| 559 | L5D/M36I | 52.3 | 3.93 × 10⁹ | 15.1 |
| 560 | L5N/L47I | 2.90 | 3.43 × 10⁹ | 13.1 |
| 561 | L6Q/N86Q | 30.8 | 6.12 × 10⁸ | 2.3 |
| 523 | WT | 32.49 | 3.08 × 10⁸ | 1.2 |
| 523 | WT | 33.56 | 3.32 × 10⁸ | 1.3 |
| 523 | WT | 26.99 | 2.03 × 10⁸ | 0.8 |
| 523 | WT | 29.90 | 2.18 × 10⁸ | 0.8 |
| 523 | WT | 27.05 | 2.05 × 10⁸ | 0.8 |
| 523 | WT | 32.01 | 2.99 × 10⁸ | 1.1 |
| 523 | WT average | 30.74 | 2.61 × 10⁸ | 1.0 |
| | nibsc | 40.6 | 2.46 × 10⁸ | |
| | nibsc | 32.22 | 3.10 × 10⁸ | |

nibsc: National Institute for Biological Standard and Controls (UK);
nd: not determined

### Example 7

### Assessment of Proteolytic Resistance

a. Treatment of IFN-β with Proteolytic Preparations

Following determination by ELISA of the amount of IFN-β produced (for native as well as for each mutant IFN-β), up to 150 μl of supernatant containing varying concentrations of native IFN-β or variants (0 to 1000 pg/ml) were treated with a mixture of proteases at 1% w/w of total proteins in the supernatant (e.g., 1% serum, as the concentration of total protein in 1% serum is 600 μg/ml, the 1% proteases was based on the total amount of proteins and not only in the amount of IFN-β). Modified IFN-β polypeptides were treated with proteases in order to identify resistant molecules. The resistance of the modified IFN-β molecules compared to wild-type IFN-β was determined by exposure (120 min, 25° C.) to a mixture of proteases (containing 1.5 pg of each of the following proteases (1% wt/wt, Sigma): α-chymotrypsin, carboxypeptidase, endoproteinase Arg-C, endoproteinase Asp-N, endoproteinase Glu-C, endoproteinase Lys-C, and trypsin). At the end of the incubation time, 10 μl of anti-protease complete medium containing mini EDTA free tablets (Roche) diluted 1/1000 in 10 ml DMEM was added to each reaction in order to inhibit protease activity. Treated samples were then used to determine residual anti-viral or anti-proliferation activities as described above in Example 5. Native IFN-β incubated in the absence of protease exhibited anti-viral activity beginning at concentrations greater than 10 pg/ml which dose-dependently increased until a concentration of 100 pg/ml where the concentration of IFN-β required to achieve maximal anti-viral activity reached a plateau. Incubation of native IFN-β for 2 hours with proteases resulted in no detectable residual anti-viral activity, even at concentrations as high as 800 pg/ml of IFN-β polypeptide. Modified IFN-β polypeptides exhibiting improved resistance to proteolysis were those assessed to have residual anti-viral activity following incubation with proteases comparable to native IFN-β incubated in the absence of proteases. The residual activity of modified IFN-β polypeptides was similar following incubation in the presence or absence of protease, demonstrating that the IFN-β LEAD is resistant to proteolysis.

b. Kinetic Analysis

The percent of residual IFN-β activity over time of exposure to proteases was evaluated by a kinetic study using 1.5 pg of protease mixture. Incubation times were: 0 h, 0.5 h, 2 h, 4 h, 8 h, 12 h, 24 h and 48 h. Briefly, 20 μl of each proteolytic sample (proteases, serum, blood) was added to 100 μl of IFN-β at 400 and 800 pg/ml and incubated for variable times, as indicated. At the appropriate time points, 10 μl of anti-proteases complete medium containing mini EDTA-free tablets (Roche; one tablet dissolved in 10 ml of DMEM and then diluted 1/500) was added to each well in order to stop proteolysis reactions. Biological activity assays were then performed as described above in Example 5 for each sample in order to determine the residual activity at each time point. IFN-β polypeptides tested included native IFN-β and exemplary candidate LEADs and SuperLEADs including L6Q, M36I, L5N, Q10D, L5Q, L6Q, L5E/Q10D, L5E/K108S, L6Q/L47I, L5D/K108S, L5N/L6E, L5Q/K108S, L5N/Q10D, L6Q/M36I, L5D/N86Q, L5N/K108S, L5D/L6Q, L6E/Q10D, L5Q/N86Q, L6E/K108S, L5D/L47I, L6Q/K108S, L5N/L6Q, L6E/N86Q, L5Q/L6Q, L5D/M36I, L5N/L47I, and L6Q/N86Q. All exemplary candidate LEADs and SuperLeads tested exhibited improved protease resistance compared to native IFN-β. Native IFN-β began to exhibit reduced residual anti-viral activity following incubation with protease for less than 1 hour which declined over time such that native IFN-β exhibited no detectable residual anti-viral activity following incubation with protease for 6 hours and greater. The time of incubation of native IFN-β with proteases required to give 50% of total anti-viral activity was 2 hours. In a kinetic analysis of protease resistance where incubation times tested were 0, 0.5, 1, 2.5, 4, 6, and 8, modified IFN-β polypeptides Q10D, L5N, L6Q, and L5Q exhibited about 50% residual anti-viral activity up to 8 hours incubation with protease compared to the activity of the respective polypeptides in the absence of incubation with protease. The double mutants containing a combination of the LEAD mutations, L5N/Q10D and L5Q/L6Q, exhibited residual activity up to 8 hours following incubation with protease that was similar to the anti-viral activity of the respective polypeptide in the absence of incubation with protease. Similarly, modified IFN-β polypeptides L6Q and M36I exhibited about 50% and 20%, respectively, residual anti-viral activity up to 8 hours incubation with protease compared to the activity of the respective polypeptides in the absence of incubation with protease. The double mutant containing a combination of the LEAD mutations, L6Q/M36I, exhibited residual activity up to 8 hours following incubation with protease that was similar to its anti-viral activity in the absence of incubation with protease.

In some experiments, the time of incubation with proteases required to give 50% of total activity (anti-viral or anti-proliferative) compared to the absence of incubation with proteases was determined. Table 19 below depicts the results of kinetic analysis of residual activity of exemplary non-limiting IFN-β SuperLeads following treatment with protease and the time of incubation with protease required to give 50% of total anti-viral activity. Also depicted in Table 19 is the rate of increased proteolysis which is a ratio of time at 50% activity of the modified IFN-β polypeptide compared to a wildtype or native IFN-β.

TABLE 19
IFN-β SuperLEADS Exhibiting Increased Resistance to
Proteolysis

| | | Proteolysis Resistance | |
|---|---|---|---|
| NEMO | | Time at 50% Total | Rate: |
| Code | Mutant | Activity | Mutant/Wild-type |
| 525 | L5E/Q10D | 16 | 8 |
| 528 | L5E/K108S | 24 | 12 |
| 532 | L6Q/L47I | 24 | 12 |
| 533 | L5D/K108S | 24 | 12 |
| 534 | L5N/L6E | 24 | 12 |
| 538 | L5Q/K108S | 16 | 8 |
| 540 | L5N/Q10D | 24 | 12 |
| 541 | L6Q/M36I | 16 | 8 |
| 542 | L5D/N86Q | 20 | 10 |
| 543 | L5N/K108S | 10 | 5 |
| 544 | L5D/L6Q | 10 | 5 |
| 545 | L6E/Q10D | 24 | 12 |
| 547 | L5Q/N86Q | 16 | 8 |
| 548 | L6E/K108S | 20 | 10 |
| 551 | L5D/L47I | 20 | 10 |
| 553 | L6Q/K108S | 16 | 8 |
| 554 | L5N/L6Q | 24 | 12 |
| 557 | L6E/N86Q | 24 | 12 |
| 558 | L5Q/L6Q | 20 | 10 |
| 559 | L5D/M36I | 24 | 12 |
| 560 | L5N/L47I | 24 | 12 |
| 561 | L6Q/N86Q | 24 | 12 |
| 523 | WT | 2.0 | 1.0 |
| 523 | WT | 2.0 | 1.0 |
| 523 | WT | 2.0 | 1.0 |
| 523 | WT | 2.0 | 1.0 |
| 523 | WT | 2.0 | 1.0 |
| 523 | WT | 2.0 | 1.0 |
| 523 | WT Average | 2.0 | 1.0 |

c. Additional Kinetic Analyses of IFN-β LEAD Proteins

In a separate experiment, WT and IFN-β mutant LEAD proteins were tested for biological activity and resistance to proteolysis. In this experiment LEAD proteins were produced in human embryonic kidney cell line, 293-EBNA, using Dubelcco's modified Eagle's medium supplemented with glucose (4.5 g/L; Gibco-BRL), glutamine (2 mM, Gibco-BRL), Geneticin (0.25 g/l, Gibco-BRL) and fetal bovine serum (10%, Hyclone). Cells were transiently transfected with the plasmids encoding the IFN-β mutants as follows: 15×10⁶ cells were seeded into triple flasks and grown for 48 h before transfection. Cells, at about 70% confluence, were transfected with 45 μg of plasmid (from the library of pNAUT-IFN-β mutants, see above) by using polyethyleneimine (PEI, Sigma) solution. Briefly, plasmid DNAs were mixed with 150 mM NaCl solution (Sigma) and then, a 10 mM PEI mixture diluted into 150 mM NaCl solution was added dropwise to plasmid DNA mixture. The DNA/PEI mixture was incubated for 15 minutes at room temperature before addition to each of the flasks containing 25 ml of culture medium supplemented with 1% serum. After gently shaking the flasks, cells were incubated at 37° C. in 10% CO₂ atmosphere and supernatants containing IFN-β proteins are collected 48 hours after transfection into 1 liter bottle aliquots at 4° C. Normalization of IFN-β concentration from culture supernatants was performed by enzyme-linked immunoabsorbent assay (ELISA) using two commercial kits (PBL Biomedical Laboratories and TFB, Fujirebio Inc.) according to the manufacturer's instructions. IFN-β concentrations from wild-type, and mutant samples were estimated by using an international reference standard provided by the National Institute for Biological Standard and Controls (NIBSC, United Kingdom).

For the biological activity assay, the protocol described in Example 5a was employed with the following modifications. Serial dilutions of each mutant protein were performed to produce 12 concentrations of protein in the range of 1.42 pg/ml to 3000 pg/ml. The proteins were added to HeLa cells as described in Example 5a, and incubated for 16 hours at 37° C. in an atmosphere of 5% CO₂. Following treatment of the HeLa cells with the IFN-β mutant LEAD proteins, the HeLa cells were infected with EMC-virus. At 48 hour post-infection, the resistance to viral infection was assayed by determination of the number of living cells by staining with methylene blue followed by OD measurement. The absorbance of the dye was measured using an ELISA plate reader (Spectramax). The anti-viral activity of IFN-β samples (expressed as EC₅₀ average, pg/ml) was determined as the concentration of IFN-β needed for 50% protection of the cells against EMC virus-induced cytopathic effects.

For the resistance to proteases assay, WT and IFN-β mutant LEAD proteins (400 pg) were treated with a mixture of proteases for different lengths of time between 30 minutes and 24 hours prior to addition to HeLa cells, as described in Example 7b. The percent of residual IFN-β activity over time of exposure to proteases was evaluated using 1.5 pg of the protease mixture (approximately 1% of total proteins in assay, Example 7a). Incubation times were: 0 h, 2 h, 4 h, 8 h, 16 h, and 24 h. Following protease exposure, the proteolysis reaction was inhibited by the addition of protease inhibitors. The treated WT and IFN-β mutant LEAD proteins were then tested for antiviral activity as described in Example 5 in order to determine the residual activity at each time point. After 16 hours of incubation with the HeLa cells with the IFN-β mutant LEAD proteins, the HeLa cells were infected with EMC-virus. At 48 hours post-infection, the resistance to viral infection was assayed by determination of the number of living cells by staining with methylene blue followed by OD measurement as described above.

Data for an exemplary selection of mutant IFN-β LEAD polypeptides are shown in Table 19b.

Tested polypeptides included native IFN-β and exemplary candidate LEADs including L5Q, M36I, L47I, K105S, K108S, K108H, L5D, L5E, L5N, L6E, L6Q, L6R, L6S, L9N, Q10D, S13D, C17N, C17T, N86Q, N86T, Q94D, Q94S, H97D, and N90C. The exemplary candidate LEADs and SuperLeads tested exhibited improved protease resistance compared to native IFN-β. Native IFN-β began to exhibit reduced residual anti-viral activity following incubation with protease for less than 1 hour which declined over time such that native IFN-β exhibited no detectable residual anti-viral activity following incubation with protease for 6 hours and greater. In a kinetic analysis of protease resistance where incubation times tested were 0 h, 2 h, 4 h, 8 h, 16 h, and 24 h, modified IFN-β polypeptides L5E, L5D, L5Q, M36I, and L5N exhibited about 50% or more residual anti-viral activity up to 8 hours incubation with proteases compared to the activity of the respective polypeptides in the absence of incubation with proteases.

In some experiments, the time of incubation with proteases required to give 50% of total activity (anti-viral) compared to incubation in the absence of proteases was determined. Table 19b below depicts the results of kinetic analysis of residual activity of exemplary non-limiting IFN-β Leads following treatment with protease and the time of incubation with protease required to give 50% of total anti-viral activity. Also depicted in Table 19b is the rate of increased proteolysis which is a ratio of time at 50% activity of the modified IFN-β polypeptide compared to a wildtype or native IFN-β.

TABLE 19b
IFN-β LEADS Exhibiting Increased Resistance to Proteolysis

| | | Proteolysis Resistance | |
|---|---|---|---|
| NEMO | | Time at 50% Total | Rate: |
| Code | Mutant | Activity | Mutant/Wild-type |
| 9 | L5Q | 10.00 | 5 |
| 58 | M36I | 16.00 | 8 |
| 72 | L47I | 8.00 | 4 |
| 104 | K105S | 9.00 | 4.5 |
| 110 | K108S | 8.00 | 4 |
| 111 | K108H | 9.00 | 4.5 |
| 169 | L5D | 12.00 | 6 |
| 170 | L5E | 14.00 | 7 |
| 247 | L5N | 10.00 | 5 |
| 250 | L6E | 10.00 | 5 |
| 253 | L6Q | 8.00 | 4 |
| 254 | L6R | 10.00 | 5 |
| 255 | L6S | 12.00 | 6 |
| 257 | L9N | 9.00 | 4.5 |
| 259 | Q10D | 22.00 | 11 |
| 266 | S13D | 10.00 | 5 |
| 286 | C17N | 22.00 | 11 |
| 290 | C17T | 8.00 | 4 |
| 299 | N86Q | 12.00 | 6 |
| 301 | N86T | 6.00 | 3 |
| 321 | Q94D | 4.00 | 2 |
| 326 | Q94S | 8.00 | 4 |
| 336 | H97D | 11.00 | 5.5 |
| 369 | N90C | 24.00 | 12 |
| | WT | 2.0 | 1.0 |

### Example 8

### Assessment of Resistance to Gelatinase B

Native IFN-β polypeptide or modified IFN-β polypeptides (15 μg per sample) were treated with gelatinase B (SIGMA) at ratio 10:1 w/w. Samples were collected at various time points between 5 minutes and 20 hours of incubation, and the protease reaction was stopped by adding 50 μl of anti-proteases solution (Roche). Samples were stored at −20° C. At the appropriate time points, 10 ml of anti-proteases complete medium containing mini EDTA-free tablets (Roche; one tablet dissolved in 10 ml of DMEM and then diluted 1/500) was added to each well in order to stop proteolysis reactions. Biological activity assays were then performed as described for each sample in order to determine the residual activity at each time point. IFN-β polypeptides tested included native IFN-β and exemplary candidate LEADs and SuperLEADs including L6E/K108S, L5D/M36I, L5D, L5E, L5N, Q94D, L5Q/K108S, L5D/K108S, L5D/L6Q, L6E/Q10D, L5D/L47I, L5S, Q10D, L5E/K108S, L5N/Q10D, and L5N/K108S. After incubation with gelatinase B for less than 100 minutes, native IFN-β exhibiting no detectable residual activity in an anti-viral assay. All candidate LEADs tested demonstrated improved resistance to proteolysis by gelatinase B compared to native IFN-β, although at varying levels. Following incubation with gelatinase B for 200 minutes, Q94D, L5N, L5E, L5E/K108S, L5S, L5N/K108S, and L5N/Q10D showed residual anti-viral activity comparable to incubation in the absence of protease, which decreased to no or low residual activity following incubation with gelatinase B for approximately 400 minutes. L5D/K108S, L5D/M36I, L5D, L6E/K108S, L5Q/K108S, L6E/Q10D, L5D/L47I, L5D/L6Q, and Q10D showed almost complete resistance to gelatinase B as assessed by residual anti-viral activity following incubation with gelatinase B up to the maximal incubation time tested, 20 hours. L5D/K108S, L5D/M36I, L5D, L6E/L6Q, L5D/L47I, and L5D/L6Q were the most resistant with no loss in residual activity following incubation with gelatinase B for 20 h.

### Example 9

### Assessment of Conformational Stability: Thermal Tolerance Assay

After determination by ELISA of the amount of proteins produced (for each individual IFN-β variant and for native IFN-β), 0.4 ng of native IFN-β or modified IFN-β was added to 250 μl of DMEM serum free medium supplemented with 1× anti-protease cocktail mixture (mini EDTA free, Roche) and incubated at 37° C. in a deep-well plate. To assess the kinetics of thermal tolerance, individual IFN-β polypeptides (wildtype or variant) were incubated for increasing time at 37° C. At increasing time-points (0, 2, 4, 6, 8, 12, 24, 36, 48 hours), 380 μl of DMEM medium supplemented with 5% SVF was added to 20 μl aliquots (final concentration 12000 pg/ml of IFN-β). Samples are immediately frozen and stored at −20° C. Biological activity assays were then performed as described for each sample in order to determine the residual activity at each time point. The time of incubation at a temperature of 37° C. required to give 50% of total activity (anti-viral or anti-proliferative) compared to incubation at room temperature was determined. Table 20 below depicts the results of kinetic analysis of residual activity of exemplary non-limiting IFN-β SuperLeads following treatment with increased temperature. Also depicted in Table 20 is the rate of increased thermal stability which is a ratio of time at 50% activity of the modified IFN-β polypeptide compared to a wildtype or native IFN-β.

TABLE 20
IFN-β SuperLEADS Exhibiting Increased Thermal Tolerance

| NEMO | | Temperature Stability | Rate: |
|---|---|---|---|
| Code | Mutant | Time at 50% Total Activity | mutant/wild-type |
| 525 | L5E/Q10D | 48 | 16.0 |
| 528 | L5E/K108S | 24 | 8.0 |
| 532 | L6Q/L47I | 30 | 10.0 |
| 533 | L5D/K108S | 24 | 8.0 |
| 534 | L5N/L6E | 48 | 16.0 |
| 538 | L5Q/K108S | 24 | 8.0 |
| 540 | L5N/Q10D | 48 | 16.0 |
| 541 | L6Q/M36I | 48 | 16.0 |
| 542 | L5D/N86Q | 48 | 16.0 |
| 543 | L5N/K108S | 24 | 8.0 |
| 544 | L5D/L6Q | 48 | 16.0 |
| 545 | L6E/Q10D | 48 | 16.0 |
| 547 | L5Q/N86Q | 48 | 16.0 |
| 548 | L6E/K108S | 40 | 13.3 |
| 551 | L5D/L47I | 48 | 160 |
| 553 | L6Q/K108S | 48 | 16.0 |
| 554 | L5N/L6Q | 48 | 16.0 |
| 557 | L6E/N86Q | 48 | 16.0 |
| 558 | L5Q/L6Q | 30 | 10.0 |
| 559 | L5D/M36I | 48 | 16.0 |
| 560 | L5N/L47I | 48 | 16.0 |
| 561 | L6Q/N86Q | 36 | 12.0 |
| 523 | WT | 3.0 | 1.0 |
| 523 | WT | 3.0 | 1.0 |
| 523 | WT | 3.0 | 1.0 |
| 523 | WT | 3.0 | 1.0 |
| 523 | WT | 3.0 | 1.0 |
| 523 | WT | 3.0 | 1.0 |
| 523 | WT Average | 3.0 | 1.0 |

Table 20b below depicts the results of kinetic analysis of residual activity of exemplary non-limiting IFN-β Leads following treatment with increased temperature. Also depicted in Table 20b is the rate of increased thermal stability which is a ratio of time at 50% activity of the modified IFN-β polypeptide compared to a wildtype or native IFN-β. The polypeptides tested were prepared as described in Example 7c. The polypeptides were subjected to the thermal tolerance assay as described above followed by assessment of IFN-β viral activity as described in Example 7c.

TABLE 20b
IFN-β SuperLEADS Exhibiting Increased Thermal Tolerance

| NEMO | | Temperature Stability | Rate: |
|---|---|---|---|
| Code | Mutant | Time at 50% Total Activity | mutant/wild-type |
| 9 | L5Q | 32 | 6.4 |
| 58 | M36I | 16 | 3.2 |
| 72 | L47I | 6 | 1.2 |
| 104 | K105S | 8 | 1.6 |
| 110 | K108S | 6 | 1.2 |
| 111 | K108H | 6 | 1.2 |
| 169 | L5D | 22 | 4.4 |
| 170 | L5E | 56 | 11.2 |
| 247 | L5N | 32 | 6.4 |
| 250 | L6E | 56 | 11.2 |
| 253 | L6Q | 54 | 10.8 |
| 254 | L6R | 10 | 2 |
| 255 | L6S | 54 | 10.8 |
| 257 | L9N | 48 | 9.6 |
| 259 | Q10D | 23 | 4.6 |
| 266 | S13D | 30 | 6 |
| 286 | C17N | 10 | 2 |
| 290 | C17T | 10 | 2 |
| 321 | Q94D | 8 | 1.6 |
| 326 | Q94S | 54 | 10.8 |
| 336 | H97D | 36 | 7.2 |
| 369 | N90C | 32 | 6.4 |
| | WT | 5 | 1.0 |

### Example 10

### In Vivo Pharmacokinetics (PK)

a. Intravenous Administration

Residual anti-viral (biological) activity of candidate LEADs and native (wild-type, unmodified) IFN-β was measured in plasma samples by assaying protection of HeLa cells from EMCV infection. Briefly, IFN-β wt and mutant polypeptides (generated in 293 EBNA cells and partially purified) were administrated by intravenous (IV) injection route in a volume of 10 mL/kg in 12 male mice (3 mice per group) (22.5 μg/kg dosage). Blood samples were drawn on day 1 between 0.08 and 24 hours post-dose administration. 30 μl of plasma was diluted into 1 mL with culture medium, followed by serial dilution and addition to HeLa cells. After 16 hrs of treatment of HeLa cells with the samples of plasma, the HeLa cells were infected with EMC-Virus. At 48 hrs post-infection, the number of living cells was determined by Methylene blue staining and OD measurement. EC₅₀ and specific activity were calculated for each mutant protein using NEMO (software), see Example 6. Each data point is the outcome of 36 wells (36 points); i.e. serial dilution (12 dilutions), each dilution was made and tested in triplicate. Each data point for PK represents the corresponding EC₅₀. In each experiment, the modified IFN-β polypeptides exhibited increased residual anti-viral activity over a longer period of time compared to the wild-type polypeptide. Table 21 provides exemplary data for the average residual anti-viral activity (U/mL) over time for modified and wild-type IFN-β polypeptides.

TABLE 21

| | Average Residual Anti-Viral Activity (U/mL) | | | | | | | | |
|---|---|---|---|---|---|---|---|---|---|
| Polypeptide | 0.08 hr | 0.25 hr | 0.5 hr | 1 hr | 1.5 hrs | 2 hrs | 3 hrs | 4 hrs | 6 hrs |
| L5D | 28.76 | 17.36 | 7.0 | 4.54 | 4.16 | 2.51 | 1.88 | 1.8 | 0.53 |
| L5E | 45.49 | 25.37 | 12.91 | 9.53 | 3.78 | 2.51 | 2.81 | 2.44 | 1.06 |
| L5N | 23.12 | 11.70 | 10.69 | 6.42 | 3.18 | 1.87 | 2.20 | 1.29 | 0.79 |
| L6Q | 14.98 | 23.06 | 11.79 | 7.31 | 4.98 | 3.82 | 2.54 | 2.33 | 1.90 |
| L5Q | 33.66 | 17.83 | 8.73 | 6.34 | 2.87 | 1.69 | 0.82 | 0.69 | 0.21 |
| K108S | 29.63 | 17.74 | 9.65 | 7.94 | 2.39 | 1.72 | 0.96 | 0.70 | 0.46 |
| Wild- | 17.99 | 6.06 | 2.19 | 0.43 | 0.17 | 0 | 0 | 0 | 0 |
| type* | | | | | | | | | |

*Average anti-viral activity of the wild-type IFN-β polypeptide was only measurable for 1.5 hours after IV administration of the native protein into the mice.

In Table 22, the pharmacokinetic parameters Ci, AUC and half-life for following IV administration of exemplary IFN-β LEADs in mice are presented. Data are expressed as a ratio between mutant and wild-type IFN-β values. PK parameters were determined using PK solutions® (version 2) software.

TABLE 22
Pharmacokinetic Profile of Exemplary IFN-β LEADS and
(Intravenous Administration)

| | Half-life | C initial (iv) | AUC |
|---|---|---|---|
| Mutant | Mutant/WT | Mutant/WT | Mutant/WT |
| L6Q | 6.1 | 1.01 | 6.2 |
| L5E | 5.3 | 0.95 | 4.9 |
| L5N | 5.6 | 0.85 | 4.7 |
| Q10D | 2.1 | 2.08 | 4.4 |
| L5D | 4.9 | 0.73 | 3.6 |
| L5Q | 4.0 | 0.89 | 3.5 |
| K108S | 4.5 | 0.69 | 3.1 |
| L6E | 1.8 | 1.65 | 2.9 |
| M36I | 2.2 | 1.26 | 2.7 |
| N86Q | 3.0 | 0.84 | 2.5 |
| Q94D | 2.3 | 1.09 | 2.4 |
| K108H | 1.4 | 1.36 | 2.1 |
| L116T | 2.0 | 0.72 | 1.4 |
| C17T | 1.0 | 1.32 | 1.3 |
| H97D | 1.8 | 0.60 | 1.1 |
| K136Q | 1.0 | 1.11 | 1.1 |

b. Subcutaneous Administration

Residual anti-viral (biological) activity of candidate LEADs and SuperLEADs and native (wild-type, unmodified) IFN-β also was measured in plasma samples, following subcutaneous (SC) injection, by assaying protection of HeLa cells from EMCV infection. Briefly, IFN-β wt and mutant polypeptides (produced in 293 EBNA cells and partially purified) were administrated by SC injection route in a volume of 10 mL/kg in 12 male mice (3 mice per group) (220 μg/kg dosage). Blood samples were drawn on day 1 between 0.08 and 48 hours post-dose administration. 30 μl of plasma was diluted into 1 mL with culture medium, followed by serial dilution and addition to HeLa cells. After 16 hrs of treatment of HeLa cells with the samples of plasma, the HeLa cells were infected with EMC-Virus. At 48 hrs post-infection, the number of living cells was determined by Methylene blue staining and OD measurement. EC₅₀ and specific activity were calculated for each mutant protein using NEMO (software), see Example 6. Each data point is the outcome of 36 wells (36 points); i.e. serial dilution (12 dilutions), each dilution was made and tested in triplicate. Each data point for PK represents the corresponding EC₅₀. Average anti-viral activity of the wild-type, native IFN-β polypeptide was only measurable for 6 hours after SC administration of the native protein into the mice. In each experiment, the modified IFN-β polypeptides exhibited increased residual anti-viral activity over a longer period of time compared to the wild-type polypeptide.

In Table 23, the pharmacokinetic parameters, half-life, AUC and Tₘₐₓ, following SC administration of exemplary IFN-β LEADS and Super-LEADs in mice are presented. Data for half-life and AUC are expressed as a ratio between mutant and wild-type IFN-β values. PK parameters were determined using PK solutions® (version 2) software.

TABLE 23
Pharmacokinetic Profile of Exemplary IFN-β LEADS
and Super-LEADs (Subcutaneous Administration)

| | Half-life | AUC_{(0-∞)} | Tₘₐₓ |
|---|---|---|---|
| Mutant | Mutant/WT | Mutant/WT | (h) |
| L5D/L47I | 2.0 | 70.9 | 1 |
| L5D/K108S | 1.4 | 49.6 | 1 |
| L6Q/K108S | 8.1 | 16.7 | 1 |
| K108S | 2.3 | 13.4 | 1 |
| N90C | 5.1 | 10.4 | 1 |
| L6E | 2.3 | 9.1 | 1 |
| L5D/L6Q | 5.0 | 8.4 | 2 |
| K105S | 1.4 | 7.5 | 1 |
| L5Q/L47I | 1.5 | 4.7 | 1 |
| L5N/Q10D | 2.5 | 4.7 | 1 |
| L5N | 1.7 | 4.3 | 1 |
| L5Q/K108S | 1.3 | 3.9 | 1 |
| K108H | 3.4 | 3.9 | 1 |
| L5S | 1.5 | 3.5 | 1 |
| Q94S | 2.4 | 3.2 | 1 |

### Example 11

### IFN-β Induction of Gene Expression

The ability of IFN-β LEAD and Super-LEAD polypeptides to induce expression of known IFN-β inducible genes in HT-1080 cells was assessed. HT-1080 cells were cultured using DMEM (Invitrogen) supplemented with 10% fetal bovine serum. Cells were plated into 96-well plates at 1×10⁴ cells/well and incubated overnight at 37° C. in a humid atmosphere with a composition of 5% CO2/95% air, before being treated with 1500 pg of IFN-β wild-type (WT) and mutant proteins. After treatment for 16 hours, cells were lysed and total RNA was isolated using RNA extraction kit (Qiagen RNeasy 96 RNA preparation kit, Qiagen) according to the manufacturer's protocol. RNA samples were aliquoted in 96-well plates and stored at −80° C. until use. MxA (myxovirus resistance 1 interferon-inducible protein p78), p69 (2′5′-oligoadenylate synthetase (2′5′ OAS)) and β-R1 (SCYB11) mRNA levels were determined by real time quantitative PCR using an ABI 7900 sequence detector (Applied Biosystems). Quantification of mRNA was measured by using the one step reverse transcription polymerase chain reaction (RT-PCR) kit (First-strand synthesis kit, Applied Biosystems) according to procedure using the comparative Delta Ct method (Perkin Elmer (1997) user bulletin no 2) with GAPDH (glyceraldehyde-3-phosphate dehydrogenase) RNA probe as an endogenous reference (Applied Biosystems). The primers and probe sequences used for each gene are presented below. The data were normalized by expressing the ratio of either MxA, 2′5′ OAS p69 or β-R1 mRNA relative to GAPDH RNA.

For MxA:
Primer MxA-1:

(SEQ ID NO: 661)

5′-AAGGAATGGGAATCAGTCATGAG-3′
Primer MxA-2:

(SEQ ID NO: 662)

5′-TCTATTAGAGTCAGATCCGGGACAT-3′
RT PCR Probe MxA sequence:

(SEQ ID NO: 667)

5′-TCACCCTGGAGATCAGCTCCCGA-3′
For β-R1:
Primers BR1-1:

(SEQ ID NO: 663)

5′-AGGACGCTGTCTTTGCATAGG-3′
Primers BR1-2:

(SEQ ID NO: 664)

5′-ACAGTTGTTACTTGGGTACATTATGGA-3′
RT PCR Probe β-R1 sequence:

(SEQ ID NO: 668)

5′-AAAAGCAGTGAAAGTGGCAGATATTGAGAAAGC-3′
For p69:
Primer P69OAS-1:

(SEQ ID NO: 665)

5′-ATCTCGTCGTGTTCCATAACTCACT-3′
Primer P69OAS-2:

(SEQ ID NO: 666)

5′-GTTCATGGATTTCCTTGACGATTT-3′
RT PCR Probe 2′5′ OAS p69 sequence:

(SEQ ID NO: 669)

5′-CTACACCTCCCAAAAAAACGAGCGGC-3′

Data for the induction of MxA, β-R1, and p69 gene expression are presented in Table 24 as increased (+), highly increased (+/+), decreased (−), highly decreased (−/−) or equivalent (=) induction of gene expression compared to induction by wild-type IFN-β. All IFN-β LEADs and all IFN-β Super-LEADs except three mutants exhibited strong induction of β-R1 gene expression, with some exemplary LEADs and SuperLEADs exhibiting higher induction of β-R1 compared to induction by wild-type IFN-β as shown in Table 24. All IFN-β LEADs and all IFN-β Super-LEADs exhibited strong induction of MxA and p69 gene expression with some exemplary LEADs and SuperLEADs exhibiting higher induction MxA and p69 compared to induction by wild-type IFN-β as shown in Table 24.

TABLE 24
Induction of MxA, p69, and β-R1 gene expression
by IFN-β LEADs and Super-Leads

| | Comparison with | | | | Comparison with | | |
|---|---|---|---|---|---|---|---|
| | native IFNβ | | | | native IFNβ | | |
| Mutants | BR1 | MxA | P69 | Mutants | BR1 | MxA | P69 |
| L5T | = | = | = | V91C | = | = | = |
| L5Q | + | = | = | L5D/L6E | = | = | − |
| M36I | − | + | + | L5E/Q10D | = | + | = |
| D39G | = | = | = | L5Q/M36I | −/− | = | −/− |
| L47I | = | = | = | L5E/L6E | = | = | − |
| K105S | + | +/+ | + | L5D/Q10D | = | = | = |
| K108S | = | + | = | L5N/M36I | = | + | = |
| K108H | = | = | − | L5D/K108S | = | = | = |
| L116T | = | = | = | L5N/L6E | −/− | − | − |
| K136Q | = | = | = | L5Q/Q10D | +/+ | − | = |
| E137H | + | = | −/− | L6E/M36I | = | = | = |
| L5D | +/+ | − | = | L5E/N86Q | +/+ | − | = |
| L5E | + | = | = | L5Q/K108S | −/− | − | −/− |
| L5N | = | = | = | L5Q/L6E | −/− | − | − |
| L5S | = | = | − | L6Q/M36I | = | = | = |
| L6E | = | = | − | L5D/N86Q | = | + | = |
| L6Q | = | = | − | L5N/K108S | = | + | = |
| L6R | = | + | + | L5D/L6Q | = | + | = |
| L6S | = | = | = | L6E/K108S | = | = | = |
| L9N | = | = | = | L5E/L6Q | = | − | − |
| Q10D | + | = | = | L5D/L47I | −/− | = | = |
| S13D | = | = | = | L5N | + | = | = |
| C17N | = | = | = | L6Q/K108S | = | = | = |
| N86Q | = | = | = | L5N/L6Q | = | = | = |
| N86T | + | = | − | L5E/M36I | = | = | = |
| Q94D | = | +/+ | + | L6E/N86Q | −/− | − | − |
| Q94S | = | = | = | L5D/M36I | +/+ | = | = |
| H97D | − | − | − | L5N/L47I | = | = | = |
| V101S | = | = | = | L6Q/N86Q | = | = | = |
| V101C | = | = | = | NIBSC-IFNα | −/− | = | = |
| N90C | = | = | = | NIBSC-IFNβ | = | − | − |

NIBSC: National Institute for Biological Standard and Controls (UK)

### Example 12

### Stat 3 Phosphorylation Assay

The ability of IFN-β LEAD and Super-LEAD polypeptides to induce Stat3 phosphorylation in HT-1080 cells was assessed. HT-1080 cells were cultured using DMEM (Invitrogen) supplemented with 10% fetal bovine serum. Cells were plated into 96-well plates at 1×10⁴ cells/well and incubated overnight at 37° C. in a humid atmosphere with a composition of 5% CO₂/95% air, before being treated with 6000 pg of IFN-β wild-type (WT) and mutant proteins. After treatment for 30 minutes, media was removed and cells were rinsed once with ice-cold PBS. Cell lysates were obtained by adding 100 μl of ice-cold cell lysis buffer plus 1 mM phenylmethylsulfonyl fluoride (PMSF) to each well and the plates were incubated on ice for 5 minutes. Cells were scraped off the plate and transfer to a new 96-well-plate. An additional step of cell disruption was performed by several cycles of freezing/thawing. The cell lysate was centrifugated for 10 minutes at 4° C., and supernatant aliquoted and stored at −80° C. until use. Phosphorylation of Stat3 was detected by ELISA kit (PathScan Phospho-Stat3 Sandwitch Elisa Kit, Cell Signalling) according to manufacturer protocol.

Exemplary data for induction of Stat3 phosphorylation is presented in Table 25 as increased (+), decreased (−), or equivalent (=) induction of Stat3 phosphorylation compared to induction by wild-type IFN-β. All IFN-β LEADs and all IFN-β Super-LEADs exhibited strong induction of STAT3 phosphorylation with some exemplary LEADs and SuperLEADs exhibiting higher phosphorylation of STAT3 compared to induction by wild-type IFN-β as shown in Table 25.

TABLE 25
Induction of STAT3 Phosphorylation by IFN-β LEADs and Super-Leads

| | Comparison | | Comparison |
|---|---|---|---|
| | with native IFNβ | | with native IFNβ |
| Mutants | Stat3 phosphorylation | Mutants | Stat3 phosphorylation |
| L5T | + | V91C | = |
| L5Q | + | L5D/L6E | = |
| M36I | + | L5E/Q10D | = |
| D39G | = | L5Q/M36I | = |
| L47I | = | L5E/L6E | = |
| K105S | = | L5D/Q10D | = |
| K108S | = | L5N/M36I | = |
| K108H | + | L5D/K108S | = |
| L116T | = | L5N/L6E | = |
| K136Q | = | L5Q/Q10D | = |
| E137H | = | L6E/M36I | = |
| L5D | + | L5E/N86Q | = |
| L5E | + | L5Q/K108S | = |
| L5N | = | L5Q/L6E | = |
| L5S | = | L6Q/M36I | = |
| L6E | = | L5D/N86Q | = |
| L6Q | = | L5N/K108S | = |
| L6R | + | L5D/L6Q | = |
| L6S | = | L6E/K108S | = |
| L9N | + | L5E/L6Q | = |
| Q10D | = | L5D/L47I | = |
| S13D | = | L5N | = |
| C17N | = | L6Q/K108S | = |
| N86Q | + | L5N/L6Q | = |
| N86T | = | L5E/M36I | = |
| Q94D | = | L6E/N86Q | = |
| Q94S | = | L5D/M36I | = |
| H97D | = | L5N/L47I | = |
| V101S | = | L6Q/N86Q | = |
| V101C | = | NIBSC-IFNα | = |
| N90C | = | NIBSC-IFNβ | nd |

NIBIC: National Institute for Biological Standard and Controls (UK)

Since modifications will be apparent to those of skill in this art, it is intended that this invention be limited only by the scope of the appended claims.

1. A modified IFN-β polypeptide, comprising:
one or more amino acid modifications in an unmodified IFN-β polypeptide, wherein:
the one or more amino acid modifications correspond to modifications selected from among Y3I, Y3H, L6I, L6V, L6H, L6A, R11D, Q18S, Q18N, Q18H, Q18T, K19N, L20I, L20V, L20H, L20A, L21I, L21V, L21T, L21Q, L21H, L21A, Q23H, Q23S, Q23T, Q23N, L24I, L24V, L24T, L24Q, L24H, L24A, E29N, K33N, D34N, D34Q, D34G, F38I, F38V, D39N, P41A, P41S, E42N, E43N, E43K, E43Q, E43H, K45D, K45N, Q48H, Q48S, Q48T, Q48N, Q49H, Q49S, Q49T, Q49N, F50I, F50V, Q51H, Q51S, Q51T, Q51N, K52D, K52N, E53N, E53R, E53Q, E53H, D54K, D54N, D54G, D54Q, L57I, L57T, L57Q, L57H, Y60I, Y60H, E61K, E61H, E61N, E61Q, M62I, M62V, M62T, M62Q, L63I, L63V, L63T, L63Q, L63H, L63A, Q64H, Q64S, Q64T, Q64N, F70I, F70V, Q72H, Q72S, Q72T, Q72N, D73N, W79H, W79S, E81K, E81N, E85N, E85K, L87I, L87V, L87H, L87A, L88I, L88V, L88T, L88Q, L88H, L88A, L98I, L98V, L98H, L98A, K99N, L102I, L102V, L102T, L102Q, L102H, L102A, E103N, E103K, E104N, E104R, K105D, K105N, L106I, L106V, L106T, L106Q, L106H, L106A, E107N, E107R, K108D, K108N, E109R, E109N, D110K, D110N, R113E, K115D, K115N, K115S, K115H, K115Q, M117I, M117V, M117T, M117Q, M117A, L122I, L122V, L122T, L122Q, L122H, L122A, K123N, R124D, R124E, Y125I, Y125H, Y126I, Y126H, Y132I, Y132H, L133I, L133V, L133T, L133Q, L133H, L133A, K134N, K136N, E137N, W143H, W143S, R147H, R147Q, E149H, E149N, E149Q, L151I, L151V, L151T, L151Q, L511H, L151A, R152D, F154V, F154I, F156I, L160I, L160V, L160T, L160Q, L160H, L160A, L164I, L164V, L164T, L164Q, L164H, L164A, and R165D in the mature IFN-β polypeptide set forth in SEQ ID NO:1.

2. A modified IFN-β polypeptide of claim 1, wherein the one or more amino acid modifications correspond to modifications selected from among Y3I, Q18S, Q18N, K19N, L20I, L20V, K33N, D34N, P41A, P41S, E42N, E43N, K45D, K45N, Q48H, Q48S, Q48T, Q49H, Q49S, Q49T, F50I, F50V, Q51H, Q51S, Q51T, Q51N, K52D, K52N, E53N, D54K, D54N, D54G, L57I, Y60I, E61K, E61H, E61N, L63I, Q64H, Q64S, Q64T, F70I, F70V, Q72H, Q72S, E85N, L88I, L88V, L98I, L98V, K99N, E103N, E104N, K105D, K105N, L106I, L106V, E107N, E109N, K115D, K115N, K115S, K115H, K123N, Y125I, Y126I, Y132I, K134N, K136N, R147H, R147Q, E149H, E149N, L151I, and F154V in the mature IFN-β polypeptide set forth in SEQ ID NO:1.

3. The modified IFN-β polypeptide of claim 1 that retains at least one in vivo activity of an IFN-β polypeptide.

4. The modified IFN-β polypeptide of claim 1 that has 1, 2, 3, 4, 5, 6, 7, 8, 9, 10, 11, 12, 13, 14, 15, 16, 17, 18, 19 or 20 amino acid modifications.

5. The modified IFN-β polypeptide of claim 1, that is a mature polypeptide.

6. The modified IFN-β polypeptide of claim 1, that is a precursor polypeptide.

7. A modified IFN-β polypeptide of claim 1, wherein the unmodified IFN-β polypeptide comprises the sequence of amino acids set forth in SEQ ID NO:1 or SEQ ID NO:3.

8. A modified IFN-β polypeptide of claim 1, comprising:
one or more amino acid modifications in an unmodified IFN-β polypeptide comprising the sequence of amino acids set forth in SEQ ID NO:3, wherein:
the one or more amino acid modifications correspond to modifications selected from among Y3I, Q18S, Q18N, K19N, L20I, L20V, L21I, L21V, K33N, D34N, P41A, P41S, E42N, E43N, K45D, K45N, Q48H, Q48S, Q48T, Q49H, Q49S, Q49T, F50I, F50V, Q51H, Q51S, Q51T, Q51N, K52D, K52N, E53N, D54K, D54N, D54G, L57I, Y60I, E61K, E61H, E61N, M62I, M62V, Q64H, Q64S, Q64T, F70I, F70V, Q72H, Q72S, E85N, L88I, L88V, L98I, L98V, K99N, E103N, E104N, K105D, K105N, L106I, L106V, E107N, E109N, K115D, K115N, K115S, K115H, M117I, M117V, L122I, L122V, K123N, Y125I, Y126I, Y132I, K134N, Y136N, R147H, R147Q, E149H, E149N, L151I, L154V, and L160V in the mature IFN-β polypeptide set forth in SEQ ID NO:1.

9. A modified IFN-β polypeptide of claim 1, wherein the unmodified IFN-polypeptide is an allelic or species variant of the polypeptide whose sequence is set forth in SEQ ID NO:1.

10. The modified IFN-β polypeptide of claim 9, wherein the allelic or species variant has 40%, 50%, 60%, 70%, 80%, 90%, 91%, 92%, 93%, 94%, 95%, 96%, 97%, 98%, or 99% sequence identity to the polypeptide whose sequence is set forth in SEQ ID NO:1 excluding the one or more amino acid modification(s).

11. A modified IFN-β polypeptide of claim 1, comprising a sequence of amino acids set forth in any one of SEQ ID NOS: 4-68, 71-82, 84-87, 134-153, 519, 520, 534-557, 559, 560, 562-564, 566-606 and 608-650.

12. A modified IFN-β polypeptide of claim 1, wherein only the primary sequence is modified and the polypeptide exhibits increased protein stability.

13. A modified IFN-β polypeptide of claim 1, wherein the polypeptide:
comprises only one amino acid modification; and
exhibits increased protein stability.

14. A modified IFN-β polypeptide of claim 1 that exhibits increased protease resistance or increased conformational stability or a combination thereof compared to the unmodified IFN-β polypeptide and retains one or more activities of the unmodified IFN-β polypeptide.

15. The modified IFN-β polypeptide of claim 14 that exhibits increased protease resistance.

16. A modified IFN-β polypeptide of claim 14 wherein:
the one or more amino acid modifications correspond to modifications selected from among Y3H, Y3I, L6I, L6V, L6H, L6A, K19N, Q18S, Q18N, Q18H, Q18T, L20I, L20V, L20H, L20A, L21I, L21V, L21T, L21Q, L21H, L21A, Q23H, Q23S, Q23T, Q23N, L24I, L24V, L24T, L24Q, L24H, L24A, K33N, E29N, D34N, D34Q, D34G, F38I, F38V, D39N, P41A, P41S, E42N, E43Q, E43H, E43N, K45N, Q48N, Q48H, Q48S, Q48T, Q49N, Q49H, Q49S, Q49T, F50I, F50V, Q51H, Q51S, Q51T, Q51N, K52N, E53N, E53Q, E53H, D54N, D54Q, D54G, L57I, L57V, L57T, L57Q, L57H, Y60I, Y60H, E61H, E61N, E61Q, M62I, M62V, M62T, M62Q, L63I, L63V, L63T, L63Q, L63H, Q64N, Q64H, Q64S, Q64T, F70I, F70V, Q72H, Q72S, Q72T, Q72N, D73N, W79H, W79S, E81N, E85N, L87I, L87V, L87H, L87A, L88I, L88V, L88T, L88Q, L88H, L88A, L98I, L98V, L98H, L98A, K99N, L102I, L102V, L102T, L102Q, L102H, L102A, E103N, E104N, K105N, L106I, L106V, L106T, L106Q, L106H, L106A, E107N, K108N, E109N, D110N, K115N, K115S, K115H, K115Q, M117I, M117V, M117T, M117Q, M117A, L122I, L122V, L122T, L122Q, L122H, L122A, K123N, Y125I, Y125H, Y126I, Y126H, Y132I, Y132H, L133I, L133V, L133T, L133Q, L133H, K134N, K136N, E137N, W143H, W143S, R147H, R147Q, E149H, E149N, E149Q, L151I, L151V, L151T, L151Q, L151H, L151A, F154I, F154V, F156I, L160I, L160V, L160T, L160Q, L160H, L160A, L164T, L164Q, L164H, L164A, L164I, and L164V in the mature IFN-β polypeptide set forth in SEQ ID NO:1.

17. A modified IFN-β polypeptide of claim 14, wherein the polypeptide exhibits increased resistance to one or more proteases selected from among pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, luminal pepsin, microvillar endopeptidase, dipeptidyl peptidase, enteropeptidase, hydrolase, NS3, factor Xa, Granzyme B, thrombin, plasmin, urokinase, tPA and PSA.

18. A modified IFN-β polypeptide of claim 17, wherein the protease is gelatinase B and the amino acid modification is at positions containing any one or more of an amino acid selected from among Phenylalanine (F), Leucine (L), Glutamic Acid (E), Tyrosine (Y), and Glutamine (Q).

19. A modified IFN-β polypeptide of claim 18, wherein:
the one or more amino acid modifications correspond to modifications selected from among Y3I, Y3H, L6I, L6V, L6H, L6A, Q18S, Q18N, Q18H, Q18T, L20I, L20V, L20H, L20A, L21I, L21V, L21T, L21Q, L21H, L21A, Q23H, Q23S, Q23T, Q23N, L24I, L24V, L24T, L24Q, L24H, L24A, E29N, F38I, F38V, E42N, E43N, E43Q, E43H, Q48H, Q48S, Q48T, Q48N, Q49H, Q49S, Q49T, Q49N, F50I, F50V, Q51H, Q51S, Q51T, Q51N, E53Q, E53H, E53N, L57I, L57V, L57T, L57Q, L57H, Y60H, Y60I, E61H, E61N, E61Q, L63I, L63V, L63T, L63Q, L63H, Q64H, Q64S, Q64T, Q64N, F70I, F70V, Q72H, Q72S, Q72T, Q72N, E81N, E85N, L87I, L87V, L87H, L87A, L88I, L88V, L88T, L88Q, L88H, L88A, L98I, L98V, L98H, L98A, L102I, L102V, L102T, L102Q, L102H, L102A, E103N, E104N, L106I, L106V, L106T, L106Q, L106H, L106A, E107N, E109N, Y125I, Y125H, Y126I, Y126H, Y132I, Y132H, L133I, L133V, L133T, L133Q, L133H, E137N, E149H, E149N, E149Q, L151I, L151V, L151T, L151Q, L151H, L151A, F154I, F154V, F156I, L160I, L160V, L160T, L160Q, L160H, L160A, L164I, L164V, L164H, L164A, L164T, and L164Q in the mature IFN-β polypeptide set forth in SEQ ID NO:1.

20. A modified IFN-β polypeptide of claim 14 that exhibits increased conformational stability, wherein:
the one or more amino acid modifications correspond to modifications selected from among R11D, E43K, K45D, K52D, E53R, D54K, E61K, E81K, E85K, E103K, E104R, K105D, E107R, E109R, D110K, R113E, K115Q, K15D, R124D, R124E, R152D, and R165D.

21. A modified IFN-β polypeptide of claim 14, wherein:
the one or more amino acid modifications increase the isoelectric point of the polypeptide; and
the amino acid modification is replacement by one or more amino acid residues selected from among a Glutamic Acid (E) or an Aspartic Acid (D) to a Lysine (K) or to an Arginine (R); or
the one or more amino acid modifications decrease the isoelectric point of the polypeptide; and
the one or more amino acid modification(s) is replacement by one or more amino acid residues selected from among a Lysine (K) or an Arginine (R) to a Glutamine (Q), to a Glutamic Acid (E) or to an Aspartic Acid (D).

22. A modified IFN-β polypeptide of claim 21 wherein:
the one or more amino acid modifications increase the isoelectric point; and
the one or more amino acid modifications correspond to modifications selected from among E43K, E53R, D54K, E61K, E81K, E85K, E103K, E104R, E107R, E109R, and D110K in the mature IFN-β polypeptide set forth in SEQ ID NO:1.

23. A modified IFN-β polypeptide of claim 21, wherein:
the one or more amino acid modifications decrease the isoelectric point; and
the one or more amino acid modifications correspond to modifications selected from among K115Q, R11D, K45D, K52D, K105D, K108D, R113E, K115D, R124D, R124E, R152D, and R165D in the mature IFN-β polypeptide set forth in SEQ ID NO:1.

24. A modified IFN-β polypeptide of claim 1, comprising one or more further amino acid modification(s) at one or more positions corresponding to amino acid positions selected from among M1, Y3, L5, L6, F8, L9, Q10, R11, S12, S13, N14, F15, Q16, C17, Q18, K19, L20, L21, W22, Q23, L24, N25, R27, L28, E29, Y30, L32, K33, D34, R35, M36, F38, D39, E42, E43, K45, L47, Q48, Q49, K52, E53, D54, L57, Y60, E61, M62, L63, Q64, F67, R71, Q72, D73, G78, W79, N80, E81, T82, I83, E85, N86, L87, L88, A89, N90, V91, Y92, Q94, I95, H97, L98, K99, V101, L102, E103, E104, K105, L106, E107, K108, E109, D110, F111, R113, K115, L116, M117, L120, L122, K123, R124, Y125, Y126, R128, L130, Y132, L133, K134, N136, E137, Y138, W143, E149, L151, R152, F154, Y155, F156, R159, L160, Y163, L164, and R165 in the mature IFN-β polypeptide set forth in SEQ ID NO:1.

25. A modified IFN-β polypeptide of claim 24, wherein the one or more further amino acid modification(s) correspond to modifications selected from among M1C, M1D, M1E, M1K, M1N, M1R, M1S, M1V, M1I, M1T, M1A, M1Q, Y3H, L5V, L5I, L5T, L5Q, L5H, L5A, L5D, L5E, L5K, L5R, L5N, L5S, L6I, L6V, L6H, L6A, L6D, L6E, L6K, L6N, L6Q, L6R, L6S, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, R11D, R11H, R11Q, S12D, S12E, S12K, S12R, S13D, S13E, S13K, S13N, S13Q, S13R, S13T, S13C, N14D, N14E, N14K, N14Q, N14R, N14S, N14T, F15D, F15E, F15K, F15R, F15I, F15V, Q16D, Q16E, Q16K, Q16N, Q16R, Q16S, Q16C, Q16T, C17D, C17E, C17K, C17N, C17Q, C17R, C17S, C17T, Q18H, Q18T, K19Q, K19T, K19S, K19H, L20H, L20A, L20N, L20Q, L20R, L20S, L20T, L20D, L20E, L20K, L21I, L21V, L21T, L21Q, L21H, L21A, W22D, W22E, W22K, W22R, W22S, W22H, Q23H, Q23S, Q23T, Q23N, Q23D, Q23E, Q23K, Q23R, L24I, L24V, L24T, L24Q, L24H, L24A, L24D, L24E, L24K, L24R, N25H, N25S, N25Q, R27H, R27Q, L28V, L28I, L28T, L28Q, L28H, L28A, E29N, E29Q, E29H, Y30H, Y30I, L32V, L32I, L32T, L32Q, L32H, L32A, K33Q, K33T, K33S, K33H, D34Q, D34G, R35H, R35Q, M36V, M36I, M36T, M36Q, M36A, F38I, F38V, D39N, D39Q, D39H, D39G, E42Q, E42H, E43K, E43Q, E43H, K45Q, K45T, K45S, K45H, L47V, L47I, L47T, L47Q, L47H, L47A, Q48N, Q49N, K52Q, K52T, K52S, K52H, E53R, E53Q, E53H, D54Q, L57V, L57T, L57Q, L57H, L57A, Y60H, E61Q, M62I, M62V, M62T, M62Q, M62A, L63V, L63T, L63Q, L63H, L63A, Q64N, F67I, F67V, R71H, R71Q, Q72N, D73N, D73H, D73G, D73Q, G78D, G78E, G78K, G78R, W79H, W79S, W79D, W79E, W79K, W79R, N80D, N80E, N80K, N80R, E81K, E81N, E81Q, E81H, T82D, T82E, T82K, T82R, I83D, I83E, I83K, I83R, I83N, I83Q, I83S, I83T, E85K, E85Q, E85H, N86D, N86E, N86K, N86R, N86Q, N86S, N86T, L87I, L87V, L87H, L87A, L87D, L87E, L87K, L87R, L87N, L87Q, L87S, L87T, L88T, L88Q, L88H, L88A, A89D, A89E, A89K, A89R, N90D, N90E, N90K, N90Q, N90R, N90S, N90T, N90C, V91D, V91E, V91K, V91N, V91Q, V91R, V91S, V91T, V91C, Y92H, Y92I, Q94D, Q94E, Q94K, Q94N, Q94R, Q94S, Q94T, Q94C, I95D, I95E, I95K, I95N, I95Q, I95R, I95S, I95T, H97D, H97E, H97K, H97N, H97Q, H97R, H97S, H97T, H97C, L98H, L98A, L98D, L98E, L98K, L98N, L98Q, L98R, L98S, L98T, L98C, K99Q, K99T, K99S, K99H, V101D, V101E, V101K, V101N, V101Q, V101R, V101S, V101T, V101C, L102I, L102V, L102T, L102Q, L102H, L102A, E103K, E103Q, E103H, E104R, E104Q, E104H, K105Q, K105T, K105S, K105H, L106T, L106Q, L106H, L106A, E107R, E107Q, E107H, K108D, K108N, K108Q, K108T, K108S, K108H, E109R, E109Q, E109H, D110K, D110N, D110Q, D110H, D110G, F111I, F111V, R113E, R113H, R113Q, K115Q, L116V, L116I, L116T, L116Q, L116H, L116A, M117I, M117V, M117T, M117Q, M117Q, M117A, L120V, L120I, L120T, L120Q, L120H, L120A, L122I, L122V, L122T, L122Q, L122H, L122A, K123Q, K123T, K123S, K123H, R124D, R124E, R124H, R124Q, Y125H, Y126H, R128H, R128Q, L130V, L130I, L130T, L130Q, L130H, L130A, Y132H, L133I, L133V, L133T, L133Q, L133H, L133A, K134Q, K134T, K134S, K134H, K136Q, K136T, K136S, K136H, E137N, E137Q, E137H, Y138H, Y138I, W143H, W143S, E149Q, L151V, L151T, L151Q, L151H, L151A, R152D, R152H, R152Q, F154I, Y155H, Y155I, F156I, F156V, R159H, R159Q, L160I, L160V, L160T, L160Q, L160H, L160A, Y163H, Y163I, L164I, L164V, L164T, L164Q, L164H, L164A, R165D, R165Q and R165H in the mature IFN-β polypeptide set forth in SEQ ID NO:1.

26. A modified IFN-β polypeptide of claim 24, wherein the one or more further amino acid modification(s) at one or more positions corresponding to amino acid positions is selected from among M1, Y3, L5, L6, F8, L9, Q10, R11, S12, S13, N14, F15, Q16, C17, Q18, K19, L20, L21, W22, Q23, L24, N25, R27, L28, E29, Y30, L32, K33, D34, R35, M36, F38, D39, E42, E43, K45, L47, Q48, Q49, K52, E53, D54, L57, Y60, E61, M62, L63, Q64, F67, R71, Q72, D73, G78, W79, N80, E81, T82, I83, E85, N86, L87, L88, A89, N90, V91, Y92, Q94, I95, H97, L98, K99, V101, L102, E103, E104, K105, L106, E107, K108, E109, D110, F111, R113, K115, L116, M117, L122, K123, R124, Y125, Y126, R128, L130, Y132, L133, K134, K136, E137, Y138, W143, E149, L151, R152, Y155, F156, R159, L160, Y163, L164, and R165 in the mature IFN-β polypeptide set forth in SEQ ID NO:1; and
the modified IFN-β polypeptide exhibits increased protease resistance compared to an IFN-β polypeptide that does not include the modification(s).

27. A modified IFN-β polypeptide of claim 26, wherein the one or more further amino acid modification(s) correspond to modifications selected from among M1V, M1I, M1T, M1A, M1Q, M1D, M1E, M1K, M1N, M1R, M1S, M1C, Y3H, L5V, L5I, L5T, L5Q, L5H, L5A, L5D, L5E, L5K, L5R, L5N, L5S, L6H, L6A, L6I, L6V, L6D, L6E, L6K, L6N, L6Q, L6R, L6S, L6T, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, R11H, R11Q, S12D, S12E, S12K, S12R, S13D, S13E, S13K, S13N, S13Q, S13R, S13T, S13C, N14D, N14E, N14K, N14Q, N14R, N14S, N14T, F15I, F15V, F15D, F15E, F15K, F15R, Q16D, Q16E, Q16K, Q16N, Q16R, Q16S, Q16T, Q16C, C17D, C17E, C17K, C17N, C17R, C17S, C17T, Q18H, Q18T, K19Q, K19T, K19S, K19H, L20H, L20A, L20N, L20Q, L20R, L20S, L20T, L20D, L20E, L20K, L21I, L21V, L21T, L21Q, L21H, L21A, W22S, W22H, W22D, W22E, W22K, W22R, Q23H, Q23S, Q23T, Q23N, Q23D, Q23E, Q23K, Q23R, L24T, L24Q, L24H, L24I, L24V, L24D, L24E, L24K, L24R, N25H, N25S, N25Q, R27H, R27Q, L28V, L28I, L28T, L28Q, L28H, L28A, E29N, E29Q, E29H, Y30H, Y30I, L32V, L32I, L32T, L32Q, L32H, L32A, K33Q, K33T, K33S, K33H, D34Q, D34G, R35H, R35Q, M36V, M36I, M36T, M36Q, M36A, F38I, F38V, D39N, D39Q, D39H, D39G, E42Q, E42H, E43Q, E43H, K45Q, K45T, K45S, K45T, L47V, L47I, L47T, L47Q, L47H, L47A, Q48N, Q49N, K52Q, K52T, K52S, K52H, E53Q, E53H, D54Q, L57T, L57Q, L57H, L57A, L57V, Y60H, E61Q, M62I, M62V, M62T, M62Q, M62A, L63T, L63Q, L63H, L63A, L63V, Q64N, F67I, F67V, R71H, R71Q, Q72T, Q72N, D73N, D73Q, D73H, D73G, G78D, G78E, G78K, G78R, W79H, W79S, N80D, N80E, N80K, N80R, E81N, E81Q, E81H, T82D, T82E, T82K, T82R, I83D, I83E, I83K, I83R, I83N, I83Q, I83S, I83T, E85 Q, E85H, N86D, N86E, N86K, N86R, N86Q, N86S, N86T, L87H, L87A, L88T, L88Q, L88H, L88A, L87I, L87V, L87D, L87E, L87K, L87R, L87N, L87Q, L87S, L87T, A89D, A89E, A89K, A89R, N90D, N90E, N90K, N90Q, N90R, N90S, N90T, N90C, V91D, V91E, V91K, V91N, V91Q, V91R, V91S, V91T, V91C, Y92H, Y92I, Q94D, Q94E, Q94K, Q94N, Q94R, Q94S, Q94T, Q94C, I95D, I95E, I95K, I95N, I95Q, I95R, I95S, I95T, H97D, H97E, H97K, H97N, H97Q, H97R, H97S, H97T, H97C, L98H, L98A, L98D, L98E, L98K, L98N, L98Q, L98R, L98S, L98T, L98C, K99Q, K99T, K99S, K99H, V101D, V101E, V101K, V101N, V101Q, V101R, V101S, V101T, V101C, L102T, L102Q, L102H, L102A, L102I, L102V, E103Q, E103H, E104Q, E104H, K105Q, K105T, K105S, K105H, L106T, L106Q, L106H, L106A, E107H, K108N, K108Q, K108T, K108S, K108H, E109H, E109Q, D110N, D100Q, D110H, D110G, F111I, F111V, R113H, R113Q, K115Q, L116V, L116I, L116T, L116Q, L116H, L116A, M117I, M117V, M117T, M117Q, M117A, L122I, L122V, L122T, L122Q, L122H, L122A, K123Q, K123T, K123S, K123H, R124H, R124Q, Y125H, Y126H, R128H, R128Q, L130V, L130I, L130T, L130Q, L130H, L130A, L133T, L133Q, L133H, L133A, Y132I, K134Q, K134T, K134S, K134H, K136Q, K136T, K136S, K136H, E137N, E137Q, E137H, Y138H, Y138I, W143H, W143S, E149Q, L151T, L151Q, L151H, L151A, L151V, R152H, R152Q, F154V, F154I, Y155H, Y155I, F156I, F156V, R159H, R159Q, L160V, L160T, L160Q, L160H, L160A, L160I, Y163H, Y163I, L164T, L164Q, L164H, L164A, L164I, L164V, R165H, and R165Q in the mature IFN-β polypeptide set forth in SEQ ID NO:1.

28. A modified IFN-β polypeptide of claim 24, wherein the one or more further amino acid modification(s) at one or more positions corresponding to amino acid positions is selected from among Y3, L5, L6, F8, L9, Q10, F15, Q16, Q18, L20, L21, Q23, L28, E29, Y30, L32, F38, E42, E43, L47, Q48, Q49, E53, L57, Y60, E61, L63, Q64, F67, Q72, E81, E85, L87, L88, Y92, Q94, L98, L102, E103, E104, L106, E107, E109, F111, L116, L120, Y125, Y126, L130, Y132, L133, E137, Y138, E149, L151, F154, F156, L160, Y163, and L164 in the mature IFN-β polypeptide set forth in SEQ ID NO:1; and
the modified IFN-β polypeptide exhibits increased resistance to gelatinase B proteolysis compared to an IFN-β polypeptide that does not include the modification(s).

29. A modified IFN-β polypeptide of claim 28, wherein the one or more further amino acid modification(s) correspond to modifications selected from among Y3H, L5V, L5I, L5T, L5Q, L5H, L5A, L5D, L5E, L5K, L5R, L5N, L5S, L6I, L6V, L6H, L6A, L6D, L6E, L6K, L6N, L6Q, L6R, L6S, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, F15I, F15V, F15D, F15E, F15K, F15R, Q16D, Q16E, Q16K, Q16N, Q16R, Q16S, Q16T, Q16C, Q18H, Q18T, L20H, L20A, L20N, L20Q, L20R, L20S, L20T, L20D, L20E, L20K, L21I, L21V, L21T, L21Q, L21H, L21A, Q23H, Q23S, Q23T, Q23N, Q23D, Q23E, Q23K, Q23R, L28V, L28I, L28T, L28Q, L28H, L28A, E29N, E29Q, E29H, Y30H, Y30I, L32V, L32I, L32T, L32Q, L32H, L32A, F38I, F38V, E42Q, E42H, E43Q, E43H, L47V, L47I, L47T, L47Q, L47H, L47A, Q48N, Q49N, E53Q, E53H, L57V, L57T, L57Q, L57H, L57A, Y60H, E61Q, L63V, L63T, L63Q, L63H, L63A, Q64N, F67I, F67V, Q72T, Q72N, E81N, E81Q, E81H, E85Q, E85H, L87I, L87V, L87H, L87A, L87D, L87E, L87, L87R, L87N, L87Q, L87S, L87T, L88T, L88Q, L88H, L88A, Y92H, Y92I, Q94D, Q94E, Q94K, Q94N, Q94R, Q94S, Q94T, Q94C, L98H, L98A, L98D, L98E, L98K, L98N, L98Q, L98R, L98S, L98T, L98C, L102I, L102V, L102T, L102Q, L102H, L102A, E103Q, E103H, E104Q, E104H, L106T, L106Q, L106H, L106A, E107Q, E107H, E109H, E109Q, F111I, F111V, L116V, L116I, L116T, L116Q, L116H, L116A, L116V, L116I, L116T, L116Q, L116H, L116A, Y125H, Y126H, L130V, L130I, L130T, L130Q, L130H, L130A, Y132H, L133I, L133V, L133T, L133Q, L133H, L133A, E137N, E137Q, E137H, Y138H, Y138I, E149Q, L151V, L151T, L151Q, L151H, L151A, F154I, F156I, F156V, L160I, L160V, L160T, L160Q, L160H, L160A, Y163H, Y163I, L164I, L164V, L164T, L164Q, L164H, and L164A in the mature IFN-β polypeptide set forth in SEQ ID NO:1.

30. A modified IFN-β polypeptide of claim 24, wherein the one or more further amino acid modification(s) is at one or more positions corresponding to amino acid positions is selected from among R11, K45, K52, K105, K108, R113, K115Q, R124, R152, and R165 in the mature IFN-β polypeptide set forth in SEQ ID NO:1; and
the modified IFN-β polypeptide exhibits a decreased isoelectric point compared to an IFN-β polypeptide that does not include the modification(s).

31. A modified IFN-β polypeptide of claim 30, wherein the one or more further amino acid modification(s) correspond to modifications selected from among R11Q, R11D, K45Q, K52Q, K105Q, K108Q, K108D, R113Q, R113E, K115Q, R124Q, R124D, R124E, R152Q, R152D, R165Q, and R165D in the mature IFN-β polypeptide set forth in SEQ ID NO:1.

32. A modified IFN-β polypeptide of claim 1, further comprising one or more amino acid modifications contributing to one or more of deimmunization, glycosylation, and/or PEGylation of the polypeptide.

33. A modified IFN-β polypeptide of claim 32, wherein the polypeptide is glycosylated and/or conjugated to a polyethylene glycol (PEG) moiety.

34. A modified IFN-β polypeptide, comprising:
two or more amino acid modifications in an unmodified IFN-β polypeptide, wherein:
the two or more amino acid modifications are at two or more positions corresponding to amino acid positions selected from among Y3, L6, R11, Q18, K19, L20, L21, Q23, L24, E29, K33, D34, F38, D39, P41, E42, E43, K45, Q48, Q49, F50, Q51, K52, E53, D54, L57, Y60, E61, M62, L63, Q64, F70, Q72, D73, W79, E81, E85, L87, L88, L98, K99, L102, E103, E104, K105, L106, E107, K108, E109, D110, R113, K115, M117, L122, K123, R124, Y125, Y126, Y132, L133, K134, K136, E137, W143, R147, E149, L151, R152, F154, F156, L160, L164, and R165 in the mature IFN-β polypeptide set forth in SEQ ID NO:1.

35. The modified IFN-β polypeptide of claim 34 that retains at least one in vivo activity of an IFN-β polypeptide.

36. The modified IFN-β polypeptide of claim 34 that has 1, 2, 3, 4, 5, 6, 7, 8, 9, 10, 11, 12, 13, 14, 15, 16, 17, 18, 19 or 20 amino acid modifications.

37. The modified IFN-β polypeptide of claim 34 that is a mature polypeptide.

38. The modified IFN-β polypeptide of claim 34 that is a precursor polypeptide.

39. A modified IFN-β polypeptide of claim 34, wherein the two or more amino acid modification(s) correspond to modifications selected from among Y3I, Y3H, L6I, L6V, L6H, L6A, R11D, Q18H, Q18S, Q18T, Q18N, K19N, L20I, L20V, L20H, L20A, L21I, L21V, L21T, L21Q, L21H, L21A, Q23H, Q23S, Q23T, Q23N, L24I, L24V, L24T, L24Q, L24H, L24A, E29N, K33N, D34N, D34Q, D34G, F38I, F38V, D39N, P41A, P41S, E42N, E43K, E43Q, E43H, E43N, K45D, K45N, Q48H, Q48S, Q48T, Q48N, Q49H, Q49S, Q49T, Q49N, F50I, F50V, Q51H, Q51S, Q51T, Q51N, K52D, K52N, E53R, E53Q, E53H, E53N, D54G, L57I, L57V, L57T, L57Q, L57H, L57A, Y60H, Y60I, E61K, E61Q, E61H, E61N, M62I, M62V, M62T, M62Q, M62H, M62A, L63I, L63V, L63T, L63Q, L63H, L63A, Q64H, Q64S, Q64T, Q64N, F70I, F70V, Q72H, Q72S, Q72T, Q72N, D73N, W79H, W79S, E81K, E81N, E85K, E85N, L87I, L87V, L87H, L87A, L88I, L88V, L88T, L88Q, L88H, L88A, L98I, L98V, L98H, L98A, K99N, L102I, L102V, L102T, L102Q, L102H, L102A, E103K, E103N, E104R, E104N, K105D, K105N, L106I, L106V, L106T, L106Q, L106H, L106A, E107R, E107N, K108D, K108N, E109R, E109N, D110K, D110N, R113E, K115D, K115Q, K115N, K115S, K115H, M117I, M117V, M117T, M117Q, M117A, L122I, L122V, L122T, L122Q, L122H, L122A, K123N, R124D, R124E, Y125H, Y125I, Y126H, Y126I, Y132H, Y132I, L133I, L133V, L133T, L133Q, L133H, L133A, K134N, K136N, E137N, W143H, W143S, R147H, R147Q, E149Q, E149H, E149N, L151I, L151V, L151T, L151Q, L151H, L151A, R152D, F154I, F154V, F156I, F156V, L160I, L160V, L160T, L160Q, L160H, L160A, L164I, L164V, L164T, L164Q, L164H, L164A, R165D in the mature IFN-β polypeptide set forth in SEQ ID NO:1.

40. The modified IFN-β polypeptide of claim 34, wherein the unmodified IFN-β polypeptide contains a sequence of amino acids set forth in SEQ ID NOS: 1 or 3.

41. The modified IFN-β polypeptide of claim 34, wherein the unmodified IFN-β polypeptide is an allelic or species variant of the polypeptide set forth in SEQ ID NO:1.

42. The modified IFN-β polypeptide of claim 41, wherein the allelic or species variant has 75%, 80%, 85%, 90%, 91%, 92%, 93%, 94%, 95%, 96%, 97%, 98%, or 99% sequence identity to the polypeptide set forth in SEQ ID NO:1 excluding the amino acid modification(s).

43. The modified IFN-β polypeptide of claim 34, wherein only the primary sequence is modified and the polypeptide exhibits increased protein stability.

44. The modified IFN-β polypeptide of claim 34 that exhibits increased protease resistance compared to the unmodified IFN-β polypeptide and retains one or more activities of the unmodified IFN-β polypeptide

45. A modified IFN-β polypeptide of claim 34, comprising one or more further amino acid modifications at one or more positions corresponding to amino acid positions selected from among M1, L5, L6, F8, L9, Q10, R11, S12, S13, N14, F15, Q16, C17, K19, L20, W22, Q23, L24, N25, R27, L28, E29, Y30, L32, K33, R35, M36, D39, E42, K45, L47, K52, F67, R71, D73, G78, W79, N80, E81, T82, I83, E85, N86, L87, A89, N90, V91, Y92, Q94, I95, H97, L98, K99, V101, E103, E104, K105, E107, K108, E109, D110, F111, R113, L116, L120, K123, R124, R128, L130, K134, K136, E137, Y138, R152, Y155, R159, Y163, and R165 in the mature IFN-β polypeptide set forth in SEQ ID NO:1.

46. A modified IFN-β polypeptide of claim 45, wherein the one or more further amino acid modification(s) correspond to modifications selected from among M1V, M1I, M1T, M1A, M1Q, M1D, M1E, M1K, M1N, M1R, M1S, M1C, L5V, L5I, L5T, L5Q, L5H, L5A, L5D, L5E, L5K, L5R, L5N, L5S, L6D, L6E, L6K, L6N, L6Q, L6R, L6S, L6T, L6T, L6C, F8I, F8V, F8D, F8E, F8K, F8R, L9V, L9I, L9T, L9Q, L9H, L9A, L9D, L9E, L9K, L9N, L9R, L9S, Q10D, Q10E, Q10K, Q10N, Q10R, Q10S, Q10T, Q10C, R11H, R11Q, S12D, S12E, S12K, S12R, S13D, S13E, S13K, S13N, S13Q, S13R, S13T, S13C, N14D, N14E, N14K, N14Q, N14R, N14S, N14T, F15I, F15V, F15D, F15E, F15K, F15R, Q16D, Q16E, Q16K, Q16N, Q16R, Q16S, Q16T, Q16C, C17D, C17E, C17K, C17N, C17R, C17S, C17T, K19Q, K19T, K19S, K19H, L20N, L20Q, L20R, L20S, L20T, L20D, L20E, L20K, W22S, W22H, W22D, W22E, W22K, W22R, Q23D, Q23E, Q23K, Q23R, L24D, L24E, L24K, L24R, N25H, N25S, N25Q, R27H, R27Q, L28V, L28I, L28T, L28Q, L28H, L28A, E29Q, E29H, Y30H, Y30I, L32V, L32I, L32T, L32Q, L32H, L32A, K33Q, K33T, K33S, K33H, R35H, R35Q, M36V, M36I, M36T, M36Q, M36A, D39Q, D39H, D39G, E42Q, E42H, K45Q, K45T, K45S, K45T, L47V, L47I, L47T, L47Q, L47H, L47A, K52Q, K52T, K52S, K52H, F67I, F67V, R71H, R71Q, D73Q, D73H, D73G, G78D, G78E, G78K, G78R, N80D, N80E, N80K, N80R, E81Q, E81H, T82D, T82E, T82K, T82R, I83D, I83E, I83K, I83R, I83N, I83Q, I83S, I83T, E85Q, E85H, N86D, N86E, N86K, N86R, N86Q, N86S, N86T, L87D, L87E, L87K, L87R, L87N, L87Q, L87S, L87T, A89D, A89E, A89K, A89R, N90D, N90E, N90K, N90Q, N90R, N90S, N90T, N90C, V91D, V91E, V91K, V91N, V91Q, V91R, V91S, V91T, V91C, Y92H, Y92I, Q94D, Q94E, Q94K, Q94N, Q94R, Q94S, Q94T, Q94C, I95D, I95E, I95K, I95N, I95Q, I95R, I95S, I95T, H97D, H97E, H97K, H97N, H97Q, H97R, H97S, H97T, H97C, L98D, L98E, L98K, L98N, L98Q, L98R, L98S, L98T, L98C, K99Q, K99T, K99S, K99H, V101D, V101E, V101K, V101N, V101Q, V101R, V101S, V101T, V101C, E103Q, E103H, E104Q, E104H, K105Q, K105T, K105S, K105H, E107Q, E107H, K108Q, K108T, K108S, K108H, E109H, E109Q, D110Q, D110H, D110G, F111I, F111V, R113H, R113Q, L116V, L116I, L116T, L116Q, L116H, L116A, L120V, L120I, L120T, L120Q, L120H, L120A, K123Q, K123T, K123S, K123H, R124H, R124Q, R128H, R128Q, L130V, L130I, L130T, L130Q, L130H, L130A, K134Q, K134T, K134S, K134H, K136Q, K136T, K136S, K136H, E137Q, E137H, Y138H, Y138I, R152H, R152Q, Y155H, Y155I, R159H, R159Q, Y163H, Y163I, R165H, and R165Q in the mature IFN-β polypeptide.

47. A modified IFN-β polypeptide of claim 34, further-comprising one or more amino acid modifications contributing to one or more of deimmunization, glycosylation and PEGylation of the polypeptide.

48. A modified IFN-β polypeptide of claim 47, wherein the polypeptide is glycosylated or conjugated to a polyethylene glycol (PEG) moiety.

49. A modified IFN-β polypeptide, comprising:
two or more amino acid modifications in an unmodified IFN-β polypeptide, wherein:
the two or more amino acid modifications are at two or more positions corresponding to amino acid positions selected from among M1, L5, L6, F8, L9, Q10, R11, S12, S13, N14, F15, Q16, C17, L20, W22, Q23, L24, E43, K45, K52, E53, D54, E61, G78, W79, N80, E81, T82, I83, E85, N86, L87, A89, N90, V91, Q94, I95, H97, L98, V101, E103, E104, K105, E107, K108, E109, D110, R113, K115, R124, R152, and R165 in the mature IFN-β polypeptide set forth in SEQ ID NO:1;
the modified IFN-β polypeptide exhibits increased protein stability manifested as increased conformational stability compared to the unmodified IFN-β polypeptide; and
the modified IFN-β polypeptide retains one or more activities of the unmodified IFN-β polypeptide.

50. The modified IFN-β polypeptide of claim 49 that has 1, 2, 3, 4, 5, 6, 7, 8, 9, 10, 11, 12, 13, 14, 15, 16, 17, 18, 19 or 20 amino acid modifications.

51. A modified IFN-β polypeptide of claim 49 that is a mature IFN-β polypeptide.

52. A modified IFN-β polypeptide of claim 49 that is a precursor IFN-β polypeptide.

53. A modified IFN-β polypeptide of claim 49, wherein the two or more amino acid modification(s) correspond to modifications selected from among M1E, M1D, M1K, M1R, M1N, M1Q, M1S, M1T, M1C, L5E, L5D, L5K, L5R, L5N, L5Q, L5S, L5T, L6C, F8E, F8D, F8K, F8R, L9E, L9D, L9K, L9R, L9N, L9Q, L9S, L9T, Q10C, Q10E, Q10D, Q10K, Q10R, Q10N, Q10S, Q10T, R11Q, R11D, S12E, S12D, S12K, S12R, S13E, S13D, S13K, S13R, S13N, S13Q, S13T, S13C, N14E, N14D, N14K, N14R, N14Q, N14S, N14T, F15E, F15D, F15K, F15R, Q16E, Q16D, Q16K, Q16R, Q16N, Q16S, Q16T, Q16C, C17E, C17D, C17K, C17R, C17N, C17Q, C17S, C17T, L20E, L20D, L20K, L20R, L20N, L20Q, L20S, L20T, W22E, W22D, W22K, W22R, Q23E, Q23D, Q23K, Q23R, L24E, L24D, L24K, L24R, E43K, K45Q, K45D, K52Q, K52D, E53R, D54K, E61K, G78E, G78D, G78K, G78R, W79E, W79D, W79K, W79R, N80E, N80D, N80K, N80R, E81K, T82E, T82D, T82K, T82R, I83E, I83D, I83K, I83R, I83N, I83Q, I83S, I83T, E85K, N86E, N86D, N86K, N86R, N86Q, N86S, N86T, L87E, L87D, L87K, L87R, L87N, L87Q, L87S, L87T, A89E, A89D, A89K, A89R, N90E, N90D, N90K, N90R, N90Q, N90S, N90T, N90C, V91E, V91D, V91K, V91R, V91N, V91Q, V91S, V91T, V91C, Q94E, Q94D, Q94K, Q94R, Q94N, Q94S, Q94T, Q94C, I95E, I95D, I95K, I95R, I95N, I95Q, I95S, I95T, H97E, H97D, H97K, H97R, H97N, H97Q, H97S, H97T, H97C, L98E, L98D, L98K, L98R, L98N, L98Q, L98S, L98T, L98C, V101C, V101E, V101D, V101K, V101R, V101N, V101Q, V101S, V101T, E103K, E104R, K105Q, K105D, E107R, K108Q, K108D, E109R, D110K, R113Q, R113E, K115Q, K115D, R124Q, R124D, R124E, R152Q, R152D, R165Q, and R165D in the mature IFN-β polypeptide set forth in SEQ ID NO:1.

54. The modified IFN-β polypeptide of claim 49, wherein the unmodified IFN-β polypeptide has a sequence of amino acids set forth in SEQ ID NO:1 or SEQ ID NO:3.

55. The modified IFN-β polypeptide of claim 49, wherein the unmodified IFN-β polypeptide is an allelic or species variant of the polypeptide set forth in SEQ ID NO:1.

56. The modified IFN-β polypeptide of claim 55, wherein the allelic or species variant has 75%, 80%, 85%, 90%, 91%, 92%, 93%, 94%, 95%, 96%, 97%, 98%, or 99% sequence identity to the polypeptide set forth in SEQ ID NO:1 excluding the amino acid modification(s).

57. The modified IFN-β polypeptide of claim 49, wherein only the primary sequence is modified and the polypeptide exhibits increased conformational stability.

58. A modified IFN-β polypeptide of claim 49, wherein:
the two or more amino acid modification(s) correspond to modifications selected from among L5E, L5D, L5K, L5R, F8E, F8D, F8K, F8R, L9E, L9D, L9K, L9R, S12E, S12D, S12K, S12R, F15E, F15D, F15K, F15R, Q16E, Q16D, Q16K, Q16R, L20E, L20D, L20K, L20R, W22E, W22D, W22K, W22R, Q23E, Q23D, Q23K, Q23R, L24E, L24D, L24K, L24R, G78E, G78D, G78K, G78R, W79E, W79D, W79K, W79R, N80E, N80D, N80K, N80R, T82E, T82D, T82K, T82R, I83E, I83D, I83K, I83R, N86E, N86D, N86K, N86R, L87E, L87D, L87K, L87R, A89E, A89D, A89K, and A89R in the mature IFN-β polypeptide set forth in SEQ ID NO:1; and
the amino acid modifications contribute to increased conformational stability due to the addition of charges to regions in helices A and C.

59. A modified IFN-β polypeptide of claim 49, wherein:
the two or more amino acid modification(s) correspond to modifications selected from among M1E, M1D, M1K, M1R, M1N, M1Q, M1S, M1T, L5E, L5D, L5K, L5R, L5N, L5Q, L5S, L5T, L6E, L6D, L6K, L6R, L6N, L6Q, L6S, L6T, L9E, L9D, L9K, L9R, L9N, L9Q, L9S, L9T, Q10E, Q10D, Q10K, Q10R, Q10N, Q10S, Q10T, S13E, S13D, S13K, S13R, S13N, S13Q, S13T, N14E, N14D, N14K, N14R, N14Q, N14S, N14T, Q16E, Q16D, Q16K, Q16R, Q16N, Q16S, Q16T, C17E, C17D, C17K, C17R, C17N, C17Q, C17S, C17T, L20E, L20D, L20K, L20R, L20N, L20Q, L20S, L20T, I83E, I83D, I83K, I83R, I83N, I83Q, I83S, I83T, N86E, N86D, N86K, N86R, N86Q, N86S, N86T, L87E, L87D, L87K, L87R, L87N, L87Q, L87S, L87T, N90E, N90D, N90K, N90R, N90Q, N90S, N90T, V91E, V91D, V91K, V91R, V91N, V91Q, V91S, V91T, Q94E, Q94D, Q94K, Q94R, Q94N, Q94S, Q94T, I95E, I95D, I95K, I95R, I95N, I95Q, I95S, I95T, H97E, H97D, H97K, H97R, H97N, H97Q, H97S, H97T, L98E, L98D, L98K, L98R, L98N, L98Q, L98S, L98T, V101E, V101D, V101K, V101R, V101N, V101Q, V101S, and V101T in the mature IFN-β polypeptide set forth in SEQ ID NO:1; and
the amino acid modifications contribute to increased conformational stability due to increased polar interactions between helices A and C.

60. A modified IFN-β polypeptide of claim 49, wherein:
the two or more amino acid modification(s) correspond to modifications selected from among M1C, L6C, Q10C, S13C, Q16C, N90C, V91C, Q94C, H97C, L98C, and V101C in the mature IFN-β polypeptide set forth in SEQ ID NO:1; and
the amino acid modifications result in introduction of a disulfide bridge in the IFN-β polypeptide.

61. A modified IFN-β polypeptide of claim 60, wherein the disulfide bridge formed between positions corresponds to amino acid positions C1-C101, C6-C98, C16-C90, C10-C97, C10-C98, or C13-C94 in the mature IFN-β polypeptide set forth in SEQ ID NO:1.

62. A modified IFN-β polypeptide of claim 49, wherein:
the two or more amino acid modification(s) correspond to modifications selected from among E43K, E53R, D54K, E61K, E81K, E85K, E103K, E104R, E107R, E109R, and D110K in the mature IFN-β polypeptide set forth in SEQ ID NO:1; and
the amino acid modifications contribute to increased conformational stability due to an increase in the isoelectric point.

63. A modified IFN-β polypeptide of claim 49, wherein:
the two or more amino acid modification(s) correspond to modifications selected from among R11D, R11Q, K45D, K45Q, K52D, K52Q, K105D, K105Q, K108D, K108Q, R113E, R113Q, K115D, K115Q, R124D, R124Q, R124E, R152D, R152Q, R165Q, and R165D in the mature IFN-β polypeptide set forth in SEQ ID NO:1

64. A modified IFN-β polypeptide of claim 49, further comprising one or more amino acid modifications contributing to one or more of deimmunization, glycosylation, and PEGylation of the polypeptide.

65. A modified IFN-β polypeptide of claim 64, wherein the polypeptide is glycosylated or conjugated to a polyethylene glycol (PEG) moiety.

66. A modified IFN-β polypeptide of any of claims 1, 34 and 49, wherein the one or more amino acid modifications are selected from natural amino acids, non-natural amino acids and a combination of natural and non-natural amino acids.

67. The modified IFN-β polypeptide of any of claims 1, 34 and 49 that is a naked polypeptide chain.

68. The modified IFN-β polypeptide of any of claims 1, 34 and 49 that is pegylated, albuminated and/or glycosylated.

69. A nucleic acid molecule, comprising a sequence of nucleotides encoding a modified IFN-β polypeptide of any of claims 1, 34 and 49.

70. A vector, comprising the nucleic acid molecule of claim 69.

71. A cell, comprising the vector of claim 70.

72. The cell of claim 71 that is a eukaryotic cell.

73. The cell of claim 71 that is a prokaryotic cell.

74. A method for production of a modified IFN-β polypeptide, comprising introducing a nucleic acid molecule of claim 69 into a cell, and culturing the cell under conditions whereby the encoded modified IFN-β polypeptide is expressed.

75. A method for production of a modified IFN-β polypeptide, comprising introducing a nucleic acid molecule of claim 69 into a cell-free system, whereby the encoded modified IFN-β polypeptide is expressed.

76. The method of claim 74, wherein the modified IFN-β polypeptide is glycosylated.

77. A pharmaceutical composition, comprising a modified IFN-β polypeptide of any of claims 1, 34 and 49 in a pharmaceutically acceptable excipient.

78. A pharmaceutical composition, consisting essentially of a modified IFN-β polypeptide of any of claims 1, 34 and 49.

79. The pharmaceutical composition of claim 77 that is formulated for oral, nasal or pulmonary administration.

80. The pharmaceutical composition of claim 79, wherein:
the IFN-β polypeptide exhibits increased protease resistance; and
the composition that is formulated for oral administration.

81. The pharmaceutical composition of claim 80, where the polypeptide exhibits increased resistance to one or more proteases selected from among pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, luminal pepsin, microvillar endopeptidase, dipeptidyl peptidase, enteropeptidase, hydrolase, NS3, factor Xa, Granzyme B, thrombin, plasmin, urokinase, tPA and PSA.

82. The pharmaceutical composition of claim 77, wherein the pharmaceutical composition does not contain exogenously added protease inhibitors.

83. The pharmaceutical composition of claim 77 that is formulated as a liquid, a pill, a tablet, a lozenge or a capsule.

84. The pharmaceutical composition of claim 83, wherein the lozenge delivers the modified IFN-β polypeptide to the mucosa of the mouth, throat, or gastrointestinal tract.

85. The pharmaceutical composition of claim 77, wherein the pharmaceutical composition is formulated for controlled-release of the modified IFN-β polypeptide.

86. The pharmaceutical composition of claim 83, wherein the pill, tablet, or capsule is coated with an enteric coating.

87. A method, comprising treating a subject by administering the pharmaceutical composition of claim 77, wherein the subject has a disease or condition that is responsive to administration of IFN-β.

88. The method of claim 87, wherein the disease or condition is selected from among a viral infection, a proliferative disorder, an autoimmune disease, and an inflammatory disorder.

89. The method of claim 88, wherein the autoimmune disease is multiple sclerosis, rheumatoid arthritis, chronic viral hepatitis, hepatitis A, hepatitis B, and myocardial viral infection.

90. The method of claim 88, wherein the proliferative disease is a cancer or bone disorder.

91. The method of claim 90, wherein the cancer is selected from among uveal melanoma, colon cancer, liver cancer, and a metastatic cancer.

92. The method of claim 90, wherein the bone disorder is osteoporosis or osteopenia.

93. The method of claim 88, wherein the inflammatory disorder is selected from among asthma, Guillain-Barre syndrome, and an inflammatory bowel disease.

94. The method of claim 93, wherein the inflammatory bowel disease is ulcerative colitis or Crohn's disease.

95. The method of claim 88, wherein the viral infection is chronic viral hepatitis or myocardial viral infection.

96. A method of treating multiple sclerosis, comprising administering a modified IFN-β polypeptide that exhibits increased resistance to cleavage by gelatinase B.

97. The method of claim 96, wherein the modified IFN-β polypeptide has a sequence of amino acids set forth in any one of SEQ ID NOS: 4-11, 16, 17, 20-27, 30-36, 39-42, 45-54, 61-70, 75-87, 157, 158, 163-168, 173, 174, 180-185, 190-193, 198, 199, 204, 205, 209, 210, 213-224, 233-238, 247-250, 266-279, 282, 283, 295-310, 328-358, 377-387, 396-403, 408-411, 447-454, 474-479, 497-504, 540-542, 547, 551, 555-558, 562-576, 578-583, 585-589, 591, 604-607, 610-614, 616-650, 652, 653, 655, 656, and 658.
